(19) United States
(12) Patent Application Publication
(10) Pub. No.: US 2024/0100172 A1
(43) Pub. Date: Mar. 28, 2024

(54) METHODS AND COMPOUNDS FOR TARGETED AUTOPHAGY

(71) Applicant: Hangzhou Jijing Pharmaceutical Technology Limited, Hangzhou, Zhejiang (CN)

(72) Inventors: Nan Ji, Arlington, MA (US); Ning Yin, Lexington, MA (US)

(21) Appl. No.: 18/268,447

(22) PCT Filed: Dec. 20, 2021
(86) PCT No.: PCT/US2021/064327

§ 371 Date: Jun. 20, 2023

Related U.S. Application Data
(60) Provisional application No. 63/155,832, filed on Mar. 3, 2021.
(60) Provisional application No. 63/128,612, filed on Dec. 21, 2020.

(51) Int. Cl. A61K 47/55; A61K 47/54; A61P 35/00

(52) U.S. Cl. CPC A61K 47/55; A61K 47/545; A61P 35/00

(57) ABSTRACT

Disclosed are bifunctional targeted protein autophagy degraders that comprise: 1) a small-molecule targeting a protein of interest; 2) a linker; and 3) a small-molecule recruiter of the autophagy adapter LC3B. These degraders target specific proteins for degradation through the autophagy-lysosomal degradation pathway.

### RELATED APPLICATIONS

This application claims the benefit of the filing date, under 35 U.S.C. § 119(e), of U.S. Provisional Application No. 63/128,612, filed on Dec. 21, 2020 and U.S. Provisional Application No. 63/155,832, filed on Mar. 3, 2021, the entire contents of each of which is incorporated herein by reference.

### BACKGROUND

Autophagy is central to the maintenance of organismal homeostasis in both physiological and pathological situations. It is an essential, conserved lysosomal degradation pathway that controls the quality of the cytoplasm by eliminating aggregated proteins and damaged organelles. Accordingly, alterations in autophagy have been linked to a wide range of diseases and conditions, including aging, cancer, metabolic disorders, and neurodegenerative diseases. Autophagy begins with double-membraned autophagosomes which engulf portions of the cytoplasm, which is followed by fusion of these vesicles with lysososomes and degradation of the autophagic contents. This pathway is dysregulated across many human disorders, including metabolic conditions, neurodegenerative diseases, cancers, and infectious diseases. Autophagosome formation is a multistep process that includes the biogenesis of the phagophore, followed by its elongation and closure. More than 15 autophagy-related ATG proteins, as well as class III PI3 kinases, are required to construct the autophagosome, including the only transmembrane ATG protein ATG9, along with membranes from multiple cellular sources. The proteins ATG8 and microtubule-associated protein 1 light-chain 3 (LC3) are involved in expansion and fusion of phagophore edges, and recruit adaptor proteins such as ubiquitin-binding protein p62 and NBR1 to autophagosomes via their LC3-interacting region (LIR) domains. In turn, autophagic adaptors enable the selective degradation of aged or damaged cellular structures, protein aggregates, and microorganisms.

Most neurodegenerative diseases are associated with intracytoplasmic deposition of aggregate-prone proteins in neurons and with mitochondrial dysfunction. Autophagy is a powerful process for removing such proteins and for maintaining mitochondrial homeostasis. Over recent years, evidence has accumulated to demonstrate that upregulation of autophagy is protective against neurodegeneration. Numerous studies have demonstrated that aggregate-prone proteins at the heart of neurodegenerative disease toxicity are autophagy substrates and that pharmacological upregulators of autophagy can be beneficial in both cell and animal models of these diseases, in which they are able to reduce both intracytoplasmic aggregates and associated cell death.

Developing a strategy to specifically target aggregated proteins to autophagy and lysosomal degradation would enable clearing of toxic protein aggregates and prevent neurodegeneration.

### SUMMARY

Disclosed are bifunctional targeted protein autophagy degraders that comprise: 1) a small-molecule targeting a protein of interest; 2) a linker; and 3) a small-molecule recruiter of an autophagy adapter such as LC3B. These degraders target specific proteins for degradation through the autophagy-lysosomal degradation pathway. For example, the disclosed degraders are effective at reducing KRAS levels in NCI-H2030 cells (see Table 2), and are effective at reducing BTK levels in NAMALWA cells (see Table 3).

The bifunctional targeted protein autophagy degraders are represented by the following structural formula:

CCB-L-(LC3B-Binder)

wherein: CCB is a cellular component binder; L is a covalent bind or linker group and LC3B binder is a moiety that binds LC3B protein.

Also disclosed is a pharmaceutical composition comprising: i) a disclosed bifunctional targeted protein autophagy degrader; and ii) a pharmaceutically acceptable carrier, excipient or diluent.

Another embodiment of the invention is a method of treating a subject with a neurodegenerative disease, cancer, a metabolic disease, an autoimmune disease, an inflammatory disease, or infectious disease, comprising administering to the subject an effective amount of a disclosed bifunctional targeted protein autophagy degrader.

### DETAILED DESCRIPTION

The invention is directed to bifunctional targeted protein autophagy degraders that comprise: 1) a small-molecule targeting a protein of interest; 2) a linker; and 3) a small-molecule recruiter of an autophagy adapter such as LC3B. These degraders target specific proteins for degradation through the autophagy-lysosomal degradation pathway.

LC3B is a member of the MAP1LC3 (microtubule-associated protein-1 light chain 3) family of proteins, which play a crucial role in autophagy. LC3B can be prepared recombinantly by procedures disclosed in Lv et al., Protein Cell 8(1):25 2017 and Li et al., Nature 575:203 (2019); and can be isolated by procedures disclosed in Atkinson et al., J. Biol. Chem., 294(38):14033 (2019). Autophagy-targeting chimeras (AUTACs) have been developed to degrade proteins by targeting LC3, as described by L. Ouyang, J. Pei, X. Pan, A. Wang, W. Shuai, F. Bu, P. Tang, S. Zhang, Y. Zhang and G. Wang, Chem. Commun., 2021, DOI: 10.1039/D1CC04661F. Autophagy-tethering compounds (ATTECs) have also been developed to degrade non-protein biomolecules using lipid droplets (LDs) as an exemplar target, as described by Y. Fu, N. Chen, Z. Wang, S. Luo, Y. Ding and B. Lu, Cell Research (2021) 0:1-15. The entire teachings of these references are incorporated herein by reference.

“LC3B binder” refers to a compound which binds to LC3B. When the LC3B binder moiety of a disclosed bifunctional targeted protein autophagy degrader binds to LC3B, the binding is with sufficient affinity such that the targeted cellular component is brought within sufficient proximity to LC3B to accelerate degradation by autophagy of the targeted cellular component. Exemplary LC3B binders and assays for identifying additional LC3B binders are disclosed in WO2020182144; WO2020182143; Lv et al., Protein Cell 8(1):25 2017; Li et al., Nature 575:203 (2019); and Atkinson et al., J. Biol. Chem., 294(38):14033 (2019). The entire teachings of these references are incorporated herein by reference. In one embodiment, the LC3B binders bind to LC3B non-covalently. Alternatively, the LC3B binder is not capable of forming a covalent bond with cysteine. In another embodiment, the LC3B binder does not interact or contact a cysteine residue in LC3B.

The term “cellular component binder” as used herein refers to a substance (e.g., a biomolecule, macromolecule, or compound) which is capable of binding a cellular component. In embodiments, the cellular component binder is capable of binding a protein (e.g., KRAS, KRAS^{G12C} or BTK). In embodiments, the cellular component binder is capable of binding a protein aggregate. In embodiments, the cellular component binder is a protein (e.g., antibody, antibody fragment, or receptor), nucleic acid (e.g., siRNA, antisense nucleic acid), aptamer, or compound).

“Cellular component” refers to matter contained inside a cell (i.e., intracellular). Cellular components include matter naturally inside the cell (i.e., on the interior of the cell's lipid bilayer) as well as originally foreign agents (e.g., microorganisms, viruses, asbestos, or compounds or extracellular origin) that exist inside the cell. Non-limiting examples of a cellular component includes a protein or a derivative, fragment, or homolog thereof, ion (e.g., Na⁺, Mg⁺, Cu⁺, Cu²⁺, Zn²⁺, Mn²⁺, Fe²⁺, and Co²⁺), polysaccharides, lipids (e.g., fats, waxes, sterols, fat-soluble vitamins such as vitamins A, D, E, and K, monoglycerides, diglycerides, triglycerides, or phospholipids), nucleic acids (e.g., DNA or RNA), nucleotides, amino acids, particles (e.g., nanoparticle), fibers (e.g., asbestos fibers), organelles (e.g., mitochondria, peroxisome, plastid, endoplasmic reticulum, flagellum, or Golgi apparatus), cellular compartments, microorganisms (e.g., bacterium, virus, or fungus), viruses, vesicles (e.g., lysosome, peroxisome), small molecules, protein complexes, protein aggregates, or macromolecules. In one embodiment, the cellular component is a biomolecule.

Preferably, the cellular component is a biomolecule whose inhibition or degradation results in a desirable therapeutic effect. Examples of such cellular components include is a protein aggregate, intracellular protein, soluble protein, midbody ring, damaged mitochodria, peroxisomes, intracellular bacteria, phagocytic membrane remnants, or viral capsid proteins. Non-limiting examples of intracellular proteins include BRD4, KRAS, MYC, YAP, TAZ, CTNNB1, APP, HTT, SNCA, NRF2, and MAPT. In another alternative, the cellular component is a protein aggregate (e.g., HTT, APP, SNCA, or MAPT). In another alternative, the cellular component is PINK1, ATG32, ESYT, PI3KC3, RAB10, or ATGL. In another alternative, the cellular component is a microorganism. In yet another alternative, the cellular component is a bacterial cell-surface glycan or bacterial cell surface protein.

In one embodiment, the cellular component is an intracellular protein selected from ERK5 (MAPK7); BTK; ALK; EGFR; RAF1; KRAS; MDM2; STAT3; HIF1A; NTRK1; IRAK4; AR; ABL1; KDR; CDK4; CDK6; CDK7; MAP3K11; MET; PDGFRA; ESR1; IGF1R; and TERT. Alternatively, the cellular component is an intracellular protein selected from BTK, BRD4, KRAS, MYC, YAP, TAZ, CTNNB1, APP, HTT, SNCA, NRF2, MAPT, PINK1, ATG32, ESYT, PI3KC3, RAB10 and ATGL.

In another alternative, the cellular component is selected from KRAS, KRAS with a G12C mutation or KRAS with a G12D mutation. Cellular component binders which bind to KRAS, KRAS with a G12C mutation or KRAS with a G12D mutation are disclosed in WO2103155223, WO2014152588, WO2104143659, WO2015054572, WO2016049524, WO2016164675, WO2016168540, WO2017015562, WO2017058728, WO2017058768, WO2017058792, WO2017058805, WO2017058807, WO2017058902, WO2017058915, WO2017087528, WO2017100546, WO2018064510, WO2018068017, WO2018140512, WO2018140513, WO2018140514, WO2018140598, WO2018140599, WO2018140600, WO2018218069, WO2018218070, WO2018218071, WO2018119183, WO2018217651, WO2019051291, WO2018143315, WO20182065, WO2019110751, WO2014160200, WO2019141250, WO2017201161, WO2019099524, WO2019150305 and WO2019155399, the entire teachings of which are incorporated herein by reference.

In another alternative, the cellular component is BTK. Cellular component binders which bind to BTK include Ibrutinib, Acalabrutinib, Evobrutinib, Fenebrutinib, Vecabrutinib and spebrutinib. Others are disclosed in WO2017046604, US20160200730, WO2015140566, WO2015132799, WO2015048689, WO2012158764, WO2014188173, WO2014039899, US20140275014, WO2014116504, WO2015022926, WO2011119663, WO2018233655, WO2018090792, WO2017041180, CN201710309938, WO2011152351, WO2015002894, WO2017066014, WO2018156901, WO2018002958, WO2014078578, WO201809539, WO2018196757, WO2018001331, WO2017077507, WO2016196418, WO2016109223, WO2016109221, WO2016109219, WO2016109215, WO2016109220, WO2016109216, WO2016109217, WO2016109222, US20140206681, WO2013010380, US20150353565, CN201810076251, CN201810002152, WO2018130213, WO2018133151, WO2018035061, WO2015095099, WO2015095102, WO2016210165, WO2010126960, WO2016112637, WO2018092047, WO2017127371, WO2017007987, WO2015006754, WO2015006492, WO2014100748, WO2014040555, WO2015170266, WO2012170976, WO2016079669, CN201810299255, CN201810020706, WO2014169710, WO2014089913, GB2516303, CN201710258020, WO2017063103, WO2014025486, WO2014135473, WO2010068810, CN201410478741, WO2013152135, WO2018208132, WO2018004306, WO2018169373, WO2015151006, WO2018039310, WO2018088780, WO2014064131, GB2515785, WO2015017502, US20160002241, WO2017096100, WO2018035080, WO2018191577, US20180194762, WO2014161799, WO2013100631, WO2018145525, CN201710244485, WO2017100662, WO2018103058, U.S. Pat. No. 9,630,968, WO2013067264, WO2014135470, WO2015050703, WO2013083666, WO2012156334, WO2016164285, WO2018097234, WO2010006970, WO2014187262, WO2010006947, WO2013157022, WO2013161848, US20150064196, US20150376166, WO2014076104, WO2011140488, WO2017133341, CN201710208174, WO2017123695, WO2017106429, WO2015116485, US20150005277, WO2014068527, US20140265012, WO2017103611, WO2014173289, WO2017198050, WO2018033853, WO2015086636, WO2015086635, WO2015086642, US20140275023, WO2016164284, WO2016065222, WO2014210087, CN201510175762, US20140378475, WO2014173289, WO2014104757, WO2013148603, WO2014198960 and WO2013185084, the entire teachings of which are incorporated herein by reference.

“Protein aggregate” refers to an aberrant collection or accumulation of proteins (e.g., misfolded proteins). Protein aggregates are often associated with diseases (e.g., amyloidosis). Typically, when a protein misfolds as a result of a change in the amino acid sequence or a change in the native environment which disrupts normal non-covalent interactions, and the misfolded protein is not corrected or degraded, the unfolded/misfolded protein may aggregate. There are three main types of protein aggregates that may form: amorphous aggregates, oligomers, and amyloid fibrils. In one embodiment, protein aggregates are termed aggresomes. In one example, the protein aggregate is HTT, APP, SNCA, or MAPT. Alternatively, the protein aggregate includes the protein Beta amyloid, Amyloid precursor protein, IAPP (Amylin), Alpha-synuclein, PrPSc, PrPSc, Huntingtin, Calcitonin, Atrial natriuretic factor, Apolipoprotein AI, Serum amyloid A, Medin, Prolactin, Transthyretin, Lysozyme, Beta-2 microglobulin, Gelsolin, Keratoepithelin, Beta amyloid, Cystatin, Immunoglobulin light chain AL, or S-IBM.

In another alternative, the cellular component is mHTT, which is mutant and aberrant form of huntingtin protein associated with Huntington's disease and other neurodegenerative diseases. Exemplary cellular component binders which bind mHTT are disclosed in WO2020182144; WO2020182143; and Li et al., Nature 575:203 (2019). Thioflavin T is another exemplary cellular component binders which binds mHTT.

A “linker group” is a bivalent moiety that connects the LC3B binder with the cellular component binder.

In one embodiment, the linker group is bivalent, saturated or unsaturated, straight or branched C₁₋₅₀ hydrocarbon chain, wherein 0-6 methylene units of L are independently replaced by —C(D)(H)—, —C(D)₂-, Si(R)₂—, —Si(OH)(R)—, —Si(OH)₂—, —P(O)(OR)—, —P(O)(R)—, —P(O)(NR₂)—, -Cy-, —O—, —NR—, —S—, —OC(O)—, —C(O)O—, —C(O)—, —S(O)—, —S(O)₂—, —NRS(O)₂—, —S(O)₂NR—, —NR(O)—, —C(O)NR—, —OC(O)NR—, NRC(O)O—,

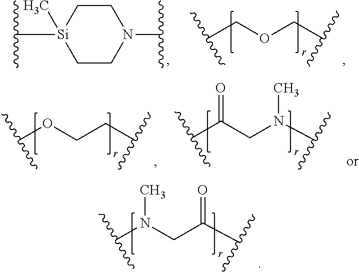

Each Cy is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur, and wherein r is 0, 1, 2, 3, 4, 5, 6, 7, 8, 9, or 10; and each R is independently hydrogen, deuterium, or an optionally substituted group selected from C1-6 aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or two R groups on the same atom are optionally taken together with their intervening atom to form an optionally substituted 4-11 membered saturated or partially unsaturated carbocyclic or heterocyclic monocyclic, bicyclic, bridged bicyclic, spiro, or heteroaryl ring having 0-3 heteroatoms, in addition to the atom to which they are attached, independently selected from nitrogen, oxygen, and sulfur.

In another embodiment, the linker is a bivalent, saturated or unsaturated, straight or branched C₁₋₅₀ hydrocarbon chain, wherein 0-6 methylene units of L are independently replaced by -Cy-, —O—, —N(R)—, —C(O)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—, wherein each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur.

The term “aliphatic”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic, that has a single point of attachment to the rest of the molecule. Unless otherwise specified, aliphatic groups contain 1-6 aliphatic carbon atoms. Examples of aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

Exemplary linkers are shown below:

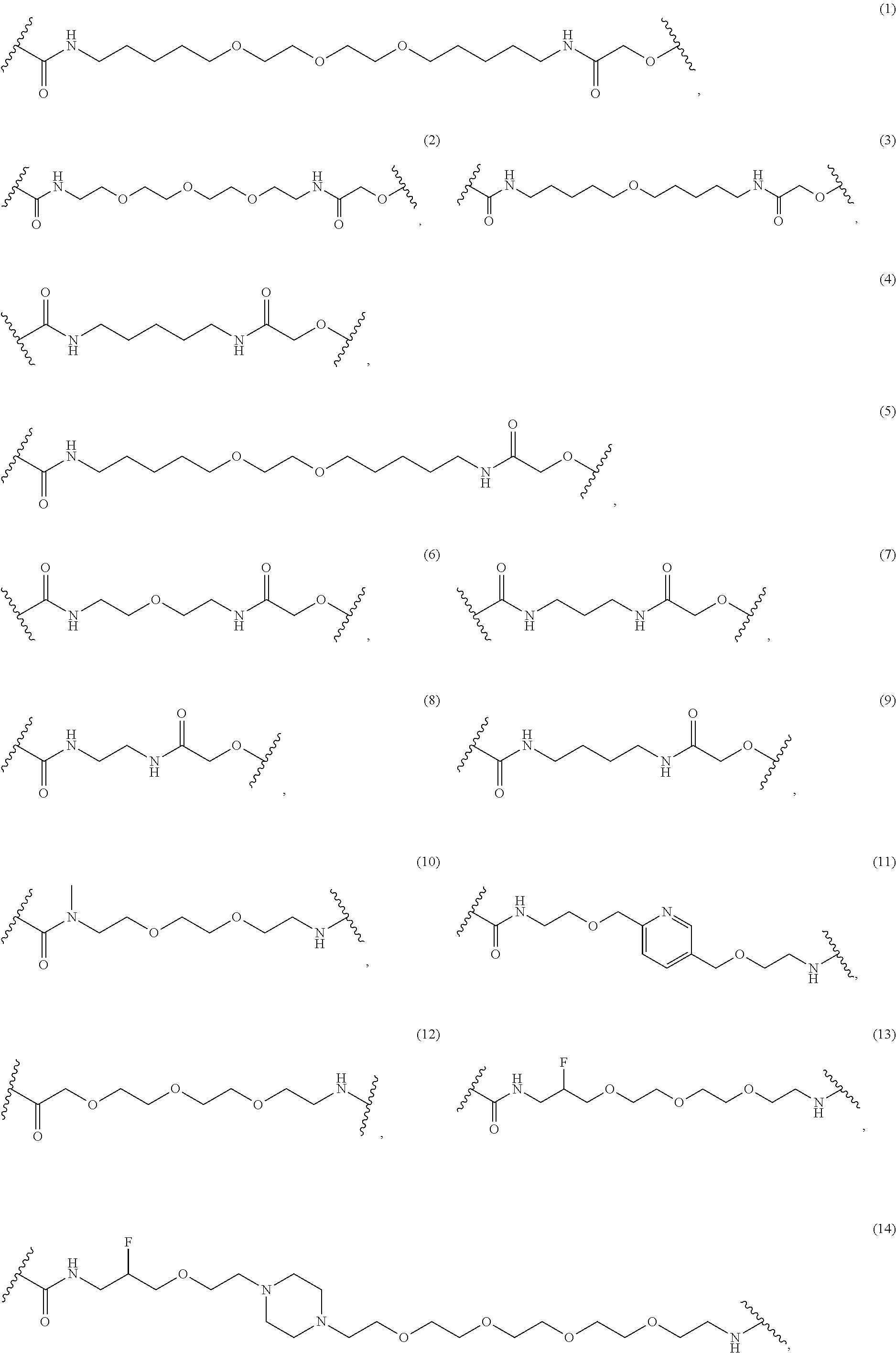

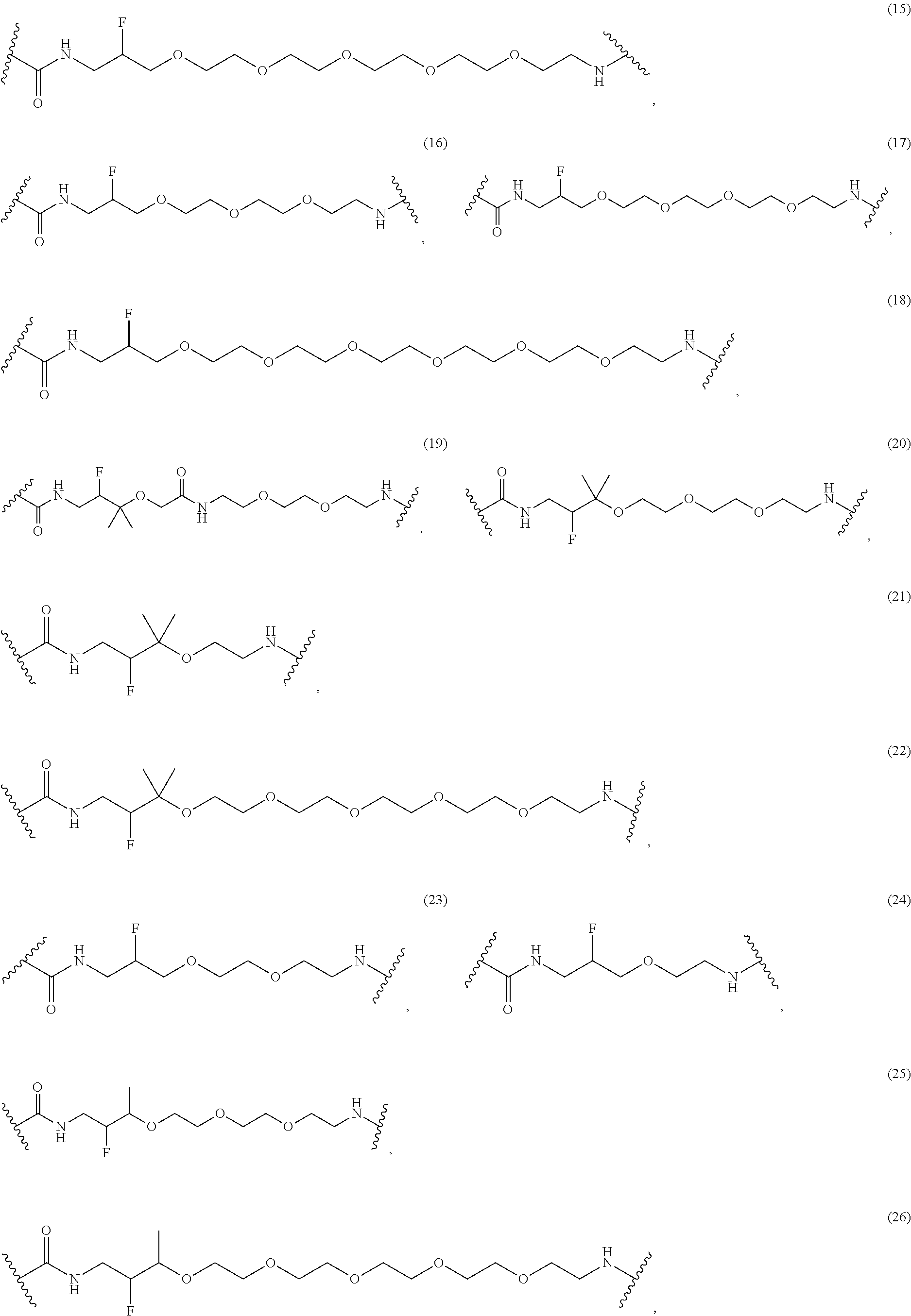

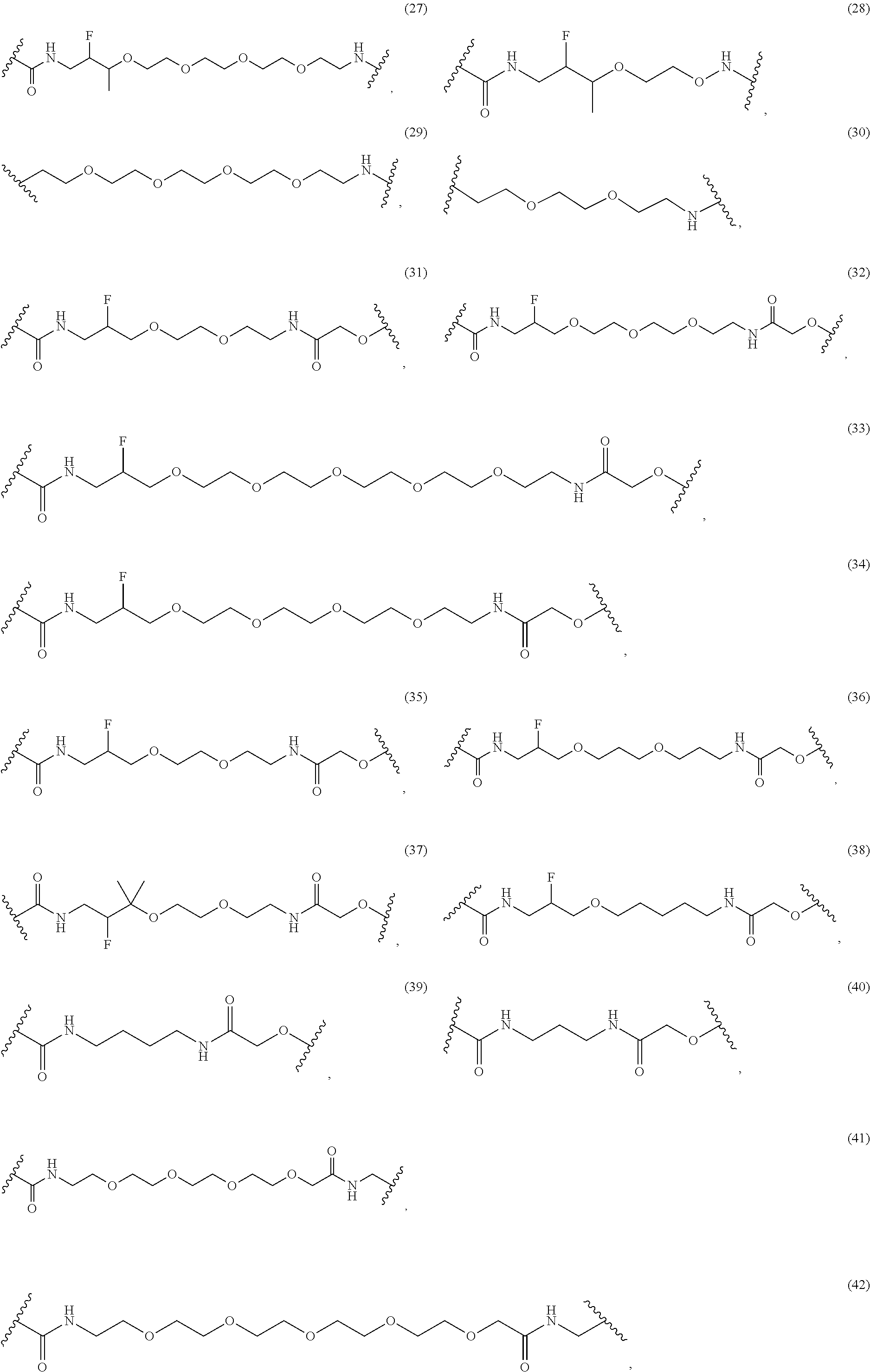

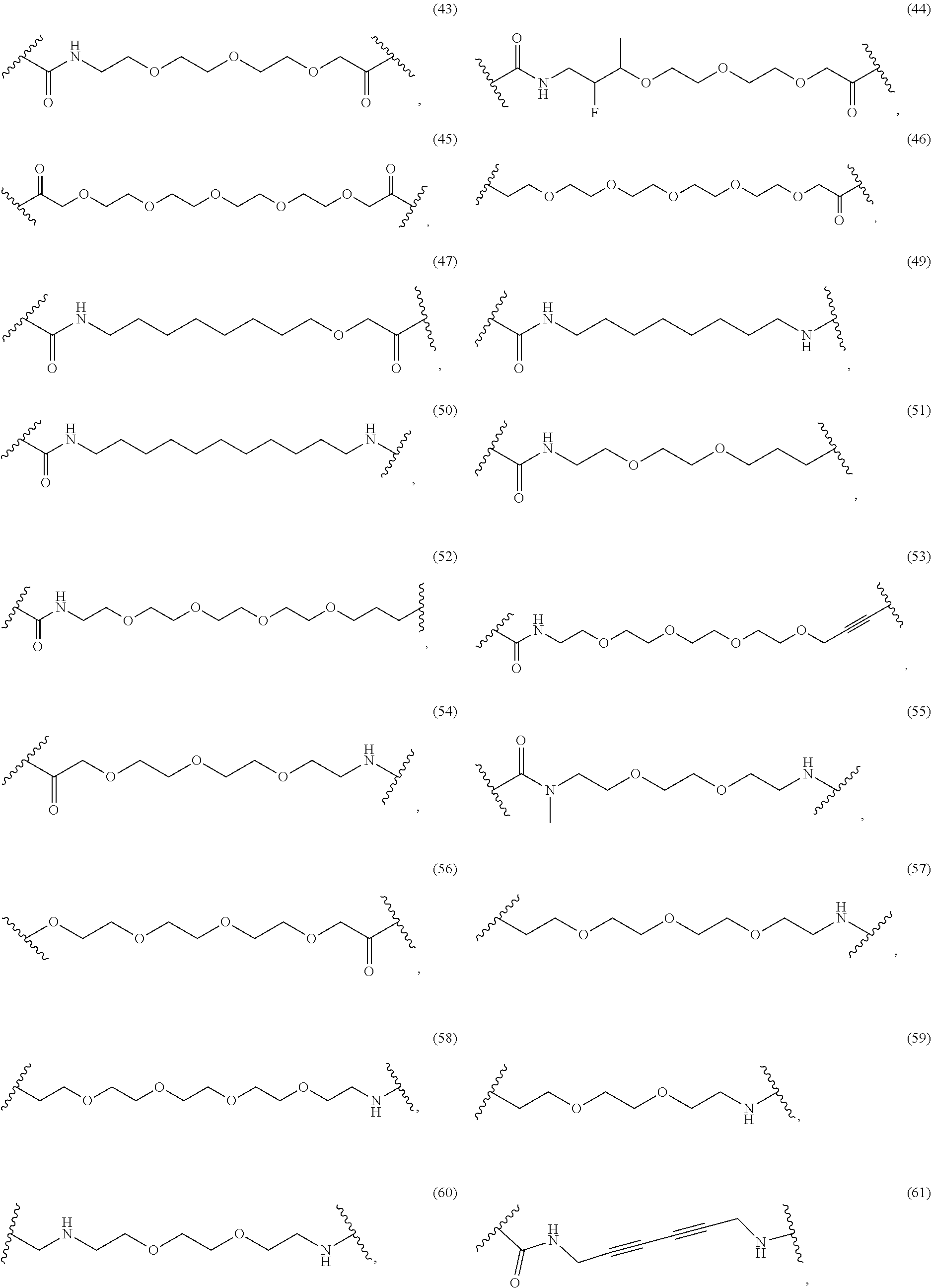

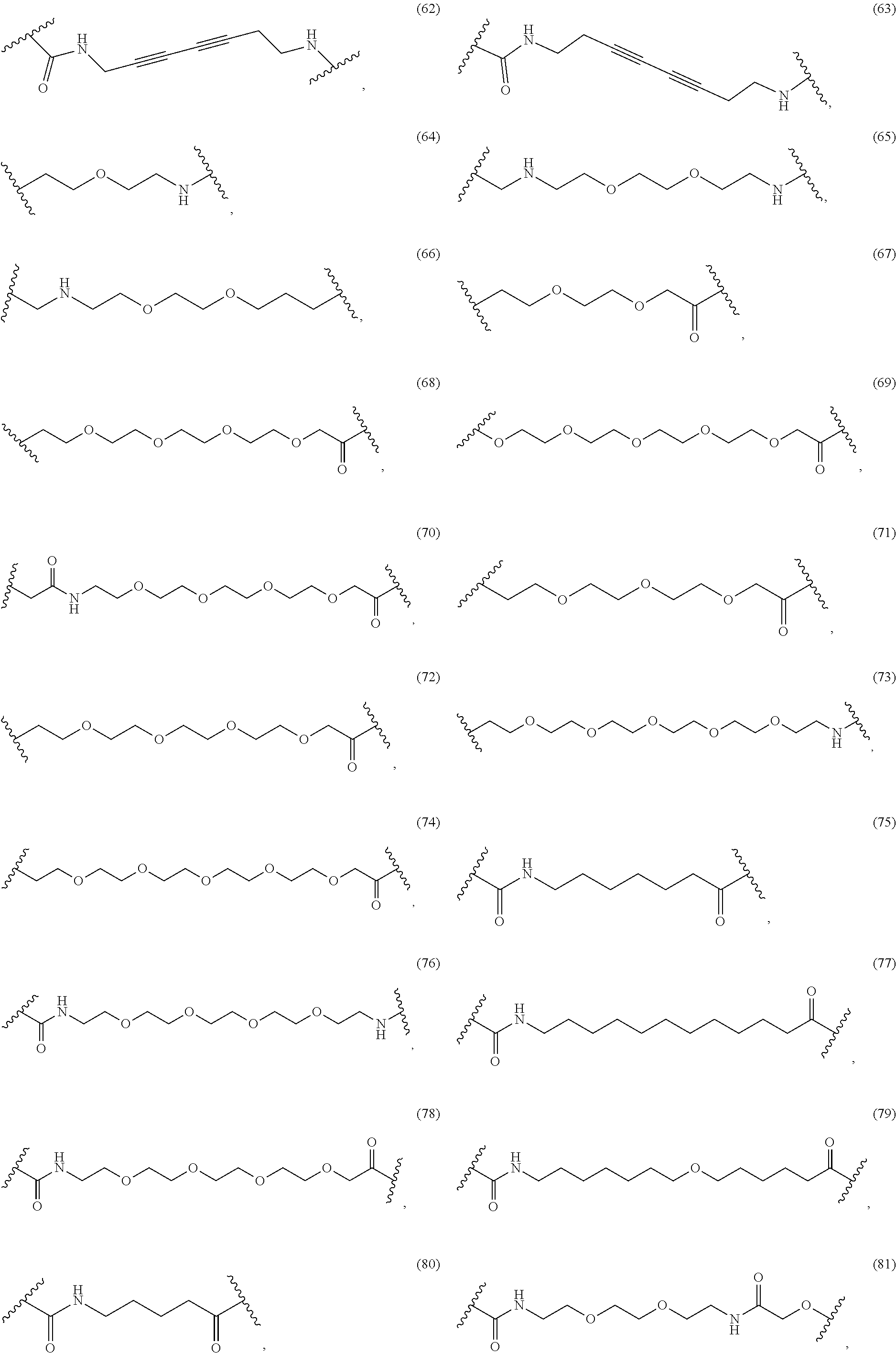

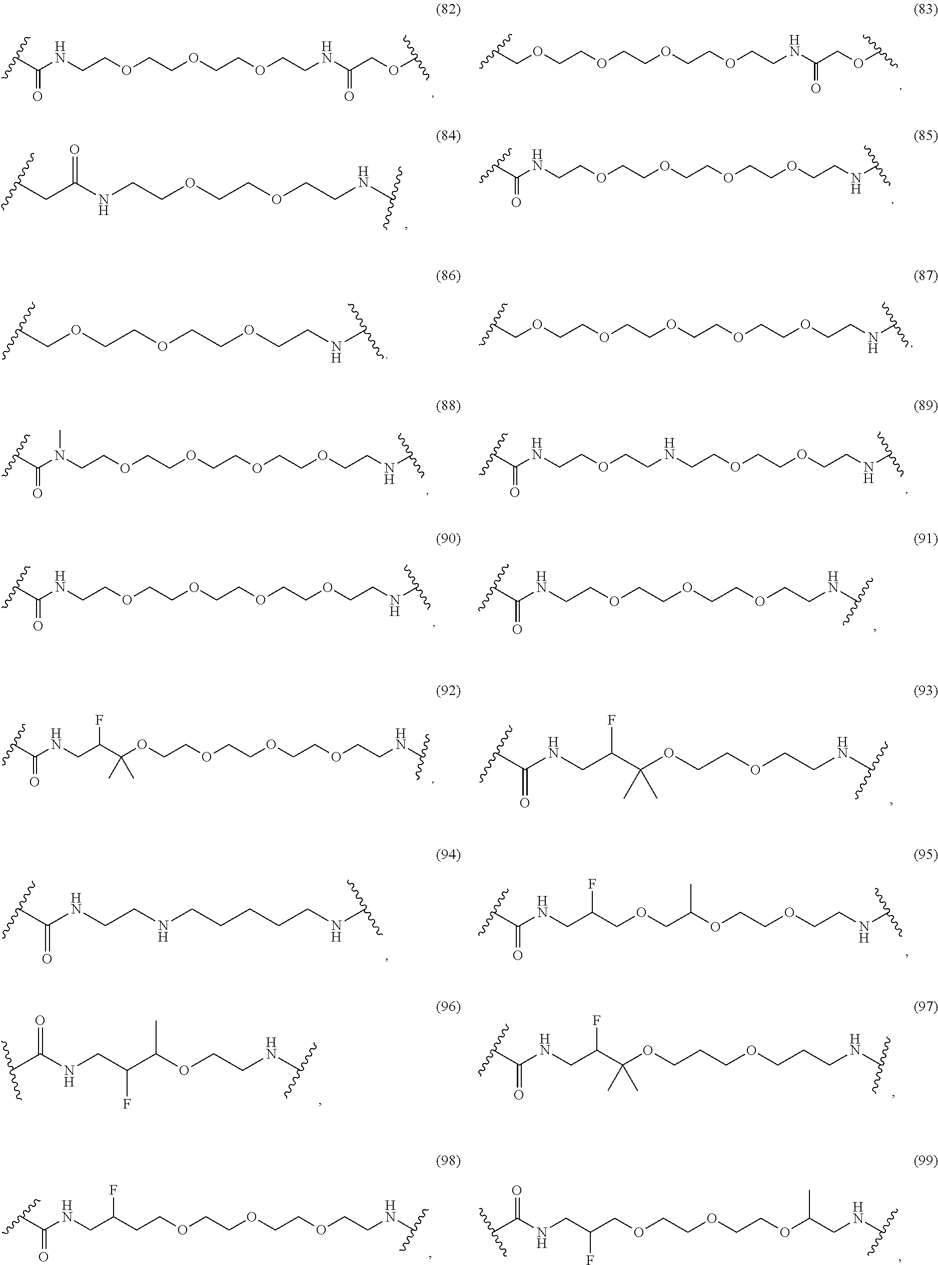

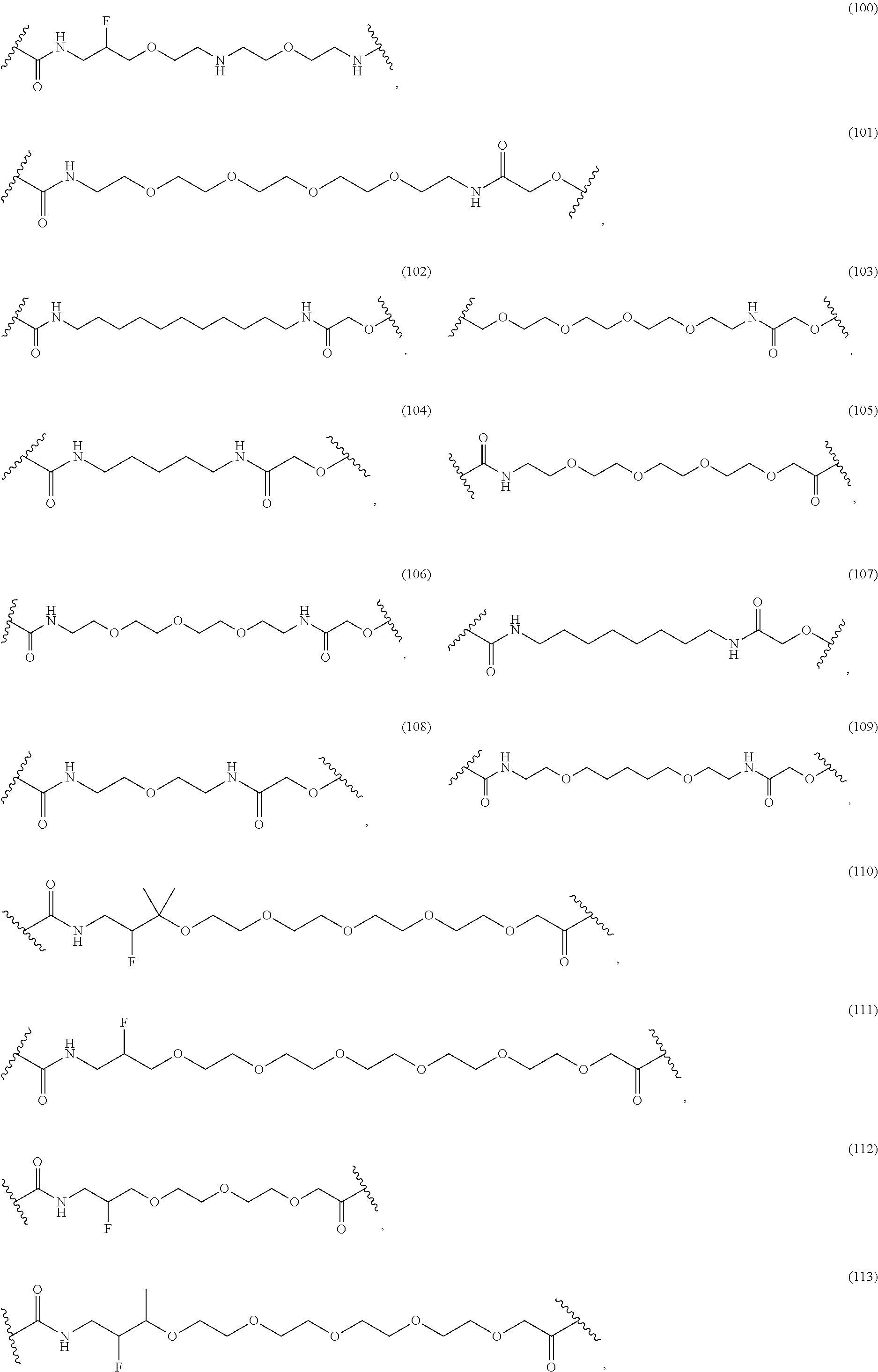

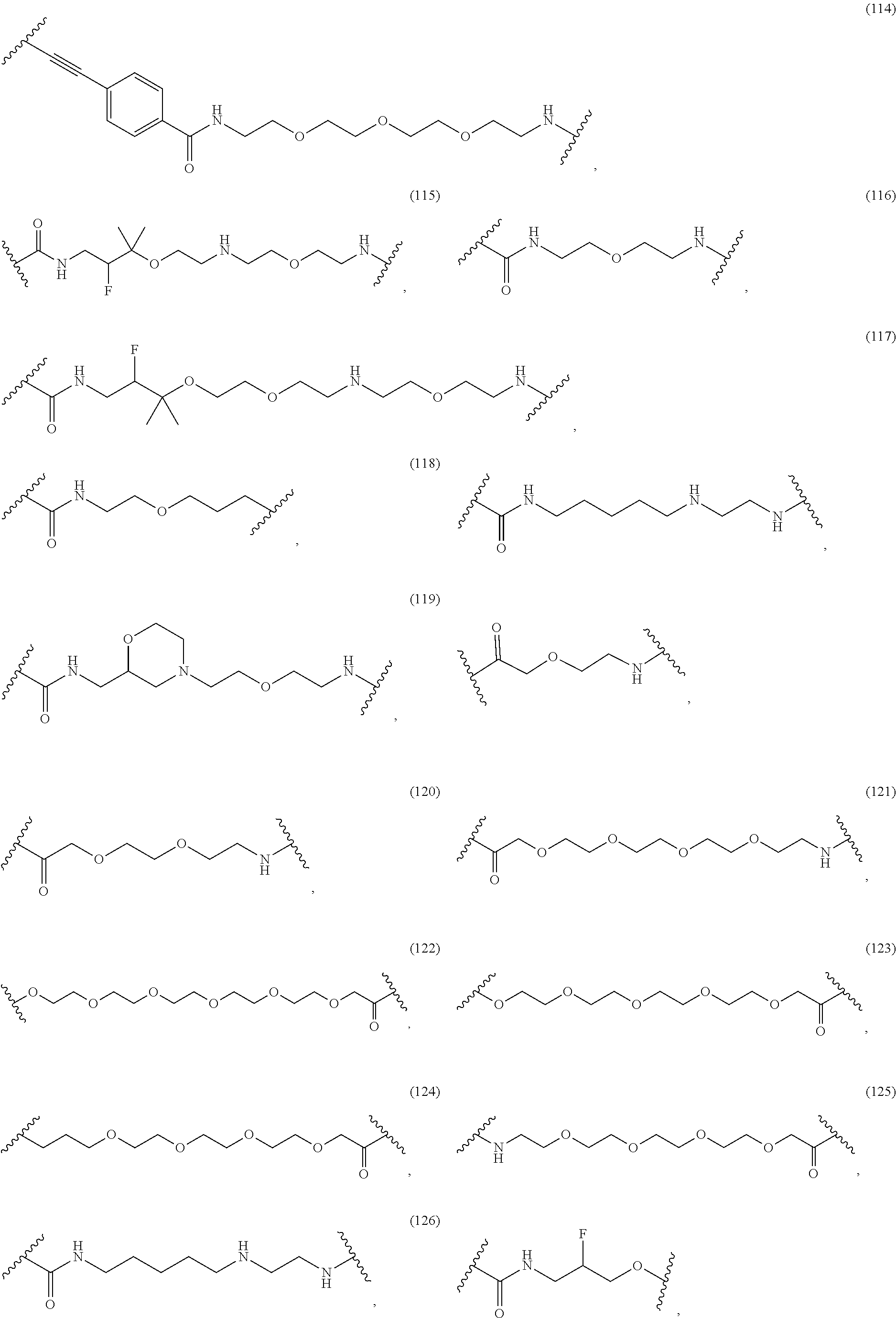

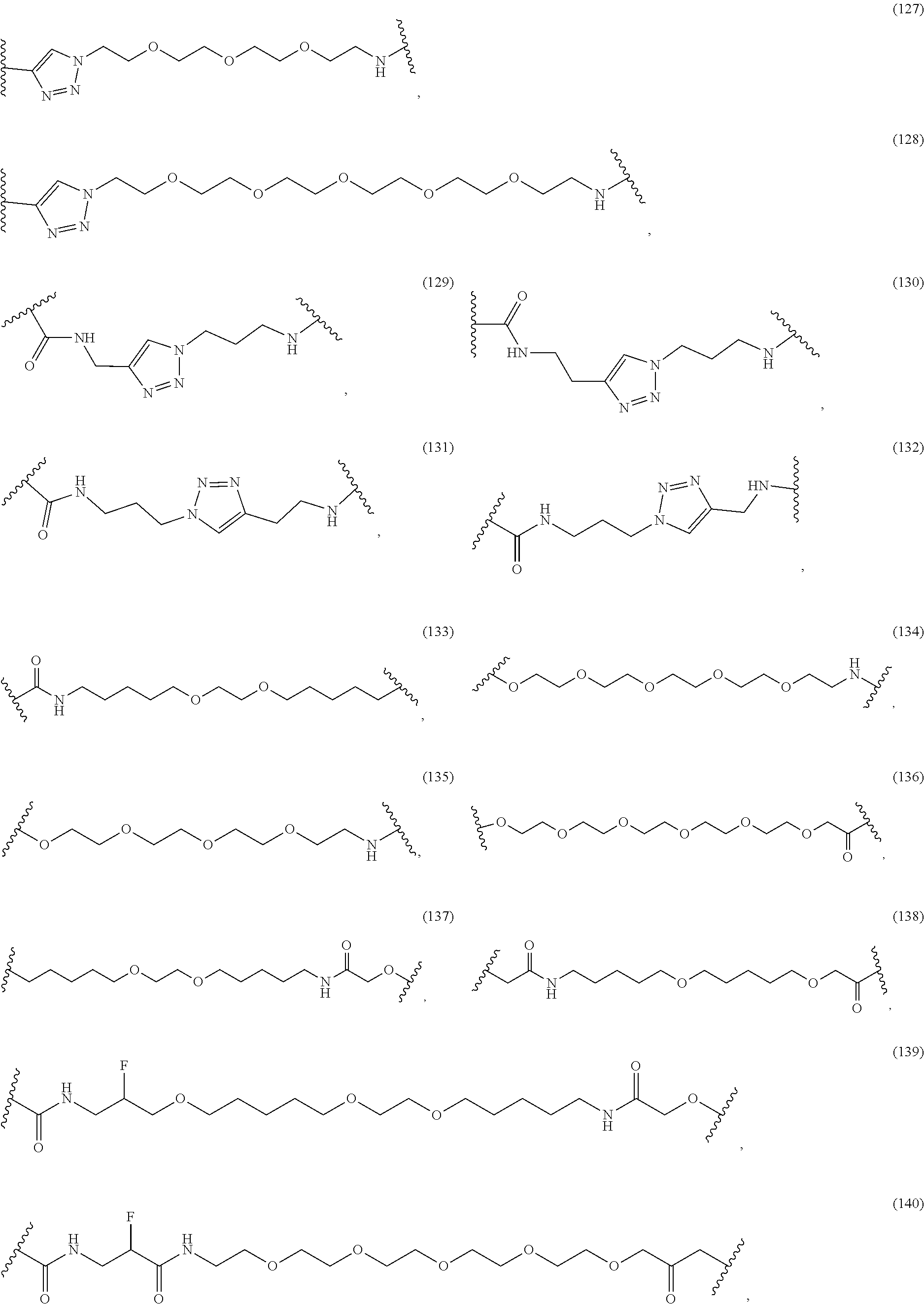

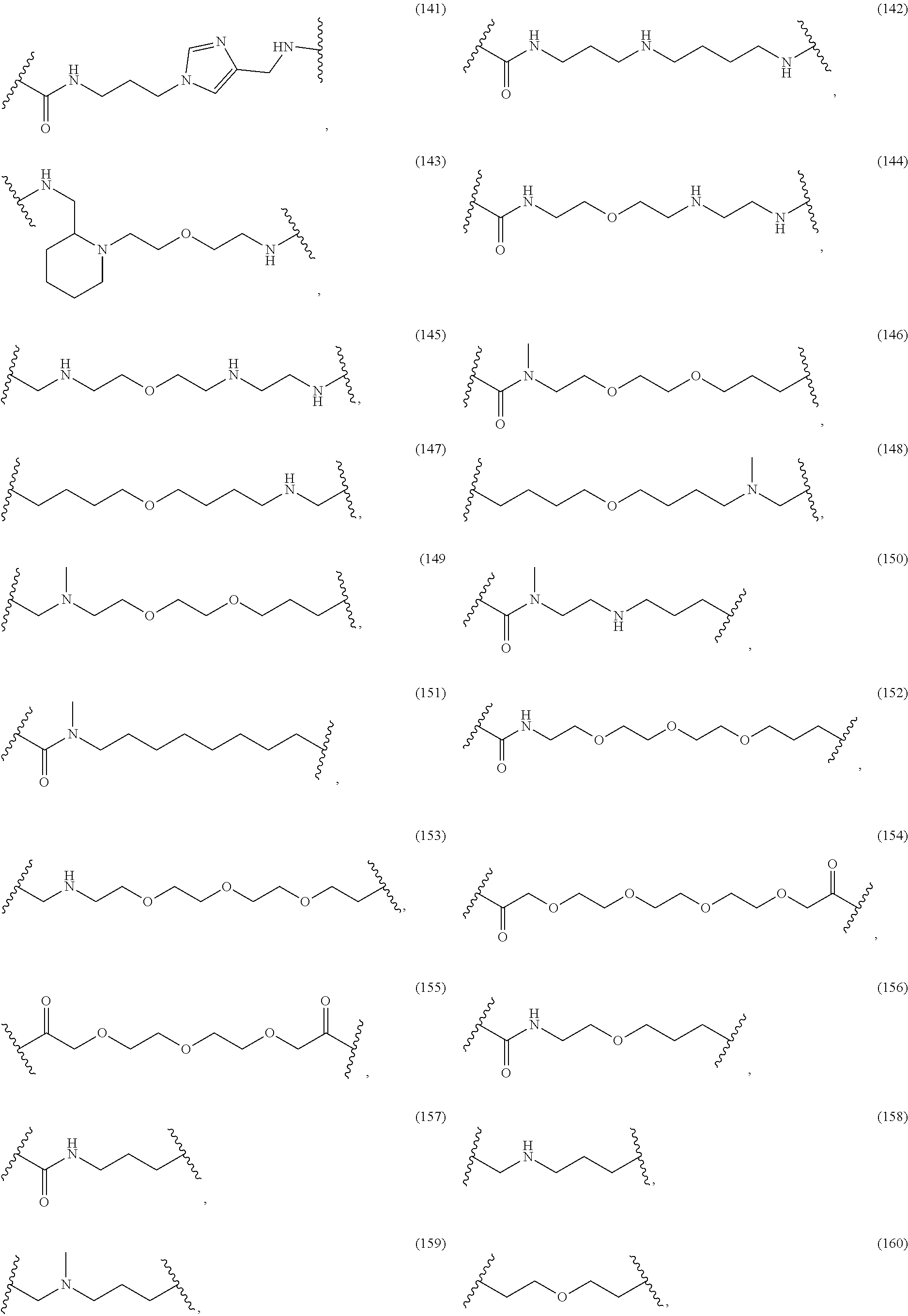

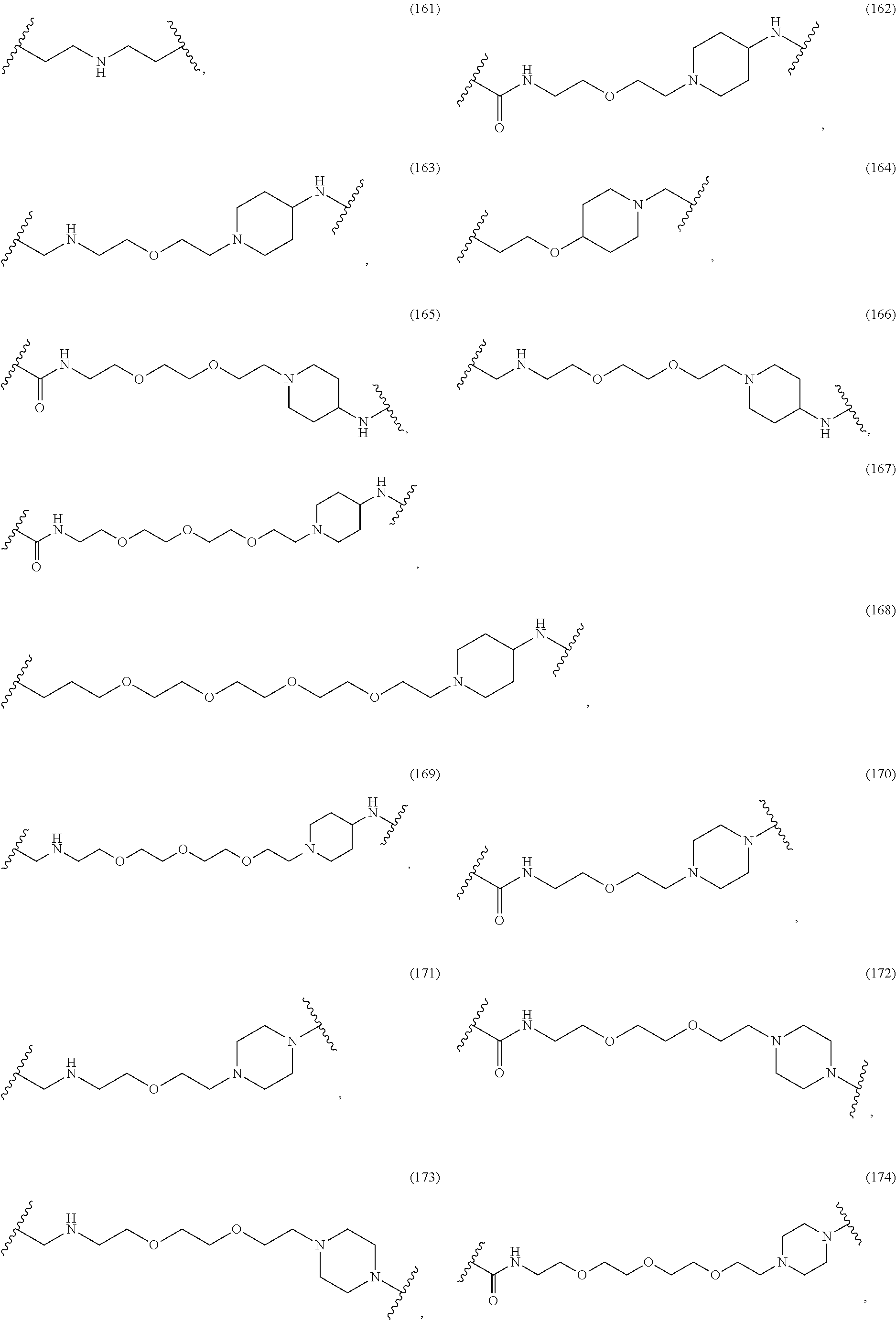

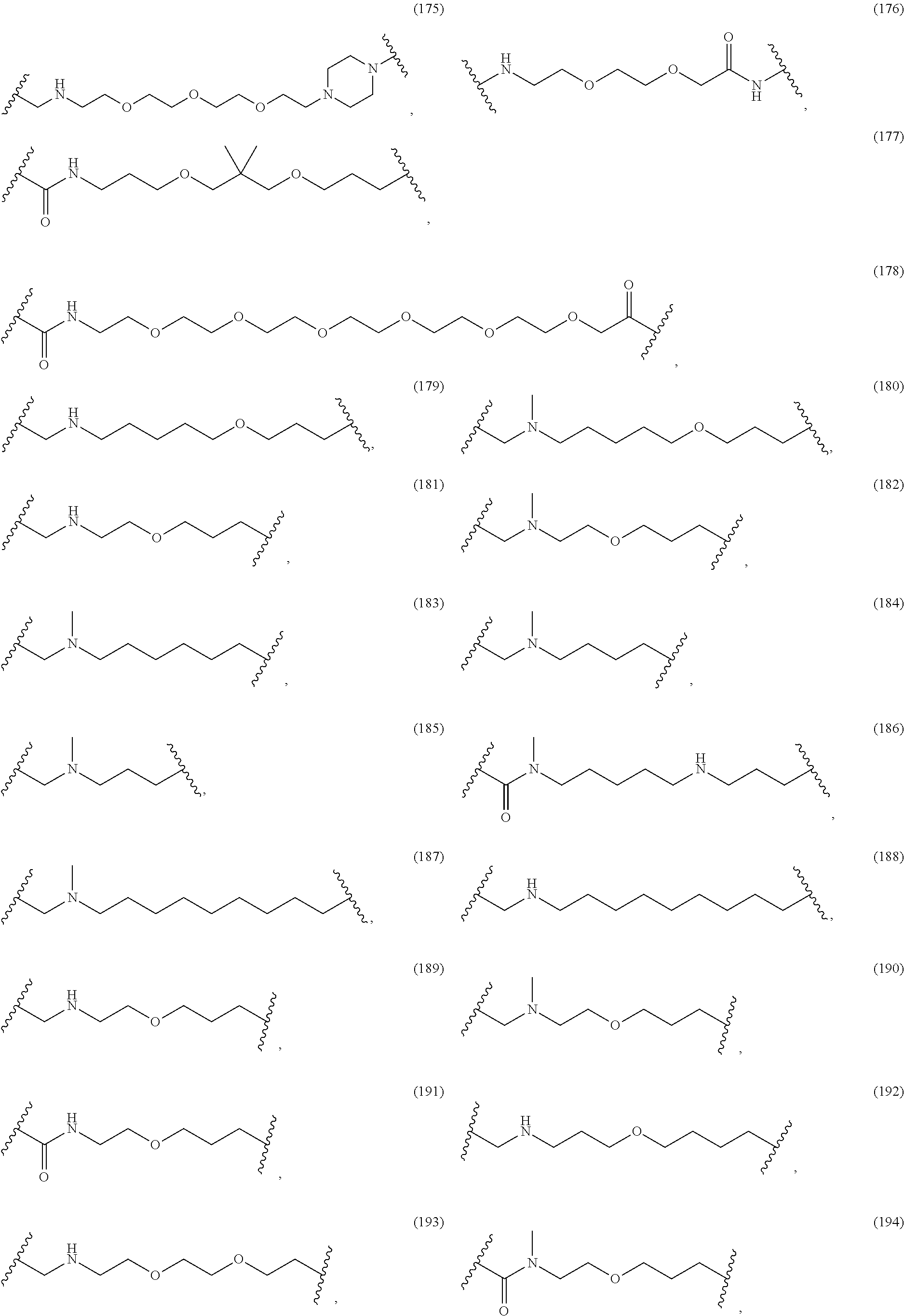

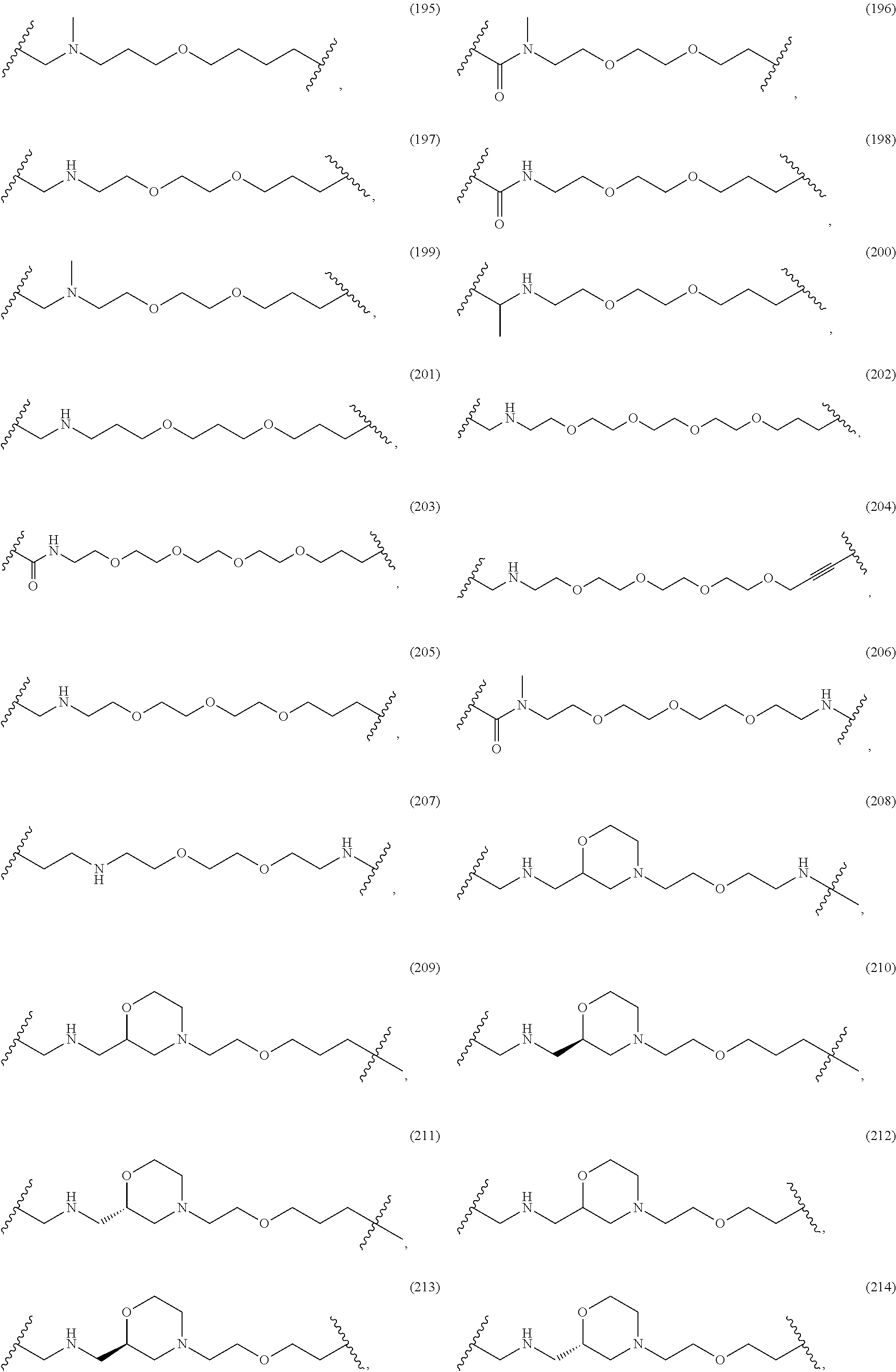

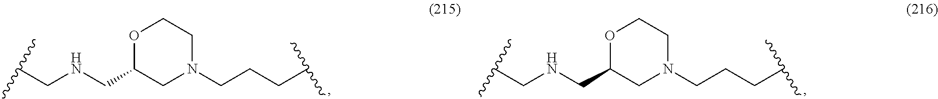 [SMILES: *CCCN1CCO[C@@H](CNC*)C1 ; *CCCN1CCO[C@H](CNC*)C1]

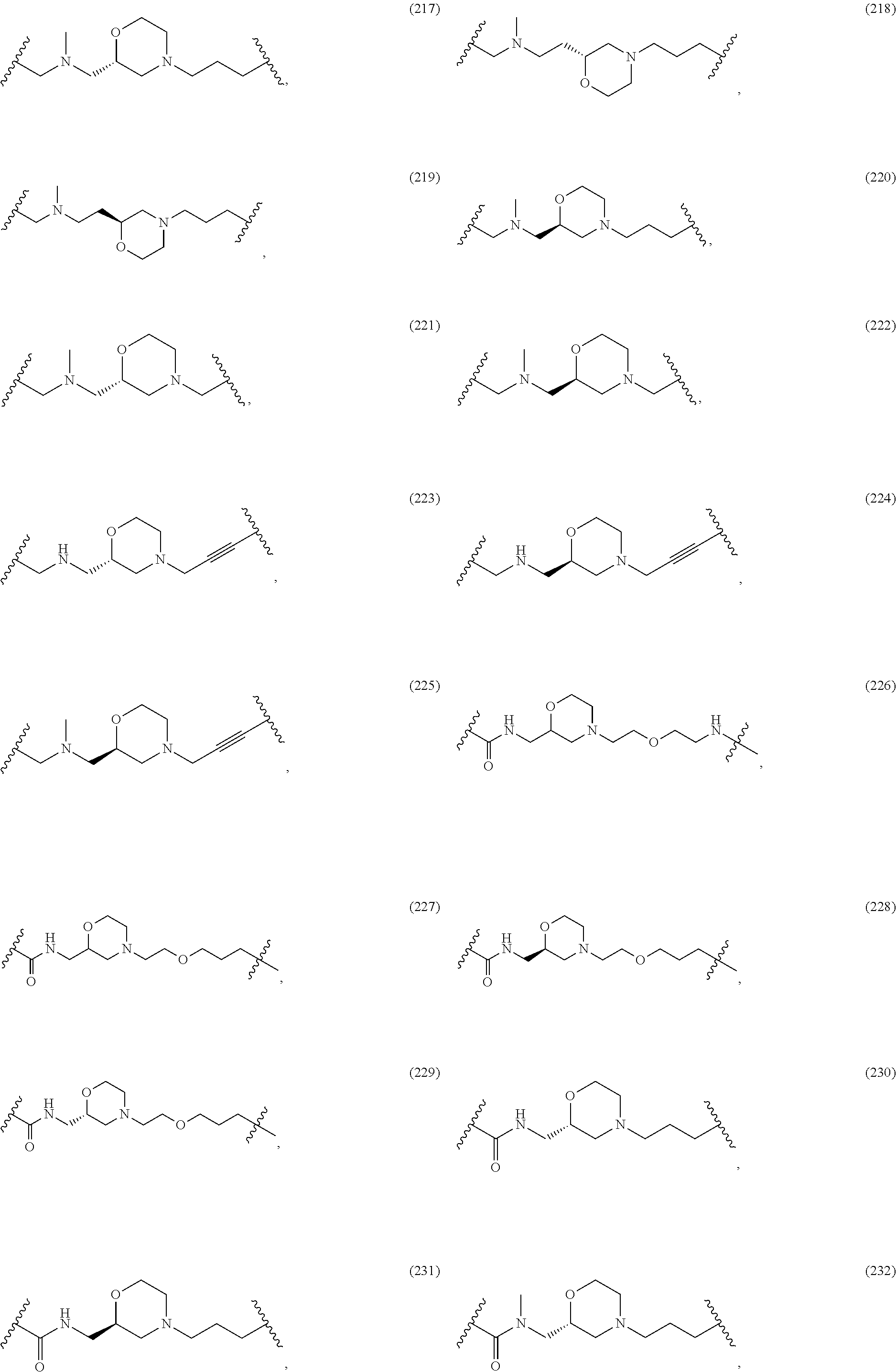

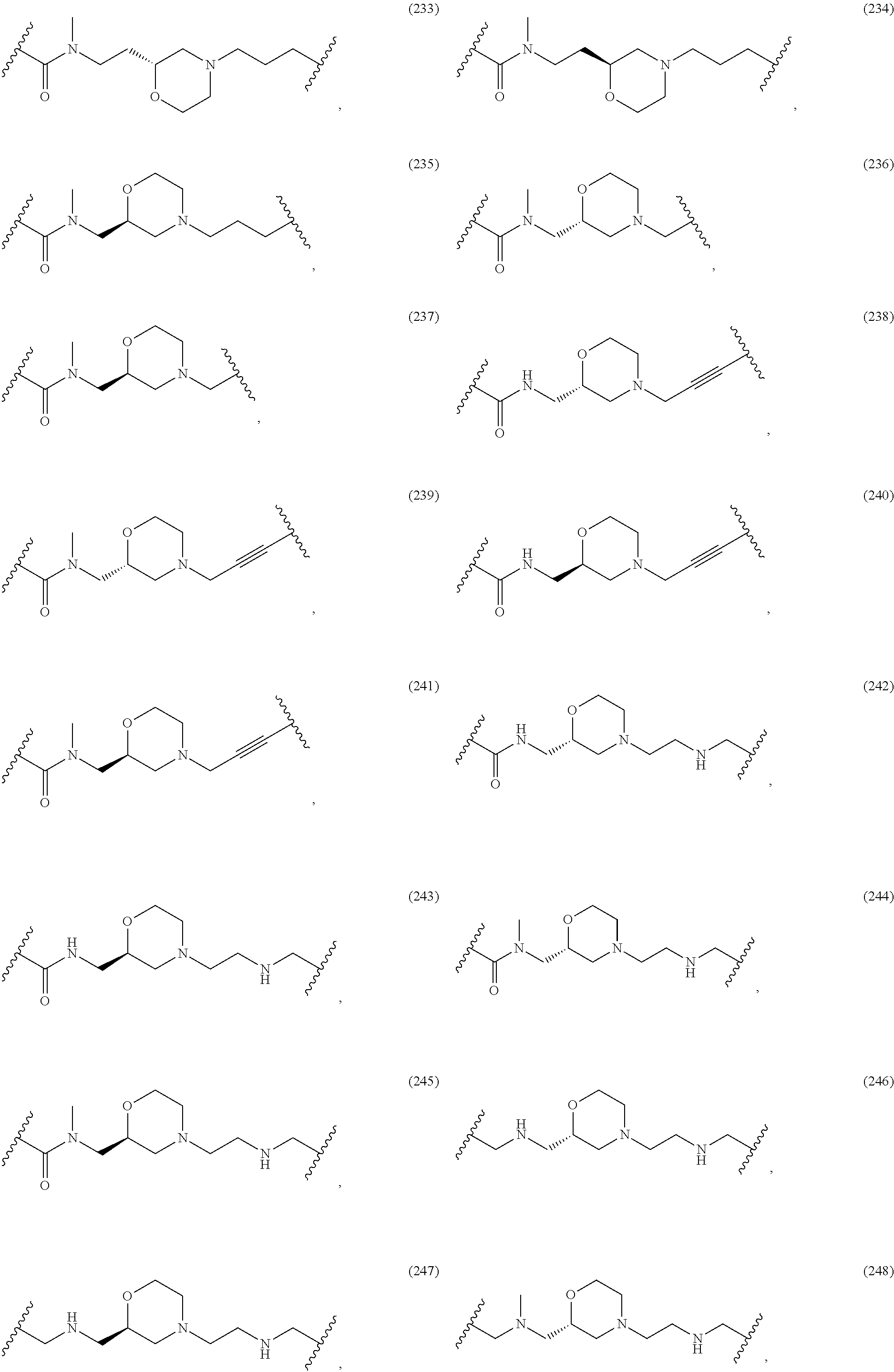

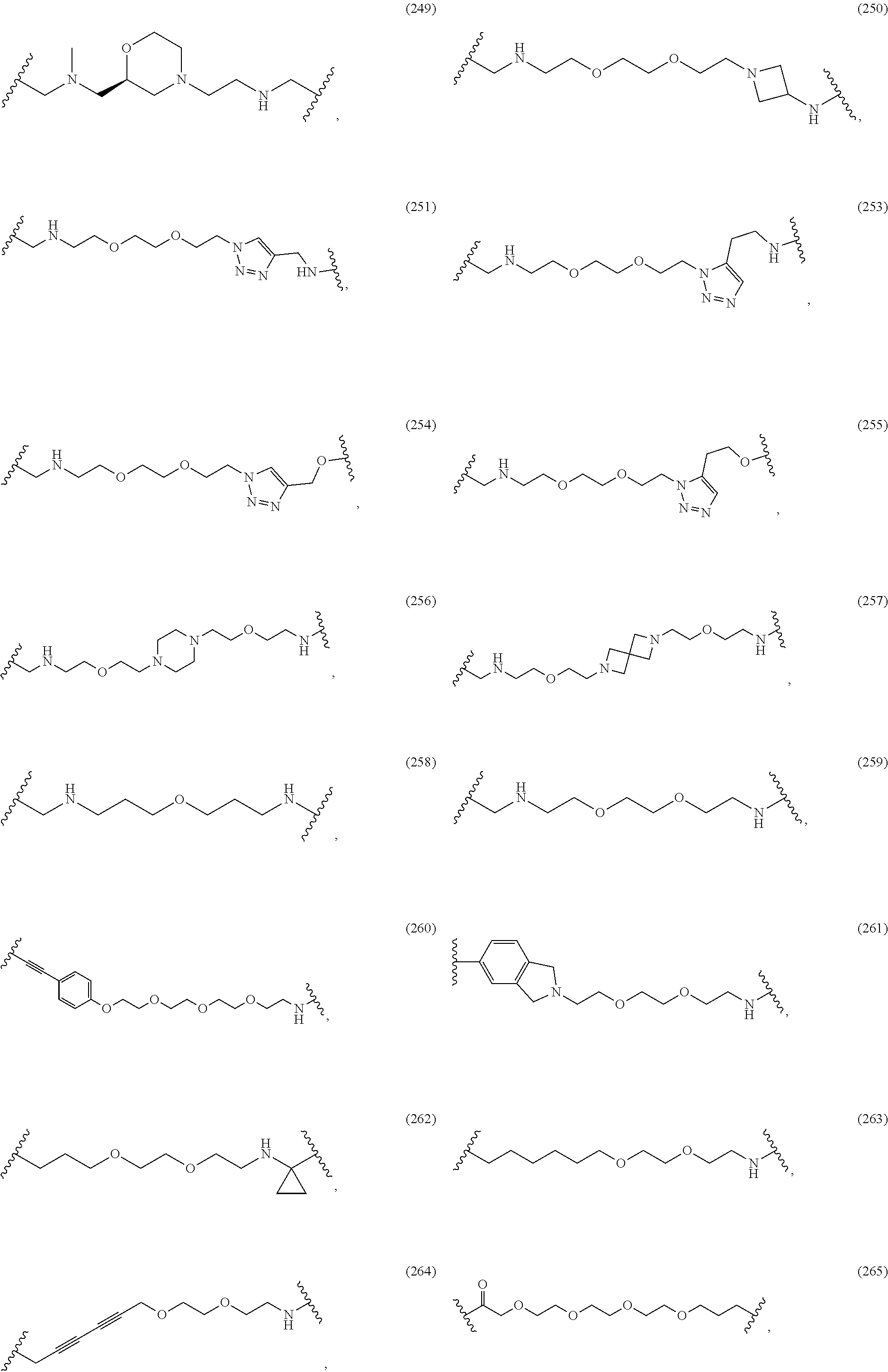

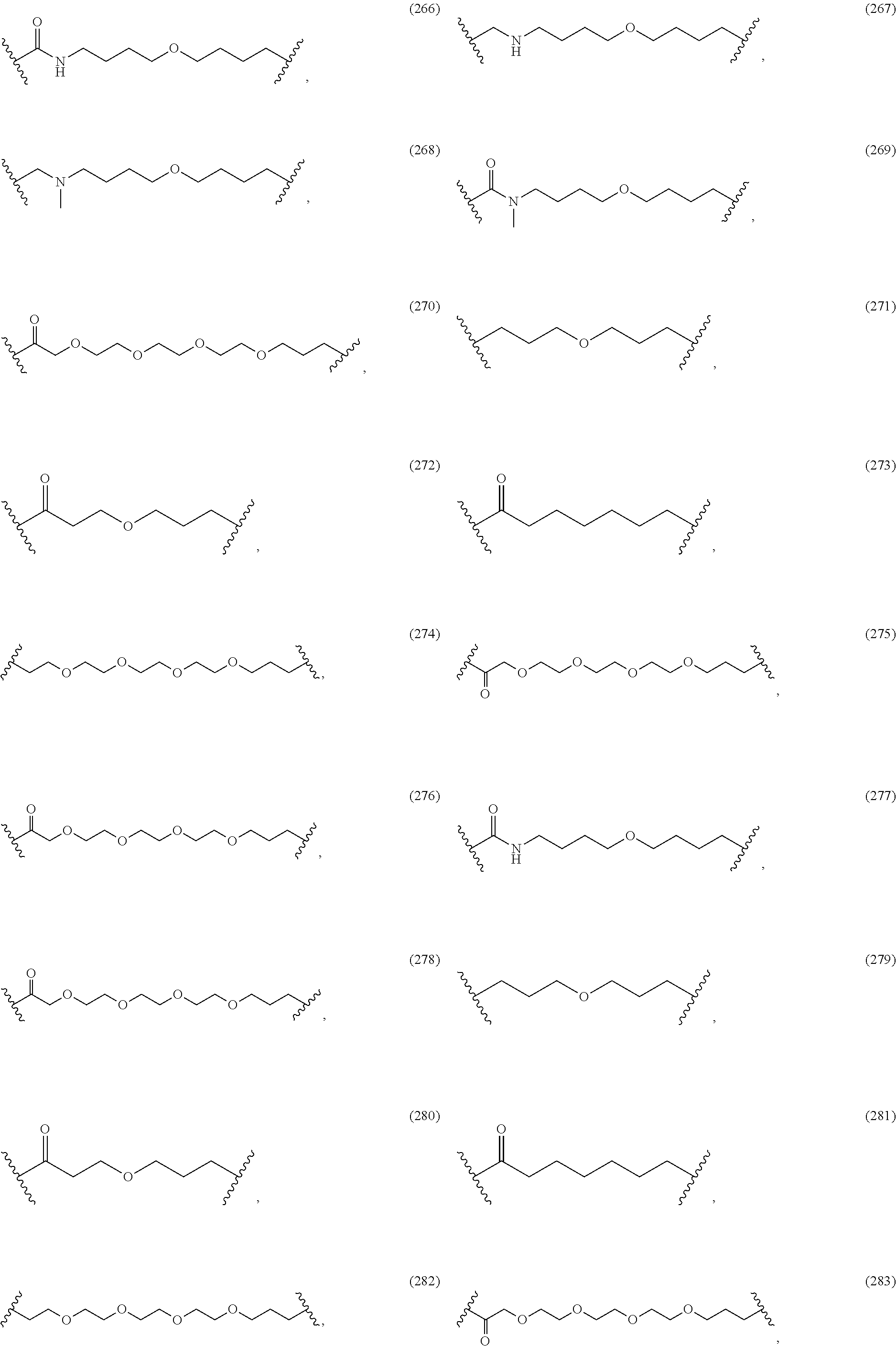

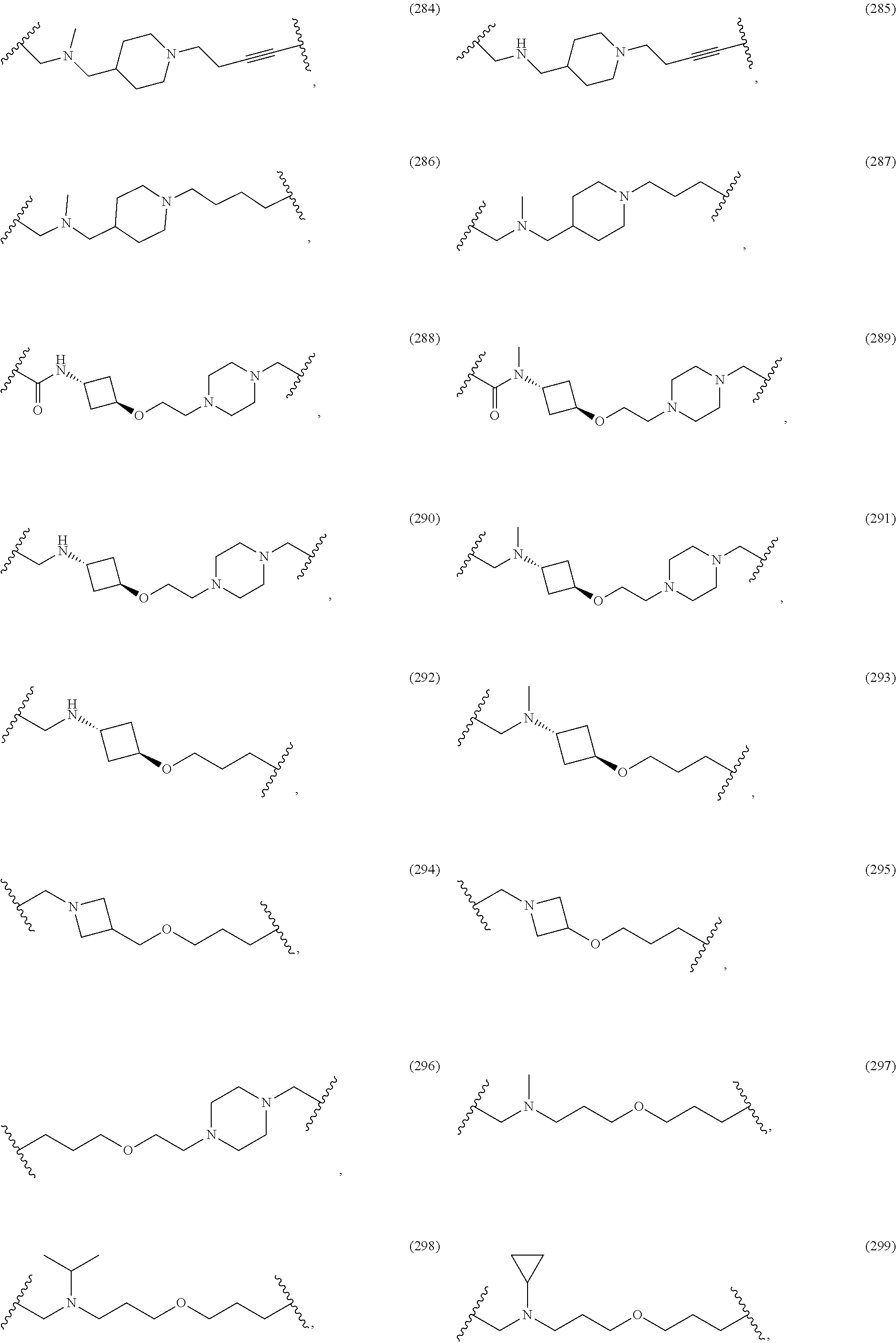

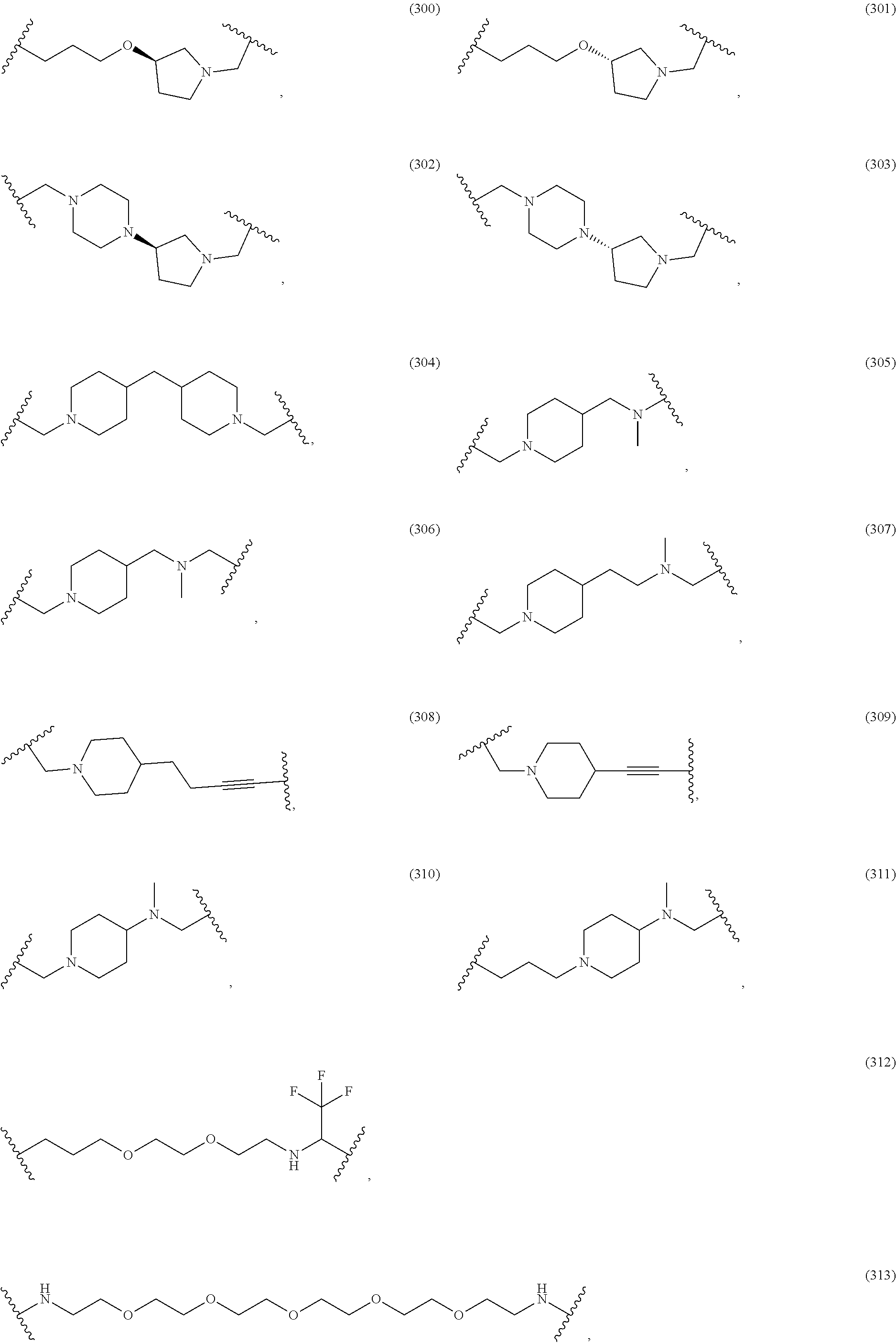

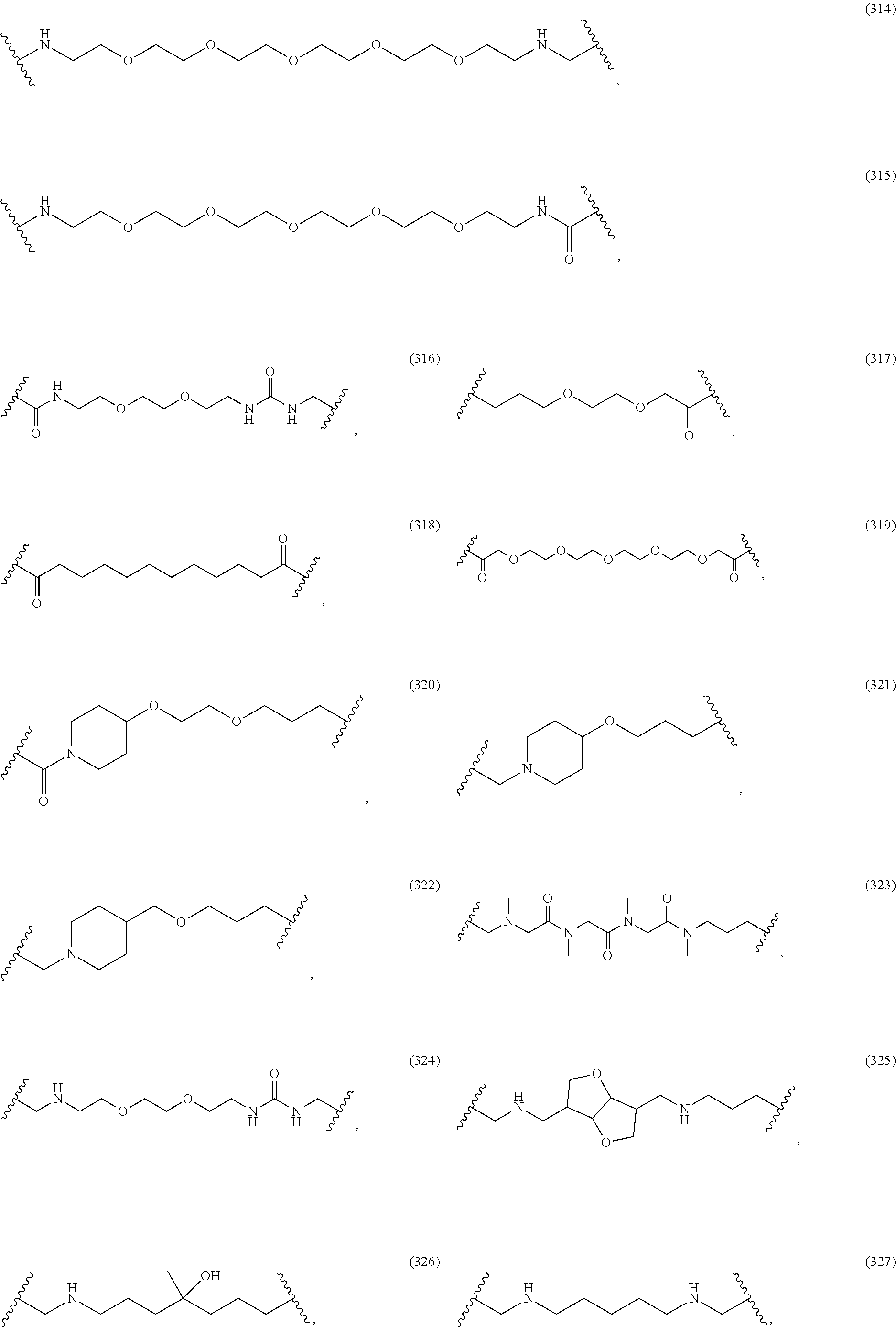

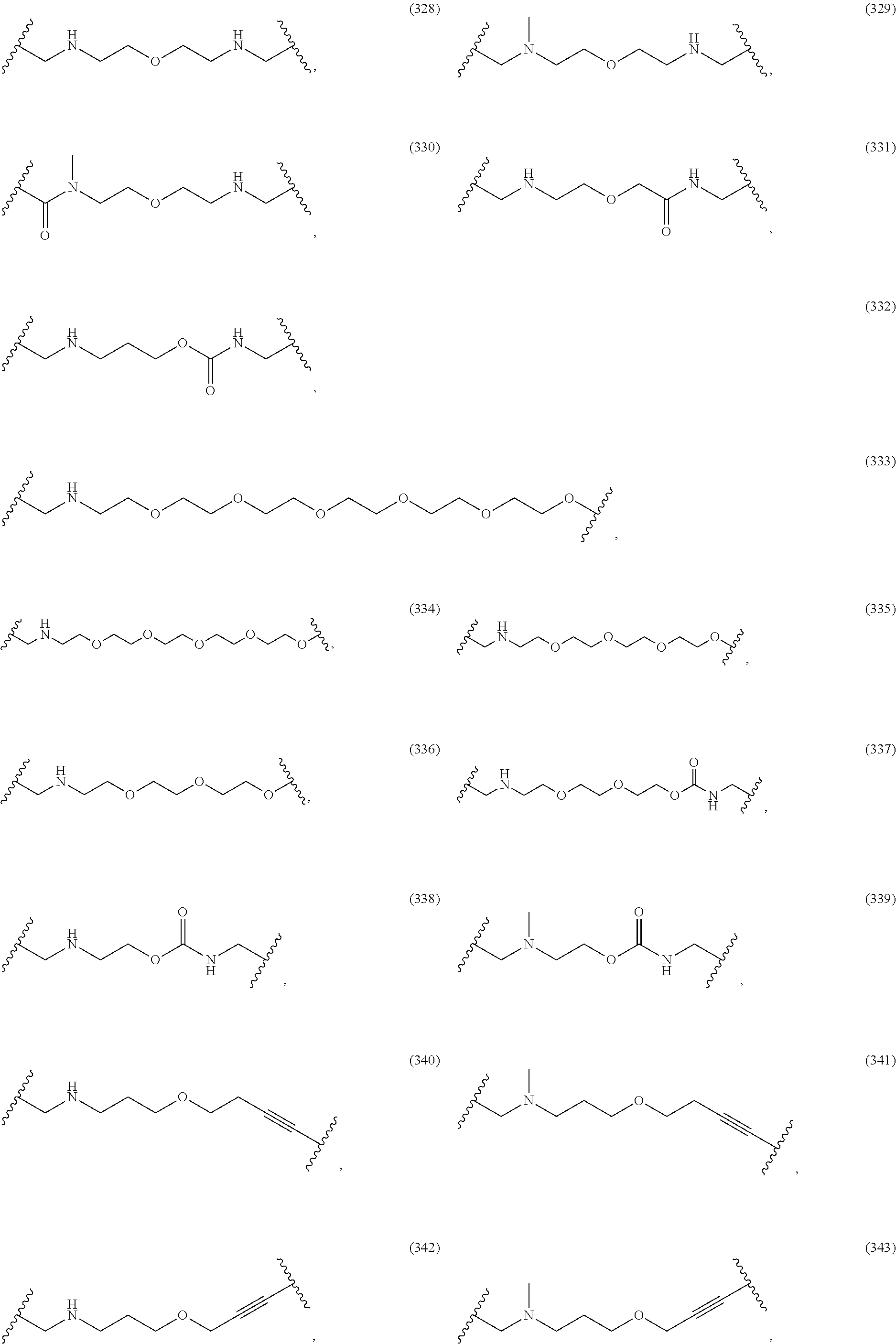

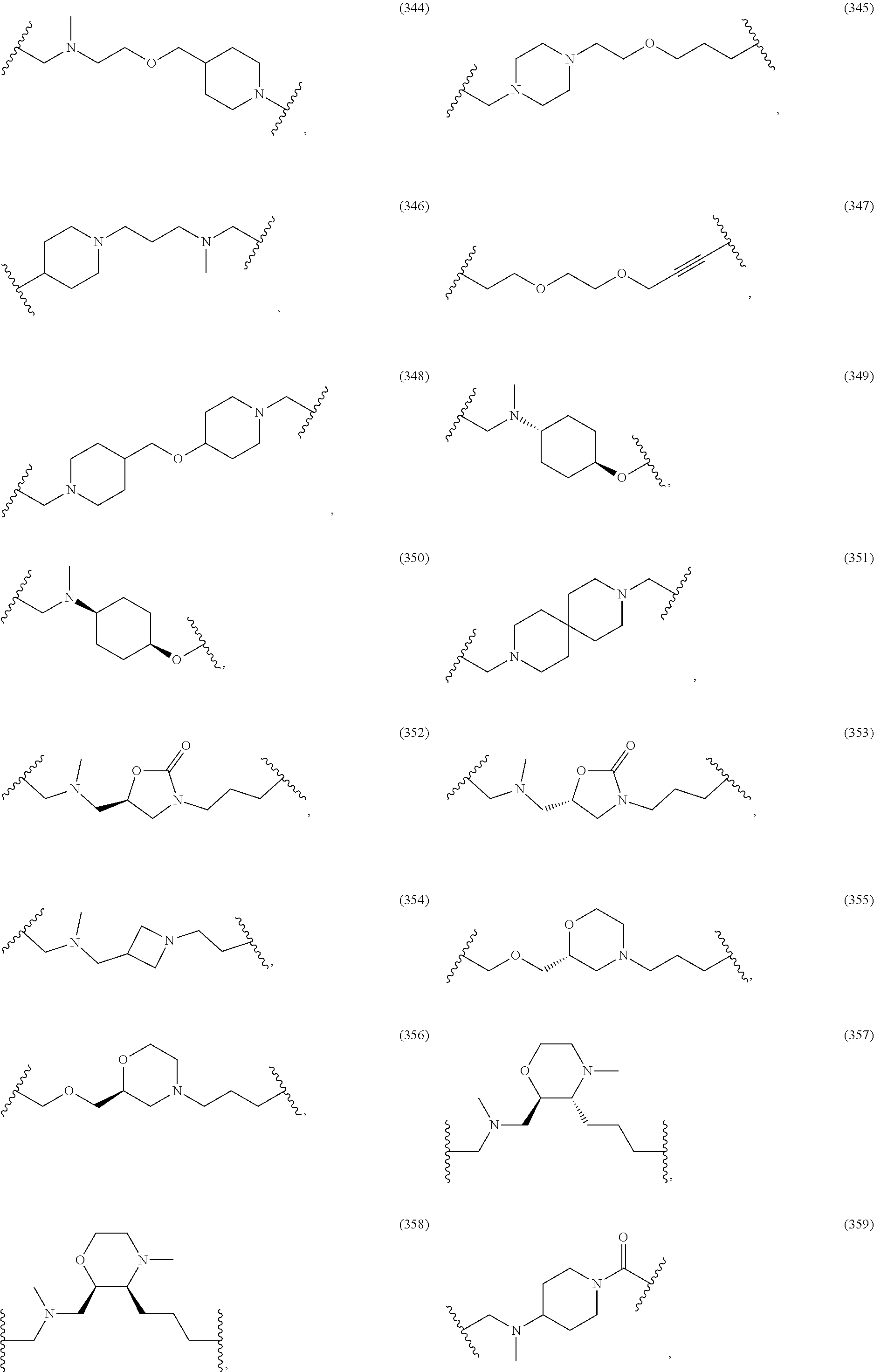

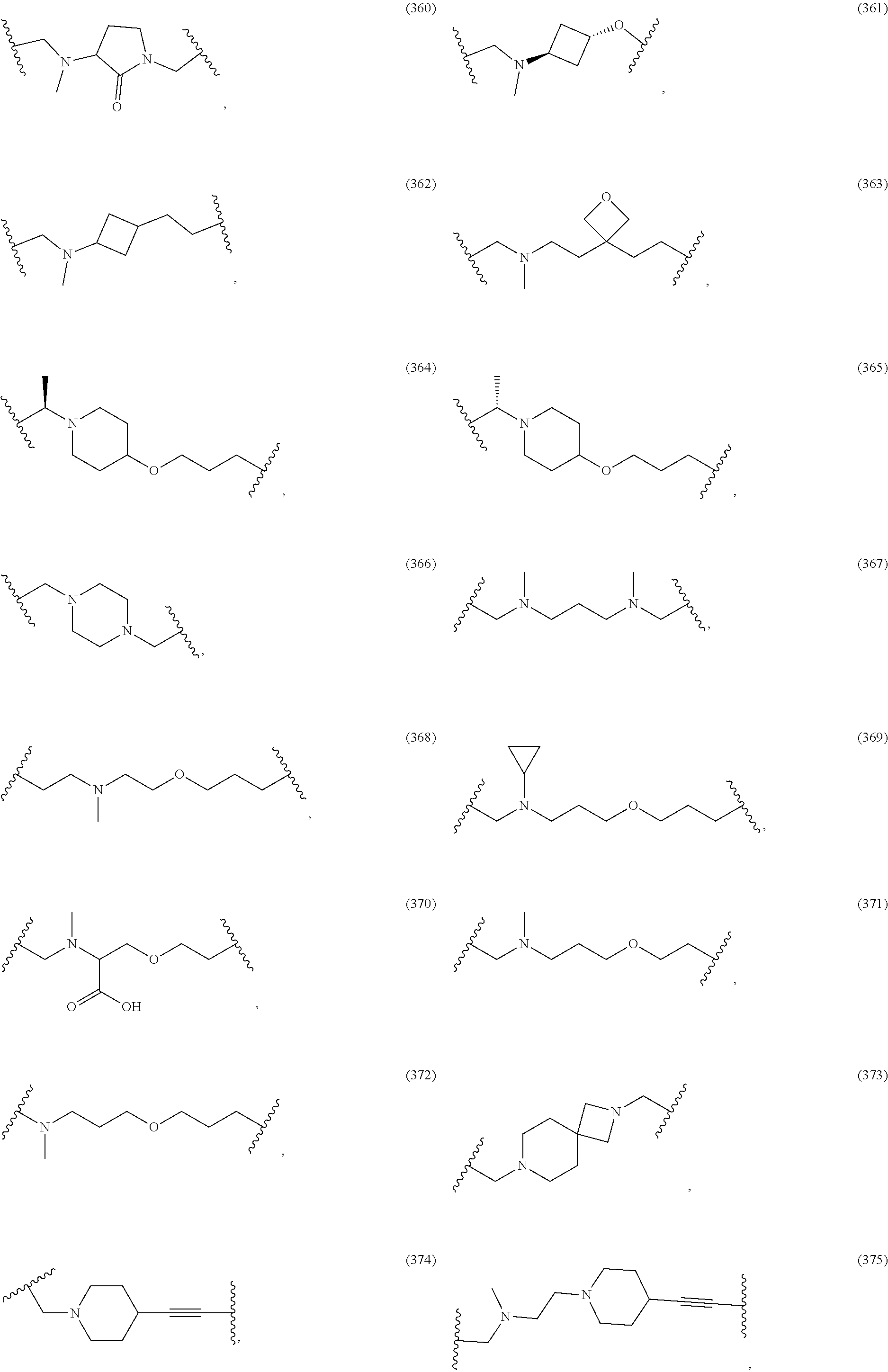

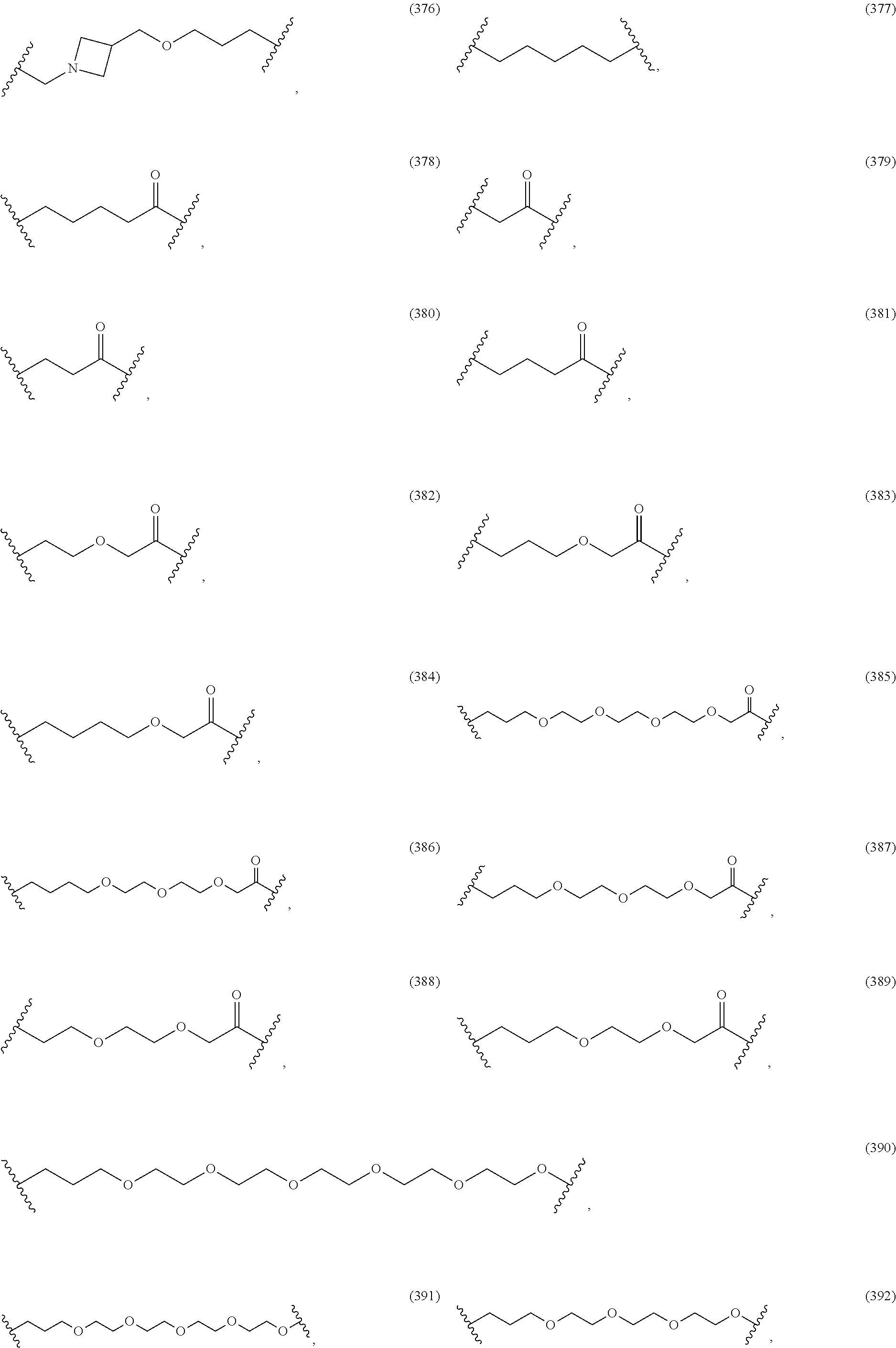

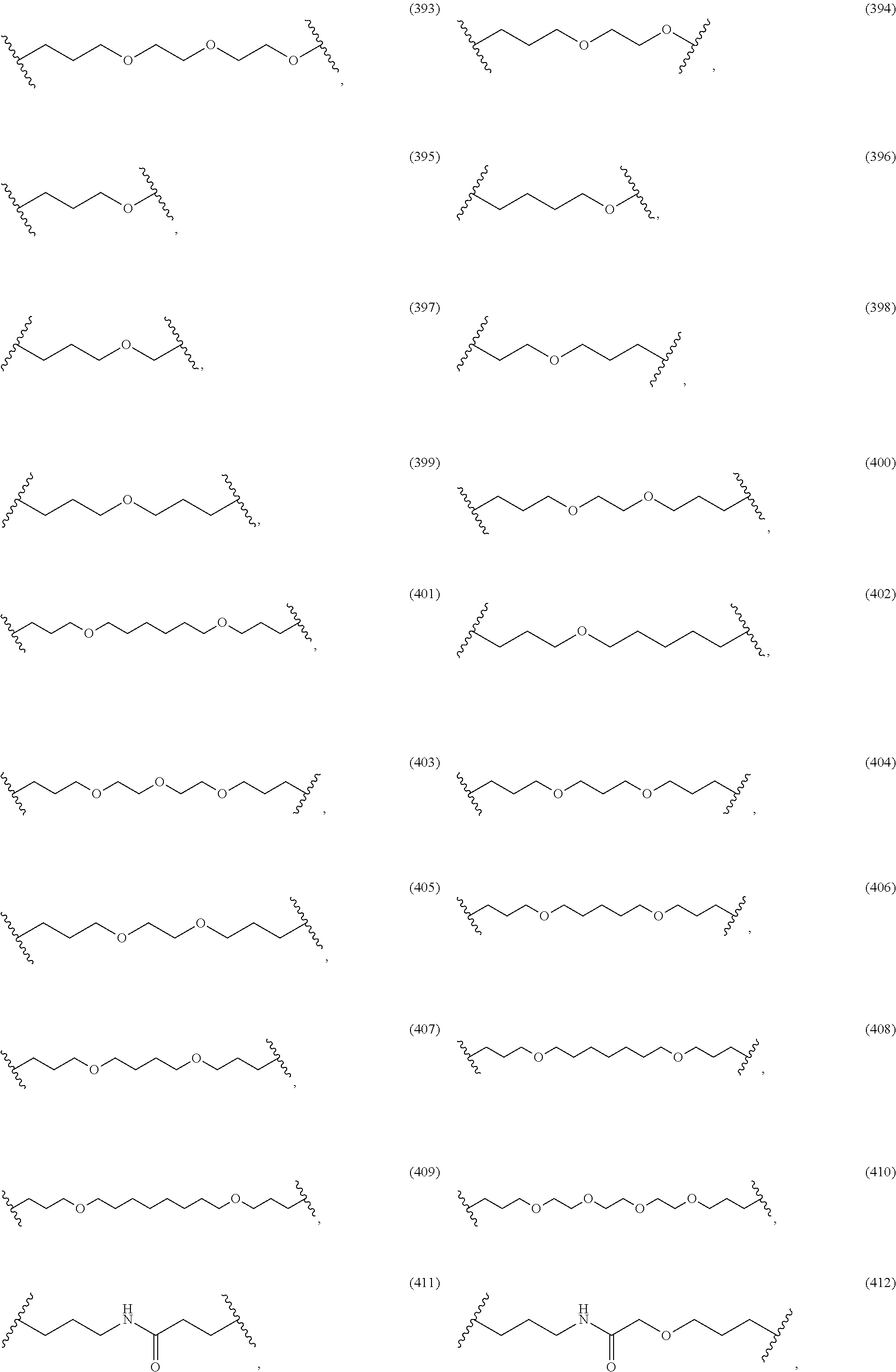

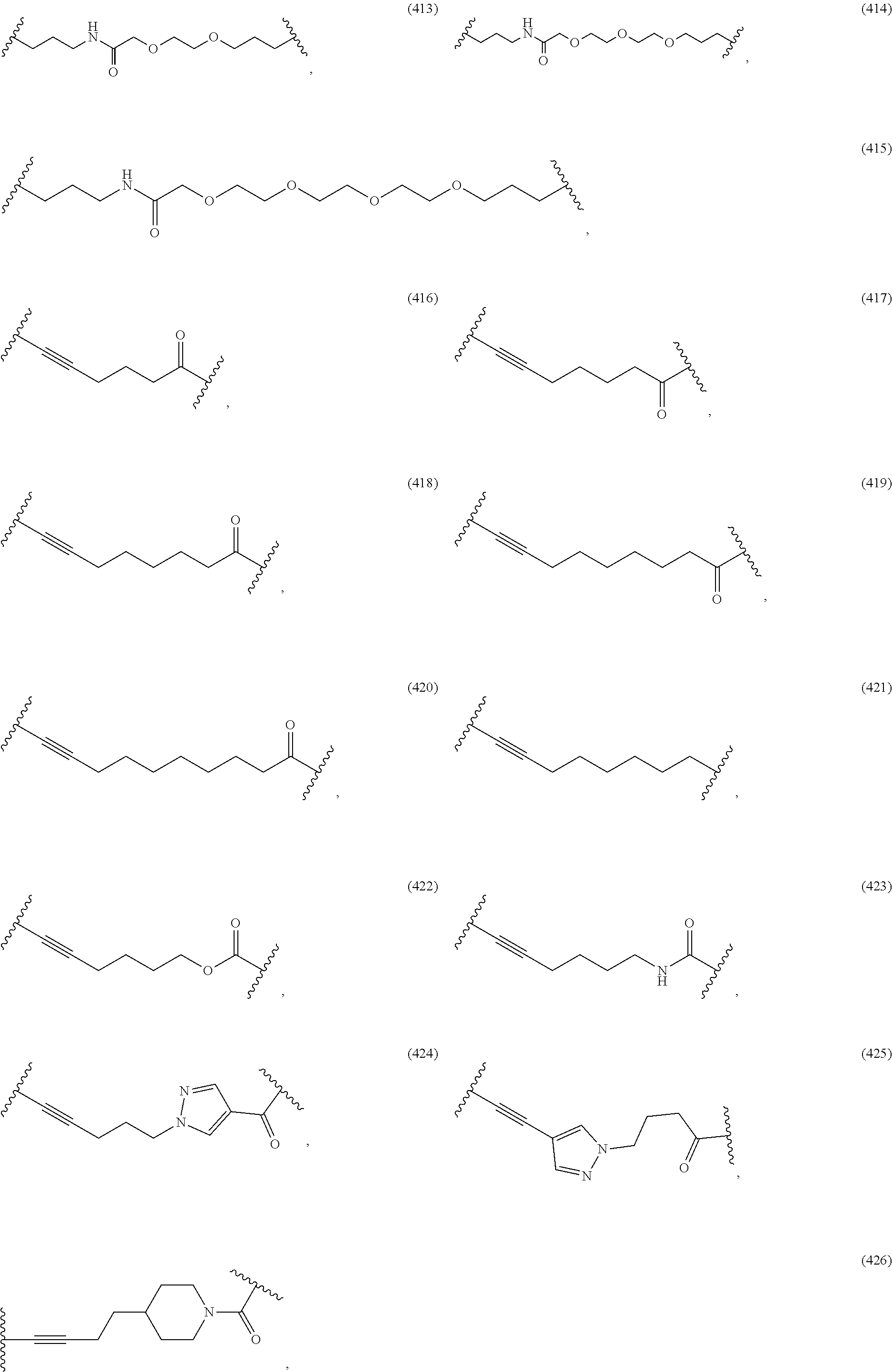

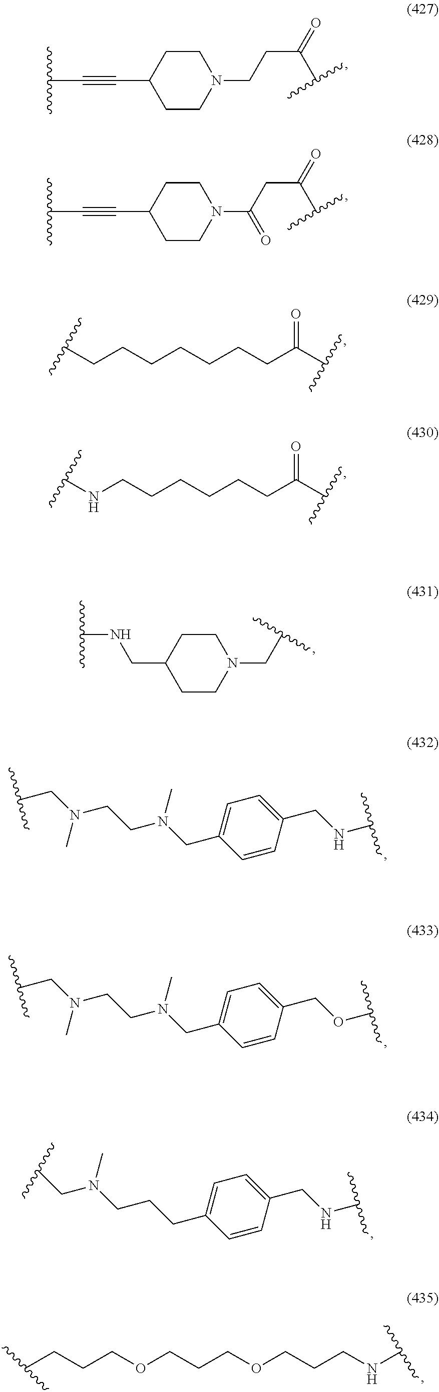

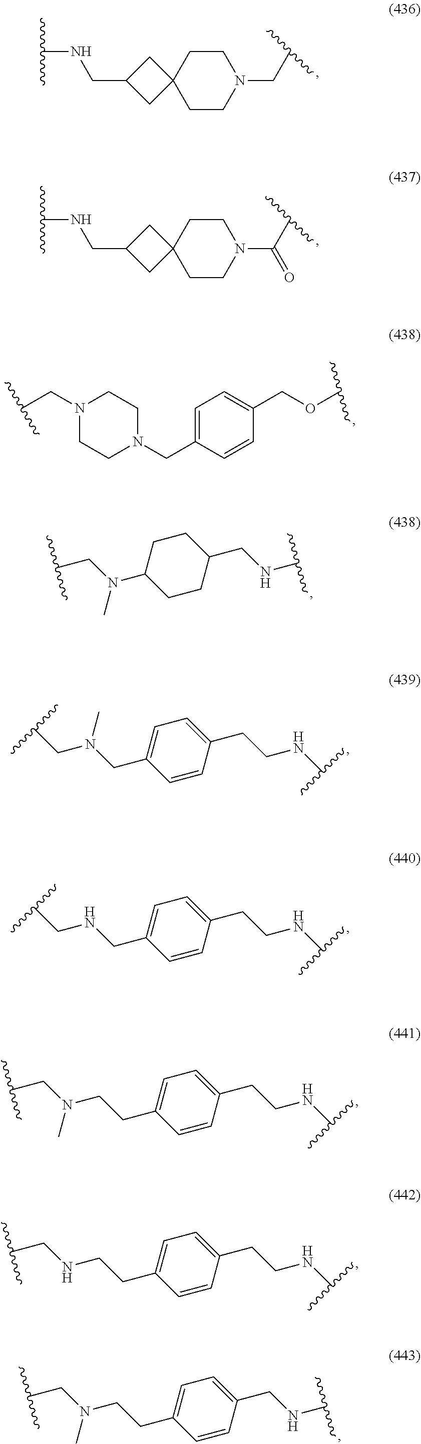

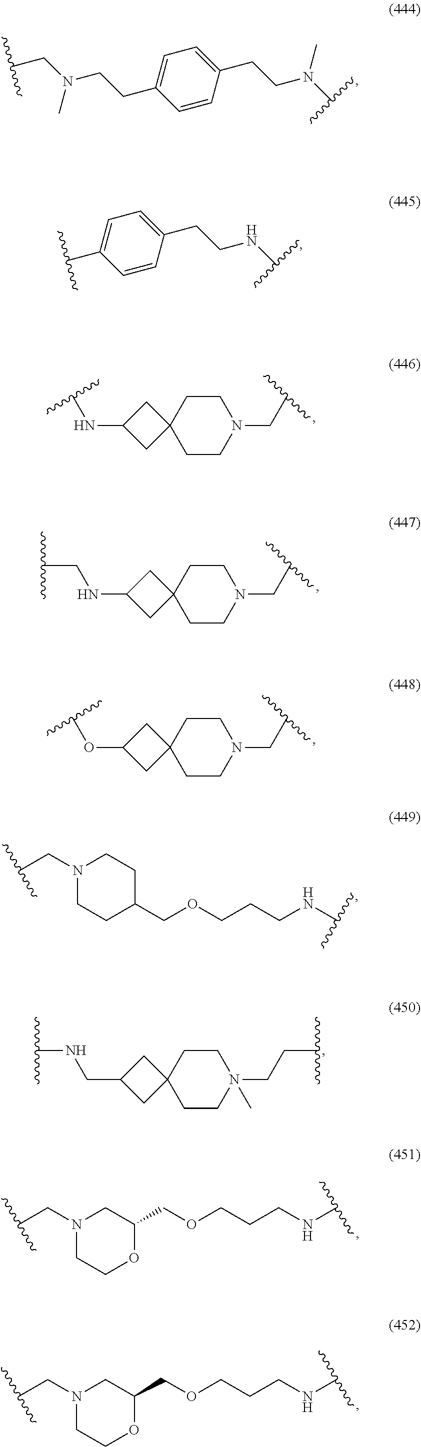

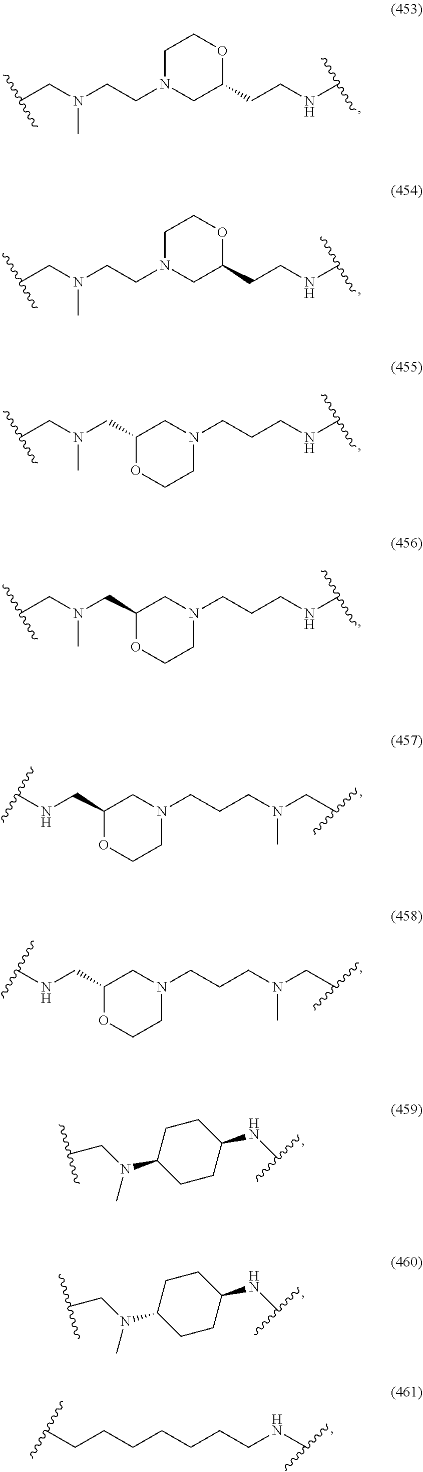

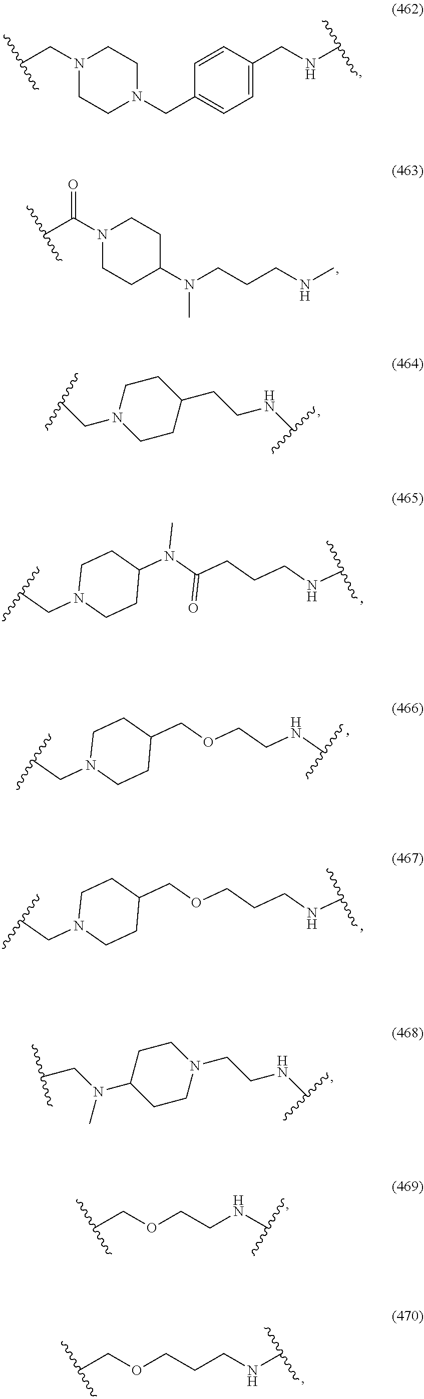

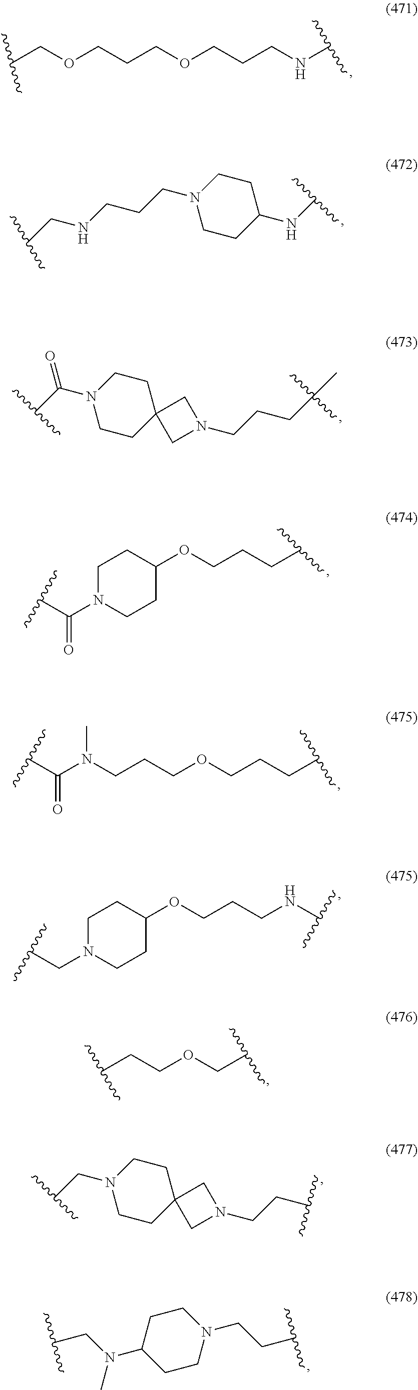

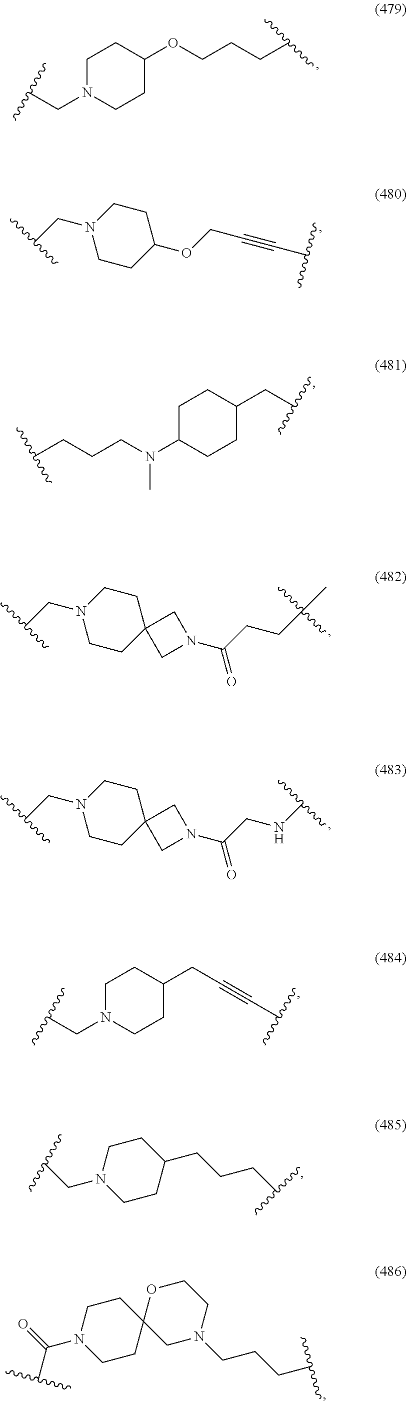

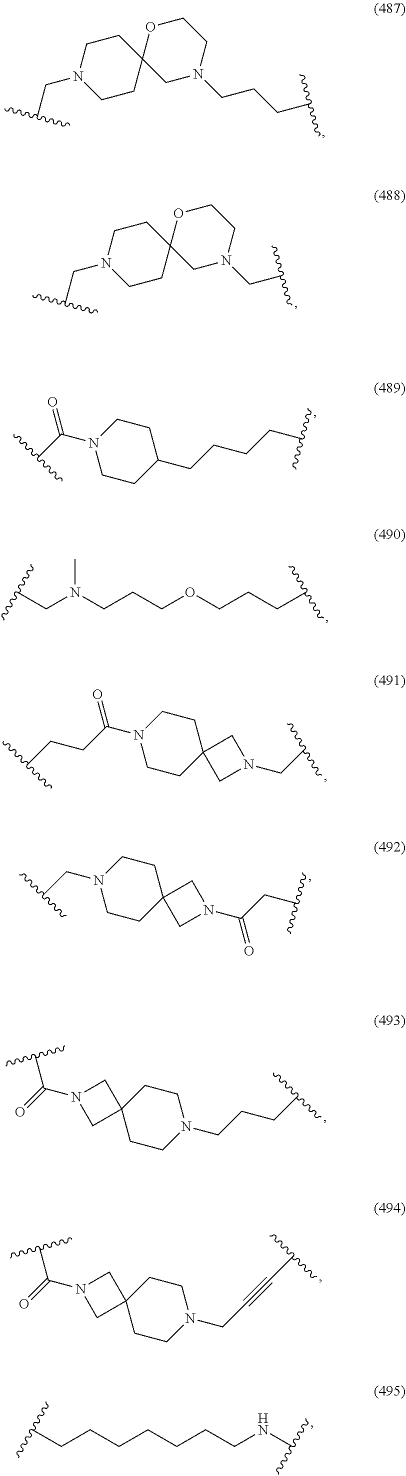

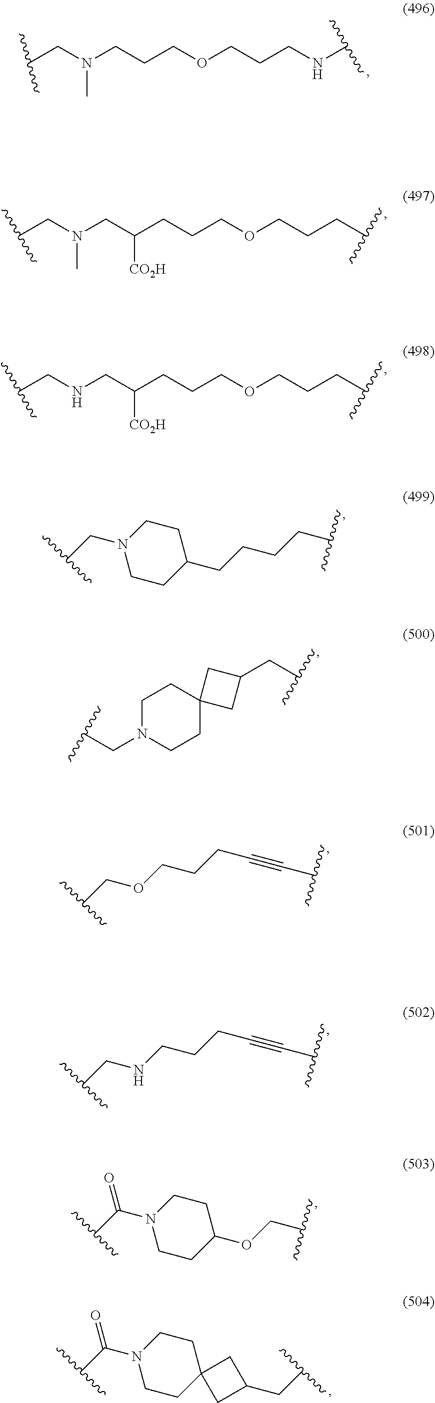

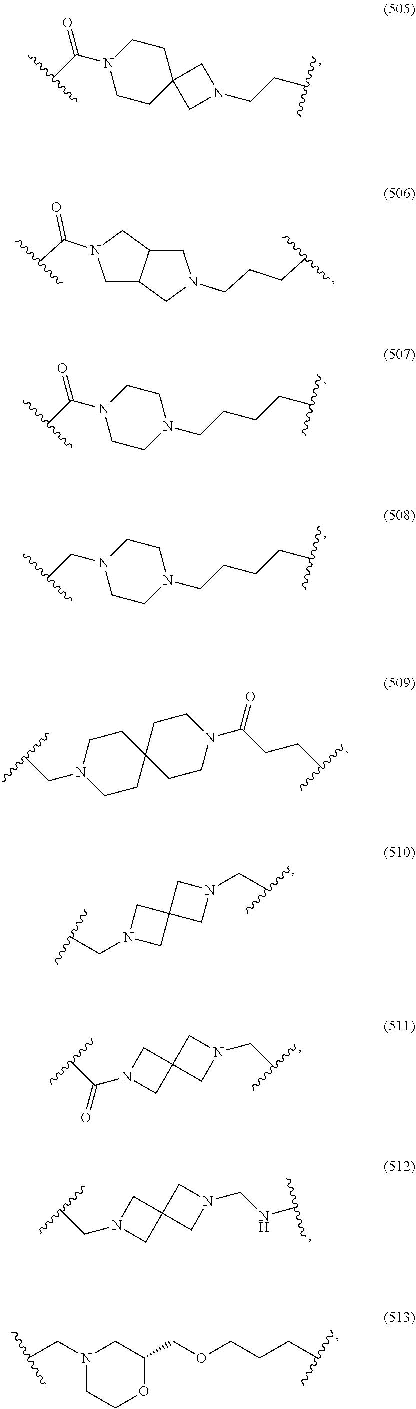

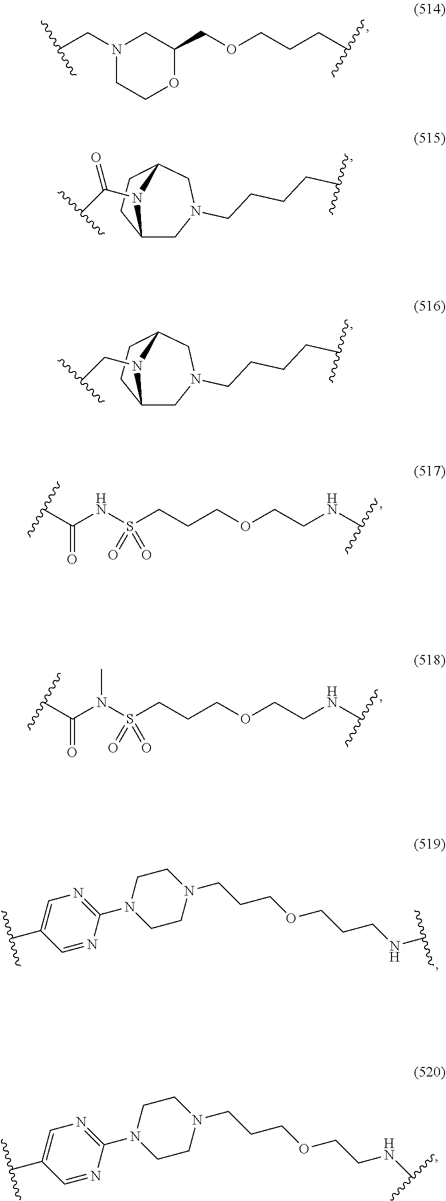

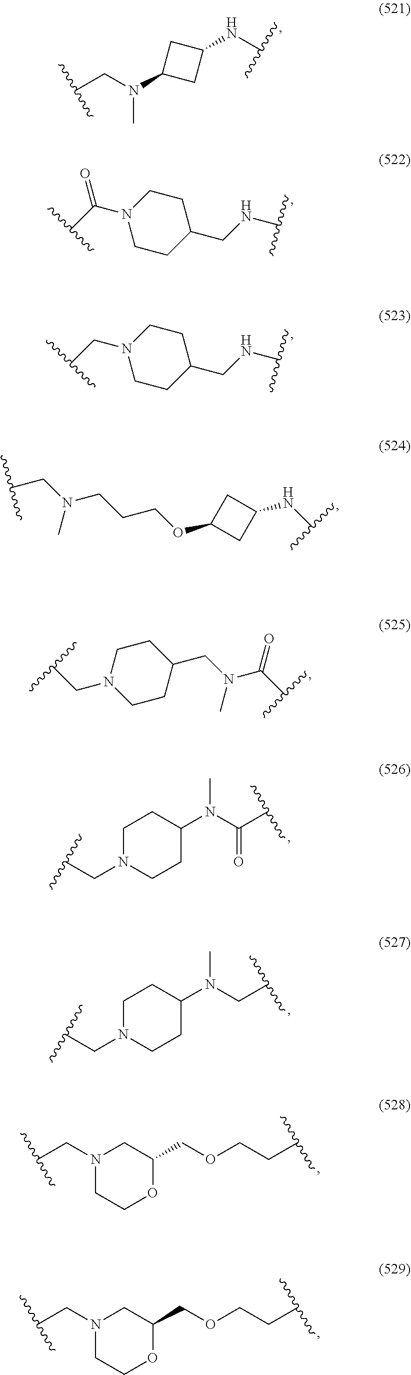

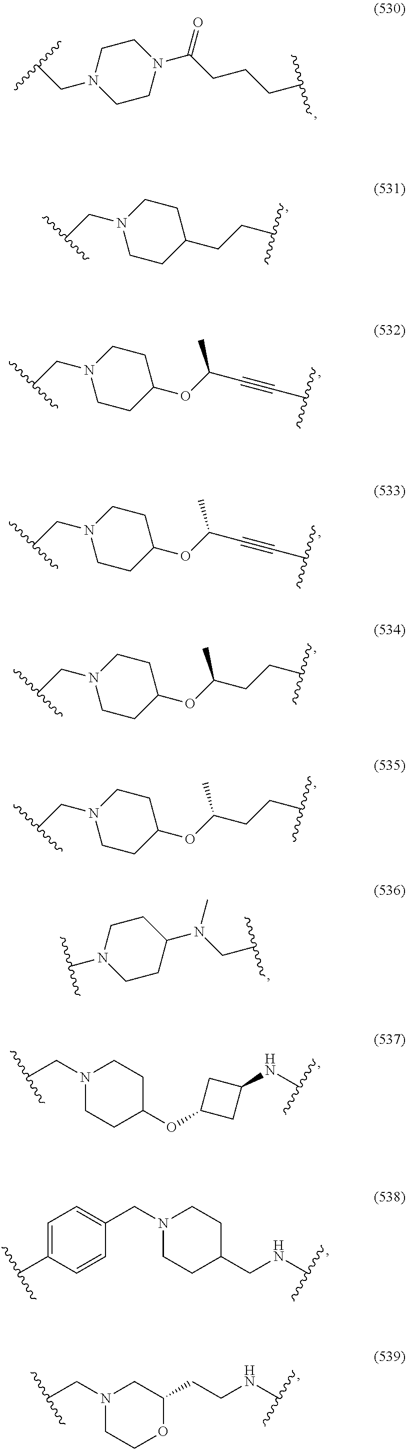

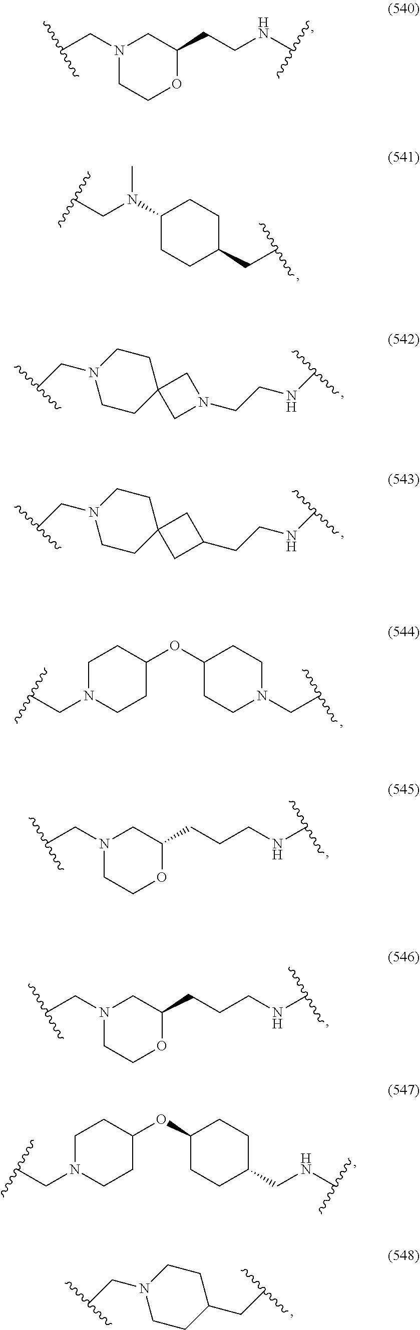

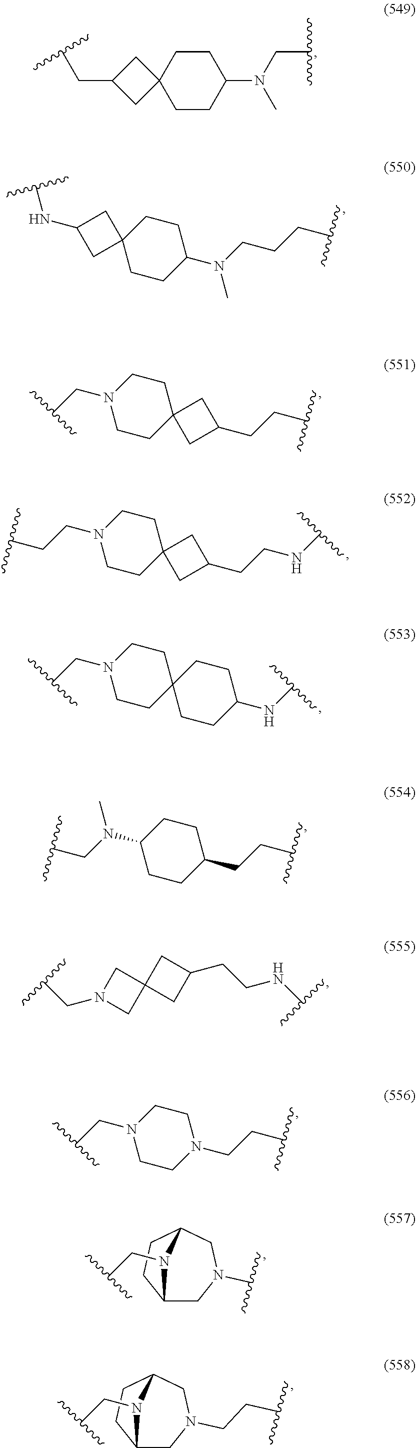

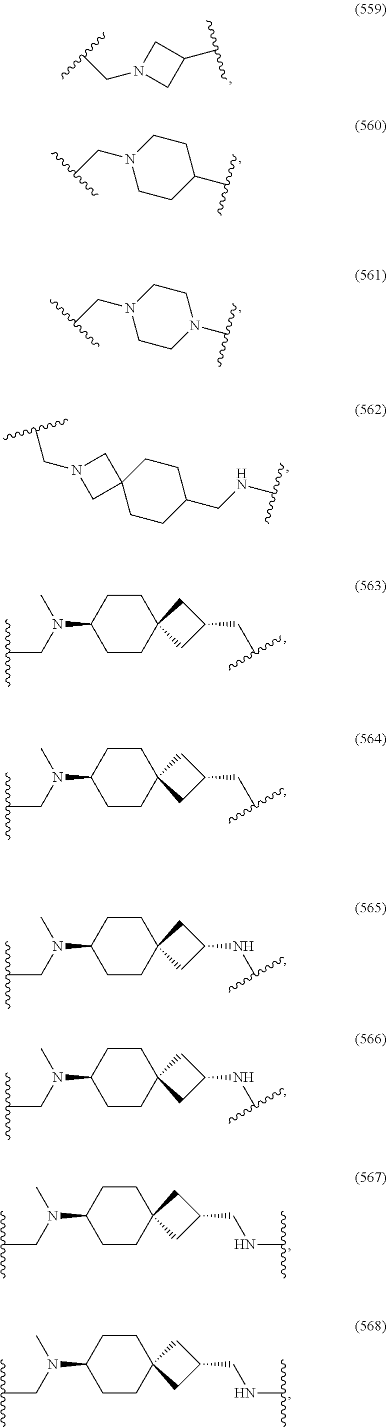

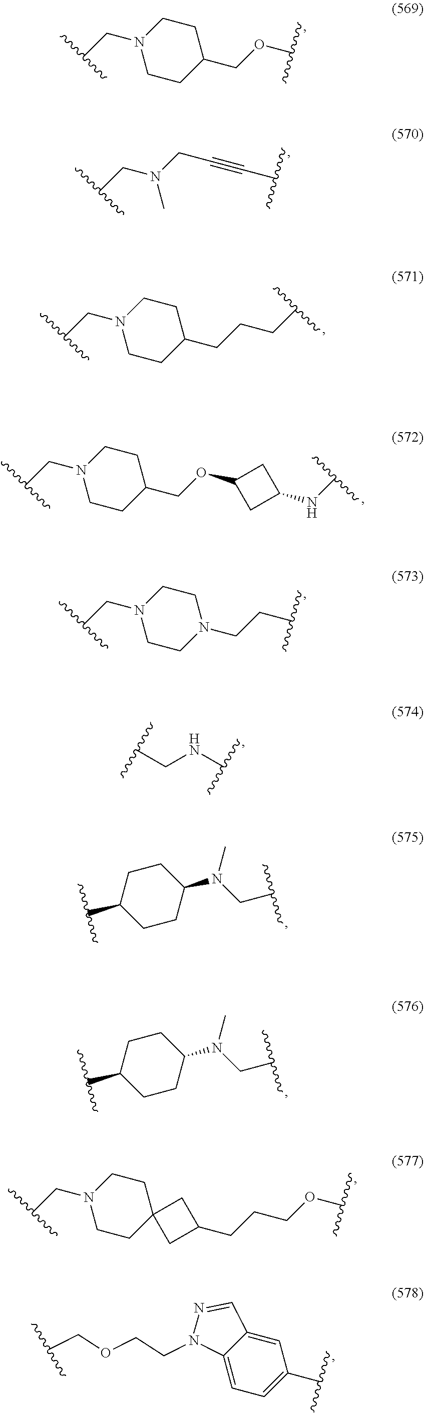

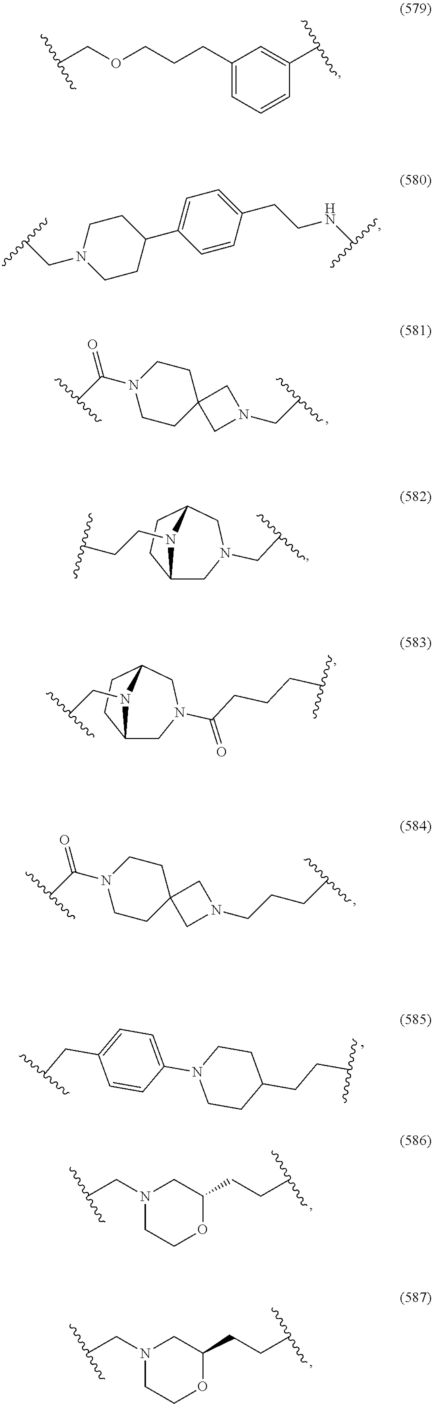

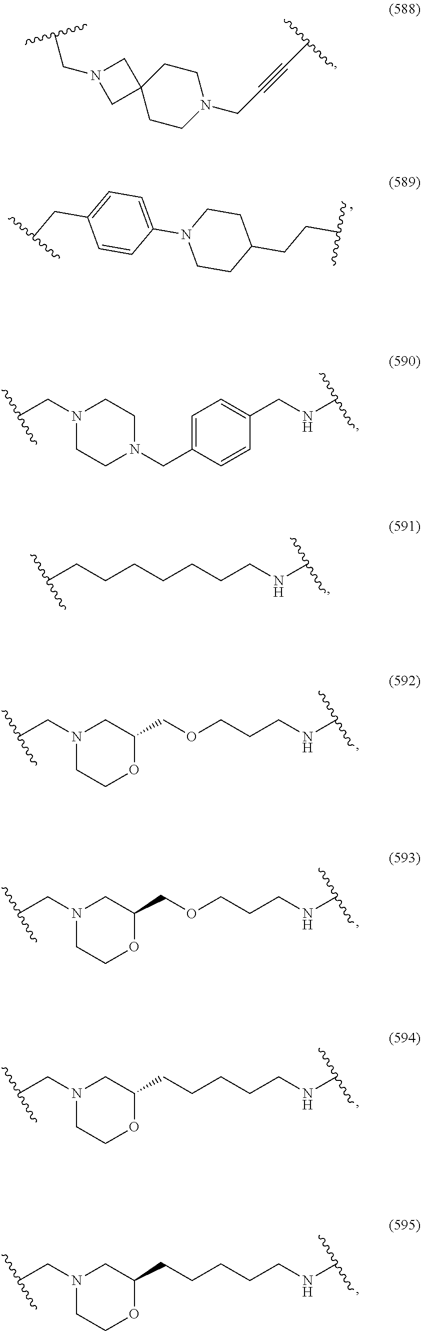

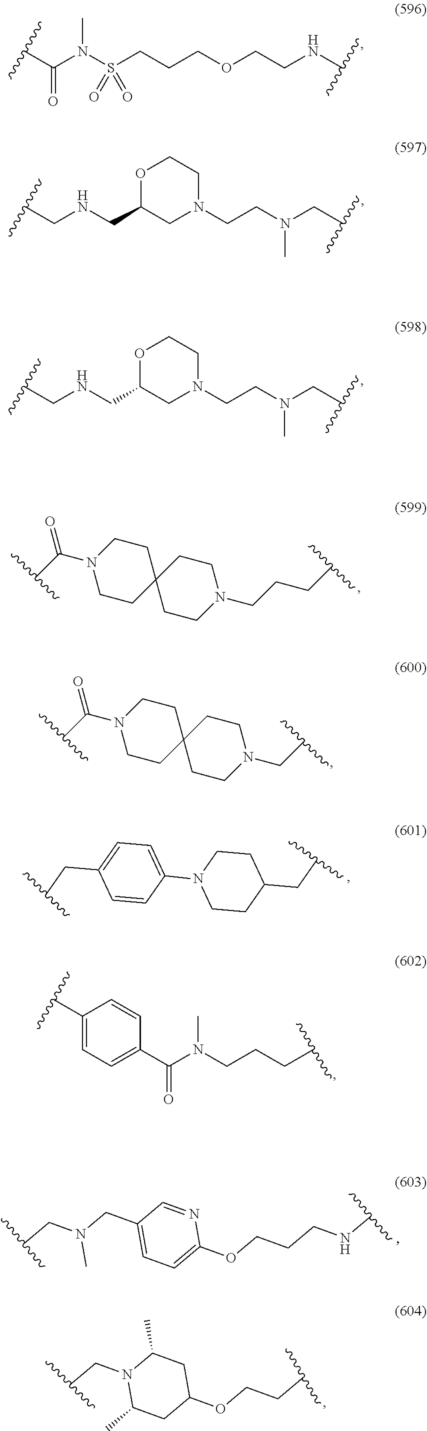

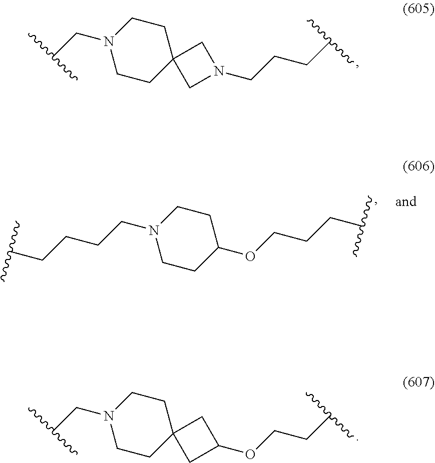 [SMILES: *CCCN1CC2(CCN(C*)CC2)C1 ; *CCCCN1CCC(OCCC*)CC1 ; *CCOC1CC2(CCN(C*)CC2)C1]

The disclosure also includes the compounds depicted in Table 1 that were prepared using the synthetic methods described in the Exemplification. The synthetic protocols used to prepare the compounds in Table 1 are summarized in Schemes 1-8 of the General Synthetic Methods and are described in more detail in the Exemplification.

TABLE 1
Exemplified Compound

| Ex. | |
|---|---|
| # | Structure |
| 1 | 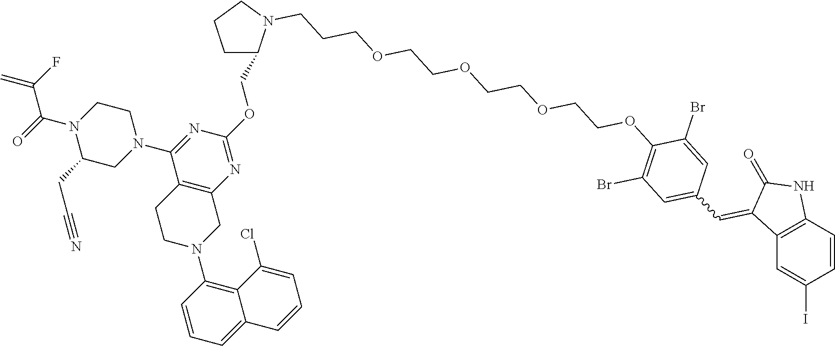 [SMILES: C=C(F)C(=O)N1CCN(c2nc(OC[C@@H]3CCCN3CCCOCCOCCOCCOc3c(Br)cc(C=C4C(=O)Nc5ccc(I)cc54)cc3Br)nc3c2CCN(c2cccc4cccc(Cl)c24)C3)C[C@@H]1CC#N] |
| | 2-((S)-4-(7-(8-chloronaphthalen-1-yl)-2-(((S)-1-(3-(2-(2-(2-(2,6-dibromo-4-((5- |
| | iodo-2-oxoindolin-3-ylidene)methyl)phenoxy)ethoxy)ethoxy)ethoxy)propyl) |
| | pyrrolidin-2-yl)methoxy)-5,6,7,8-tetrahydropyrido[3,4-d]pyrimidin-4-yl)-1-(2- |
| | fluoroacryloyl)piperazin-2-yl)acetonitrile |
| 2 | 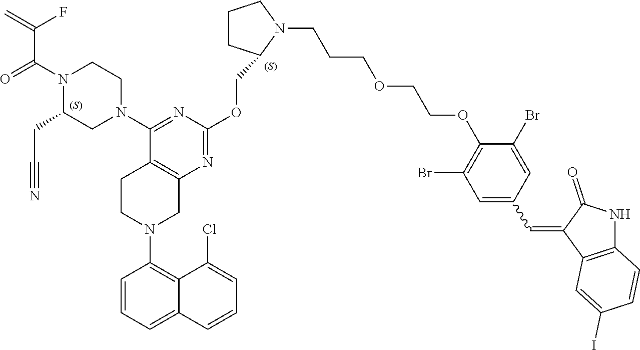 |
| | 2-((S)-4-(7-(8-chloronaphthalen-1-yl)-2-(((S)-1-(3-(2-(2,6-dibromo-4-((5-iodo-2- |
| | oxoindolin-3-ylidene)methyl)phenoxy)ethoxy)propyl)pyrrolidin-2-yl)methoxy)- |
| | 5,6,7,8-tetrahydropyrido[3,4-d]pyrimidin-4-yl)-1-(2-fluoro acryloyl)piperazin-2- |
| | yl)acetonitrile |
| 3 | 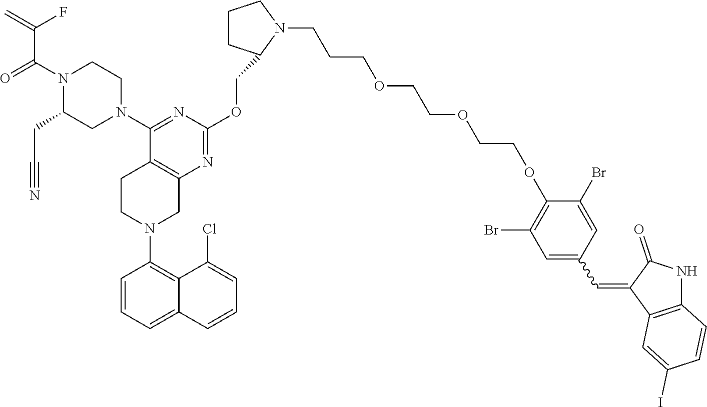 |
| | 2-((S)-4-(7-(8-chloronaphthalen-1-yl)-2-(((S)-1-(3-(2-(2-(2,6-dibromo-4-((5-iodo- |
| | 2-oxoindolin-3-ylidene)methyl)phenoxy)ethoxy)ethoxy)propyl)pyrrolidin-2- |
| | yl)methoxy)-5,6,7,8-tetrahydropyrido[3,4-d]pyrimidin-4-yl)-1-(2-fluoroacryloyl) |
| | piperazin-2-yl)acetonitrile |
| 4 | 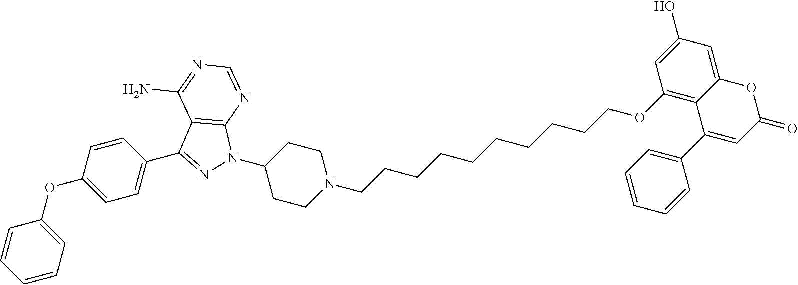 [SMILES: Nc1ncnc2c1c(-c1ccc(Oc3ccccc3)cc1)nn2C1CCN(CCCCCCCCCCOc2cc(O)cc3oc(=O)cc(-c4ccccc4)c23)CC1] |
| | 5-((10-(4-(4-Amino-3-(4-phenoxyphenyl)-1H-pyrazolo[3,4-d]pyrimidin-1- |
| | yl)piperidin-1-yl)decyl)oxy)-7-hydroxy-4-phenyl-2H-chromen-2-one |
| 5 | 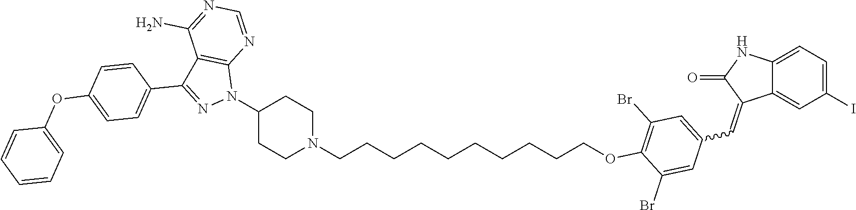 [SMILES: Nc1ncnc2c1c(-c1ccc(Oc3ccccc3)cc1)nn2C1CCN(CCCCCCCCCCOc2c(Br)cc(C=C3C(=O)Nc4ccc(I)cc43)cc2Br)CC1] |
| | 3-(4-((10-(4-(4-Amino-3-(4-phenoxyphenyl)-1H-pyrazolo[3,4-d]pyrimidin-1- |
| | yl)piperidin-1-yl)decyl)oxy)-3,5-dibromobenzylidene)-5-iodoindolin-2-one |
| 6 | 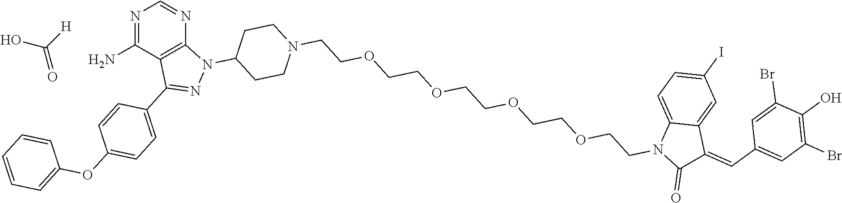 [SMILES: Nc1ncnc2c1c(-c1ccc(Oc3ccccc3)cc1)nn2C1CCN(CCOCCOCCOCCOCCN2C(=O)/C(=C/c3cc(Br)c(O)c(Br)c3)c3cc(I)ccc32)CC1 ; O=CO] |
| | 1-(14-(4-(4-amino-3-(4-phenoxyphenyl)-1H-pyrazolo[3,4-d]pyrimidin-1-yl) |
| | piperidin-1-yl)-3,6,9,12-tetraoxatetradecyl)-3-(3,5-dibromo-4-hydroxy |
| | benzylidene)-5-iodoindolin-2-one formate |
| 7 | 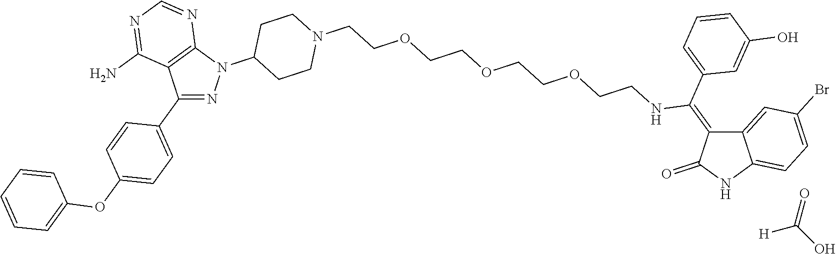 |
| | (3Z)-3-[[2-[2-[2-[2-[4-[4-amino-3-(4-phenoxyphenyl)pyrazolo[3,4-d]pyrimidin- |
| | 1-yl]-1-piperidyl]ethoxy]ethoxy]ethoxy]ethylamino]-(3-hydroxyphenyl) |
| | methylene]-5-bromo-indolin-2-one formate |
| 8 | 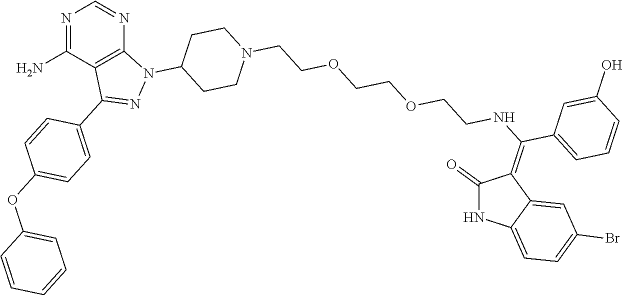 [SMILES: Nc1ncnc2c1c(-c1ccc(Oc3ccccc3)cc1)nn2C1CCN(CCOCCOCCN/C(=C2\C(=O)Nc3ccc(Br)cc32)c2cccc(O)c2)CC1] |
| | (3Z)-3-[[2-[2-[2-[4-[4-amino-3-(4-phenoxyphenyl)pyrazolo[3,4-d]pyrimidin-1- |
| | yl]-1-piperidyl]ethoxy]ethoxy]ethylamino]-(3-hydroxyphenyl)methylene]-5- |
| | bromo-indolin-2-one |

The term “bivalent ring”, as used herein, means a ring system with two points of attachment to the rest of the molecule.

The term “phenylenyl”, as used herein, refers to a phenyl group with two points of attachment to the rest of the molecule.

The term “arylenyl”, as used herein, refers to an aryl group with two points of attachment to the rest of the molecule. The term “aryl”, refers to monocyclic or bicyclic ring systems having a total of six to fourteen ring carbon atoms, wherein at least one ring in the system is aromatic and wherein each ring in the system contains 3 to 7 ring members.

The term “heteroarylenyl”, as used herein, refers to a heteroaryl group with two points of attachment to the rest of the molecule. The term “heteroaryl”, refers to monocyclic or bicyclic ring systems having a total of five to fourteen ring atoms selected from carbon atoms and one to five heteroatoms selected from nitrogen, oxygen, or sulfur, including any oxidized form of nitrogen or sulfur, and any quaternized form of a basic nitrogen, wherein at least one ring in the system is aromatic and wherein each ring in the system contains 3 to 7 ring members. One or both points of attachment could be through carbon atoms or heteroatoms. Examples of heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, pteridinyl, indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl or quinazolinyl.

The term “carbocyclylenyl”, as used herein, refers to a carbocycle group with two points of attachment to the rest of the molecule. “carbocycle” refers to a monocyclic, bicyclic, bridged bicyclic, or spirocyclic hydrocarbon that is completely saturated or that contains one or more units of unsaturation, but which is not aromatic. Examples include C3-C10 cyclkalkyl or cycloalkenyl.

The term “heterocyclylenyl” as used herein, refers to a heterocycle with two points of attachment to the rest of the molecule, wherein “heterocycle” refers to a stable 5- to 7-membered monocyclic or 7-10-membered bicyclic heterocyclic, 7-10-membered bridged bicyclic, or 7-10-membered spirocyclic moiety that is either saturated or partially unsaturated, and having, in addition to carbon atoms, one or more, preferably one to four, heteroatoms, selected from nitrogen, oxygen, or sulfur, including any oxidized form of nitrogen or sulfur, and any quaternized form of a basic nitrogen. One or both points of attachment could be through carbon atoms or heteroatoms. Examples of heterocycles include, but are not limited to morpholinyl, thiomorpholinyl, pyrrolidinonyl, pyrrolidinyl, piperidinyl, piperazinyl, hydantoinyl, valerolactamyl, oxiranyl, oxetanyl, tetrahydrofuranyl, tetrahydropyranyl, tetrahydropyrindinyl, tetrahydropyrimidinyl, tetrahydrothiophenyl, tetrahydrothiopyranyl, and the like.

In one aspect is provided a method for treating a disease associated with a cellular component (e.g., aberrant level of a cellular component), the method including contacting the cellular component with a targeted autophagy degrader (e.g., as described herein). The term “cellular component associated disease” (e.g., the cellular component may be a protein, ion, lipid, nucleic acid, nucleotide, amino acid, protein, particle, organelle, cellular compartment, microorganism, virus, vesicle, small molecule, protein complex, protein aggregate, or macromolecule) refers to a disease caused by the cellular component. Examples of a cellular component related disease include a neurodegenerative disease, cancer, a metabolic disease, autoimmune disease, inflammatory disease, or infectious disease.

In an aspect is provided a method for treating cancer, the method including administering to a subject in need thereof a therapeutically effective amount of a targeted autophagy degrader (e.g., as described herein). “Cancer” refers to all types of cancer, neoplasm or malignant tumors found in mammals (e.g., humans), including leukemia, lymphoma, carcinomas and sarcomas.

Exemplary cancers that may be treated with a compound or method provided herein include cancer of the thyroid, endocrine system, brain, breast, cervix, colon, head and neck, liver, kidney, lung, non-small cell lung, melanoma, mesothelioma, ovary, sarcoma, stomach, uterus, medulloblastoma, colorectal cancer, pancreatic cancer. Additional examples include, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

“Leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound or method provided herein include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, or undifferentiated cell leukemia.

“Lymphoma” refers to a group of cancers affecting hematopoietic and lymphoid tissues. It begins in lymphocytes, the blood cells that are found primarily in lymph nodes, spleen, thymus, and bone marrow. Two main types of lymphoma are non-Hodgkin lymphoma and Hodgkin's disease. Hodgkin's disease represents approximately 15% of all diagnosed lymphomas. This is a cancer associated with Reed-Sternberg malignant B lymphocytes. Non-Hodgkin's lymphomas (NHL) can be classified based on the rate at which cancer grows and the type of cells involved. There are aggressive (high grade) and indolent (low grade) types of NHL. Based on the type of cells involved, there are B-cell and T-cell NHLs. Exemplary B-cell lymphomas that may be treated with a compound or method provided herein include, but are not limited to, small lymphocytic lymphoma, Mantle cell lymphoma, follicular lymphoma, marginal zone lymphoma, extranodal (MALT) lymphoma, nodal (monocytoid B-cell) lymphoma, splenic lymphoma, diffuse large cell B-lymphoma, Burkitt's lymphoma, lymphoblastic lymphoma, immunoblastic large cell lymphoma, or precursor B-lymphoblastic lymphoma. Exemplary T-cell lymphomas that may be treated with a compound or method provided herein include, but are not limited to, cunateous T-cell lymphoma, peripheral T-cell lymphoma, anaplastic large cell lymphoma, mycosis fungoides, and precursor T-lymphoblastic lymphoma.

“Sarcoma” generally refers to a tumor which is made up of a substance like the embryonic connective tissue and is generally composed of closely packed cells embedded in a fibrillar or homogeneous substance. Sarcomas that may be treated with a compound or method provided herein include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

“Carcinoma” refers to a malignant new growth made up of epithelial cells tending to infiltrate the surrounding tissues and give rise to metastases. Exemplary carcinomas that may be treated with a compound or method provided herein include, for example, medullary thyroid carcinoma, familial medullary thyroid carcinoma, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma villosum.

In another aspect, there is provided a method for treating a neurodegenerative disease, the method including administering to a subject in need thereof a therapeutically effective amount of a compound described herein. In an aspect is provided a method for treating a neurodegenerative disease, the method including administering to a subject in need thereof a therapeutically effective amount of a targeted autophagy degrader (e.g., as described herein).

“Neurodegenerative disease” refers to a disease or condition in which the function of a subject's nervous system becomes impaired. Examples of neurodegenerative diseases that may be treated with a compound, pharmaceutical composition, or method described herein include Huntington Disease, Alzheimer Disease, Parkinson's Disease, Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, frontotemporal dementia, Gerstmann-Strussler-Scheinker syndrome, Huntington's disease, HIV-associated dementia, Kennedy's disease, Krabbe's disease, kuru, Lewy body dementia, Machado-Joseph disease (Spinocerebellar ataxia type 3), Multiple sclerosis, Multiple System Atrophy, Narcolepsy, Neuroborreliosis, Parkinson's disease, Pelizaeus-Merzbacher Disease, Pick's disease, Primary lateral sclerosis, Prion diseases, Refsum's disease, Sandhoffs disease, Schilder's disease, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, or Tabes dorsalis.

In an aspect is provided a method for treating a metabolic disease, the method including administering to a subject in need thereof a therapeutically effective amount of a compound described herein. In an aspect is provided a method for treating a metabolic disease, the method including administering to a subject in need thereof a therapeutically effective amount of a targeted autophagy degrader (e.g., as described herein). “Metabolic disease” or “metabolic disorder” refers to a disease or condition in which a subject's metabolism or metabolic system (e.g., function of storing or utilizing energy) becomes impaired. Examples of metabolic diseases that may be treated with a compound, pharmaceutical composition, or method described herein include diabetes (e.g., type I or type II), obesity, metabolic syndrome, or a mitochondrial disease (e.g., dysfunction of mitochondria or aberrant mitochondrial function).

In an aspect is provided a method for treating an infectious disease, the method including administering to a subject in need thereof a therapeutically effective amount of a compound described herein. In an aspect is provided a method for treating an infectious disease, the method including administering to a subject in need thereof a therapeutically effective amount of a targeted autophagy degrader (e.g., as described herein).

In an aspect is provided a method for treating an autoimmune disease, the method including administering to a subject in need thereof a therapeutically effective amount of a compound described herein. In an aspect is provided a method for treating an autoimmune disease, the method including administering to a subject in need thereof a therapeutically effective amount of a targeted autophagy degrader (e.g., as described herein). As used herein, the term “autoimmune disease” refers to a disease or condition in which a subject's immune system has an aberrant immune response against a substance that does not normally elicit an immune response in a healthy subject.

Examples of autoimmune diseases that may be treated with a compound, pharmaceutical composition, or method described herein include Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenic purpura (ATP), Autoimmune thyroid disease, Autoimmune urticaria, Axonal or neuronal neuropathies, Balo disease, Behcet's disease, Bullous pemphigoid, Cardiomyopathy, Castleman disease, Celiac disease, Chagas disease, Chronic fatigue syndrome, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenic purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenic purpura (TTP), Tolosa-Hunt syndrome, Transverse myelitis, Type 1 diabetes, Ulcerative colitis, Undifferentiated connective tissue disease (UCTD), Uveitis, Vasculitis, Vesiculobullous dermatosis, Vitiligo, or Wegener's granulomatosis (i.e., Granulomatosis with Polyangiitis (GPA).

In an aspect is provided a method for treating an inflammatory disease, the method including administering to a subject in need thereof a therapeutically effective amount of a compound described herein. In an aspect is provided a method for treating an inflammatory disease, the method including administering to a subject in need thereof a therapeutically effective amount of a targeted autophagy degrader (e.g., as described herein).

In an aspect is provided a method for treating a disease associated with a protein aggregate, the method including administering to a subject in need thereof a therapeutically effective amount of a compound described herein. In an aspect is provided a method for treating a disease associated with a protein aggregate, the method including administering to a subject in need thereof a therapeutically effective amount of a targeted autophagy degrader (e.g., as described herein). In embodiments, the disease associated with a protein aggregate is a neurodegenerative disease (e.g., Huntington's Disease, Alzheimer Disease, or Parkinson's Disease). In embodiments, the disease associated with a protein aggregate is Alzheimer's disease and the protein aggregate is an aggregate including beta amyloid. In embodiments, the disease associated with a protein aggregate is diabetes mellitus type 2 and the protein aggregate is an aggregate including IAPP. In embodiments, the disease associated with a protein aggregate is Parkinson's disease and the protein aggregate is an aggregate including alpha-synuclein. In embodiments, the disease associated with a protein aggregate is transmissible spongiform encephalopathy and the protein aggregate is an aggregate including PrP (e.g., PrP(Sc)). In embodiments, the disease associated with a protein aggregate is fatal familial insomnia and the protein aggregate is an aggregate including PrP (e.g., PrP(Sc)). In embodiments, the disease associated with a protein aggregate is Huntington's disease and the protein aggregate is an aggregate including Huntingtin. In embodiments, the disease associated with a protein aggregate is medullary carcinoma of the thyroid and the protein aggregate is an aggregate including calcitonin. In embodiments, the disease associated with a protein aggregate is cardiac arrhythmia (e.g., isolated atrial amyloidosis) and the protein aggregate is an aggregate including atrial natriuretic factor. In embodiments, the disease associated with a protein aggregate is atherosclerosis and the protein aggregate is an aggregate including apolipoprotein A1. In embodiments, the disease associated with a protein aggregate is rheumatoid arthritis and the protein aggregate is an aggregate including serum amyloid A. In embodiments, the disease associated with a protein aggregate is aortic medial amyloid and the protein aggregate is an aggregate including medin. In embodiments, the disease associated with a protein aggregate is prolactinomas and the protein aggregate is an aggregate including prolactin. In embodiments, the disease associated with a protein aggregate is familial amyloid polyneuropathy and the protein aggregate is an aggregate including transthyretin. In embodiments, the disease associated with a protein aggregate is hereditary non-neuropathic systemic amyloidosis and the protein aggregate is an aggregate including lysozyme. In embodiments, the disease associated with a protein aggregate is dialysis related amyloidosis and the protein aggregate is an aggregate including beta-2 microglobulin. In embodiments, the disease associated with a protein aggregate is Finnish amyloidosis and the protein aggregate is an aggregate including gelsolin. In embodiments, the disease associated with a protein aggregate is lattice corneal dystrophy and the protein aggregate is an aggregate including keratoepithelin. In embodiments, the disease associated with a protein aggregate is cerebral amyloid angiopathy and the protein aggregate is an aggregate including beta amyloid. In embodiments, the disease associated with a protein aggregate is cerebral amyloid angiopathy (Icelandic type) and the protein aggregate is an aggregate including cystatin. In embodiments, the disease associated with a protein aggregate is systemic AL amyloidosis and the protein aggregate is an aggregate including immunoglobulin light chain AL. In embodiments, the disease associated with a protein aggregate is sporadic inclusion body myositis and the protein aggregate is an aggregate including S-IBM. In embodiments, the disease associated with a protein aggregate is a tauopathy and the protein aggregate is an aggregate including tau protein. In embodiments, the tauopathy is primary age-related tauopathy, CTE, progressive supranuclear palsy, corticobasal degeneration, frontotemporal demential and parkinsonism linked to chromosome 17, Lytico-Bodig disease, ganglioglioma, gangliocytoma, meningioangiomatosis, postencephalitic parkinsonism, subacute sclerosing panencephalitis, lead encephalopathy, tuberous sclerosis, pantothenate kinase-associated neurodegeneration, lipofuscinosis, or Pick's disease. In embodiments, the disease associated with a protein aggregate is amyloidosis. In embodiments, the disease associated with a protein aggregate is a proteinopathy. In embodiments, the disease associated with a protein aggregate is amyotrophic lateral sclerosis and the protein aggregate is an aggregate including superoxide dismutase, TDP043, FUS, C90RF72, and/or ubiquilin-2 (UBQLN2). In embodiments, the disease associated with a protein aggregate is a trinucleotide repeat disorder.

In embodiments, the disease associated with a protein aggregate is a synucleinopathy. In embodiments, the disease associated with a protein aggregate is prion disease and the protein aggregate is an aggregate including prion protein. In embodiments, the method includes reducing the protein aggregate (e.g., reducing aggregate size, number of aggregates, or occurrence of aggregates).

In an aspect is provided a method for treating a polyglutamine disease or polyQ disease. “Polyglutamine diseases” or “polyQ diseases” refers to a group of neurodegenerative diseases caused by expanded cytosine-adenine-guanine (CAG) repeats encoding a long polyQ tract in the respective proteins. The protein including the polyQ tract may form a protein aggregate (“polyQ protein aggregate”). In Huntington's disease, the huntingtin protein may include a polyQ tract and may form a protein aggregate or “polyQ huntingtin aggregate”.

Included in the present teachings are pharmaceutically acceptable salts of the bifunctional targeted protein autophagy degraders disclosed herein. Bifunctional targeted protein autophagy degraders of the present teachings with basic groups can form pharmaceutically acceptable salts with pharmaceutically acceptable acid(s). Suitable pharmaceutically acceptable acid addition salts of the compounds described herein include salts of inorganic acids (such as hydrochloric acid, hydrobromic, phosphoric, nitric, and sulfuric acids) and of organic acids (such as acetic acid, benzenesulfonic, benzoic, methanesulfonic, and p-toluenesulfonic acids). Bifunctional targeted protein autophagy degraders of the present teachings with acidic groups can form pharmaceutically acceptable salts with pharmaceutically acceptable base(s). Suitable pharmaceutically acceptable basic salts include ammonium salts, alkali metal salts (such as sodium and potassium salts) and alkaline earth metal salts (such as magnesium and calcium salts).

As used herein, the term “pharmaceutically acceptable salt” refers to pharmaceutical salts that are, within the scope of sound medical judgment, suitable for use in contact with the tissues of humans and lower animals without undue toxicity, irritation, and allergic response, and are commensurate with a reasonable benefit/risk ratio. Pharmaceutically-acceptable salts are well known in the art. For example, S. M. Berge et al. describes pharmacologically acceptable salts in J. Pharm. Sci. (1977) 66:1-19.

The terms “administer”, “administering”, “administration”, and the like, as used herein, refer to methods that may be used to enable delivery of compositions to the desired site of biological action. These methods include, but are not limited to, intraarticular (in the joints), intravenous, intramuscular, intratumoral, intradermal, intraperitoneal, subcutaneous, orally, topically, intrathecally, inhalationally, transdermally, rectally, and the like. Administration techniques that can be employed with the agents and methods described herein are found in e.g., Goodman and Gilman, The Pharmacological Basis of Therapeutics, current ed.; Pergamon; and Remington's, Pharmaceutical Sciences (current edition), Mack Publishing Co., Easton, Pa.

A “subject” is a mammal, preferably a human, but can also be an animal in need of veterinary treatment, e.g., companion animals (e.g., dogs, cats, and the like), farm animals (e.g., cows, sheep, pigs, horses, and the like) and laboratory animals (e.g., rats, mice, guinea pigs, and the like).

The precise amount of bifunctional targeted protein autophagy degraders administered to provide an “effective amount” to the subject will depend on the mode of administration, the type, and severity of the disease or condition, and on the characteristics of the subject, such as general health, age, sex, body weight, and tolerance to drugs. The skilled artisan will be able to determine appropriate dosages depending on these and other factors. When administered in combination with other therapeutic agents, an “effective amount” of any additional therapeutic agent(s) will depend on the type of drug used. Suitable dosages are known for approved therapeutic agents and can be adjusted by the skilled artisan according to the condition of the subject, the type of condition(s) being treated and the amount of a compound of the invention being used by following, for example, dosages reported in the literature and recommended in the Physician's Desk Reference (57th ed., 2003).

The term “effective amount” means an amount when administered to the subject which results in beneficial or desired results, including clinical results, e.g., inhibits, suppresses or reduces the symptoms of the condition being treated in the subject as compared to a control. For example, a therapeutically effective amount can be given in unit dosage form (e.g., 0.1 mg to about 50 g per day, alternatively from 1 mg to about 5 grams per day; and in another alternatively from 10 mg to 1 gram per day).

The particular mode of administration and the dosage regimen will be selected by the attending clinician, taking into account the particulars of the case (e.g. the subject, the disease, the disease state involved, the particular treatment, and whether the treatment is prophylactic). Treatment can involve daily or multi-daily or less than daily (such as weekly or monthly etc.) doses over a period of a few days to months, or even years. However, a person of ordinary skill in the art would immediately recognize appropriate and/or equivalent doses looking at dosages of approved compositions for treating a mitochondria-related disease using the disclosed compounds for guidance.

The pharmaceutical composition of the invention is formulated to be compatible with its intended route of administration. In an embodiment, the composition is formulated in accordance with routine procedures as a pharmaceutical composition adapted for intravenous, subcutaneous, intramuscular, oral, intranasal, or topical administration to human beings. In preferred embodiments, the pharmaceutical composition is formulated for intravenous administration.

“Pharmaceutically acceptable excipient” and “pharmaceutically acceptable carrier” refer to a substance that aids the formulation and/or administration of an active agent to and/or absorption by a subject and can be included in the compositions of the present disclosure without causing a significant adverse toxicological effect on the subject. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, boloring, and/or aromatic substances and the like that do not deleteriously react with or interfere with the activity of the compounds provided herein. One of ordinary skill in the art will recognize that other pharmaceutical excipients are suitable for use with disclosed Bifunctional targeted protein autophagy degraders.

### General Synthetic Methods

The compounds of this invention may be prepared or isolated in general by synthetic and/or semi-synthetic methods known to those skilled in the art for analogous compounds and by methods described in detail in the Examples, herein.

In the Schemes below, where a particular protecting group, leaving group, or transformation condition is depicted, one of ordinary skill in the art will appreciate that other protecting groups, leaving groups, and transformation conditions are also suitable and are contemplated. Such groups and transformations are described in detail in March's Advanced Organic Chemistry: Reactions, Mechanisms, and Structure, M. B. Smith and J. March, 5^{th} Edition, John Wiley & Sons, 2001, Comprehensive Organic Transformations, R. C. Larock, 2^{nd} Edition, John Wiley & Sons, 1999, and Protecting Groups in Organic Synthesis, T. W. Greene and P. G. M. Wuts, 3^{rd} edition, John Wiley & Sons, 1999, the entirety of each of which is hereby incorporated herein by reference.

As used herein, the phrase “oxygen protecting group” includes, for example, carbonyl protecting groups, hydroxyl protecting groups, etc. Hydroxyl protecting groups are well known in the art and include those described in detail in Protecting Groups in Organic Synthesis, T. W. Greene and P. G. M. Wuts, 3^{rd} edition, John Wiley & Sons, 1999, the entirety of each of which is herein incorporated by reference. Examples of suitable hydroxyl protecting groups include, but are not limited to, esters, allyl ethers, ethers, silyl ethers, alkyl ethers, arylalkyl ethers, and alkoxyalkyl ethers. Examples of such esters include formates, acetates, carbonates, and sulfonates. Specific examples include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetyl), crotonate, 4-methoxy-crotonate, benzoate, p-benylbenzoate, 2,4,6-trimethylbenzoate, carbonates such as methyl, 9-fluorenylmethyl, ethyl, 2,2,2-trichloroethyl, 2-(trimethylsilyl)ethyl, 2-(phenyl sulfonyl)ethyl, vinyl, allyl, and p-nitrobenzyl. Examples of such silyl ethers include trimethylsilyl, triethylsilyl, t-butyldimethylsilyl, t-butyldiphenylsilyl, triisopropylsilyl, and other trialkylsilyl ethers. Alkyl ethers include methyl, benzyl, p-methoxybenzyl, 3,4-dimethoxybenzyl, trityl, t-butyl, allyl, and allyloxycarbonyl ethers or derivatives. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyranyl ethers. Examples of arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, and 2- and 4-picolyl.

Amino protecting groups are well known in the art and include those described in detail in Protecting Groups in Organic Synthesis, T. W. Greene and P. G. M. Wuts, 3^{rd} edition, John Wiley & Sons, 1999, the entirety of each of which is herein incorporated by reference. Suitable amino protecting groups include, but are not limited to, aralkylamines, carbamates, cyclic imides, allyl amines, amides, and the like. Examples of such groups include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, phthalimide, benzyl (Bn), fluorenylmethylcarbonyl (Fmoc), formyl, acetyl, chloroacetyl, dichloroacetyl, trichloroacetyl, phenylacetyl, trifluoroacetyl, benzoyl, and the like.

In the schemes below, where a provided compound is formed having a reactive DIM moiety (e.g., amine, alcohol, etc.), it is not shown but it is generally appreciated and well known by those having ordinary skill in the art that the reactivity of said reactive DIM moiety may be masked by employing a suitable protecting group that can thereafter be removed in situ or during a separate synthetic step.

In certain embodiments, compounds of the present invention are generally prepared according to Scheme 1 set forth below.

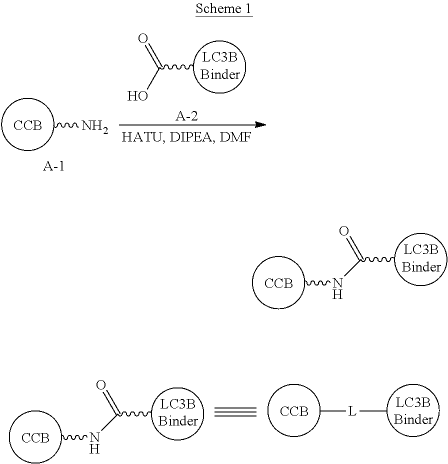

As depicted in Scheme 1, above, amine A-1 is coupled to acid A-2 using the coupling agent HATU in the presence of the base DIPEA in DMF to form a compound of the invention with a linker comprising an amide bond. The squiggly bond, [symbol] , represents the portion of the linker between cellular component binder (CCB) and the terminal amino group of A-1 or the portion of the linker between the LC3B binder and the terminal carboxyl group of A-2, respectively. Additionally, an amide bond can be formed using coupling reagents known in the art such as, but not limited to DCC, DIC, EDC, HBTU, HCTU, PyAOP, PyBrOP, BOP, BOP-Cl, DEPBT, T3P, TATU, TBTU, TNTU, TOTU, TPTU, TSTU, or TDBTU.

In certain embodiments, compounds of the present invention are generally prepared according to Scheme 2 set forth below.

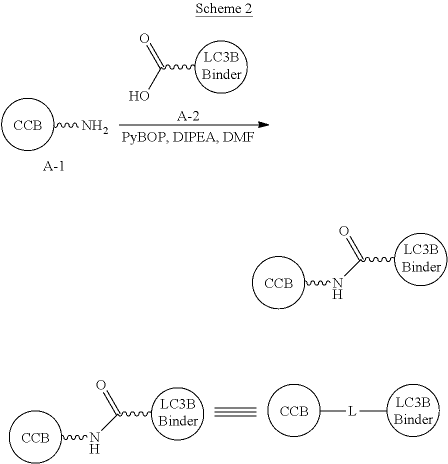

As depicted in Scheme 2, above, amine A-1 is coupled to acid A-2 using the coupling agent PyBOP in the presence of the base DIPEA in DMF to form a compound of the invention with a linker comprising an amide bond. The squiggly bond, [symbol] , represents the portion of the linker between the cellular component binder (CCB) and the terminal amino group of A-1 or the portion of the linker between the LC3B binder and the terminal carboxyl group of A-2, respectively. Additionally, an amide bond can be formed using coupling reagents known in the art such as, but not limited to DCC, DIC, EDC, HBTU, HCTU, PyAOP, PyBrOP, BOP, BOP-Cl, DEPBT, T3P, TATU, TBTU, TNTU, TOTU, TPTU, TSTU, or TDBTU.

In certain embodiments, compounds of the present invention are generally prepared according to Scheme 3 set forth below.

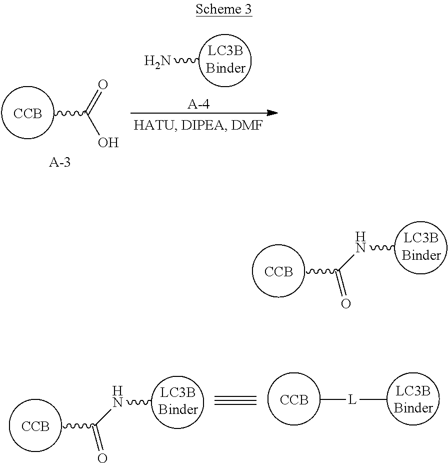

As depicted in Scheme 3, above, acid A-3 is coupled to amine A-4 using the coupling agent HATU in the presence of the base DIPEA in DMF to form a compound of the invention with a linker comprising an amide bond. The squiggly bond, [symbol] , represents the portion of the linker between cellular component binder (CCB) and the terminal carboxyl group of A-3 or the portion of the linker between the LC3B binder and the terminal amino group of A-4, respectively. Additionally, an amide bond can be formed using coupling reagents known in the art such as, but not limited to DCC, DIC, EDC, HBTU, HCTU, PyAOP, PyBrOP, BOP, BOP-Cl, DEPBT, T3P, TATU, TBTU, TNTU, TOTU, TPTU, TSTU, or TDBTU.

In certain embodiments, compounds of the present invention are generally prepared according to Scheme 4 set forth below.

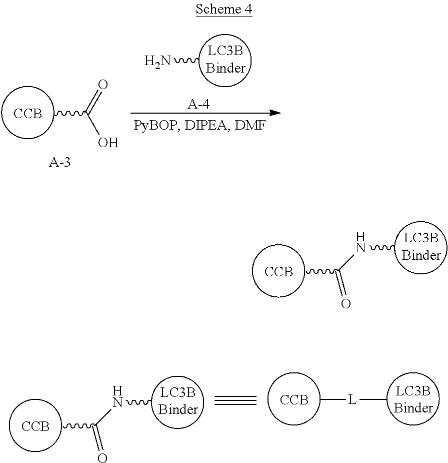

As depicted in Scheme 4, above, acid A-3 is coupled to amine A-4 using the coupling agent PyBOP in the presence of the base DIPEA in DMF to form a compound of the invention with a linker comprising an amide bond. The squiggly bond, [symbol] , represents the portion of the linker between cellular component binder (CCB) and the terminal carboxyl group of A-3 or the portion of the linker between the LC3B binder and the terminal amino group of A-4, respectively. Additionally, an amide bond can be formed using coupling reagents known in the art such as, but not limited to DCC, DIC, EDC, HBTU, HCTU, PyAOP, PyBrOP, BOP, BOP-Cl, DEPBT, T3P, TATU, TBTU, TNTU, TOTU, TPTU, TSTU, or TDBTU.

In certain embodiments, compounds of the present invention are generally prepared according to Scheme 5 set forth below.

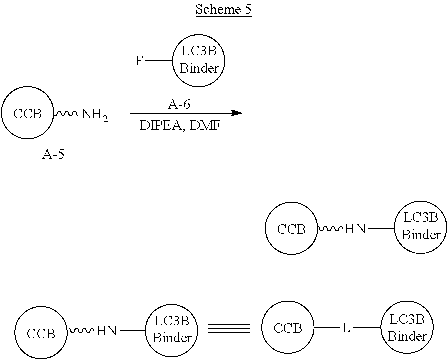

As depicted in Scheme 5, above, an S_{N}Ar displacement of fluoride A-6 by amine A-5 is effected in the presence of the base DIPEA in DMF to form a compound of the invention with a linker comprising a secondary amine. The squiggly bond, [symbol] , represents the portion of the linker between the cellular component binder (CCB) and the terminal amino group of A-5.

In certain embodiments, compounds of the present invention are generally prepared according to Scheme 6 set forth below:

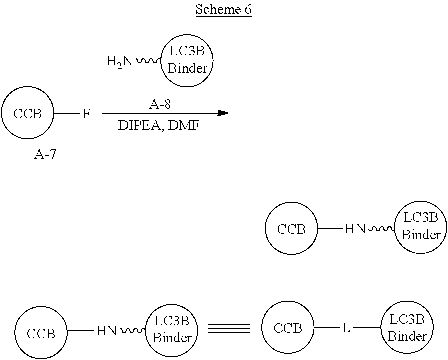

As depicted in Scheme 6, above, an SNAr displacement of fluoride A-7 by amine A-8 is effected in the presence of the base DIPEA in DMF to form a compound of the invention with a linker comprising a secondary amine. The squiggly bond, [symbol] , represents the portion of the linker between the LC3B binder and the terminal amino group of A-8.

In certain embodiments, compounds of the present invention are generally prepared according to Scheme 7 set forth below.

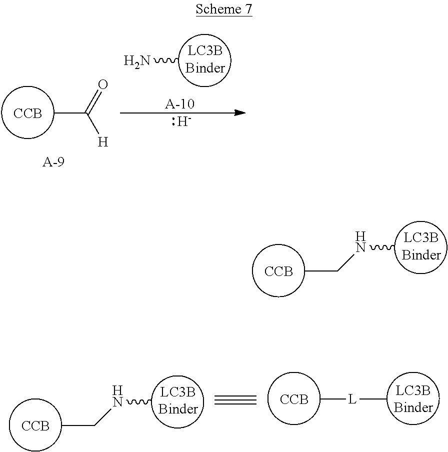

As depicted in Scheme 7, above, reductive alkylation of aldehyde A-9 by amine A-10 is effected in the presence of a mild hydride source (e.g., sodium cyanoborohydride or sodium triacetoxyborohydride) to form a provided compound with a linker comprising a secondary amine.

The squiggly bond, [symbol] , represents the portion of the linker between DIM and the terminal amino group of A-10.

In certain embodiments, compounds of the present invention are generally prepared according to Scheme 8 set forth below.

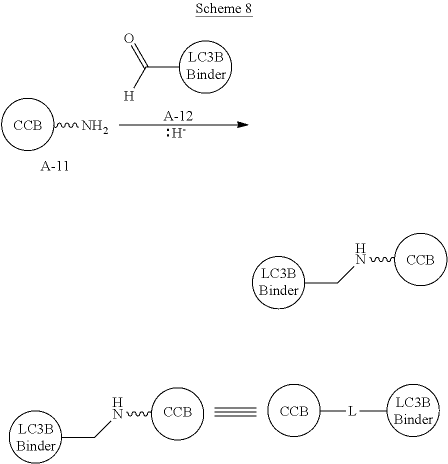

As depicted in Scheme 8, above, reductive alkylation of aldehyde A-12 by amine A-11 is effected in the presence of a mild hydride source (e.g., sodium cyanoborohydride or sodium triacetoxyborohydride) to form a provided compound with a linker comprising a secondary amine. The squiggly bond, [symbol] , represents the portion of the linker between cellular component binder (CCB) and the terminal amino group of A-11.

One of skill in the art will appreciate that various functional groups present in compounds of the invention such as aliphatic groups, alcohols, carboxylic acids, esters, amides, aldehydes, halogens and nitriles can be interconverted by techniques well known in the art including, but not limited to reduction, oxidation, esterification, hydrolysis, partial oxidation, partial reduction, halogenation, dehydration, partial hydration, and hydration. See for example, “March's Advanced Organic Chemistry”, 5^{th} Ed., Ed.: Smith, M. B. and March, J., John Wiley & Sons, New York: 2001, the entirety of each of which is herein incorporated by reference. Such interconversions may require one or more of the aforementioned techniques, and certain methods for synthesizing compounds of the invention are described below in the Exemplification.

### EXEMPLIFICATION

### A. Synthesis of Intermediate A

### Intermediate A: Tert-butyl (S)-4-(7-(8-chloronaphthalen-1-yl)-2-(((S)-pyrrolidin-2-yl)methoxy)-5,6,7,8-tetrahydropyrido[3,4-d]pyrimidin-4-yl)-2-(cyanomethyl) piperazine-1-carboxylate (Intermediate A)

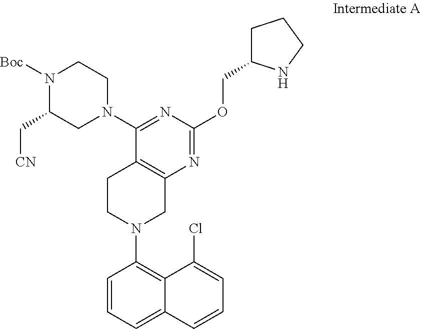 [SMILES: CC(C)(C)OC(=O)N1CCN(c2nc(OC[C@@H]3CCCN3)nc3c2CCN(c2cccc4cccc(Cl)c24)C3)C[C@@H]1CC#N]

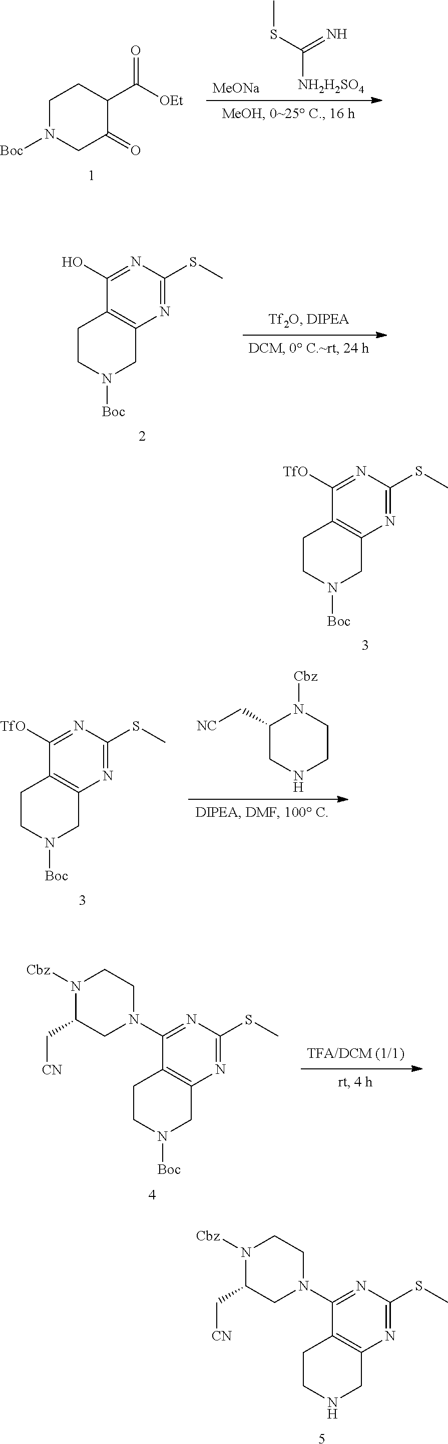

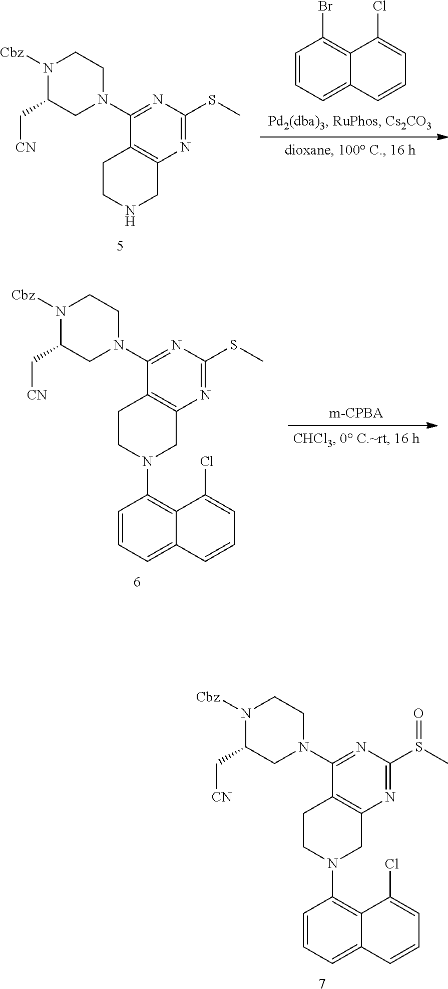

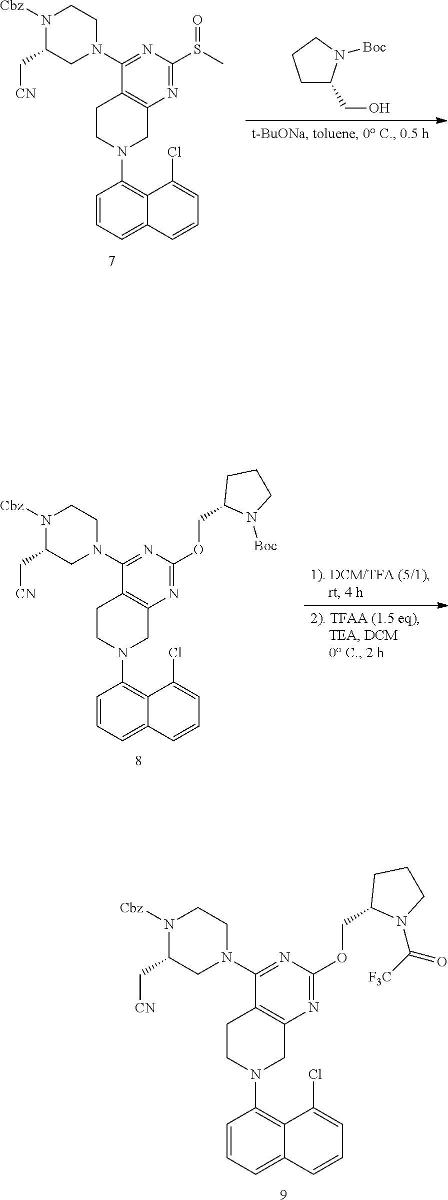

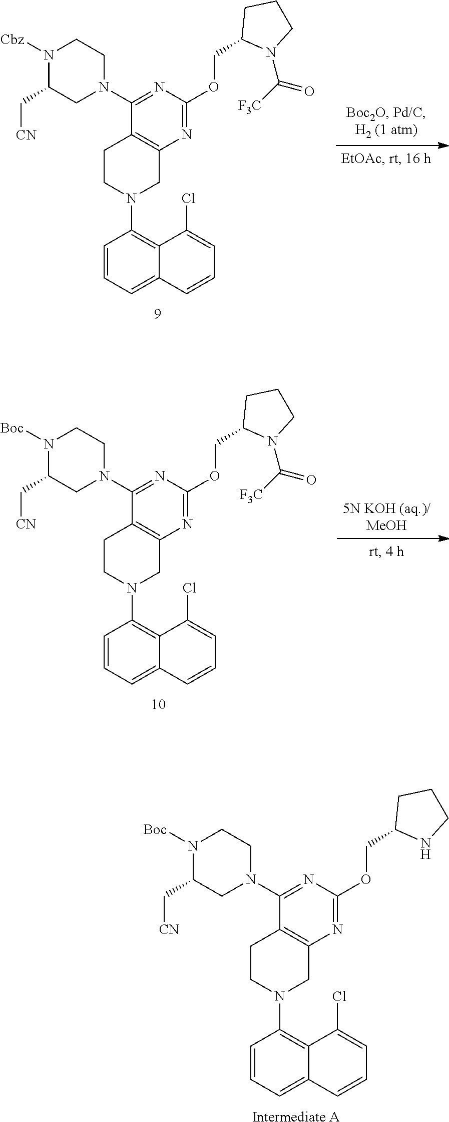

The compounds 2 to 7 below were synthesized according to the patent US 2019/0144444A1.

### Step 6: Benzyl (S)-4-(2-(((S)-1-(tert-butoxycarbonyl)pyrrolidin-2-yl)methoxy)-7-(8-chloronaphthalen-1-yl)-5,6,7,8-tetrahydropyrido[3,4-d]pyrimidin-4-yl)-2-(cyanomethyl) piperazine-1-carboxylate

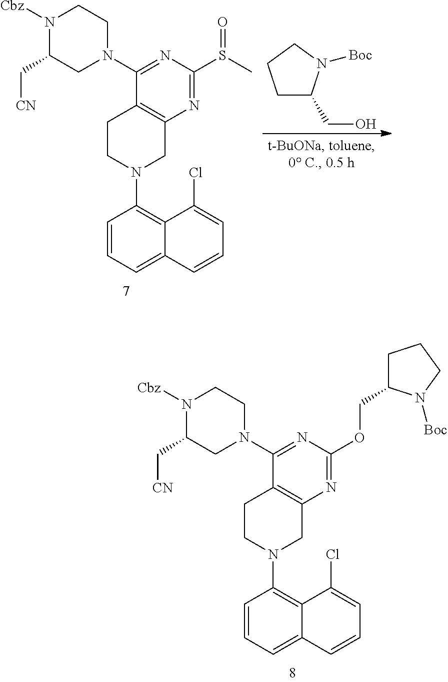

To a stirred solution of benzyl (2S)-4-(7-(8-chloronaphthalen-1-yl)-2-methanesulfinyl-5H,6H,8H-pyrido[3,4-d]pyrimidin-4-yl)-2-(cyanomethyl)piperazine-1-carboxylate (5.00 g, 8.13 mmol) in toluene (60 mL) were added tert-butyl (2S)-2-(hydroxymethyl)pyrrolidine-1-carboxylate (4.90 g, 24.38 mmol) and tert-butoxysodium (1.50 g, 16.26 mmol) at 0° C. under a nitrogen atmosphere. After stirring for additional 30 min, the resulting mixture was neutralized to pH 7 with 1 M HCl (aq.). The resulting mixture was extracted with ethyl acetate (3×100 mL). The combined organic layers were washed with brine (3×100 mL) and dried over anhydrous Na₂SO₄. After filtration, the filtrate was concentrated under reduced pressure to afford benzyl (S)-4-(2-(((S)-1-(tert-butoxycarbonyl)pyrrolidin-2-yl)methoxy)-7-(8-chloronaphthalen-1-yl)-5,6,7,8-tetrahydropyrido[3,4-d]pyrimidin-4-yl)-2-(cyanomethyl)piperazine-1-carboxylate product (2.20 g, 36%) as a yellow solid: ¹H NMR (400 MHz, DMSO-d₆) δ 7.76 (dd, J=8.1, 3.6 Hz, 2H), 7.61-7.51 (m, 4H), 7.46 (t, J=7.8 Hz, 2H), 7.40-7.30 (m, 3H), 4.48 (dt, J=11.9, 4.1 Hz, 2H), 4.38-4.29 (m, 2H), 4.24-4.15 (m, 2H), 4.01 (s, 2H), 3.89 (d, J=9.5 Hz, 6H), 2.11 (dt, J=12.5, 6.5 Hz, 2H), 1.98-1.87 (m, 4H), 1.71 (td, J=14.3, 12.8, 7.7 Hz, 2H), 1.45 (s, 13H); LC/MS (ESI, m/z): [(M+1)]⁺=752.3.

### Step 7: Benzyl (S)-4-(7-(8-chloronaphthalen-1-yl)-2-(((S)-1-(2,2,2-trifluoroacetyl) pyrrolidin-2-yl)methoxy)-5,6,7,8-tetrahydropyrido[3,4-d]pyrimidin-4-yl)-2-(cyanomethyl) piperazine-1-carboxylate

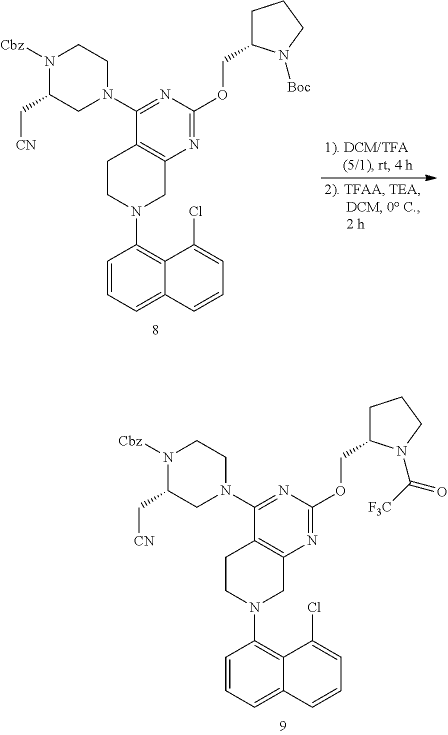 [SMILES: CC(C)(C)OC(=O)N1CCC[C@H]1COc1nc2c(c(N3CCN(C(=O)OCc4ccccc4)[C@@H](CC#N)C3)n1)CCN(c1cccc3cccc(Cl)c13)C2 ; N#CC[C@H]1CN(c2nc(OC[C@@H]3CCCN3C(=O)C(F)(F)F)nc3c2CCN(c2cccc4cccc(Cl)c24)C3)CCN1C(=O)OCc1ccccc1]

To a stirred solution of benzyl (S)-4-(2-(((S)-1-(tert-butoxycarbonyl)pyrrolidin-2-yl)methoxy)-7-(8-chloronaphthalen-1-yl)-5,6,7,8-tetrahydropyrido[3,4-d]pyrimidin-4-yl)-2-(cyanomethyl) piperazine-1-carboxylate (2.20 g, 2.93 mmol) in DCM (30 mL) was added trifluoroacetic acid (6 mL) at room temperature under a nitrogen atmosphere. The resulting mixture was stirred for 4 h at room temperature under a nitrogen atmosphere. The resulting mixture was concentrated under reduced pressure. The residue was resolved in DCM (30 mL), followed by the addition of triethylamine (0.50 g, 4.91 mmol) and trifluoroacetic anhydride (0.62 g, 2.94 mmol) at 0° C. under a nitrogen atmosphere. The resulting mixture was stirred for 2 h at room temperature. The resulting mixture was concentrated under reduced pressure. The residue was purified by silica gel column chromatography, eluted with 2%˜3% methanol in dichloromethane to afford benzyl (2S)-4-(7-(8-chloronaphthalen-1-yl)-2-(((2S)-1-(2,2,2-trifluoroacetyl)pyrrolidin-2-yl)methoxy)-5H,6H,8H-pyrido[3,4-d]pyrimidin-4-yl)-2-(cyano methyl)piperazine-1-carboxylate (1.70 g, 93%) as a light yellow solid: ¹H NMR (400 MHz, CDCl₃) δ 7.75 (dd, J=8.3, 1.5 Hz, 1H), 7.61 (t, J=8.6 Hz, 1H), 7.51 (m, 1H), 7.45 (d, J=7.7 Hz, 1H), 7.39 (d, J=10.9 Hz, 5H), 7.32 (d, J=7.7 Hz, 1H), 7.22 (d, J=7.4 Hz, 1H), 5.20 (s, 2H), 4.57 (m, 4H), 4.31 (d, J=17.3 Hz, 1H), 4.18-3.95 (m, 4H), 3.91-3.66 (m, 2H), 3.52 (td, J=11.2, 4.5 Hz, 1H), 3.40 (td, J=7.2, 3.7 Hz, 3H), 3.22-2.96 (m, 4H), 2.69 (m, 1H), 2.53 (dd, J=16.3, 12.7 Hz, 1H), 2.19-1.81 (m, 5H); LC/MS (ESI, m/z): [(M+1)]⁺=748.1.

### Step 8: Tert-butyl (S)-4-(7-(8-chloronaphthalen-1-yl)-2-(((S)-1-(2,2,2-trifluoroacetyl) pyrrolidin-2-yl)methoxy)-5,6,7,8-tetrahydropyrido[3,4-d]pyrimidin-4-yl)-2-(cyanomethyl) piperazine-1-carboxylate

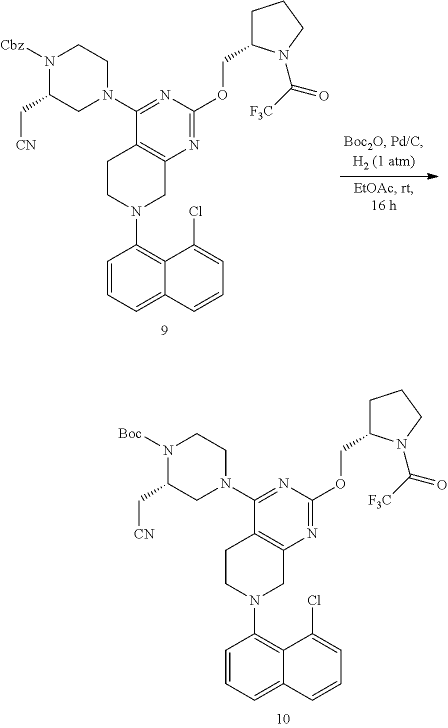 [SMILES: N#CC[C@H]1CN(c2nc(OC[C@@H]3CCCN3C(=O)C(F)(F)F)nc3c2CCN(c2cccc4cccc(Cl)c24)C3)CCN1C(=O)OCc1ccccc1 ; CC(C)(C)OC(=O)N1CCN(c2nc(OC[C@@H]3CCCN3C(=O)C(F)(F)F)nc3c2CCN(c2cccc4cccc(Cl)c24)C3)C[C@@H]1CC#N]

To a stirred solution of benzyl (2S)-4-(7-(8-chloronaphthalen-1-yl)-2-(((2S)-1-(2,2,2-trifluoroacetyl)pyrrolidin-2-yl)methoxy)-5H,6H,8H-pyrido[3,4-d]pyrimidin-4-yl)-2-(cyanomethyl) piperazine-1-carboxylate (1.70 g, 2.27 mmol) in ethyl acetate (50 mL) were added di-tert-butyl dicarbonate (0.99 g, 4.54 mmol) and Pd/C (0.24 g, 10% palladium on charcoal) at room temperature under a nitrogen atmosphere. The resulting mixture was stirred for 16 h at room temperature under a hydrogen atmosphere (2 atm). The resulting mixture was filtered and the filtered cake was washed with methanol (3×30 mL). The filtrate was concentrated under reduced pressure. The residue was purified by silica gel column chromatography, eluted with 2%˜3% methanol in dichloromethane to afford tert-butyl (2S)-4-(7-(8-chloronaphthalen-1-yl)-2-(((2S)-1-(2,2,2-trifluoroacetyl)pyrrolidin-2-yl)methoxy)-5H,6H,8H-pyrido[3,4-d]pyrimidin-4-yl)-2-(cyanomethyl)piperazine-1-carboxylate (1.50 g, 93%) as a yellow solid: ¹H NMR (400 MHz, CDCl₃) δ 7.79 (d, J=8.1 Hz, 1H), 7.70 (d, J=7.7 Hz, 1H), 7.56 (d, J=7.6 Hz, 1H), 7.47 (d, J=7.3 Hz, 1H), 7.43-7.34 (m, 2H), 4.82-4.35 (m, 8H), 4.31-3.93 (m, 4H), 3.85-3.14 (m, 10H), 2.70 (s, 2H); LC/MS (ESI, m/z): [(M+1)]⁺=714.2.

### Step 9: Tert-butyl (S)-4-(7-(8-chloronaphthalen-1-yl)-2-(((S)-pyrrolidin-2-yl)methoxy)-5,6,7,8-tetrahydropyrido[3,4-d]pyrimidin-4-yl)-2-(cyanomethyl)piperazine-1-carboxylate

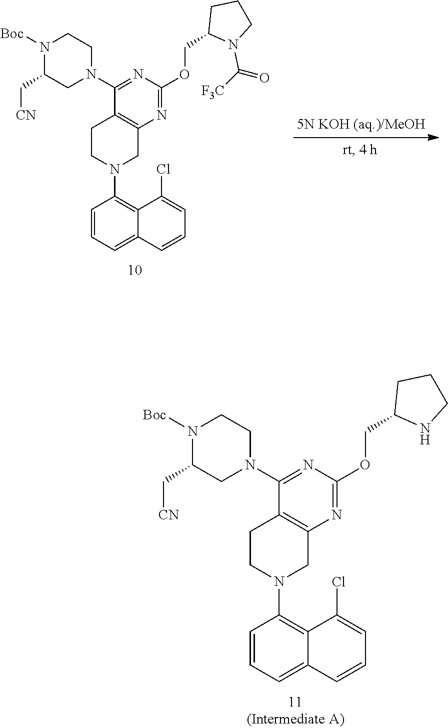 [SMILES: CC(C)(C)OC(=O)N1CCN(c2nc(OC[C@@H]3CCCN3C(=O)C(F)(F)F)nc3c2CCN(c2cccc4cccc(Cl)c24)C3)C[C@@H]1CC#N ; CC(C)(C)OC(=O)N1CCN(c2nc(OC[C@@H]3CCCN3)nc3c2CCN(c2cccc4cccc(Cl)c24)C3)C[C@@H]1CC#N]

To a stirred solution of tert-butyl (2S)-4-(7-(8-chloronaphthalen-1-yl)-2-(((2S)-1-(2,2,2-trifluoroacetyl)pyrrolidin-2-yl)methoxy)-5H,6H,8H-pyrido[3,4-d]pyrimidin-4-yl)-2-(cyanomethyl) piperazine-1-carboxylate (1.70 g, 2.38 mmol) in H₂O (2 mL) and methanol (15 mL) was added KOH (0.53 g, 9.52 mmol) in portions at room temperature under a nitrogen atmosphere. The resulting mixture was stirred for 4 h. The mixture was neutralized to pH=7 with 1 M HCl (aq.). The resulting mixture was concentrated under reduced pressure. The residue was purified by silica gel column chromatography, eluted with 2%˜4% methanol in dichloromethane to afford tert-butyl (2S)-4-(7-(8-chloronaphthalen-1-yl)-2-((2S)-pyrrolidin-2-ylmethoxy)-5H,6H,8H-pyrido[3,4-d]pyrimidin-4-yl)-2-(cyanomethyl)piperazine-1-carboxylate (1.40 g, 98%) as a yellow solid: ¹H NMR (400 MHz, CDCl₃) δ 7.81-7.72 (m, 1H), 7.63 (d, J=7.6 Hz, 1H), 7.56-7.49 (m, 1H), 7.47-7.30 (m, 3H), 5.50-5.25 (m, 1H), 4.62 (s, 2H), 4.45-3.86 (m, 6H), 3.26 (d, J=116.8 Hz, 6H), 2.67 (s, 6H), 2.07 (dd, J=64.1, 46.0 Hz, 5H), 1.53 (s, 9H); LC/MS (ESI, m/z): [(M+1)]⁺=618.3.

### B. Synthesis of Examples

### Example 1: 2-((S)-4-(7-(8-chloronaphthalen-1-yl)-2-(((S)-1-(3-(2-(2-(2-(2,6-dibromo-4-((5-iodo-2-oxoindolin-3-ylidene)methyl)phenoxy)ethoxy)ethoxy)ethoxy) propyl)pyrrolidin-2-yl)methoxy)-5,6,7,8-tetrahydropyrido[3,4-d]pyrimidin-4-yl)-1-(2-fluoro acryloyl)piperazin-2-yl)acetonitrile

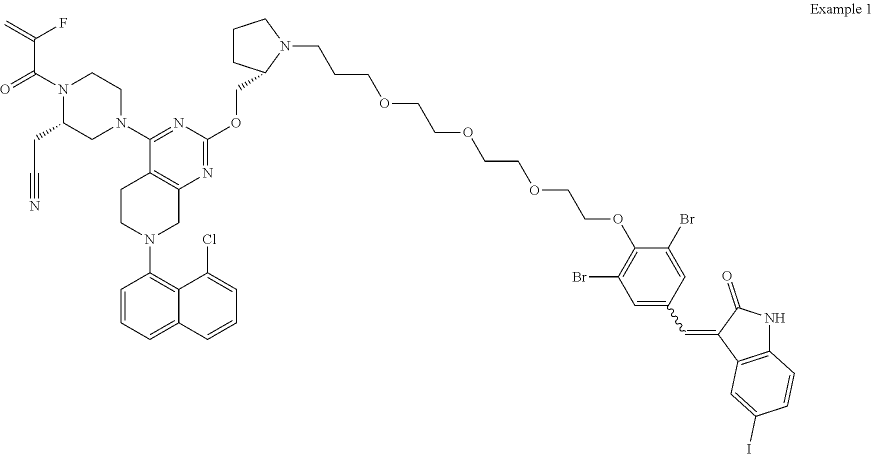 [SMILES: C=C(F)C(=O)N1CCN(c2nc(OC[C@@H]3CCCN3CCCOCCOCCOCCOc3c(Br)cc(C=C4C(=O)Nc5ccc(I)cc54)cc3Br)nc3c2CCN(c2cccc4cccc(Cl)c24)C3)C[C@@H]1CC#N]

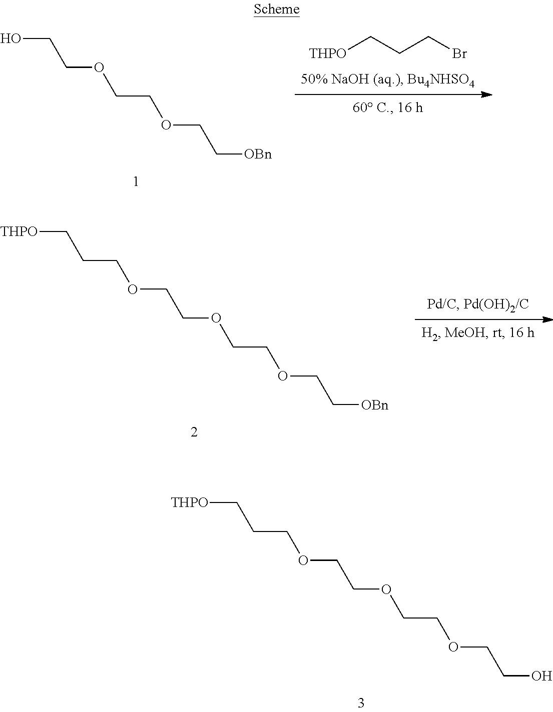

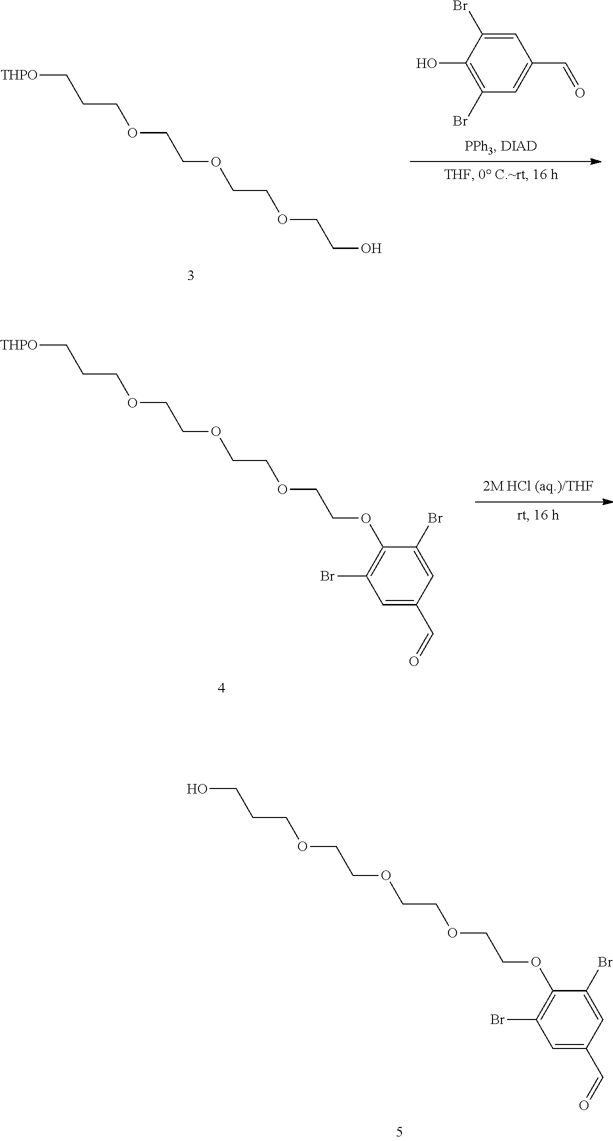

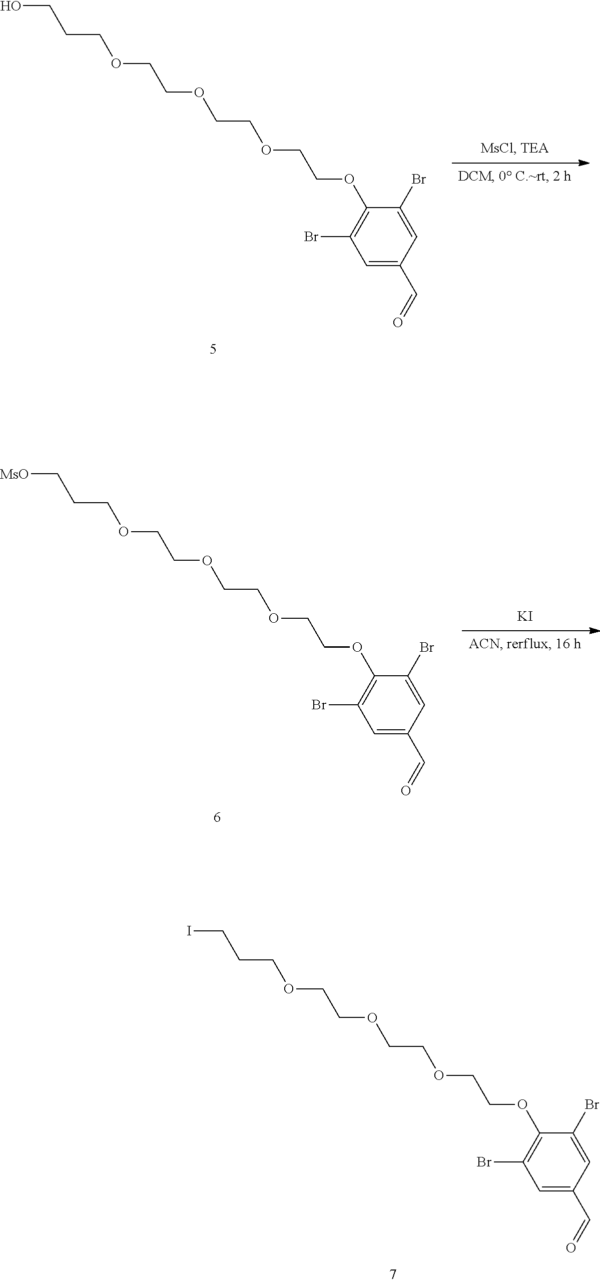

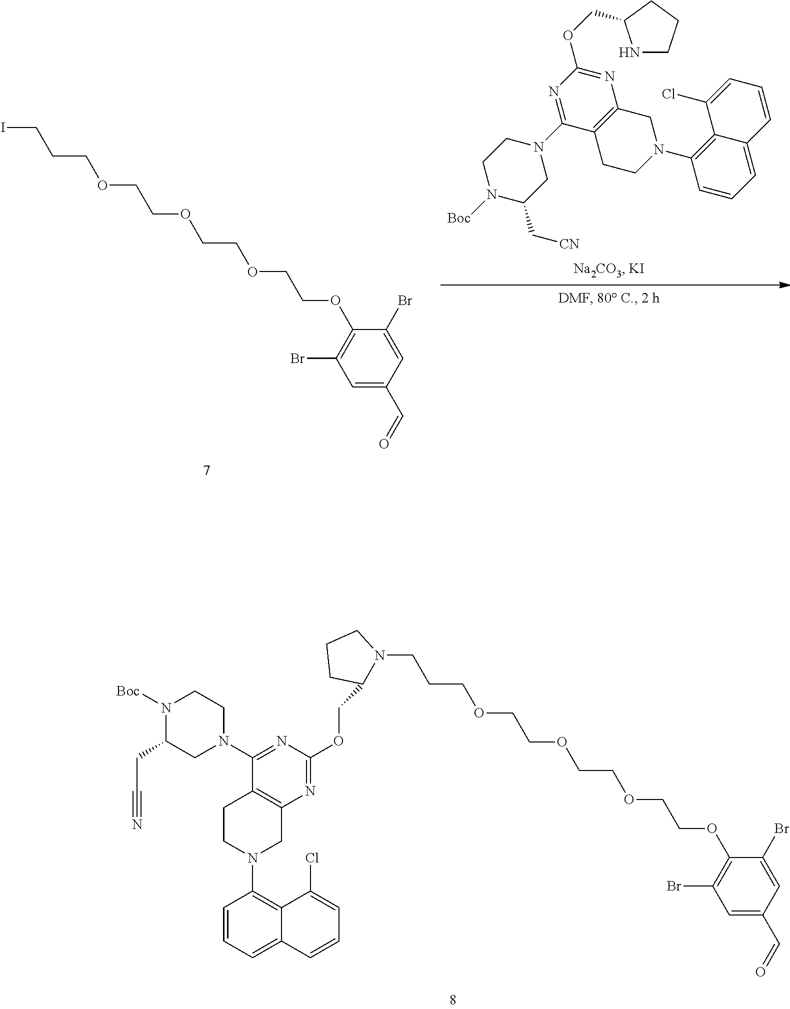

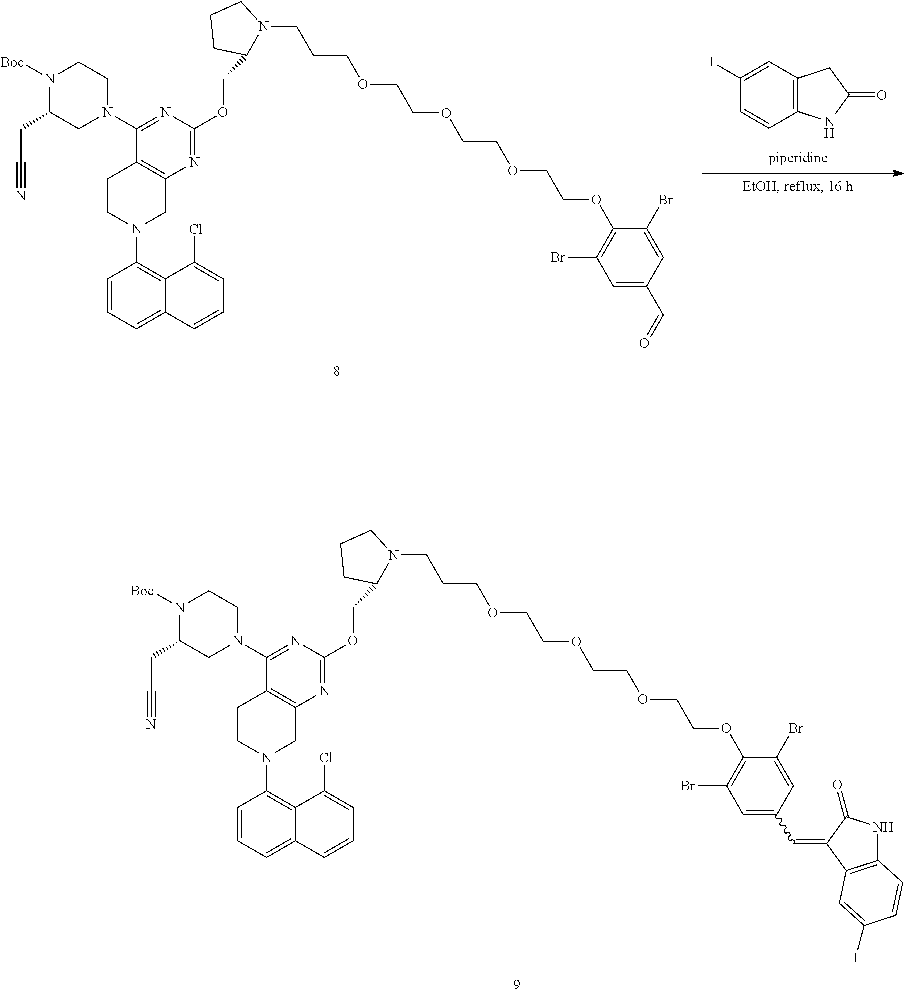

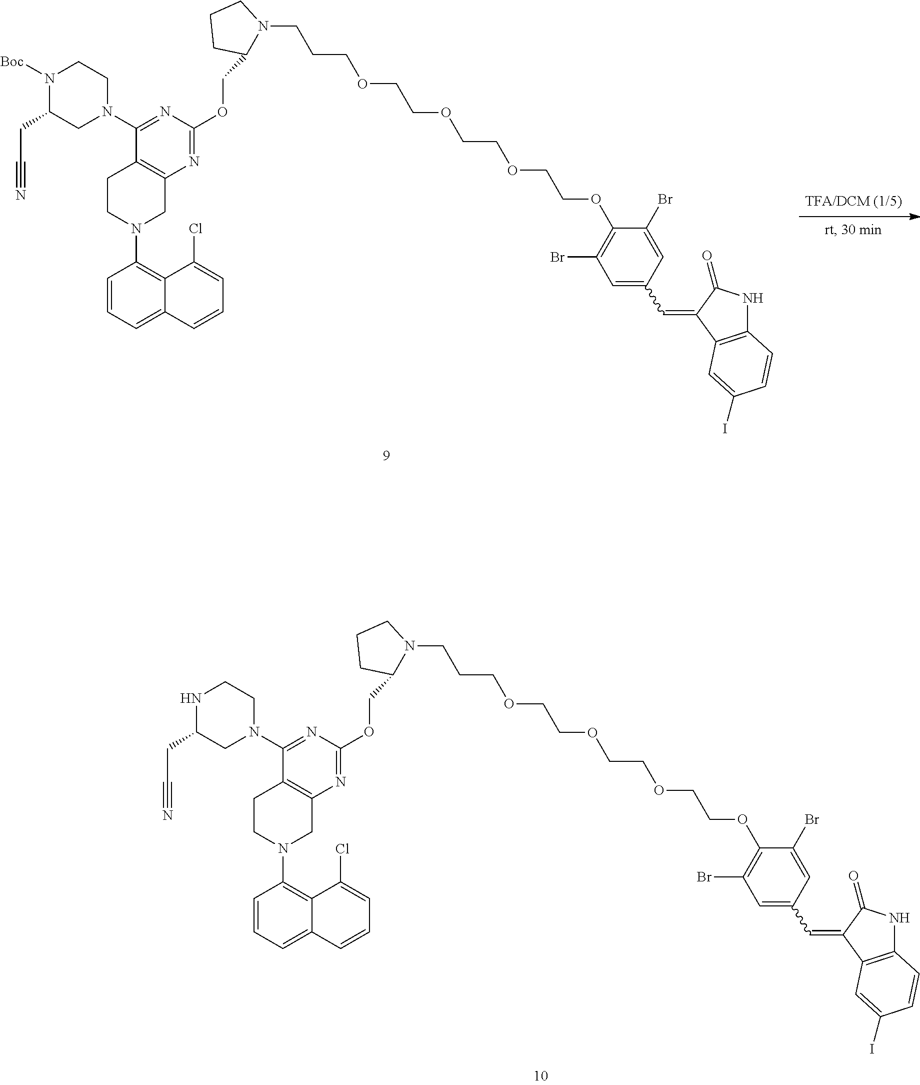

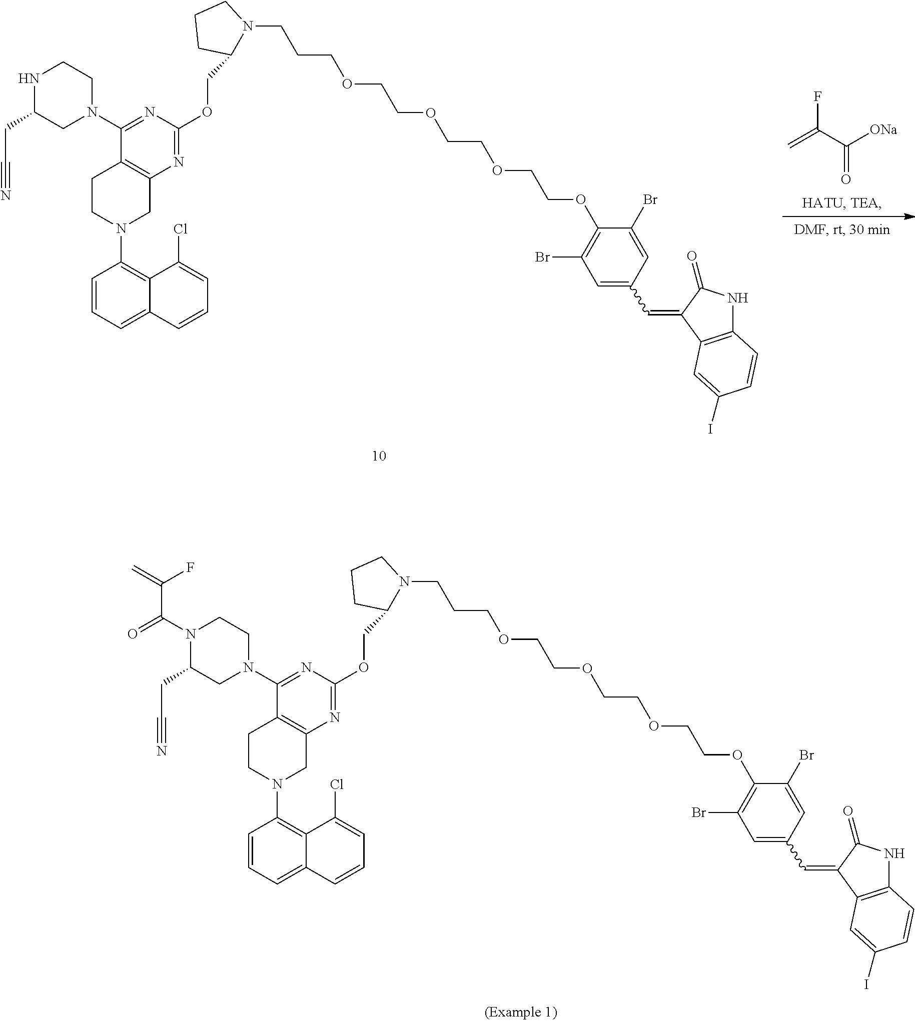

### Step 1: 2-((1-Phenyl-2,5,8,11-tetraoxatetradecan-14-yl)oxy)tetrahydro-2H-pyran

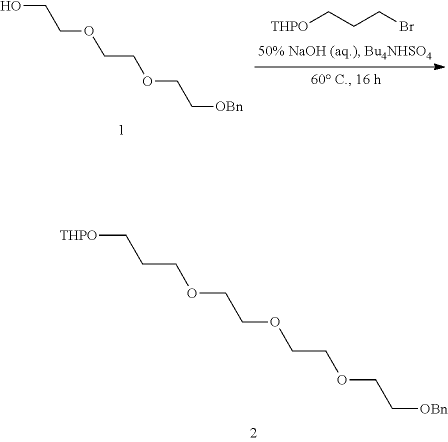

To a stirred solution of 2-(2-(2-(benzyloxy)ethoxy)ethoxy)ethan-1-ol (5.4 g, 22.47 mmol) in THF (50 mL) were added 2-(3-bromopropoxy)oxane (10.0 g, 44.94 mmol), tetrabutylammonium hydrogen sulfate (0.76 g, 2.25 mmol) and 50% NaOH(aq.) (40 mL) at room temperature. The resulting mixture was stirred for 16 h at 60° C. under a nitrogen atmosphere. After cooling down to room temperature, the resulting mixture was extracted with ethyl acetate (3×200 mL). The combined organic layers were washed with brine (3×100 mL) and dried over anhydrous Na₂SO₄. After filtration, the filtrate was concentrated under reduced pressure. The residue was purified by silica gel column chromatography, eluted with 10%˜50% ethyl acetate in petroleum ether to afford 2-((1-phenyl-2,5,8,11-tetraoxatetradecan-14-yl)oxy)tetrahydro-2H-pyran (7.7 g, 79%) as a yellow oil: ¹H NMR (400 MHz, CDCl₃) δ 7.36-7.32 (m, 4H), 7.32-7.24 (m, 1H), 4.59-4.55 (m, 3H), 3.88-3.77 (m, 2H), 3.71-3.61 (m, 10H), 3.61-3.52 (m, 4H), 3.53-3.43 (m, 2H), 1.93-1.75 (m, 3H), 1.75-1.64 (m, 1H), 1.63-1.45 (m, 4H); LC/MS (ESI, m/z): [(M+1)]+=383.2.

### Step 2: 2-(2-(2-(3-((Tetrahydro-2H-pyran-2-yl)oxy)propoxy)ethoxy)ethoxy)ethan-1-ol

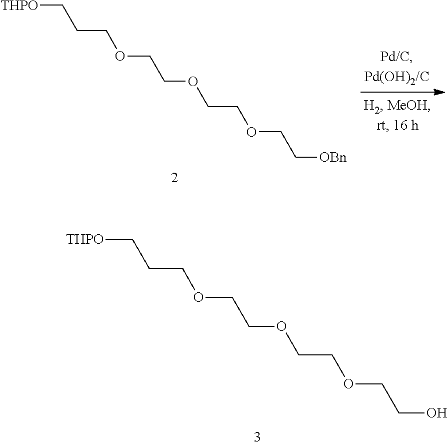

To a stirred solution of 2-((1-phenyl-2,5,8,11-tetraoxatetradecan-14-yl)oxy)tetrahydro-2H-pyran (4.00 g, 10.46 mmol) in methanol (100 mL) were added Pd(OH)₂/C (2.00 g, 10% wt) and Pd/C (2.00 g, 10% wt) at ambient temperature under a nitrogen atmosphere. The resulting mixture was stirred for 16 h at ambient temperature under a hydrogen atmosphere (2 atm). The resulting mixture was filtered and the filtered cake was washed with methanol (3×20 mL). The filtrate was concentrated under reduced pressure. The residue was purified by silica gel column chromatography, eluted with 8% methanol in dichloromethane to afford 2-(2-(2-(3-((tetrahydro-2H-pyran-2-yl)oxy)propoxy)ethoxy)ethoxy)ethan-1-ol (2.5 g, 82%) as a yellow oil. ¹H NMR (400 MHz, CDCl₃) δ 4.58 (dd, J=4.4, 2.8 Hz, 1H), 3.90-3.77 (m, 2H), 3.76-3.71 (m, 2H), 3.71-3.63 (m, 6H), 3.63-3.54 (m, 6H), 3.54-3.45 (m, 2H), 1.93-1.85 (m, 2H), 1.85-1.77 (m, 1H) 1.76-1.65 (m, 1H), 1.63-1.45 (m, 4H).; LC/MS (ESI, m/z): [(M+1)]+=293.1.

### Step 3: 3,5-Dibromo-4-(2-(2-(2-(3-((tetrahydro-2H-pyran-2-yl)oxy)propoxy)ethoxy) ethoxy)ethoxy)benzaldehyde

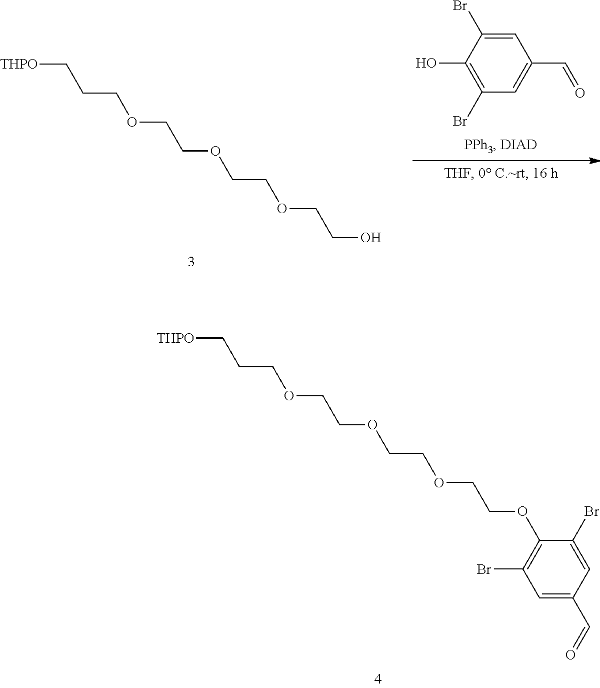

To a solution of 2-(2-(2-(3-((tetrahydro-2H-pyran-2-yl)oxy)propoxy)ethoxy)ethoxy) ethan-1-ol (2.48 g, 8.49 mmol) in THF (75 mL) were added 3,5-dibromo-4-hydroxy benzaldehyde (2.16 g, 7.72 mmol), triphenylphosphine (4.05 g, 15.43 mmol) and diisopropyl azodicarboxylate (3.12 g, 15.43 mmol) dropwise over 5 min at 0° C. The resulting mixture was stirred for 16 h at room temperature. The resulting mixture was diluted with water (100 mL). The resulting mixture was extracted with ethyl acetate (3×300 mL). The combined organic layers were washed with brine (3×100 mL) and dried over anhydrous Na₂SO₄. After filtration, the filtrate was concentrated under reduced pressure. The residue was purified by silica gel column chromatography, eluted with 0-50% ethyl acetate in petroleum ether to afford 3,5-dibromo-4-(2-(2-(2-(3-((tetrahydro-2H-pyran-2-yl)oxy)propoxy)ethoxy)ethoxy)ethoxy)benzaldehyde (4.15 g, 49%) as a yellow solid: ¹H NMR (400 MHz, CDCl₃) δ 9.85 (s, 1H), 8.02 (s, 2H), 4.57 (t, J=4.2 Hz, 1H), 4.32-4.26 (m, 2H), 3.95 (t, J=4.9 Hz, 2H), 3.87-3.73 (m, 4H), 3.70-3.62 (m, 4H), 3.62-3.54 (m, 4H) 3.54-3.44 (m, 2H), 1.93-1.76 (m, 3H) 1.76-1.70 (m, 1H), 1.69-1.46 (m, 4H); LC/MS (ESI, m/z): [(M+1)]+=554.2.

### Step 4: 3,5-Dibromo-4-(2-(2-(2-(3-hydroxypropoxy)ethoxy)ethoxy)ethoxy)benzaldehyde

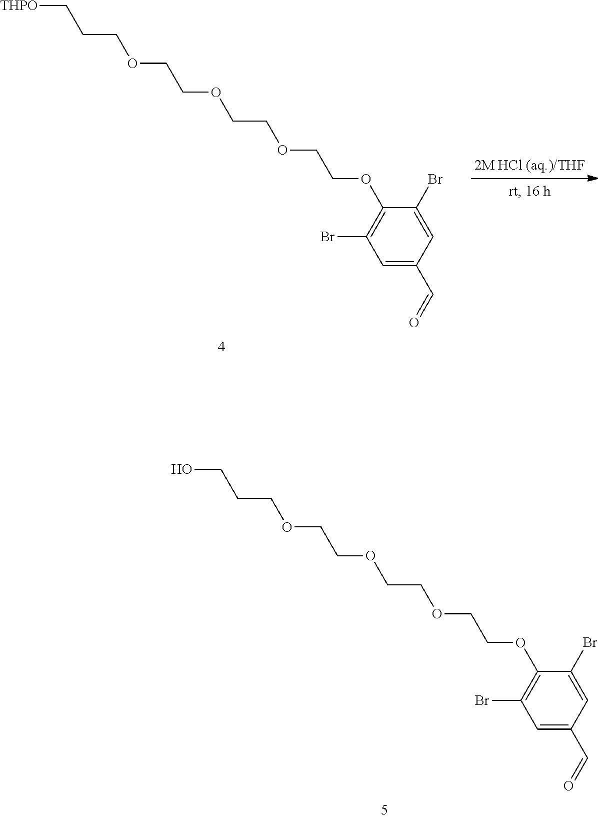

To a stirred solution of 3,5-dibromo-4-(2-(2-(2-(3-((tetrahydro-2H-pyran-2-yl)oxy) propoxy)ethoxy)ethoxy)ethoxy)benzaldehyde (4.15 g, 7.49 mmol) in THF (50 mL) was added HCl (aq.) (2 M) (50 mL) at room temperature under a nitrogen atmosphere. The resulting mixture was stirred for 16 h at room temperature under a nitrogen atmosphere. The mixture was neutralized to pH=7 with saturated NaHCO₃ (aq.). The resulting mixture was extracted with ethyl acetate (3×100 mL). The combined organic layers were washed with brine (2×20 mL) and dried over anhydrous Na₂SO₄. After filtration, the filtrate was concentrated under reduced pressure. The residue was purified by silica gel column chromatography, eluted with 0˜50% ethyl acetate in petroleum ether to afford 3,5-dibromo-4-(2-(2-(2-(3-hydroxypropoxy)ethoxy) ethoxy) ethoxy)benzaldehyde (1.6 g, 45%) as a yellow solid: ¹H NMR (300 MHz, CDCl₃) δ 9.85 (s, 1H), 8.03 (s, 2H), 4.35-4.25 (m, 2H), 4.00-3.88 (m, 2H), 3.85-3.73 (m, 4H), 3.72-3.57 (m, 8H), 1.89-1.78 (m, 2H); LC/MS (ESI, m/z): [(M+1)]⁺=470.9.

### Step 5: 3-(2-[2-[2-(2,6-Dibromo-4-formylphenoxy)ethoxy]ethoxy]ethoxy)propyl methane sulfonate

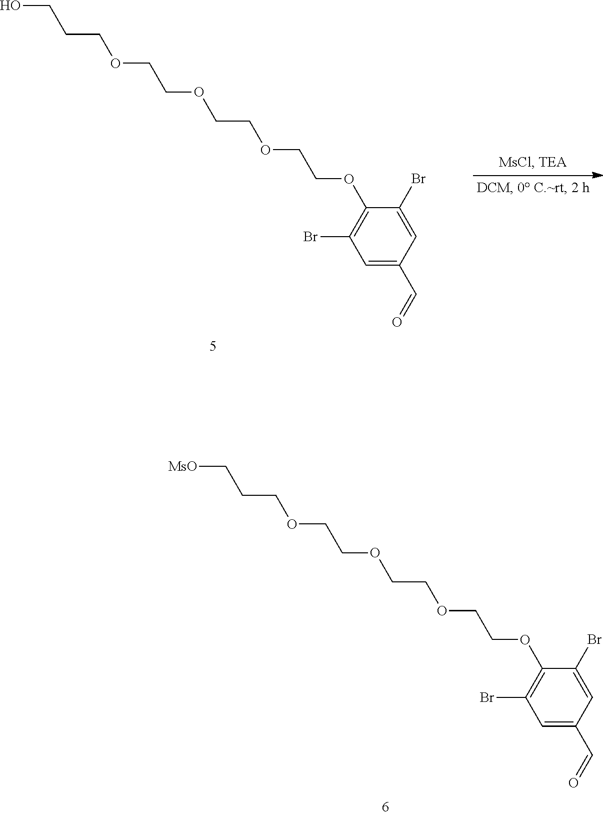 [SMILES: O=Cc1cc(Br)c(OCCOCCOCCOCCCO)c(Br)c1 ; CS(=O)(=O)OCCCOCCOCCOCCOc1c(Br)cc(C=O)cc1Br]

To a stirred solution of 3,5-dibromo-4-(2-(2-(2-(3-hydroxypropoxy)ethoxy)ethoxy) ethoxy)benzaldehyde (550 mg, 1.17 mmol) in DCM (23 mL) were added triethylamine (237 mg, 2.34 mmol) and methanesulfonyl chloride (268 mg, 2.34 mmol) at 0° C. under a nitrogen atmosphere. The resulting mixture was stirred for 2 h at room temperature under a nitrogen atmosphere. The reaction was quenched with water (50 mL). The resulting mixture was extracted with ethyl acetate (3×100 mL). The combined organic layers were washed with brine (3×100 mL) and dried over anhydrous Na₂SO₄. After filtration, the filtrate was concentrated under reduced pressure. The residue was purified by silica gel column chromatography, 8% methanol in dichloromethane to afford 3-(2-[2-[2-(2,6-dibromo-4-formylphenoxy)ethoxy]ethoxy] ethoxy)propyl methanesulfonate (618 mg, 87%) as a yellow oil: LC/MS (ESI, m/z): [(M+1)]⁺=548.2.

### Step 6: 3,5-Dibromo-4-(2-(2-(2-(3-iodopropoxy)ethoxy)ethoxy)ethoxy)benzaldehyde

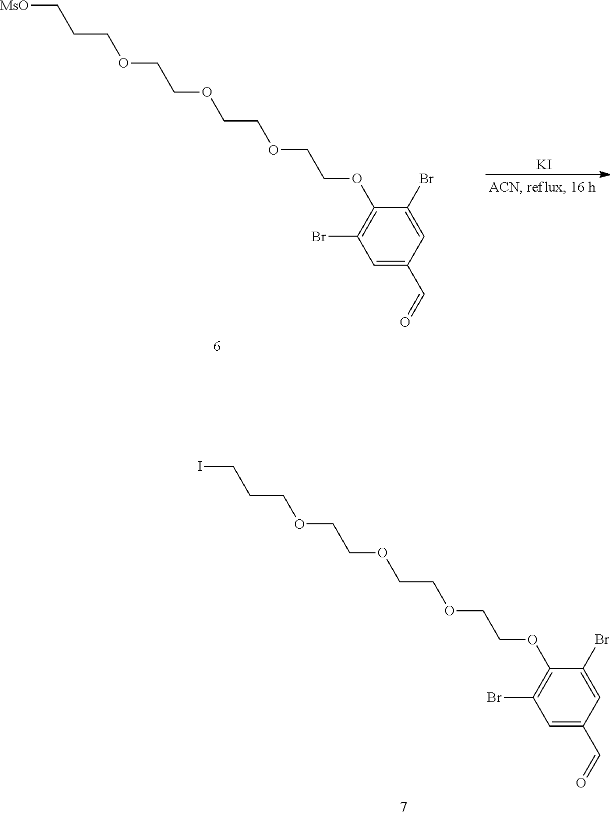 [SMILES: CS(=O)(=O)OCCCOCCOCCOCCOc1c(Br)cc(C=O)cc1Br ; "KI" ; O=Cc1cc(Br)c(OCCOCCOCCOCCCI)c(Br)c1]

To a stirred solution of 3-(2-(2-(2-(2,6-dibromo-4-formylphenoxy)ethoxy)ethoxy) ethoxy)propyl methanesulfonate (550 mg, 1.00 mmol) in acetonitrile (10 mL) was added KI (666 mg, 4.01 mmol) at room temperature under a nitrogen atmosphere. The resulting mixture was stirred for 16 h at 80° C. under a nitrogen atmosphere. The resulting mixture was cooled and concentrated under reduced pressure. The residue was purified by silica gel column chromatography, eluted with 33% ethyl acetate in petroleum ether to afford 3,5-dibromo-4-(2-(2-(2-(3-iodopropoxy)ethoxy)ethoxy)ethoxy)benzaldehyde (428 mg, 74%) as a yellow solid: ¹H NMR (300 MHz, CDCl₃) δ 9.86 (s, 1H), 8.03 (s, 2H), 4.30 (t, J=4.9 Hz, 2H), 3.95 (t, J=4.9 Hz, 2H), 3.82-3.73 (m, 2H), 3.73-3.58 (m, 6H), 3.53 (t, J=5.9 Hz, 2H), 3.28 (t, J=6.8 Hz, 2H), 2.13-2.00 (m, 2H); LC/MS (ESI, m/z): [(M+1)]⁺=580.9.

### Step 7: Tert-butyl (S)-4-(7-(8-chloronaphthalen-1-yl)-2-(((S)-1-(3-(2-(2-(2-(2,6-dibromo-4-formylphenoxy)ethoxy)ethoxy)ethoxy)propyl)pyrrolidin-2-yl)methoxy)-5,6,7,8-tetrahydro pyrido[3,4-d]pyrimidin-4-yl)-2-(cyanomethyl)piperazine-1-carboxylate

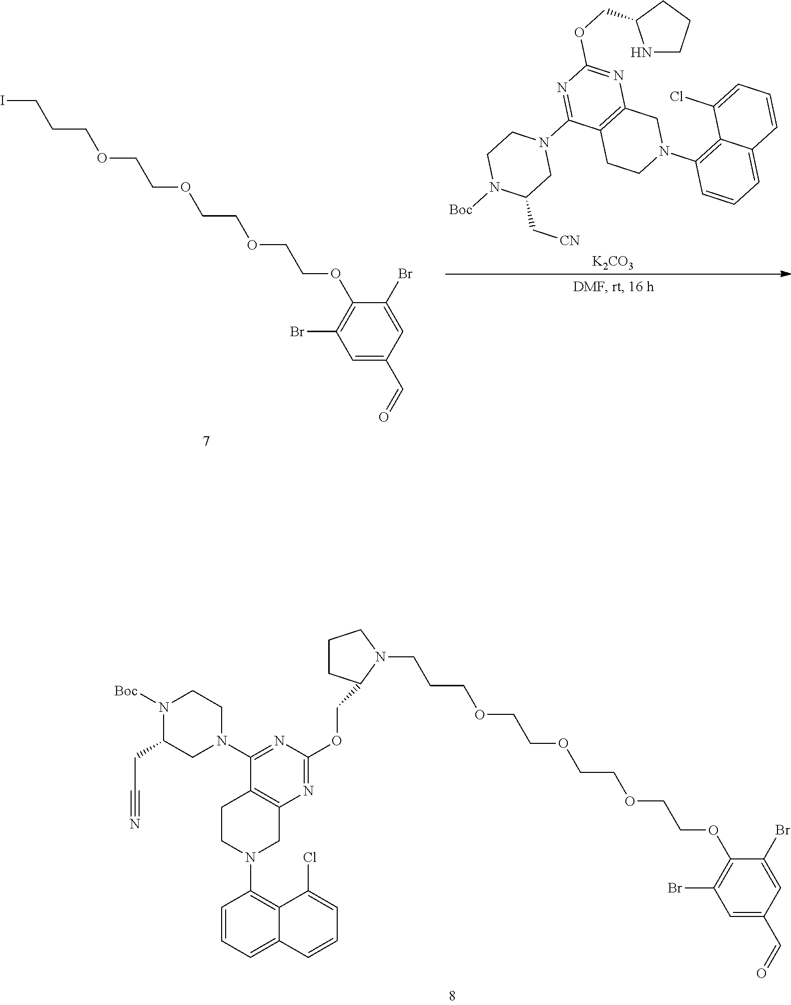

To a stirred solution of 3,5-dibromo-4-(2-(2-(2-(3-iodopropoxy)ethoxy)ethoxy)ethoxy) benzaldehyde (253 mg, 0.44 mmol) in DMF (2 mL) were added tert-butyl (2S)-4-(7-(8-chloro naphthalen-1-yl)-2-((2S)-pyrrolidin-2-ylmethoxy)-5H,6H,8H-pyrido[3,4-d]pyrimidin-4-yl)-2-(cyanomethyl)piperazine-1-carboxylate (180 mg, 0.29 mmol) and K₂CO₃ (48 mg, 0.35 mmol) at room temperature under a nitrogen atmosphere. The resulting mixture was stirred for 16 h at 25° C. under a nitrogen atmosphere. The resulting mixture was concentrated under reduced pressure. The residue was purified by Prep-TLC (CH₂Cl₂/methanol=10/1) to afford tert-butyl (S)-4-(7-(8-chloronaphthalen-1-yl)-2-(((S)-1-(3-(2-(2-(2-(2,6-dibromo-4-formylphenoxy)ethoxy)ethoxy) ethoxy)propyl)pyrrolidin-2-yl)methoxy)-5,6,7,8-tetrahydropyrido[3,4-d]pyrimidin-4-yl)-2-(cyanomethyl)piperazine-1-carboxylate (174 mg, 56%) as a yellow solid: ¹H NMR (400 MHz, CDCl₃) δ 9.84 (d, J=1.8 Hz, 1H), 8.06-7.97 (m, 2H), 7.78-7.72 (m, 1H), 7.64-7.57 (m, 1H), 7.54-7.49 (m, 1H), 7.44 (q, J=7.4 Hz, 1H), 7.33 (td, J=7.8, 2.0 Hz, 1H), 7.25-7.18 (m, 1H), 4.61 (s, 2H), 4.39 (dd, J=17.9, 12.6 Hz, 1H), 4.26 (t, J=4.9 Hz, 2H), 4.19-3.97 (m, 2H), 3.96-3.85 (m, 3H), 3.81 (d, J=18.6 Hz, 2H), 3.72 (t, J=7.3, 2H), 3.68-3.47 (m, 9H), 3.47-2.88 (m, 8H), 2.87-2.65 (m, 2H), 2.64-2.52 (m, 1H), 2.41-2.23 (m, 2H), 2.22-1.96 (m, 3H), 1.94-1.56 (m, 3H). LC/MS (ESI, m/z): [(M+1)]⁺=1070.3.

### Step 8: Tert-butyl (S)-4-(7-(8-chloronaphthalen-1-yl)-2-(((S)-1-(3-(2-(2-(2-(2,6-dibromo-4-((5-iodo-2-oxoindolin-3-ylidene)methyl)phenoxy)ethoxy)ethoxy)ethoxy)propyl)pyrrolidin-2-yl)methoxy)-5,6,7,8-tetrahydropyrido[3,4-d]pyrimidin-4-yl)-2-(cyanomethyl)piperazine-1-carboxylate

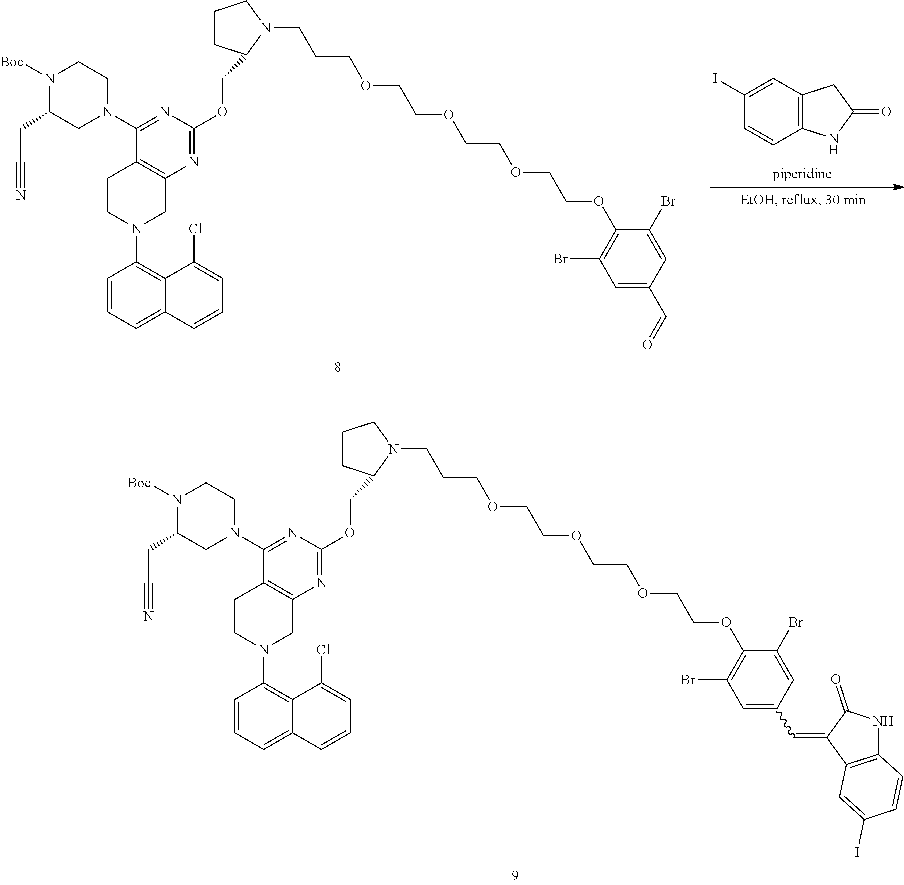

To a stirred solution of tert-butyl (S)-4-(7-(8-chloronaphthalen-1-yl)-2-(((S)-1-(3-(2-(2-(2-(2,6-dibromo-4-formylphenoxy)ethoxy)ethoxy)ethoxy)propyl)pyrrolidin-2-yl)methoxy)-5,6,7,8-tetrahydropyrido[3,4-d]pyrimidin-4-yl)-2-(cyanomethyl)piperazine-1-carboxylate (120 mg, 0.11 mmol) in ethanol (4 mL) were added 5-iodo-1,3-dihydroindol-2-one (29 mg, 0.11 mmol) and piperidine (0.9 mg, 0.01 mmol) at room temperature under a nitrogen atmosphere. The resulting mixture was stirred for 30 min at 80° C. under a nitrogen atmosphere. The resulting mixture was concentrated under reduced pressure. The residue was purified by Prep-TLC (CH₂Cl₂/methanol=15/1) to afford tert-butyl (S)-4-(7-(8-chloronaphthalen-1-yl)-2-(((S)-1-(3-(2-(2-(2-(2,6-dibromo-4-((5-iodo-2-oxoindolin-3-ylidene)methyl)phenoxy)ethoxy)ethoxy)ethoxy) propyl)pyrrolidin-2-yl)methoxy)-5,6,7,8-tetrahydropyrido[3,4-d]pyrimidin-4-yl)-2-(cyanomethyl)piperazine-1-carboxylate (120 mg, 62%) as a yellow solid: ¹H NMR (400 MHz, DMSO-d₆) δ 10.86-10.69 (m, 1H), 8.77 (s, 1H), 8.14-7.81 (m, 3H), 7.72 (d, J=8.9 Hz, 1H), 7.62-7.47 (m, 3H), 7.46-7.26 (m, 3H), 6.71 (dd, J=17.2, 8.2 Hz, 1H), 4.51 (s, 1H), 4.30-4.04 (m, 4H), 4.03-3.63 (m, 7H), 3.63-3.33 (m, 10H), 3.26-2.75 (m, 8H), 2.75-2.59 (m, 2H), 2.55-2.52 (m, 2H), 2.38-2.23 (m, 1H), 2.23-2.05 (m, 1H), 1.95-1.79 (m, 1H), 1.77-1.52 (m, 5H), 1.44 (s, 9H); LC/MS (ESI, m/z): [(M+1)]+=1311.2.

### Step 9: 2-((S)-4-(7-(8-chloronaphthalen-1-yl)-2-(((S)-1-(3-(2-(2-(2-(2,6-dibromo-4-((5-iodo-2-oxoindolin-3-ylidene)methyl)phenoxy)ethoxy)ethoxy)ethoxy)propyl)pyrrolidin-2-yl)methoxy)-5,6,7,8-tetrahydropyrido[3,4-d]pyrimidin-4-yl)piperazin-2-yl)acetonitrile

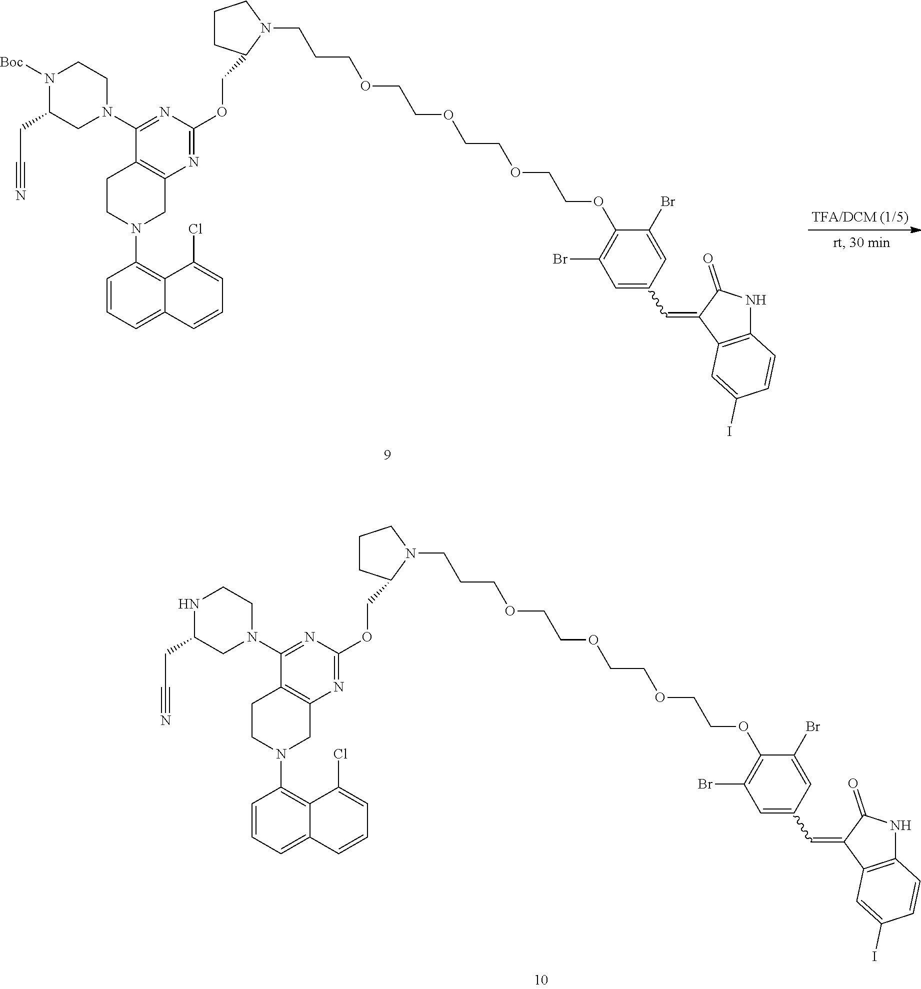

To a stirred solution of tert-butyl (S)-4-(7-(8-chloronaphthalen-1-yl)-2-(((S)-1-(3-(2-(2-(2-(2,6-dibromo-4-((5-iodo-2-oxoindolin-3-ylidene)methyl)phenoxy)ethoxy)ethoxy)ethoxy) propyl)pyrrolidin-2-yl)methoxy)-5,6,7,8-tetrahydropyrido[3,4-d]pyrimidin-4-yl)-2-(cyanomethyl) piperazine-1-carboxylate (100 mg, 0.07 mmol) in DCM (5 mL) were added trifluoroacetic acid (1 mL) dropwise at room temperature under a nitrogen atmosphere. The resulting mixture was stirred for 30 min at room temperature under a nitrogen atmosphere. The resulting mixture was concentrated under reduced pressure. The residue was purified by reverse phase flash chromatography with the following conditions: Column: Spherical C18, 20-40 μm, 120 g; Eluent A: Water (plus 10 mM NH₄HCO₃); Eluent B: ACN; Flow rate: 50 mL/min; Gradient: 70% B-95% B in 20 min; Detector: UV 220/254 nm) to afford 2-((S)-4-(7-(8-chloronaphthalen-1-yl)-2-(((S)-1-(3-(2-(2-(2-(2,6-dibromo-4-((5-iodo-2-oxoindolin-3-ylidene)methyl)phenoxy)ethoxy)ethoxy) ethoxy)propyl)pyrrolidin-2-yl)methoxy)-5,6,7,8-tetrahydropyrido[3,4-d]pyrimidin-4-yl)piperazin-2-yl)acetonitrile (50 mg, 48%) as a yellow solid: ¹H NMR (400 MHz, CDCl₃) δ 8.52-813 (m, 1H), 7.91-7.68 (m, 2H), 7.63-7.56 (m, 1H), 7.52 (t, J=8.6 Hz, 2H), 7.46-7.35 (m, 2H), 7.32 (t, J=7.8 Hz, 1H), 7.25-7.16 (m, 2H), 6.66 (t, J=10.1 Hz, 1H), 4.38 (d, J=17.8 Hz, 2H), 4.32-4.18 (m, 2H), 4.18-3.99 (m, 2H), 3.99-3.78 (m, 4H), 3.78-3.69 (m, 2H), 3.69-3.56 (m, 4H), 3.57-3.42 (m, 5H), 3.40-3.28 (m, 1H), 3.28-2.76 (m, 9H), 2.59-2.38 (m, 4H), 2.37-2.17 (m, 2H), 2.14-1.67 (m, 6H), 1.67-1.54 (m, 1H); LC/MS (ESI, m/z): [(M+1)]+=1211.4.

### Step 10: 2-((S)-4-(7-(8-chloronaphthalen-1-yl)-2-(((S)-1-(3-(2-(2-(2-(2,6-dibromo-4-((5-iodo-2-oxoindolin-3-ylidene)methyl)phenoxy)ethoxy)ethoxy)ethoxy)propyl)pyrrolidin-2-yl)methoxy)-5,6,7,8-tetrahydropyrido[3,4-d]pyrimidin-4-yl)-1-(2-fluoroacryloyl)piperazin-2-yl)acetonitrile

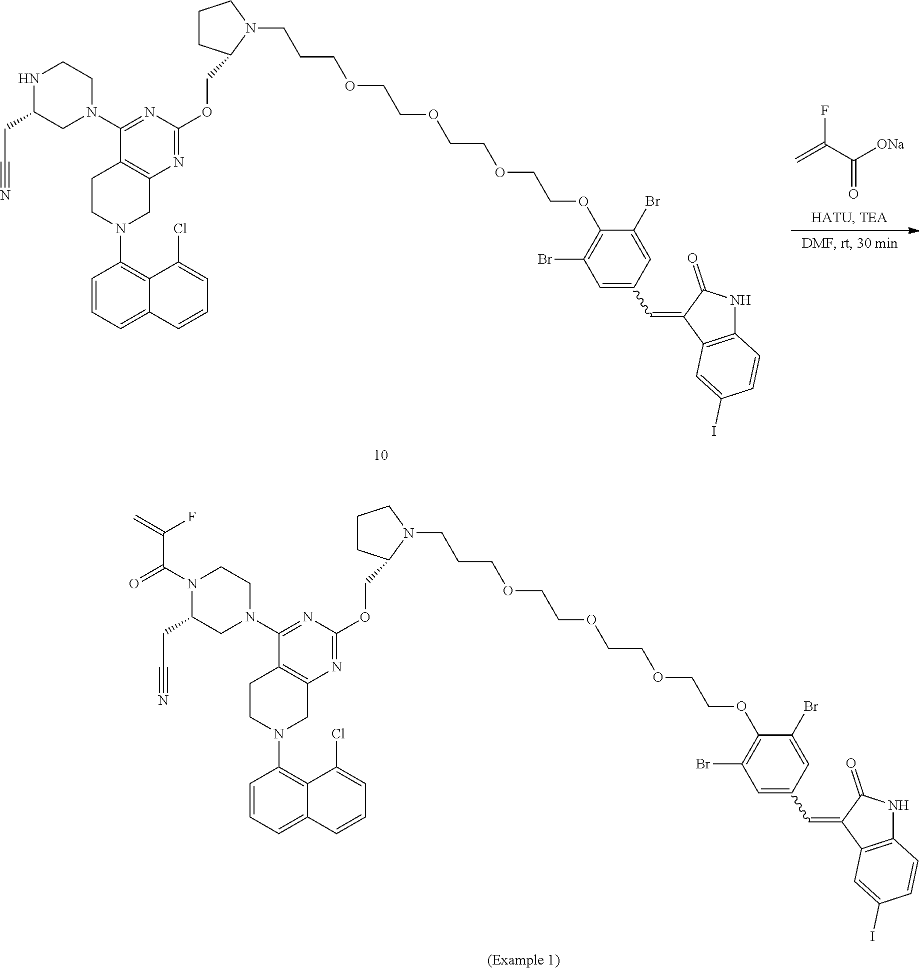

To a stirred solution of 2-((S)-4-(7-(8-chloronaphthalen-1-yl)-2-(((S)-1-(3-(2-(2-(2-(2,6-dibromo-4-((5-iodo-2-oxoindolin-3-ylidene)methyl)phenoxy)ethoxy)ethoxy)ethoxy propyl) pyrrolidin-2-yl)methoxy)-5,6,7,8-tetrahydropyrido[3,4-d]pyrimidin-4-yl)piperazin-2-yl) acetonitrile (50 mg, 0.04 mmol) in DMF (3 mL) were added sodium 2-fluoroprop-2-enoate (9 mg, 0.08 mmol), TEA (12 mg, 0.12 mmol) and HATU (23 mg, 0.06 mmol) at room temperature under a nitrogen atmosphere. The resulting mixture was stirred for 30 min at room temperature. The resulting mixture was concentrated under reduced pressure. The residue was purified by Prep-HPLC with the following conditions: Column: XSelect CSH Prep C18 OBD Column, 19*250 mm, 5 μm; Eluent A: Water (plus 10 mM NH₄HCO₃); Eluent B: ACN; Flow rate: 25 mL/min; Gradient: 80% B to 90% B in 10 min; Detector: UV 220/254 nm. The fractions containing desired product were collected, concentrated under reduced pressure and lyophilized to afford 2-((S)-4-(7-(8-chloronaphthalen-1-yl)-2-(((S)-1-(3-(2-(2-(2-(2,6-dibromo-4-((5-iodo-2-oxoindolin-3-ylidene)methyl)phenoxy)ethoxy)ethoxy)ethoxy)propyl)pyrrolidin-2-yl)methoxy)-5,6,7,8-tetrahydropyrido[3,4-d]pyrimidin-4-yl)-1-(2-fluoroacryloyl)piperazin-2-yl)acetonitrile (6.3 mg, 12%) as a yellow solid: ¹H NMR (400 MHz, DMSO-d₆) δ 10.81 (d, J=22.4 Hz, 1H), 8.78 (s, 1H), 8.07-7.80 (m, 3H), 7.75-7.69 (m, 1H), 7.59-7.48 (m, 3H), 7.44 (t, J=7.8 Hz, 1H), 7.32 (dd, J=15.8, 7.5 Hz, 1H), 6.71 (dd, J=17.2, 8.2 Hz, 1H), 5.46-5.18 (m, 2H), 4.86 (s, 1H), 4.28-4.11 (m, 4H), 4.06-3.83 (m, 4H), 3.32-3.67 (m, 3H), 3.61-3.52 (m, 2H), 3.52-3.41 (m, 5H), 3.41-3.34 (m, 4H), 3.20 (dd, J=14.0, 10.3 Hz, 1H), 3.16-2.97 (m, 4H), 2.97-2.81 (m, 2H), 2.74-2.64 (m, 2H), 2.55-2.52 (m, 2H), 2.32-2.23 (m, 1H), 2.17-2.07 (m, 1H), 1.93-1.81 (m, 1H), 1.72-1.53 (m, 5H); LC/MS (ESI, m/z): [(M+1)]⁺=1283.55.

### Example 2: 2-((S)-4-(7-(8-chloronaphthalen-1-yl)-2-(((S)-1-(3-(2-(2,6-dibromo-4-((5-iodo-2-oxoindolin-3-ylidene)methyl)phenoxy)ethoxy)propyl) pyrrolidin-2-yl)methoxy)-5,6,7,8-tetrahydropyrido[3,4-d]pyrimidin-4-yl)-1-(2-fluoro acryloyl)piperazin-2-yl)acetonitrile

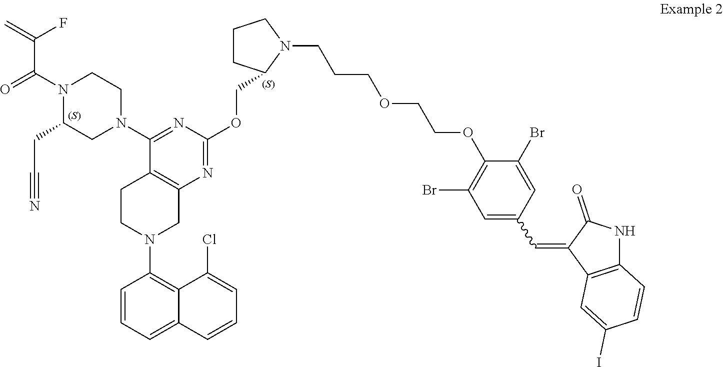

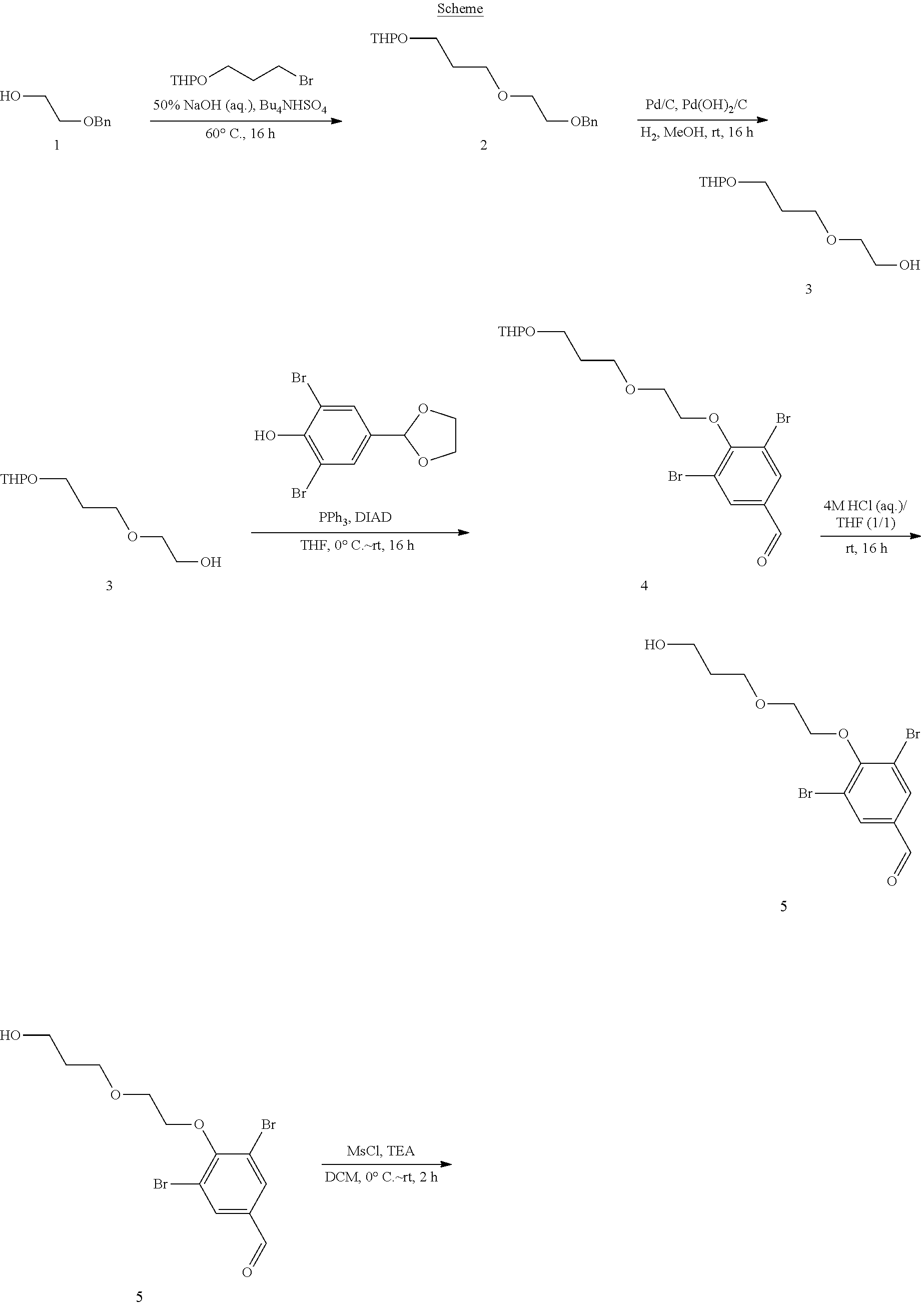

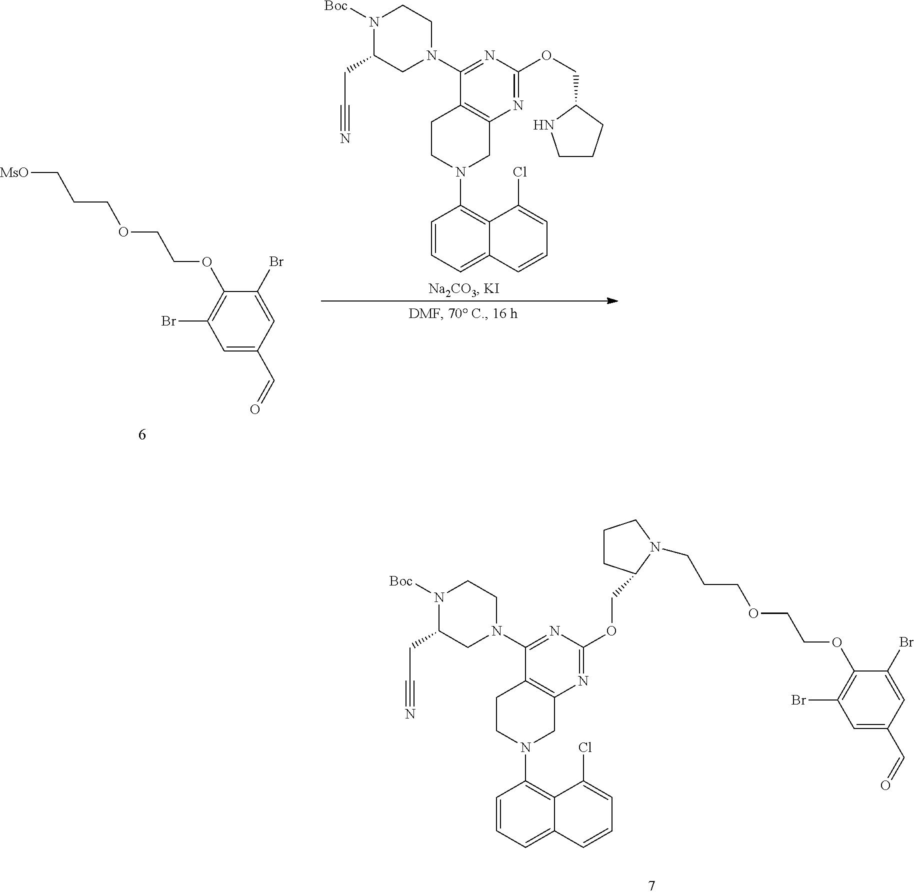

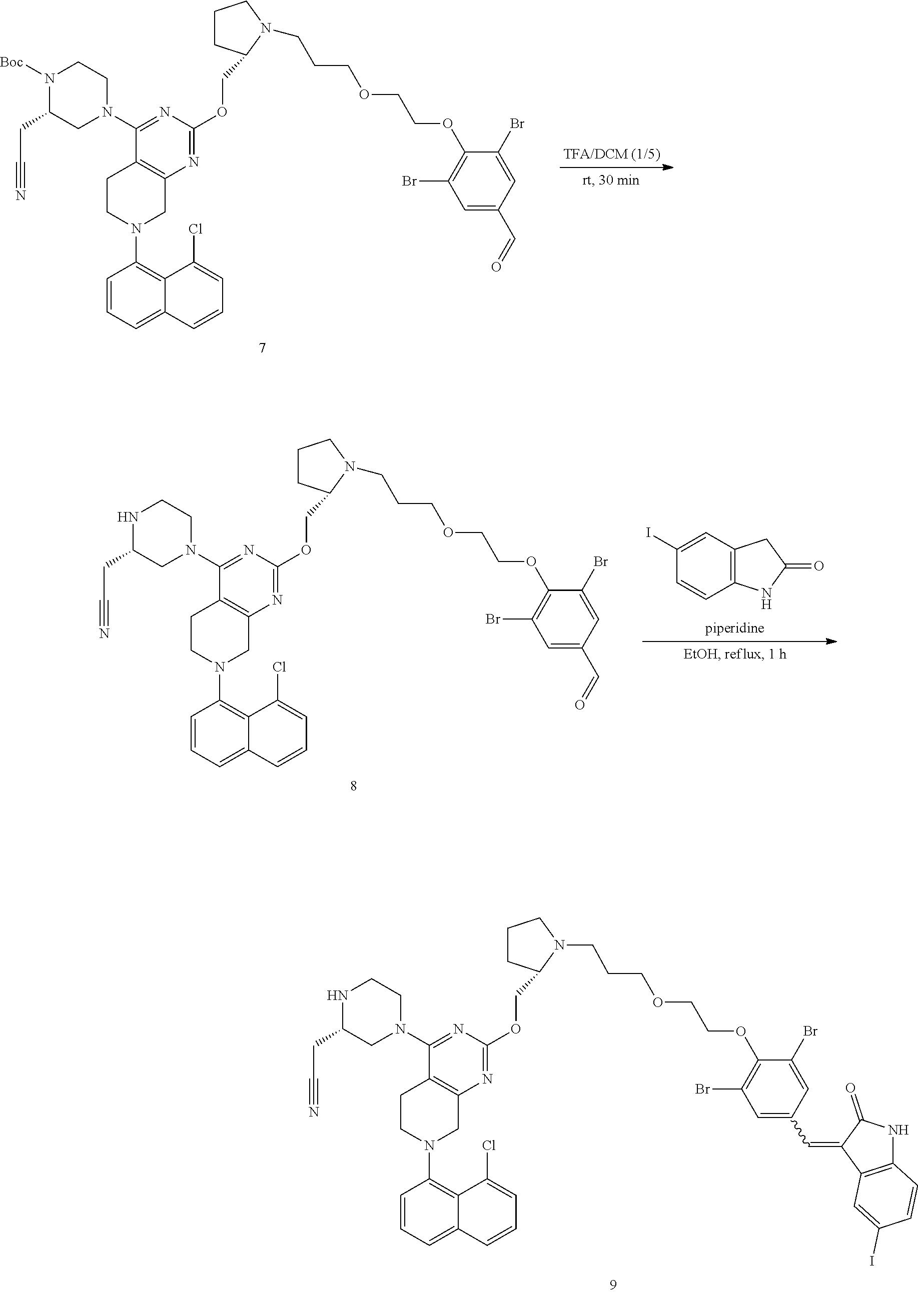 [SMILES: CC(C)(C)OC(=O)N1CCN(c2nc(OC[C@@H]3CCCN3CCCOCCOc3c(Br)cc(C=O)cc3Br)nc3c2CCN(c2cccc4cccc(Cl)c24)C3)C[C@@H]1CC#N ; N#CC[C@H]1CN(c2nc(OC[C@@H]3CCCN3CCCOCCOc3c(Br)cc(C=O)cc3Br)nc3c2CCN(c2cccc4cccc(Cl)c24)C3)CCN1 ; O=C1Cc2cc(I)ccc2N1 ; N#CC[C@H]1CN(c2nc(OC[C@@H]3CCCN3CCCOCCOc3c(Br)cc(C=C4C(=O)Nc5ccc(I)cc54)cc3Br)nc3c2CCN(c2cccc4cccc(Cl)c24)C3)CCN1]

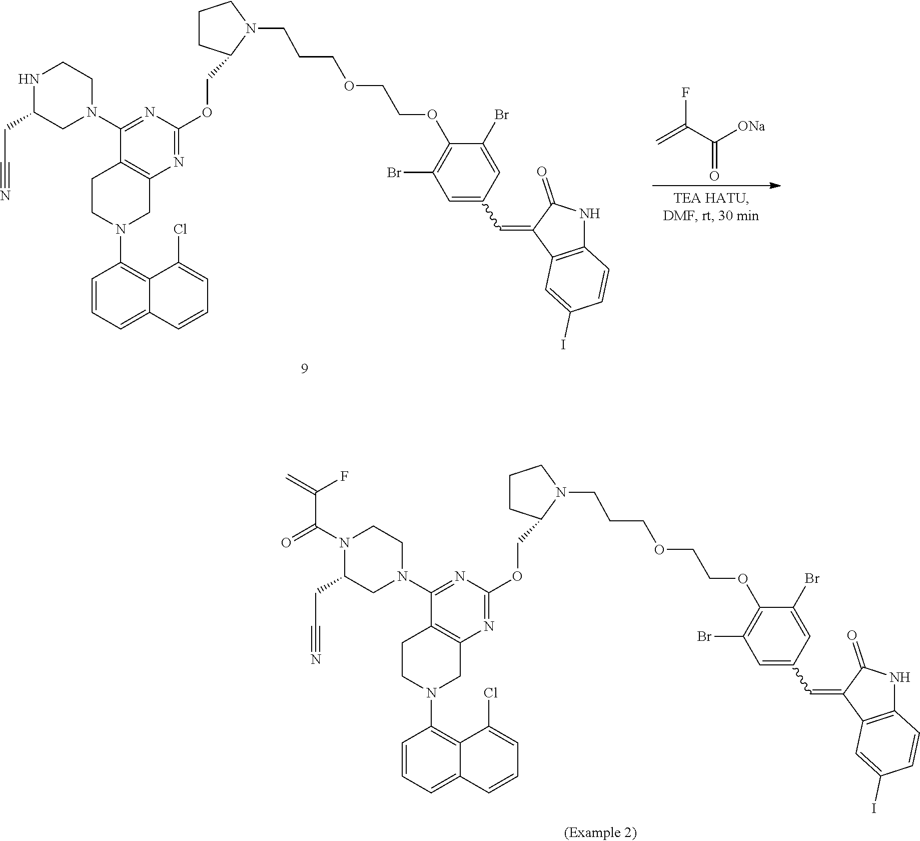

### Step 1: 2-(3-(2-(Benzyloxy)ethoxy)propoxy)-tetrahydro-2H-pyran

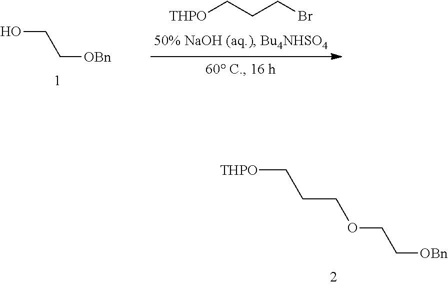

To a stirred solution of benzyl glycol (3.50 g, 23.00 mmol) in THF (30 mL) were added 2-(3-bromopropoxy)oxane (10.26 g, 45.99 mmol), tetrabutylammonium hydrogen sulfate (0.78 g, 2.30 mmol) and 50% NaOH (aq.) (40 mL) at room temperature under a nitrogen atmosphere. The resulting mixture was stirred for 16 h at 60° C. under a nitrogen atmosphere. After cooling down to room temperature, the resulting mixture was diluted with H₂O (100 mL). The resulting mixture was extracted with ethyl acetate (3×200 mL). The combined organic layers were washed with brine (3×100 mL) and dried over anhydrous Na₂SO₄. After filtration, the filtrate was concentrated under reduced pressure. The residue was purified by silica gel column chromatography, eluted with 2%˜10% ethyl acetate in petroleum ether to afford 2-(3-(2-(benzyloxy)ethoxy)propoxy)-tetrahydro-2H-pyran (5.5 g, 82%) as a yellow oil: ¹H NMR (400 MHz, CDCl₃) δ 7.38-7.30 (m, 4H), 7.30-7.24 (m, 1H), 4.59-4.56 (m, 3H), 3.90-3.78 (m, 2H), 3.62 (s, 4H), 3.61-3.55 (m, 2), 3.55-3.45 (m, 2H), 1.94-1.86 (m, 2H), 1.86-1.75 (m, 1H), 1.75-1.66 (m, 1H), 1.62-1.46 (m, 4H); LC/MS (ESI, m/z): [(M+1)]+=295.1.

### Step 2: 2-(3-(Tetrahydro-2H-pyran-2-yloxy)propoxy)ethanol

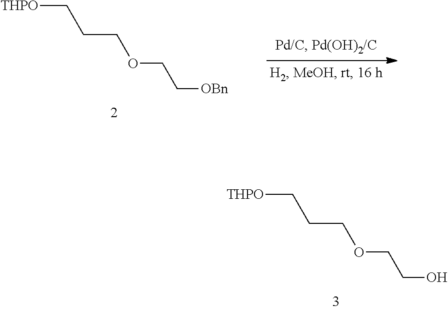

To a stirred solution of 2-(3-(2-(benzyloxy)ethoxy)propoxy)oxane (3.00 g, 10.19 mmol) in methanol (90 mL) were added Pd(OH)₂/C (1.50 g, 10% wt.) and Pd/C (1.50 g, 10% wt.) at ambient temperature under a nitrogen atmosphere. The resulting mixture was stirred for 16 h at ambient temperature under a hydrogen atmosphere (2 atm). The resulting mixture was filtered and the filtered cake was washed with methanol (3×50 mL). The filtrate was concentrated under reduced pressure. The residue was purified by silica gel column chromatography, eluted with 10% methanol in dichloromethane to afford 2-(3-(Tetrahydro-2H-pyran-2-yloxy)propoxy) ethanol (1.6 g, 77%) as a colorless oil: ¹H NMR (400 MHz, CDCl₃) δ 4.59-4.54 (m, 1H), 3.91-3.82 (m, 2H), 3.74-3.70 (m, 2H), 3.62-3.45 (m, 6H), 1.93-1.77 (m, 3H), 1.76-1.66 (m, 1H), 1.62-1.46 (m, 4H); LC/MS (ESI, m/z): [(M+1)]+=205.1.

### Step 3: 3,5-Dibromo-4-(2-(3-(tetrahydro-2H-pyran-2-yloxy)propoxy)ethoxy) benzaldehyde

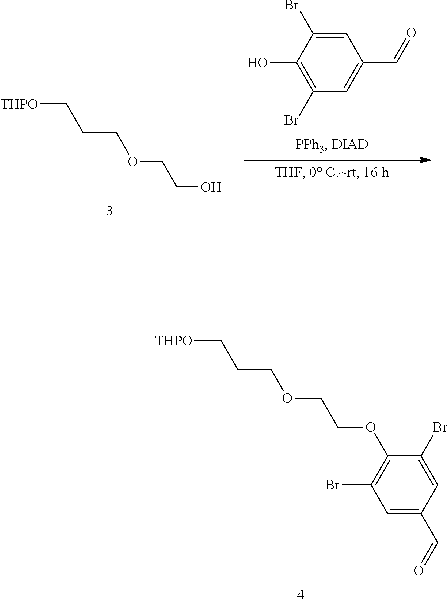

To a stirred solution of 2-(3-(tetrahydro-2H-pyran-2-yloxy)propoxy)ethanol (694 mg, 4.50 mmol) in THF (35 mL) were added 3,5-dibromo-4-hydroxybenzaldehyde (787 mg, 2.83 mmol), triphenylphosphine (1.48 g, 5.66 mmol) and diisopropyl azodicarboxylate (1.14 g, 5.66 mmol) dropwise over 5 min at 0° C. The resulting mixture was stirred for additional 16 h at ambient temperature. The resulting mixture was concentrated under reduced pressure. The residue was purified by silica gel column chromatography, eluted with 10%˜50% ethyl acetate in petroleum ether to afford 3,5-dibromo-4-(2-(3-(tetrahydro-2H-pyran-2-yloxy)propoxy)ethoxy) benzaldehyde (955 mg, 73%) as a colorless oil: ¹H NMR (400 MHz, CDCl₃) δ 9.85 (d, J=1.5 Hz, 1H), 8.03 (d, J=2.2 Hz, 2H), 4.61-4.54 (m, 1H), 4.35-4.23 (m, 2H), 3.94-3.74 (m, 4H), 3.71-3.60 (m, 2H), 3.56-3.44 (m, 2H), 1.96-1.84 (m, 2H), 1.84-1.76 (m, 1H), 1.76-1.64 (m, 1H), 1.64-1.44 (m, 4H); LC/MS (ESI, m/z): [(M+1)]+=467.9.

### Step 4: 3,5-Dibromo-4-(2-(3-hydroxypropoxy)ethoxy)benzaldehyde

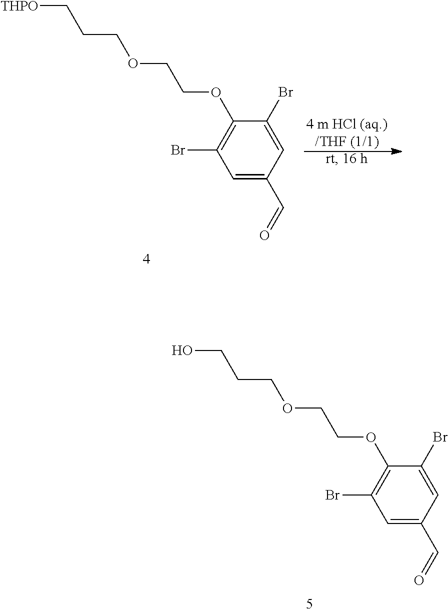

To a stirred solution of 3,5-dibromo-4-(2-(3-(tetrahydro-2H-pyran-2-yloxy)propoxy) ethoxy)benzaldehyde (900 mg, 1.93 mmol) in THF (25 mL) was added HCl (aq.) (2 M) (25 mL) at ambient temperature under a nitrogen atmosphere. The resulting mixture was stirred for 16 h at ambient temperature under a nitrogen atmosphere. The resulting mixture was extracted with ethyl acetate (3×150 mL). The combined organic layers were washed with brine (5×50 mL) and dried over anhydrous Na₂SO₄. After filtration, the filtrate was concentrated under reduced pressure. The residue was purified by silica gel column chromatography, eluted with 1%˜5% methanol in dichloromethane to afford 3,5-dibromo-4-(2-(3-hydroxypropoxy)ethoxy) benzaldehyde (620 mg, 85%) as a colorless oil: ¹H NMR (400 MHz, CDCl₃) δ 9.86 (s, 1H), 8.03 (s, 2H), 4.31-4.24 (m, 2H), 3.94-3.87 (m, 2H), 3.84-3.74 (m, 4H), 1.93-1.83 (m, 2H); LC/MS (ESI, m/z): [(M+1)]+=382.9.

### Step 5: 3-(2-(2,6-Dibromo-4-formylphenoxy)ethoxy)propyl methanesulfonate

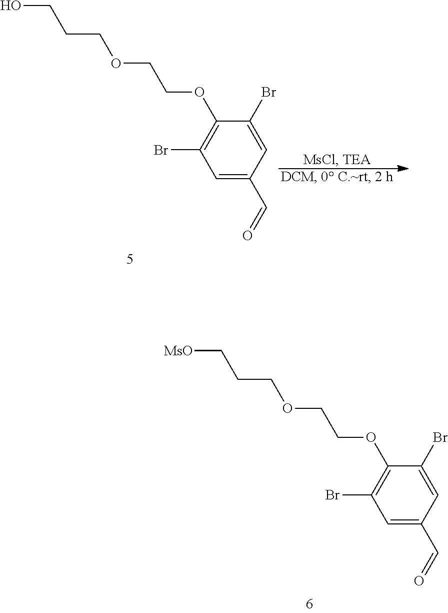 [SMILES: O=Cc1cc(Br)c(OCCOCCCO)c(Br)c1 ; CS(=O)(=O)OCCCOCCOc1c(Br)cc(C=O)cc1Br]

To a stirred solution of 3,5-dibromo-4-(2-(3-hydroxypropoxy)ethoxy)benzaldehyde (620 mg, 1.62 mmol) in DCM (20 mL) were added triethylamine (328 mg, 3.25 mmol) and methanesulfonyl chloride (370 mg, 3.24 mmol) dropwise over 5 min at 0° C. The resulting mixture was stirred for additional 2 h at ambient temperature. The reaction was quenched with water (30 mL) at 0° C. The resulting mixture was extracted with dichloromethane (3×50 mL). The combined organic layers were washed with brine (3×50 mL) and dried over anhydrous Na₂SO₄. After filtration, the filtrate was concentrated under reduced pressure. The residue was purified by silica gel column chromatography, eluted with 50% ethyl acetate in petroleum ether to afford 3-(2-(2,6-dibromo-4-formylphenoxy)ethoxy)propyl methanesulfonate (660 mg, 89%) as a colorless oil: ¹H NMR (400 MHz, CDCl₃) δ 9.86 (s, 1H), 8.04 (s, 2H), 4.35 (t, J=6.3 Hz, 2H), 4.28 (t, J=4.6 Hz, 2H), 3.90 (t, J=4.6 Hz, 2H), 3.68 (t, J=5.9 Hz, 2H), 3.02 (s, 3H), 2.08-1.99 (m, 2H); LC/MS (ESI, m/z): [(M+1)]+=460.0.

### Step 6: (S)-tert-butyl 4-(7-(8-chloronaphthalen-1-yl)-2-(((S)-1-(3-(2-(2,6-dibromo-4-formylphenoxy)ethoxy)propyl)pyrrolidin-2-yl)methoxy)-5,6,7,8-tetrahydropyrido[3,4-d]pyrimidin-4-yl)-2-(cyanomethyl)piperazine-1-carboxylate

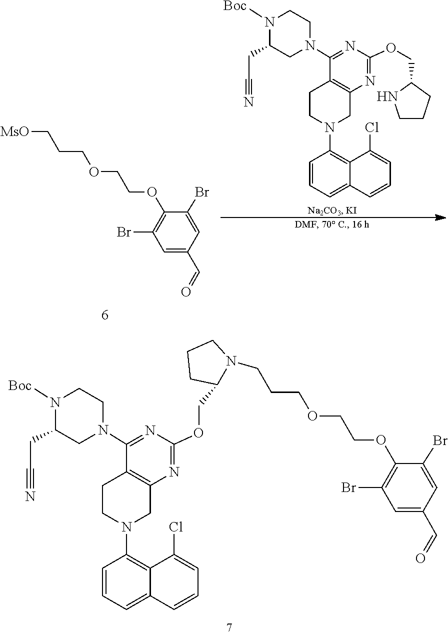 [SMILES: CS(=O)(=O)OCCCOCCOc1c(Br)cc(C=O)cc1Br ; CC(C)(C)OC(=O)N1CCN(c2nc(OC[C@@H]3CCCN3)nc3c2CCN(c2cccc4cccc(Cl)c24)C3)C[C@@H]1CC#N ; CC(C)(C)OC(=O)N1CCN(c2nc(OC[C@@H]3CCCN3CCCOCCOc3c(Br)cc(C=O)cc3Br)nc3c2CCN(c2cccc4cccc(Cl)c24)C3)C[C@@H]1CC#N]

To a stirred solution of tert-butyl (2S)-4-(7-(8-chloronaphthalen-1-yl)-2-((2S)-pyrrolidin-2-ylmethoxy)-5H,6H,8H-pyrido[3,4-d]pyrimidin-4-yl)-2-(cyanomethyl)piperazine-1-carboxylate (333 mg, 0.54 mmol) in DMF (10 mL) were added 3-(2-(2,6-dibromo-4-formylphenoxy) ethoxy)propyl methanesulfonate (273 mg, 0.59 mmol), KI (89 mg, 0.54 mmol) and Na₂CO₃ (114 mg, 1.08 mmol) at room temperature. The resulting mixture was stirred for 16 h at 70° C. After cooling down to room temperature, the resulting mixture was diluted with water (100 mL) and extracted with ethyl acetate (3×20 mL). The combined organic layers were washed with brine (3×10 mL) and dried over anhydrous Na₂SO₄. After filtration, the filtrate was concentrated under reduced pressure. The residue was purified by silica gel column chromatography, eluted with 8% methanol in dichloromethane to afford (S)-tert-butyl 4-(7-(8-chloronaphthalen-1-yl)-2-(((S)-1-(3-(2-(2,6-dibromo-4-formylphenoxy)ethoxy)propyl)pyrrolidin-2-yl)methoxy)-5,6,7,8-tetrahydropyrido[3,4-d]pyrimidin-4-yl)-2-(cyanomethyl)piperazine-1-carboxylate (290 mg, 55%) as an off-white solid: ¹H NMR (400 MHz, CDCl₃) δ 9.80 (d, J=1.9 Hz, 1H), 7.97 (d, J=1.5 Hz, 2H), 7.73 (d, J=8.3 Hz, 1H), 7.59 (t, J=7.5 Hz, 1H), 7.50 (d, J=7.5 Hz, 1H), 7.46-7.35 (m, 1H), 7.31 (t, J=7.8 Hz, 1H), 7.20 (dd, J=23.0, 7.6 Hz, 1H), 4.59 (s, 1H), 4.44-4.33 (m, 1H), 4.26-4.17 (m, 2H), 4.14-3.88 (m, 4H), 3.88-3.73 (m, 3H), 3.69-3.49 (m, 3H), 3.43-3.37 (m, 1H), 3.27-3.02 (m, 4H), 3.02-2.83 (m, 2H), 2.83-2.64 (m, 2H), 2.54 (t, J=13.4 Hz, 1H), 2.11-1.55 (m, 10H), 1.50 (s, 9H); LC/MS (ESI, m/z): [(M+1)]⁺=982.4.

### Step 7: 2-((S)-4-(7-(8-chloronaphthalen-1-yl)-2-(((S)-1-(3-(2-(2,6-dibromo-4-formyl phenoxy)ethoxy)propyl)pyrrolidin-2-yl)methoxy)-5,6,7,8-tetrahydropyrido[3,4-d]pyrimidin-4-yl)piperazin-2-yl)acetonitrile

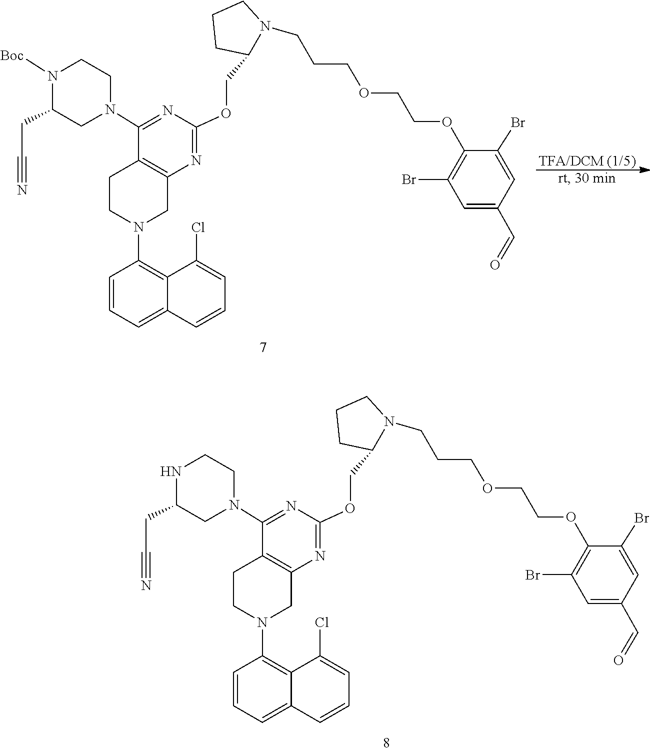 [SMILES: CC(C)(C)OC(=O)N1CCN(c2nc(OC[C@@H]3CCCN3CCCOCCOc3c(Br)cc(C=O)cc3Br)nc3c2CCN(c2cccc4cccc(Cl)c24)C3)C[C@@H]1CC#N ; N#CC[C@H]1CN(c2nc(OC[C@@H]3CCCN3CCCOCCOc3c(Br)cc(C=O)cc3Br)nc3c2CCN(c2cccc4cccc(Cl)c24)C3)CCN1]

To a stirred solution of (S)-tert-butyl 4-(7-(8-chloronaphthalen-1-yl)-2-(((S)-1-(3-(2-(2,6-dibromo-4-formylphenoxy)ethoxy)propyl)pyrrolidin-2-yl)methoxy)-5,6,7,8-tetrahydropyrido [3,4-d]pyrimidin-4-yl)-2-(cyanomethyl)piperazine-1-carboxylate (150 mg, 0.153 mmol) in DCM (5 mL) was added trifluoroacetic acid (1 mL) dropwise at ambient temperature under a nitrogen atmosphere. The resulting mixture was stirred for 30 min at ambient temperature under a nitrogen atmosphere. The resulting mixture was concentrated under reduced pressure. The residue was purified by Prep-TLC (CH₂Cl₂/methanol=8:1) to afford 2-((S)-4-(7-(8-chloro naphthalen-1-yl)-2-(((S)-1-(3-(2-(2,6-dibromo-4-formylphenoxy)ethoxy)propyl) pyrrolidin-2-yl)methoxy)-5,6,7,8-tetrahydropyrido[3,4-d]pyrimidin-4-yl)piperazin-2-yl)acetonitrile (90 mg, 67%) as a yellow solid: ¹H NMR (400 MHz, CDCl₃) δ 9.81 (s, 1H), 7.98 (d, J=1.2 Hz, 2H), 7.74 (d, J=8.1 Hz, 1H), 7.62-7.57 (m, 1H), 7.50 (d, J=7.4 Hz, 1H), 7.46-7.39 (m, 1H), 7.32 (t, J=7.8 Hz, 1H), 7.21 (t, J=7.7 Hz, 1H), 4.45-4.29 (m, 2H), 4.28-4.18 (m, 2H), 4.10-3.96 (m, 2H), 3.93-3.77 (m, 4H), 3.72 (d, J=13.0 Hz, 1H), 3.66-3.50 (m, 3H), 3.37-3.27 (m, 1H), 3.24-3.02 (m, 5H), 3.01-2.78 (m, 4H), 2.59-2.37 (m, 4H), 2.27-2.17 (m, 1H), 2.07-1.95 (m, 2H), 1.92-1.78 (m, 3H); LC/MS (ESI, m/z): [(M+1)]⁺=882.3.

### Step 8: 2-((S)-4-(7-(8-chloronaphthalen-1-yl)-2-(((S)-1-(3-(2-(2,6-dibromo-4-((5-iodo-2-oxoindolin-3-ylidene)methyl)phenoxy)ethoxy)propyl)pyrrolidin-2-yl)methoxy)-5,6,7,8-tetrahydropyrido[3,4-d]pyrimidin-4-yl)piperazin-2-yl)acetonitrile

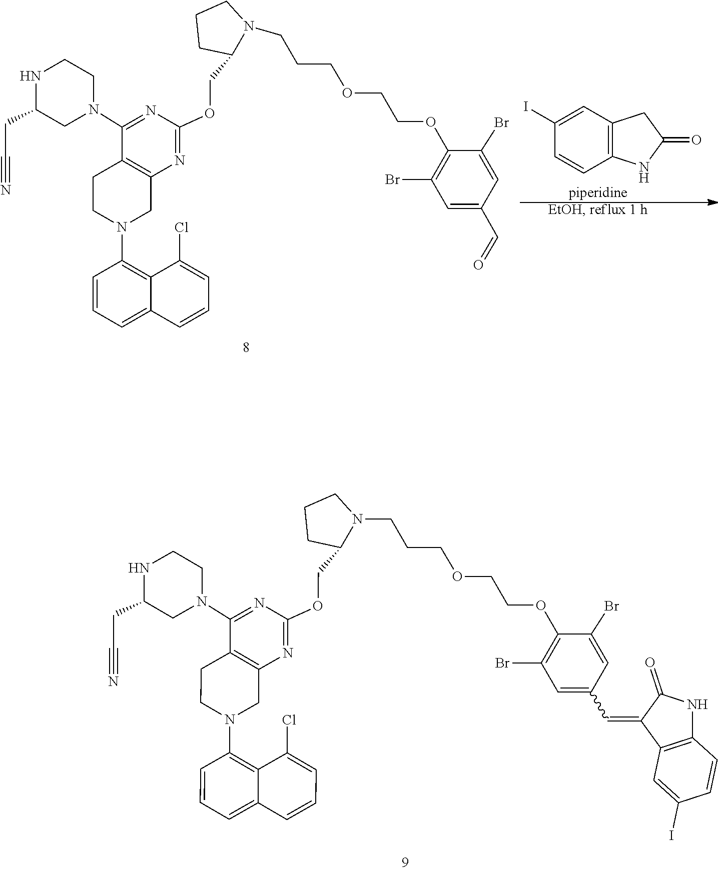

To a stirred mixture of 2-((S)-4-(7-(8-chloronaphthalen-1-yl)-2-(((S)-1-(3-(2-(2,6-dibromo-4-formylphenoxy)ethoxy)propyl)pyrrolidin-2-yl)methoxy)-5,6,7,8-tetrahydropyrido [3,4-d]pyrimidin-4-yl)piperazin-2-yl)acetonitrile (90 mg, 0.10 mmol) in ethanol (3 mL) were added 5-iodo-1,3-dihydroindol-2-one (27 mg, 0.10 mmol) and piperidine (9 mg, 0.10 mmol) at ambient temperature under a nitrogen atmosphere. The resulting mixture was stirred for 30 min at 80° C. under a nitrogen atmosphere. The resulting mixture was concentrated under reduced pressure. The residue was purified by reverse phase flash chromatography with the following conditions: Column, Spherical C18, 20˜40 μm, 80 g; Eluent A: Water (plus 10 mM NH₄HCO₃ and 0.02% NH₃·H₂O); Eluent B: ACN; Flow rate: 50 mL/min; Gradient: 80% B to 92% B in 30 min; Detector: UV 220/254 nm. The fractions containing desired product were collected and concentrated under reduced pressure to afford 2-((S)-4-(7-(8-chloronaphthalen-1-yl)-2-(((S)-1-(3-(2-(2,6-dibromo-4-((5-iodo-2-oxoindolin-3-ylidene)methyl)phenoxy)ethoxy)propyl)pyrrolidin-2-yl)methoxy)-5,6,7,8-tetrahydropyrido[3,4-d]pyrimidin-4-yl)piperazin-2-yl)acetonitrile (70 mg, 62%) as a yellow solid; ¹H NMR (400 MHz, DMSO-d₆) δ 10.78 (d, J=19.9 Hz, 1H), 8.74 (s, 1H), 8.07-7.79 (m, 3H), 7.76-7.64 (m, 1H), 7.60-7.45 (m, 3H), 7.41 (t, J=7.8 Hz, 1H), 7.33-7.24 (m, 1H), 6.71 (dd, J=16.6, 8.2 Hz, 1H), 4.44-3.79 (m, 8H), 3.78-3.56 (m, 4H), 3.56-3.38 (m, 4H), 3.16-2.75 (m, 8H), 2.75-2.59 (m, 4H), 2.01-1.85 (m, 1H), 1.85-1.56 (m, 5H); LC/MS (ESI, m/z): [(M+1)]⁺=1123.3.

### Step 9: 2-((S)-4-(7-(8-chloronaphthalen-1-yl)-2-(((S)-1-(3-(2-(2,6-dibromo-4-((5-iodo-2-oxoindolin-3-ylidene)methyl)phenoxy)ethoxy)propyl)pyrrolidin-2-yl)methoxy)-5,6,7,8-tetrahydropyrido[3,4-d]pyrimidin-4-yl)-1-(2-fluoroacryloyl)piperazin-2-yl)acetonitrile

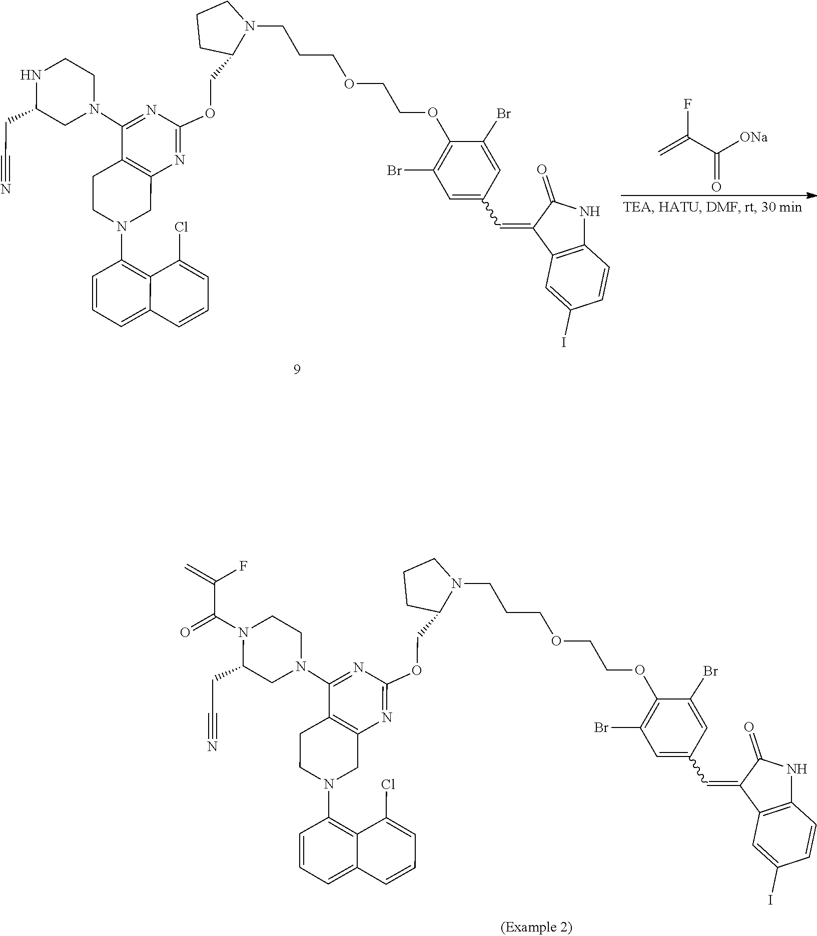

To a stirred solution of 2-((S)-4-(7-(8-chloronaphthalen-1-yl)-2-(((S)-1-(3-(2-(2,6-dibromo-4-((5-iodo-2-oxoindolin-3-ylidene)methyl)phenoxy)ethoxy)propyl)pyrrolidin-2-yl)methoxy)-5,6,7,8-tetrahydropyrido[3,4-d]pyrimidin-4-yl)piperazin-2-yl)acetonitrile (70 mg, 0.062 mmol) in DMF (3 mL) were added triethylamine (19 mg, 0.19 mmol), sodium 2-fluoro prop-2-enoate (14 mg, 0.12 mmol) and HATU (36 mg, 0.093 mmol) at room temperature under a nitrogen atmosphere. The resulting mixture was stirred for 30 min at room temperature. The resulting mixture was concentrated under reduced pressure. The residue was purified by Prep-HPLC with the following conditions: Column: XSelect CSH Prep C18 OBD Column, 19*250 mm, 5 um; Eluent A: Water (plus 10 mM NH₄HCO₃); Eluent B: ACN; Flow rate: 25 mL/min; Gradient: 70% B to 90% B in 8 min; Detector: UV 220/254 nm. The fractions containing desired product were collected and concentrated under reduced pressure and lyophilized to afford 2-((S)-4-(7-(8-chloronaphthalen-1-yl)-2-(((S)-1-(3-(2-(2,6-dibromo-4-((5-iodo-2-oxoindolin-3-ylidene)methyl)phenoxy)ethoxy)propyl)pyrrolidin-2-yl)methoxy)-5,6,7,8-tetrahydropyrido[3,4-d]pyrimidin-4-yl)-1-(2-fluoroacryloyl)piperazin-2-yl)acetonitrile (9.1 mg, 3%) as a yellow solid: ¹H NMR (400 MHz, DMSO-d₆) δ 10.81 (d, J=20.4 Hz, 1H), 8.75 (s, 1H), 8.16-7.79 (m, 3H), 7.75-7.66 (m, 1H), 7.60-7.46 (m, 3H), 7.42 (t, J=7.8 Hz, 1H), 7.30 (dd, J=14.2, 7.5 Hz, 1H), 6.75-6.66 (m, 1H), 5.46-5.12 (m, 2H), 5.02-4.48 (m, 2H), 4.34-3.82 (m, 8H), 3.83-3.57 (m, 4H), 3.57-3.40 (m, 4H), 3.29-2.79 (m, 10H), 2.01-1.82 (m, 1H), 1.82-1.56 (m, 5H); LC/MS (ESI, m/z): [(M+1)]⁺=1195.35.

### Example 3: 2-((S)-4-(7-(8-chloronaphthalen-1-yl)-2-(((S)-1-(3-(2-(2-(2,6-dibromo-4-((5-iodo-2-oxoindolin-3-ylidene)methyl)phenoxy)ethoxy)ethoxy) propyl)pyrrolidin-2-yl)methoxy)-5,6,7,8-tetrahydropyrido[3,4-d]pyrimidin-4-yl)-1-(2-fluoroacryloyl) piperazin-2-yl)acetonitrile

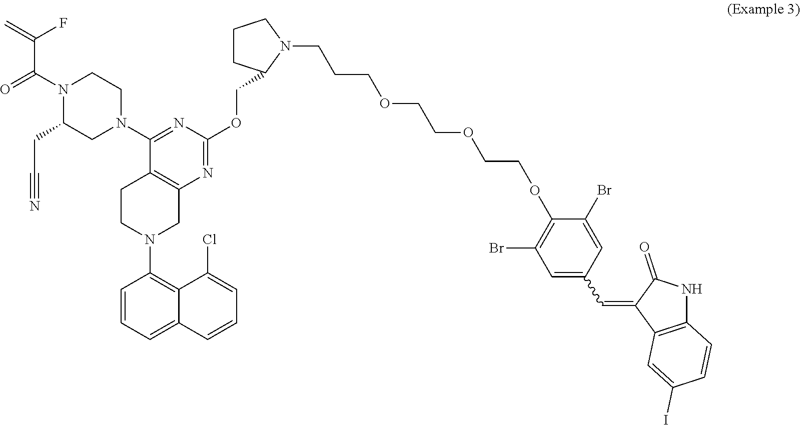

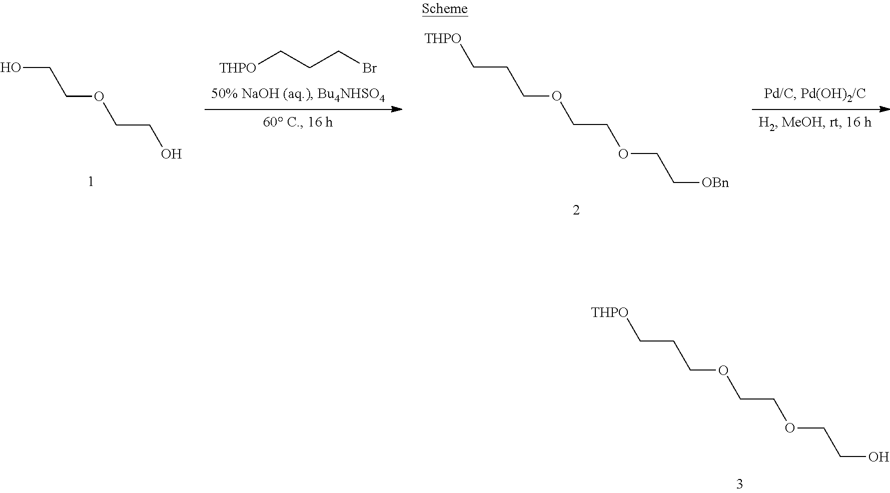

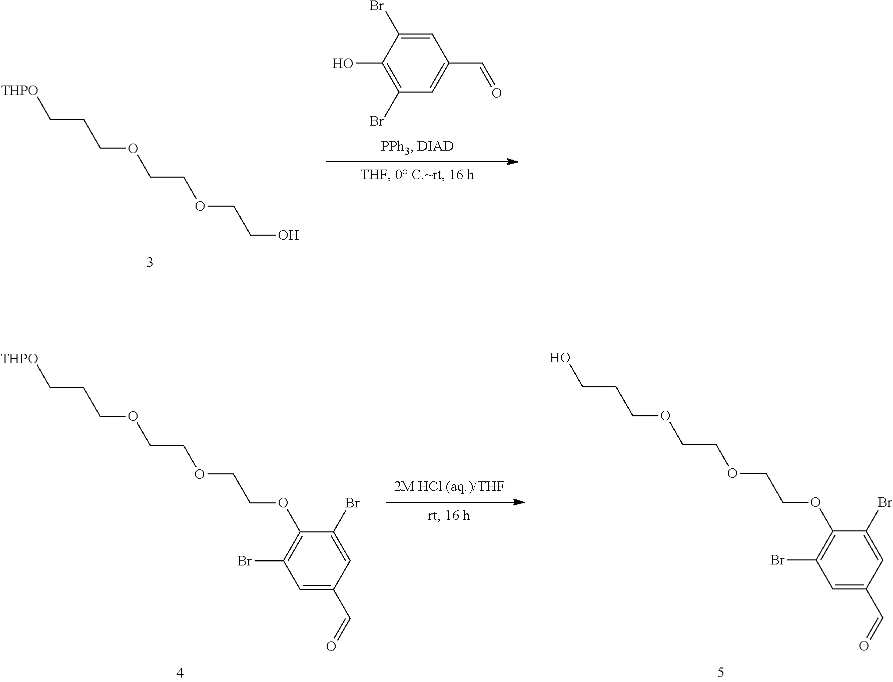

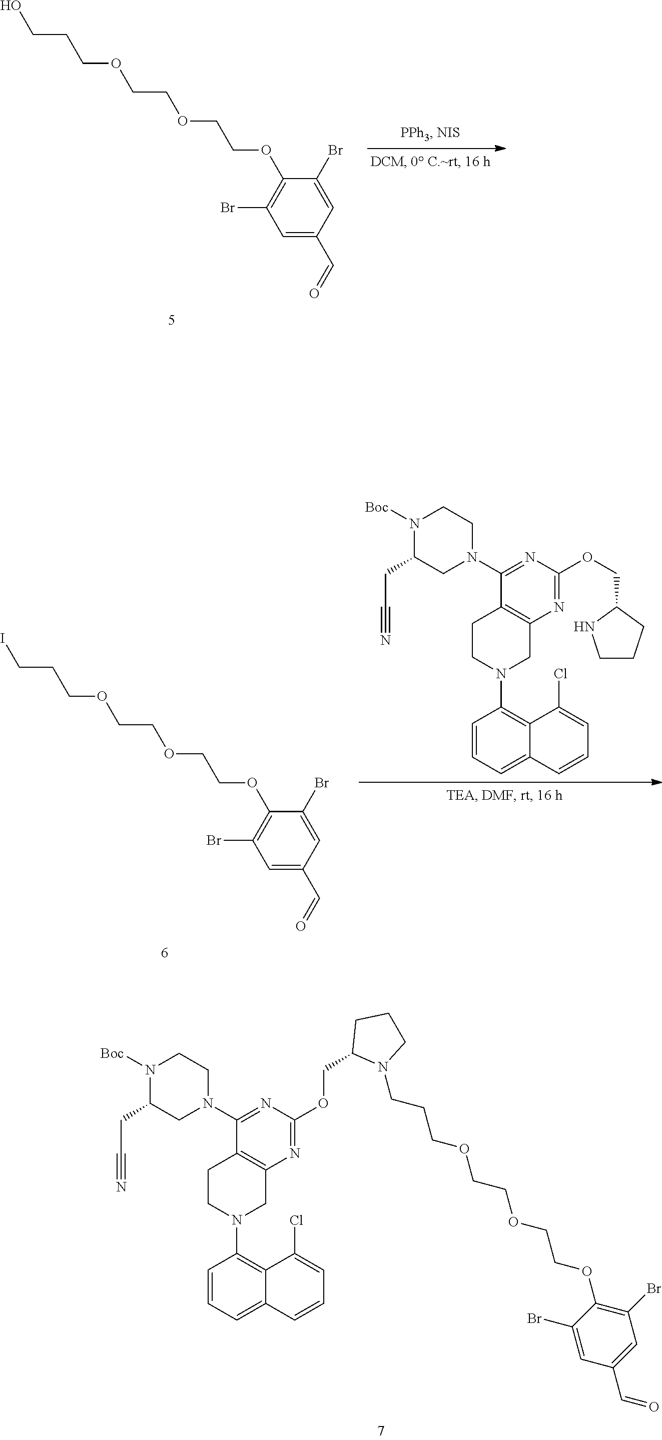

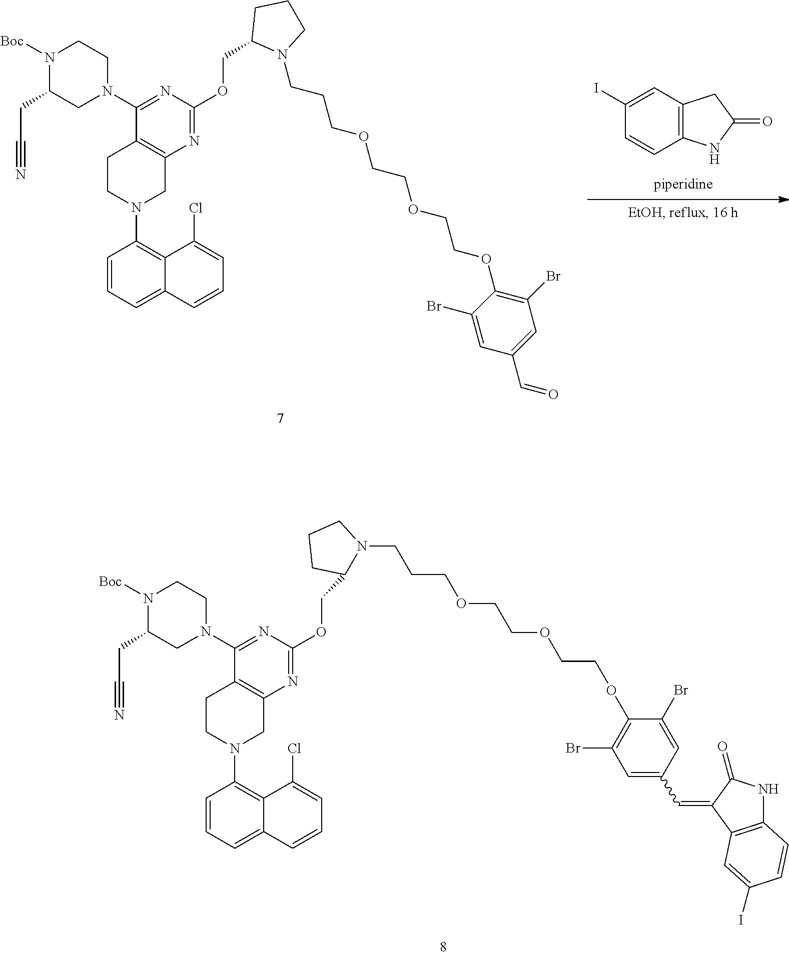

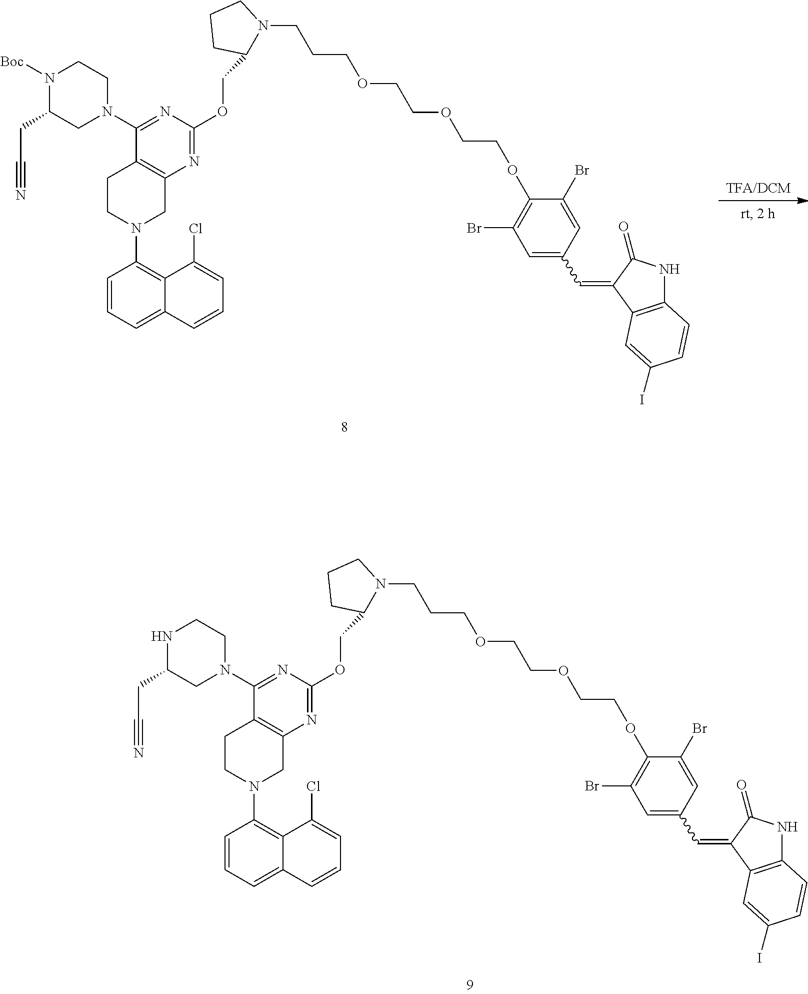

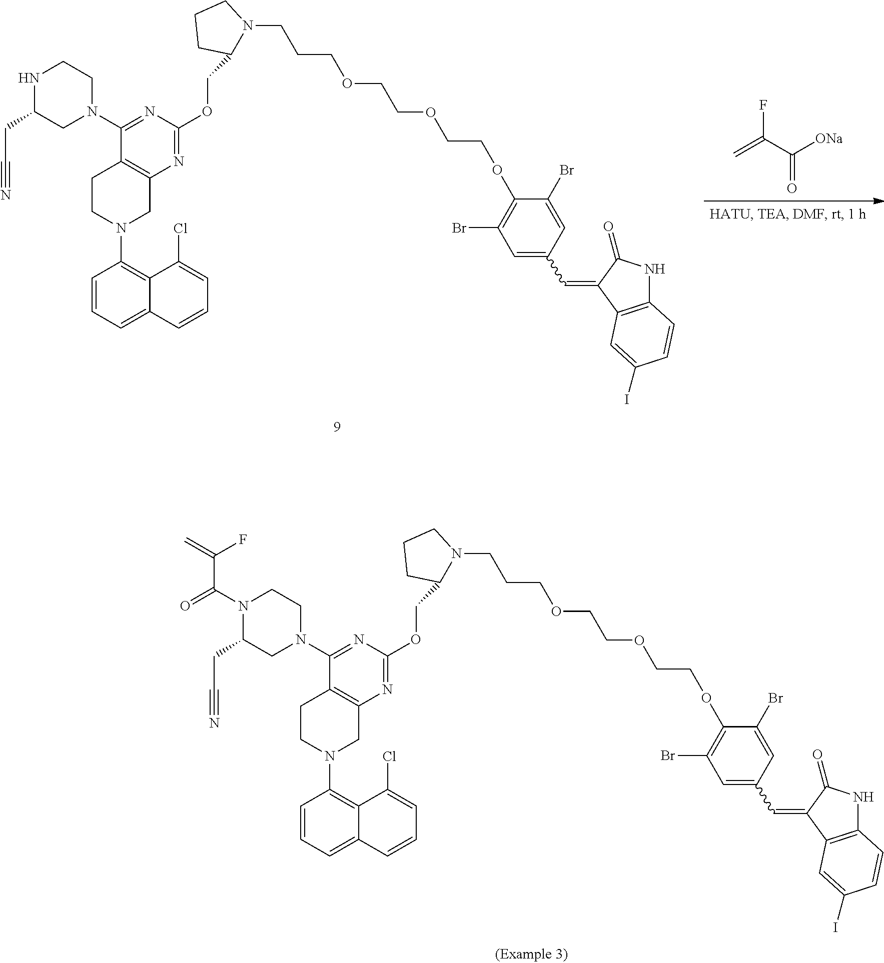

### Step 1: 2-(3-(2-(2-(Benzyloxy)ethoxy)ethoxy)propoxy)-tetrahydro-2H-pyran

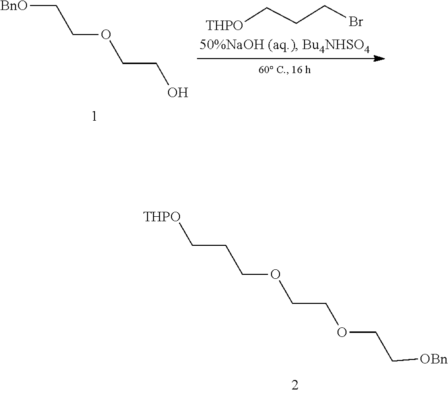

To a stirred solution of 2-(3-bromopropoxy)oxane (5.00 g, 22.41 mmol) in THF (50 mL) were added 2-(2-(benzyloxy)ethoxy)ethan-1-ol (8.80 g, 44.82 mmol), tetrabutylammonium hydrogen sulfate (0.76 g, 2.24 mmol) and 50% NaOH (aq.) (50 mL) at ambient temperature. The resulting mixture was stirred for 16 h at 60° C. under air atmosphere. After cooling down to room temperature, the resulting mixture was extracted with ethyl acetate (3×200 mL). The combined organic layers were washed with brine (3×100 mL) and dried over anhydrous Na₂SO₄. After filtration, the filtrate was concentrated under reduced pressure. The residue was purified by silica gel column chromatography, eluted with 1%˜10% ethyl acetate in petroleum ether to afford 2-(3-(2-(2-(benzyloxy)ethoxy)ethoxy)propoxy)-tetrahydro-2H-pyran (8 g, 93%) as a yellow oil: ¹H NMR (400 MHz, CDCl₃) δ 7.34 (d, J 4.4 Hz, 4H), 7.30-7.24 (i, 1H), 4.57 (s, 3H), 3.91-3.74 (m, 2H), 3.74-3.54 (m, 9H), 3.54-3.44 (m, 3H), 1.97-1.75 (m, 3H), 1.76-1.64 (m, 1H), 1.64-1.44 (in, 4H); LC/MS (ESI, m/z): [(M+1)]⁺=339.2.

### Step 2: 2-(2-(3-(Tetrahydro-2H-pyran-2-yloxy)propoxy)ethoxy)ethanol

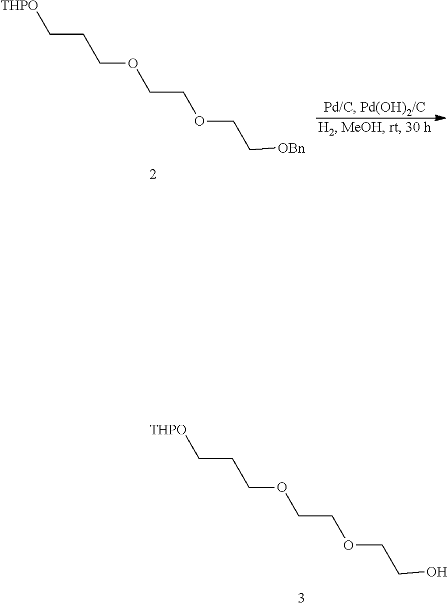

To a stirred solution of 2-(3-(2-(2-(benzyloxy)ethoxy)ethoxy)propoxy)-tetrahydro-2H-pyran (4.00 g, 13.59 mmol) in methanol (90 mL) were added Pd(OH)₂/C (0.60 g, 10% wt.) and Pd/C (0.60 g, 10% wt.) at ambient temperature under a nitrogen atmosphere. The resulting mixture was stirred for 30 h at ambient temperature under a hydrogen atmosphere (2 atm). The resulting mixture was filtered and the filtered cake was washed with methanol (3×50 mL). The filtrate was concentrated under reduced pressure. The residue was purified by silica gel column chromatography, eluted with 10% methanol in dichloromethane to afford 2-(2-(3-(tetrahydro-2H-pyran-2-yloxy)propoxy)ethoxy)ethanol (2.5 g, 91%) as a colorless oil: ¹H NMR (400 MHz, CDCl₃) δ 4.56 (t, J=3.6 Hz, 1H), 3.90-3.75 (m, 2H), 3.75-3.69 (m, 2H), 3.69-3.62 (m, 2H), 3.62-3.52 (m, 6H), 3.52-3.40 (m, 2H), 2.56 (s, 1H), 1.94-1.74 (m, 3H), 1.74-1.63 (m, 1H), 1.62-1.44 (m, 4H). LC/MS (ESI, m/z): [(M+1)]⁺=249.1.

### Step 3: 3,5-Dibromo-4-(2-(2-(3-(tetrahydro-2H-pyran-2-yloxy)propoxy)ethoxy)ethoxy) benzaldehyde

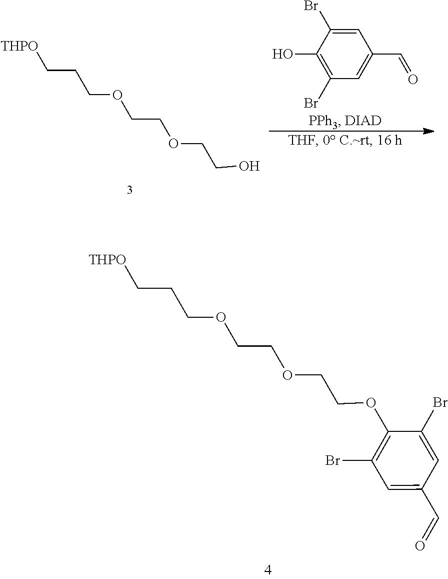

To a stirred solution of 2-(2-(3-(tetrahydro-2H-pyran-2-yloxy)propoxy)ethoxy)ethanol (2.00 g, 8.0 mmol) in THF (100 mL) were added 3,5-dibromo-4-hydroxybenzaldehyde (1.79 g, 6.40 mmol), triphenylphosphine (3.35 g, 12.8 mmol) and diisopropyl azodicarboxylate (2.59 g, 12.8 mmol) dropwise over 5 min at 0° C. The resulting mixture was stirred for 16 h at ambient temperature. The resulting mixture was concentrated under reduced pressure. The residue was purified by silica gel column chromatography, eluted with 10%˜30% ethyl acetate in petroleum ether to afford 3,5-dibromo-4-(2-(2-(3-(tetrahydro-2H-pyran-2-yloxy)propoxy)ethoxy)ethoxy) benzaldehyde (2.6 g, 80%) as an off-white solid: ¹H NMR (400 MHz, CDCl₃) δ 9.85 (s, 1H), 8.03 (s, 2H), 4.60-4.56 (m, 1H), 4.29 (dd, J=5.7, 4.0 Hz, 2H), 3.97-3.92 (m, 2H), 3.89-3.80 (m, 2H), 3.78-3.72 (m, 2H), 3.67-3.55 (m, 4H), 3.54-3.44 (m, 2H), 1.94-1.77 (m, 3H), 1.76-1.64 (m, 1H), 1.63-1.46 (m, 4H); LC/MS (ESI, m/z): [(M+1)]+=511.1.

### Step 4: 3,5-Dibromo-4-(2-(2-(3-hydroxypropoxy)ethoxy)ethoxy)benzaldehyde

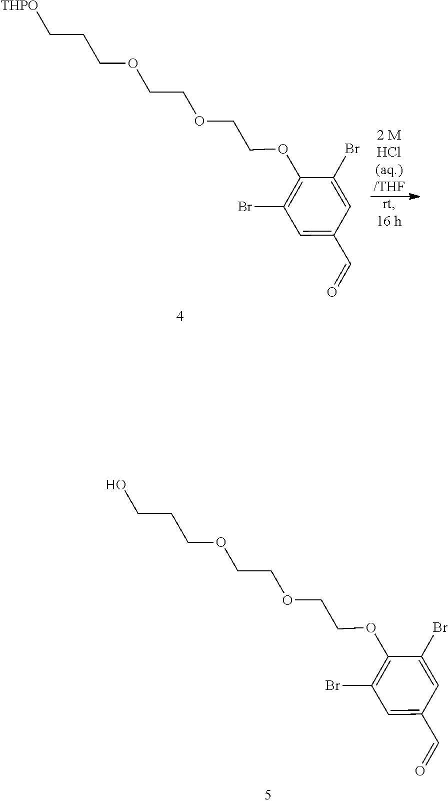

To a stirred solution of 3,5-dibromo-4-(2-(2-(3-(tetrahydro-2H-pyran-2-yloxy)propoxy) ethoxy)ethoxy)benzaldehyde (3.80 g, 7.66 mmol) in THF (80 mL) was added 2M HCl (aq.) (80 mL) at ambient temperature under air atmosphere. The resulting mixture was stirred for 16 h at ambient temperature under air atmosphere. The resulting mixture was extracted with ethyl acetate (3×150 mL). The combined organic layers were washed with brine (5×50 mL) and dried over anhydrous Na₂SO₄. After filtration, the filtrate was concentrated under reduced pressure. The residue was purified by silica gel column chromatography, eluted with 1%˜5% methanol in dichloromethane to afford 3,5-dibromo-4-(2-(2-(3-hydroxypropoxy)ethoxy) ethoxy)benzaldehyde (1.48 g, 46%) as a colorless oil: ¹H NMR (400 MHz, CDCl₃) δ 9.85 (s, 1H), 8.02 (s, 2H), 4.29 (t, J=4.8 Hz, 2H), 3.94 (t, J=4.9 Hz, 2H), 3.82-3.74 (m, 4H), 3.72-3.61 (m, 5H), 1.88-1.80 (m, 2H); LC/MS (ESI, m/z): [(M+1)]⁺=427.0.

### Step 5: 3,5-Dibromo-4-(2-(2-(3-iodopropoxy)ethoxy)ethoxy)benzaldehyde

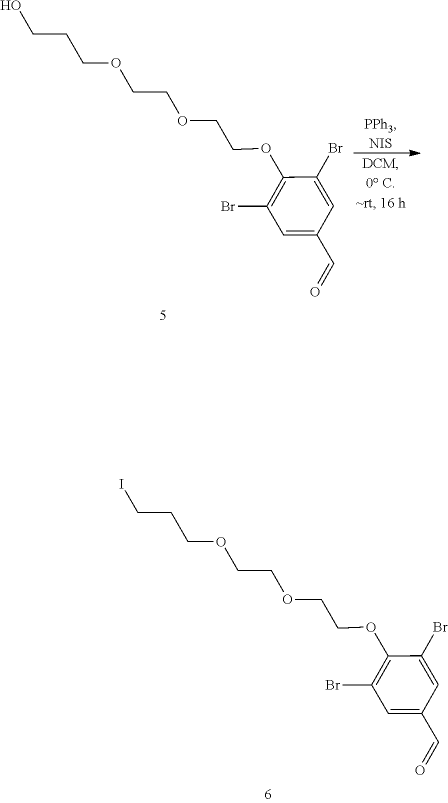 [SMILES: O=Cc1cc(Br)c(OCCOCCOCCCO)c(Br)c1 ; O=Cc1cc(Br)c(OCCOCCOCCCI)c(Br)c1]

To a stirred solution of 3,5-dibromo-4-(2-(2-(3-hydroxypropoxy)ethoxy)ethoxy) benzaldehyde (890 mg, 2.10 mmol) in DCM (80 mL) was added triphenylphosphine (1.10 g, 4.20 mmol) at ambient temperature under a nitrogen atmosphere. To the above mixture was added NIS (945 mg, 4.20 mmol) in portions over 2 min at 0° C. The resulting mixture was stirred for 16 h at ambient temperature. The resulting mixture was concentrated under reduced pressure. The residue was purified by silica gel column chromatography, eluted with 20% ethyl acetate in petroleum ether to afford 3,5-dibromo-4-(2-(2-(3-iodopropoxy)ethoxy)ethoxy)benzaldehyde (848 mg, 76%) as an off-white oil: ¹H NMR (400 MHz, CDCl₃) δ 9.86 (dd, J=5.0, 2.6 Hz, 1H), 8.03 (dd, J=5.0, 2.6 Hz, 2H), 4.30 (t, J=5.8 Hz, 2H), 3.96 (t, J=5.4 Hz, 2H), 3.79-3.72 (m, 2H), 3.64 (t, J=5.2 Hz, 2H), 3.55 (t, J=6.0 Hz, 2H), 3.32-3.24 (m, 2H), 2.12-2.01 (m, 2H); LC/MS (ESI, m/z): [(M+1)]+=536.9.

### Step 6: (S)-tert-butyl 4-(7-(8-chloronaphthalen-1-yl)-2-(((S)-1-(3-(2-(2-(2,6-dibromo-4-formyl phenoxy)ethoxy)ethoxy)propyl)pyrrolidin-2-yl)methoxy)-5,6,7,8-tetrahydropyrido[3,4-d]pyrimidin-4-yl)-2-(cyanomethyl)piperazine-1-carboxylate

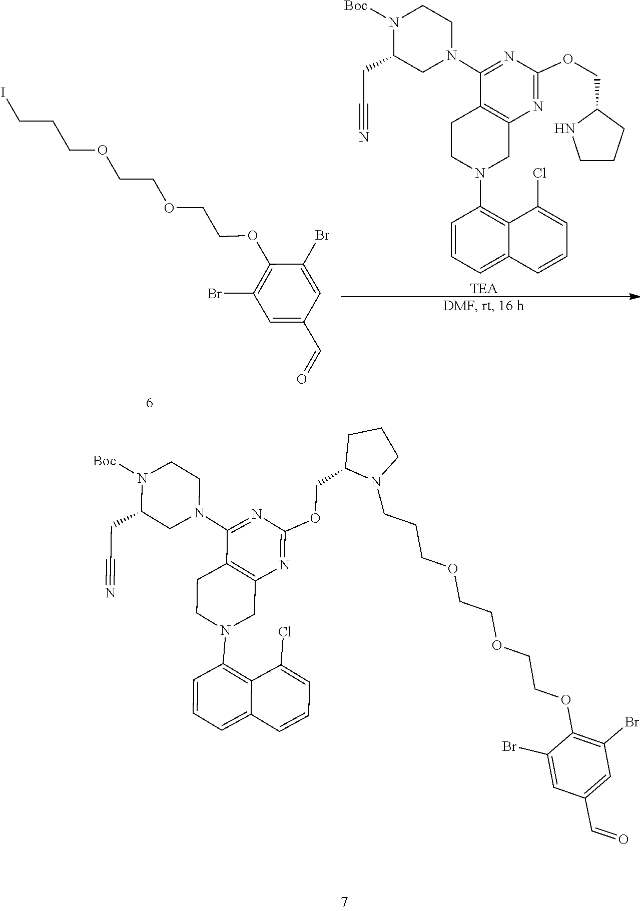 [SMILES: O=Cc1cc(Br)c(OCCOCCOCCCI)c(Br)c1 ; CC(C)(C)OC(=O)N1CCN(c2nc(OC[C@@H]3CCCN3)nc3c2CCN(c2cccc4cccc(Cl)c24)C3)C[C@@H]1CC#N ; CC(C)(C)OC(=O)N1CCN(c2nc(OC[C@@H]3CCCN3CCCOCCOCCOc3c(Br)cc(C=O)cc3Br)nc3c2CCN(c2cccc4cccc(Cl)c24)C3)C[C@@H]1CC#N]

To a solution tert-butyl (2S)-4-[7-(8-chloronaphthalen-1-yl)-2-[(2S)-pyrrolidin-2-ylmethoxy]-5H,6H,8H-pyrido[3,4-d]pyrimidin-4-yl]-2-(cyanomethyl)piperazine-1-carboxylate (336 mg, 0.54 mmol) in DMF (6 mL) were added 3,5-dibromo-4-(2-(2-(3-iodopropoxy)ethoxy) ethoxy)benzaldehyde (350 mg, 0.65 mmol) and triethylamine (165 mg, 1.63 mmol). The resulting mixture was stirred for 16 h at room temperature under a nitrogen atmosphere. The resulting mixture was diluted with water (50 mL) and extracted with ethyl acetate (3×10 mL). The combined organic layers were washed with brine (3×10 mL) and dried over anhydrous Na₂SO₄. After filtration, the filtrate was concentrated under reduced pressure. The residue was purified by silica gel column chromatography, eluted with 1%˜8% methanol in dichloromethane to afford (S)-tert-butyl 4-(7-(8-chloronaphthalen-1-yl)-2-(((S)-1-(3-(2-(2-(2,6-dibromo-4-formylphenoxy) ethoxy)ethoxy)propyl)pyrrolidin-2-yl)methoxy)-5,6,7,8-tetrahydropyrido[3,4-d]pyrimidin-4-yl)-2-(cyanomethyl)piperazine-1-carboxylate (350 mg, 63%) as a yellow oil: ¹H NMR (400 MHz, CDCl₃) δ 9.83 (s, 1H), 8.00 (s, 2H), 7.74 (d, J=8.1 Hz, 1H), 7.60 (t, J=7.8 Hz, 1H), 7.51 (d, J=7.4 Hz, 1H), 7.47-7.36 (m, 1H), 7.32 (t, J=7.8 Hz, 1H), 7.25-7.16 (m, 1H), 4.60 (s, 1H), 4.49-4.32 (m, 1H), 4.25 (t, J=4.9 Hz, 2H), 4.21-3.77 (m, 7H), 3.76-3.63 (m, 3H), 3.63-3.42 (m, 7H), 3.41-2.64 (m, 8H), 2.55 (t, J=13.1 Hz, 1H), 2.49-2.34 (m, 1H), 2.35-2.15 (m, 1H), 2.15-1.93 (m, 1H), 1.93-1.69 (m, 5H), 1.42 (s, 9H); LC/MS (ESI, m/z): [(M+1)]⁺=1026.5.

### Step 7: (S)-tert-butyl 4-(7-(8-chloronaphthalen-1-yl)-2-(((S)-1-(3-(2-(2-(2,6-dibromo-4-((5-iodo-2-oxoindolin-3-ylidene)methyl)phenoxy)ethoxy)ethoxy)propyl)pyrrolidin-2-yl)methoxy)-5,6,7,8-tetrahydropyrido[3,4-d]pyrimidin-4-yl)-2-(cyanomethyl)piperazine-1-carboxylate

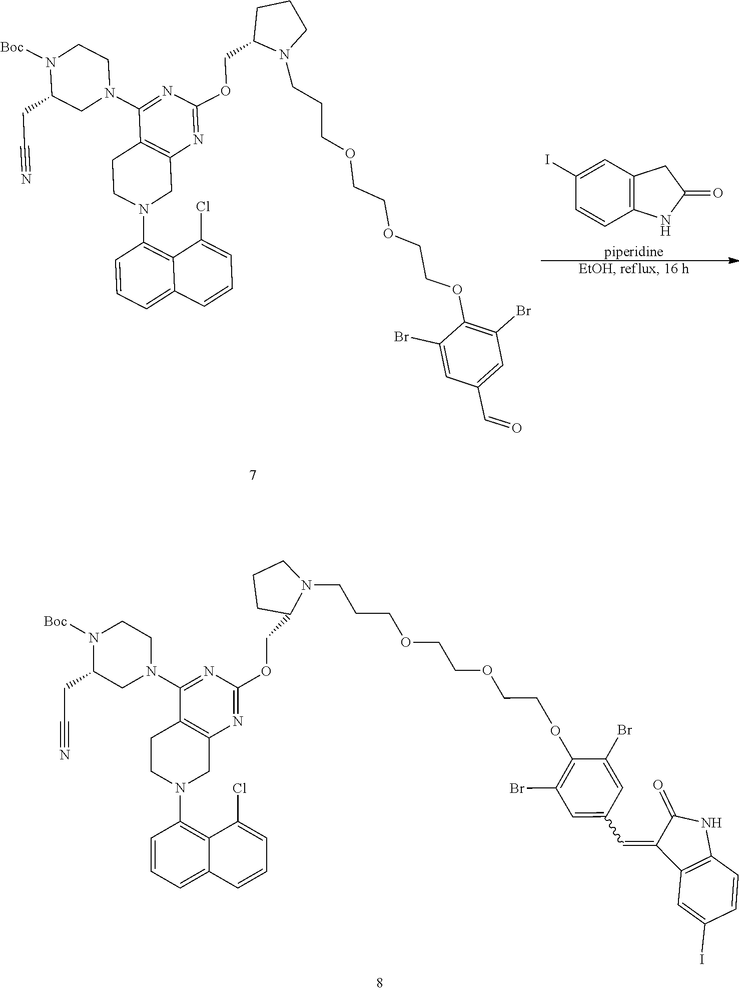

To a solution of (S)-tert-butyl 4-(7-(8-chloronaphthalen-1-yl)-2-(((S)-1-(3-(2-(2-(2,6-dibromo-4-formylphenoxy)ethoxy)ethoxy)propyl)pyrrolidin-2-yl)methoxy)-5,6,7,8-tetrahydro pyrido[3,4-d]pyrimidin-4-yl)-2-(cyanomethyl)piperazine-1-carboxylate (150 mg, 0.15 mmol) in ethanol (5 mL) were added piperidine (2 mg, 0.015 mmol) and 5-iodo-1,3-dihydroindol-2-one (39 mg, 0.16 mmol). The resulting mixture was stirred for 16 h at 80° C. under a nitrogen atmosphere. The resulting mixture was concentrated under reduced pressure. The residue was purified by Prep-TLC (CH₂Cl₂/Methanol=15/1) to afford (S)-tert-butyl 4-(7-(8-chloro naphthalen-1-yl)-2-(((S)-1-(3-(2-(2-(2,6-dibromo-4-((5-iodo-2-oxoindolin-3-ylidene)methyl) phenoxy)ethoxy)ethoxy) propyl)pyrrolidin-2-yl)methoxy)-5,6,7,8-tetrahydropyrido[3,4-d]pyrimidin-4-yl)-2-(cyanomethyl)piperazine-1-carboxylate (120 mg, 65%) as a yellow solid: ¹H NMR (400 MHz, CDCl₃) δ 8.47 (s, 1H), 7.91-7.70 (m, 3H), 7.63-7.56 (m, 2H), 7.56-7.47 (m, 2H), 7.47-7.36 (m, 1H), 7.32 (t, J=7.7 Hz, 1H), 7.25-7.14 (m, 1H), 6.64 (dd, J=15.4, 8.4 Hz, 1H), 4.61 (s, 1H), 4.46-4.20 (m, 4H), 4.15-3.77 (m, 7H), 3.77-3.42 (m, 9H), 3.38-2.64 (m, 8H), 2.62-2.32 (m, 2H), 2.33-2.14 (m, 1H), 2.10-1.92 (m, 1H), 1.92-1.58 (m, 5H), 1.51 (s, 9H); LC/MS (ESI, m/z): [(M+1)]+=1267.5.

### Step 8: 2-((S)-4-(7-(8-chloronaphthalen-1-yl)-2-(((S)-1-(3-(2-(2-(2,6-dibromo-4-((5-iodo-2-oxoindolin-3-ylidene)methyl)phenoxy)ethoxy)ethoxy)propyl)pyrrolidin-2-yl)methoxy)-5,6,7,8-tetrahydropyrido[3,4-d]pyrimidin-4-yl)piperazin-2-yl)acetonitrile

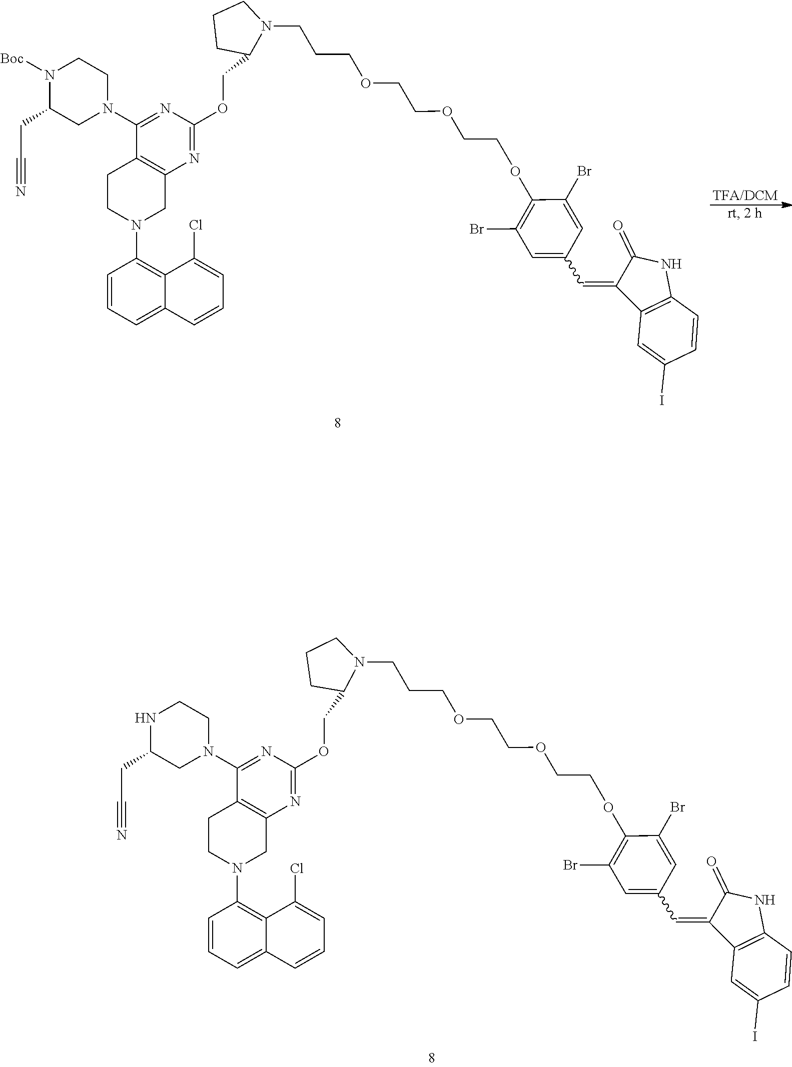

A solution of (S)-tert-butyl 4-(7-(8-chloronaphthalen-1-yl)-2-(((S)-1-(3-(2-(2-(2,6-dibromo-4-((5-iodo-2-oxoindolin-3-ylidene)methyl)phenoxy)ethoxy)ethoxy)propyl)pyrrolidin-2-yl)methoxy)-5,6,7,8-tetrahydropyrido[3,4-d]pyrimidin-4-yl)-2-(cyanomethyl)piperazine-1-carboxylate (120 mg, 0.095 mmol) in DCM (10 mL) and trifluoroacetic acid (2 mL) was stirred for 20 min at room temperature under a nitrogen atmosphere. The resulting mixture was concentrated under reduced pressure. The residue was purified by reverse phase flash chromatography with the following conditions: Column, Spherical C18, 20˜40 μm, 80 g; Eluent A: Water (plus 10 mM NH₄HCO₃); Eluent B: Methanol; Flow rate: 50 mL/min; Gradient: 90% B to 93% B in 10 min; Detector: UV 220/254 nm. Desired fractions were collected and concentrated under reduced pressure to afford 2-((S)-4-(7-(8-chloronaphthalen-1-yl)-2-(((S)-1-(3-(2-(2-(2,6-dibromo-4-((5-iodo-2-oxoindolin-3-ylidene)methyl)phenoxy)ethoxy)ethoxy)propyl) pyrrolidin-2-yl)methoxy)-5,6,7,8-tetrahydropyrido[3,4-d]pyrimidin-4-yl)piperazin-2-yl) acetonitrile (70 mg, 64%) as a yellow solid: ¹H NMR (400 MHz, DMSO-d₆) δ 10.78 (d, J=22.4 Hz, 1H), 8.76 (s, 1H), 8.07-7.80 (m, 3H), 7.70 (d, J=7.9 Hz, 1H), 7.60-7.37 (m, 5H), 7.33-7.27 (m, 1H), 6.71 (dd, J=16.7, 8.2 Hz, 1H), 4.28-4.20 (m, 1H), 4.19-4.09 (m, 3H), 4.01-3.63 (m, 6H), 3.59-3.32 (m, 8H), 3.14-2.75 (m, 8H), 2.74-2.62 (m, 4H), 2.32-2.22 (m, 1H), 2.18-2.05 (m, 1H), 1.92-1.79 (m, 1H), 1.72-1.52 (m, 5H); LC/MS (ESI, m/z): [(M+1)]⁺=1167.4.

### Step 9: 2-((S)-4-(7-(8-chloronaphthalen-1-yl)-2-(((S)-1-(3-(2-(2-(2,6-dibromo-4-((5-iodo-2-oxoindolin-3-ylidene)methyl)phenoxy)ethoxy)ethoxy)propyl)pyrrolidin-2-yl)methoxy)-5,6,7,8-tetrahydropyrido[3,4-d]pyrimidin-4-yl)-1-(2-fluoroacryloyl)piperazin-2-yl)acetonitrile

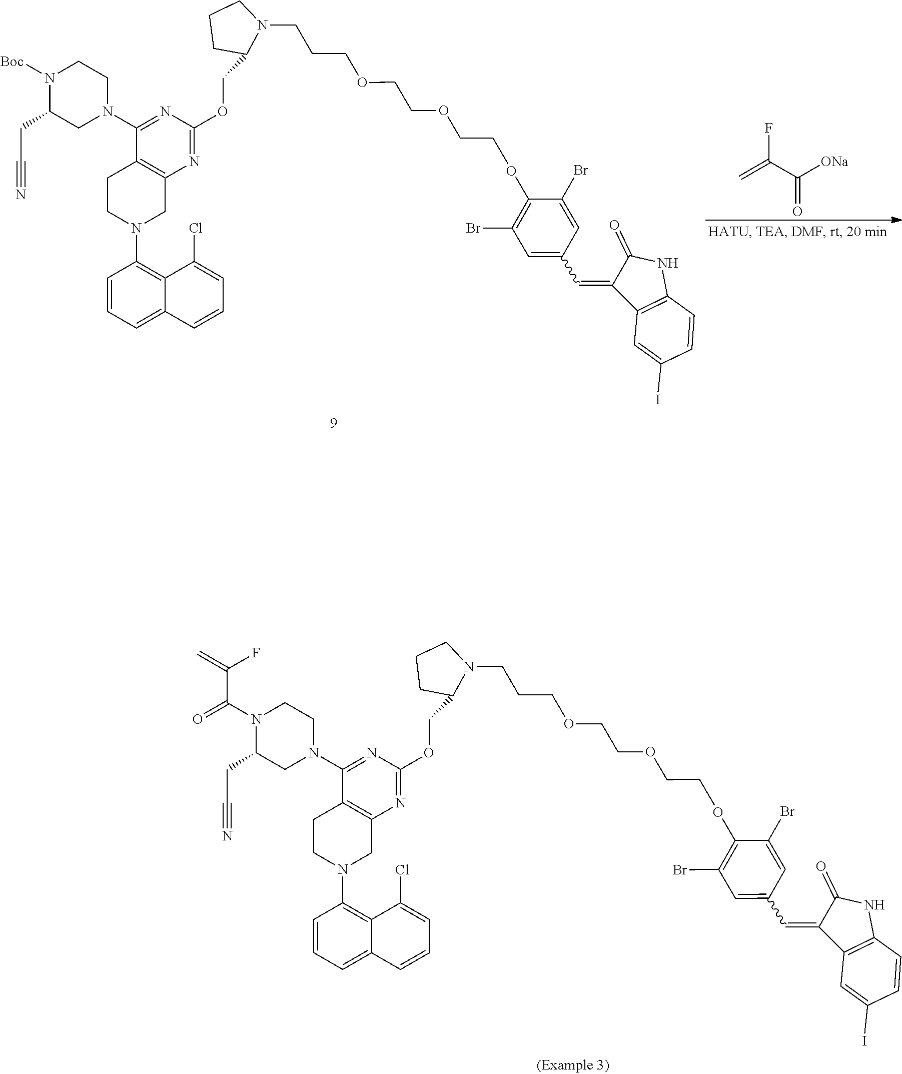

To a stirred solution of 2-((S)-4-(7-(8-chloronaphthalen-1-yl)-2-(((S)-1-(3-(2-(2-(2,6-dibromo-4-((5-iodo-2-oxoindolin-3-ylidene)methyl)phenoxy)ethoxy)ethoxy)propyl)pyrrolidin-2-yl)methoxy)-5,6,7,8-tetrahydropyrido[3,4-d]pyrimidin-4-yl)piperazin-2-yl)acetonitrile (70 mg, 0.06 mmol) in DMF (3 mL) were added triethylamine (19 mg, 0.18 mmol), sodium 2-fluoroprop-2-enoate (14 mg, 0.12 mmol) and HATU (35 mg, 0.09 mmol) at room temperature under a nitrogen atmosphere. The resulting mixture was stirred for 20 min at room temperature. The resulting mixture was concentrated under reduced pressure. The residue was purified by Prep-HPLC with the following conditions: Column: XSelect CSH Prep C18 OBD Column, 19*250 mm, 5 μm; Eluent A: Water (plus 10 mM NH₄HCO₃); Eluent B: ACN; Flow rate: 25 mL/min; Gradient: 70% B to 90% B in 8 min; Detector: UV 220/254 nm. Desired fractions were collected, concentrated under reduced pressure and lyophilized to afford 2-((S)-4-(7-(8-chloro naphthalen-1-yl)-2-(((S)-1-(3-(2-(2-(2,6-dibromo-4-((5-iodo-2-oxoindolin-3-ylidene)methyl) phenoxy)ethoxy)ethoxy)propyl)pyrrolidin-2-yl)methoxy)-5,6,7,8-tetrahydropyrido[3,4-d]pyrimidin-4-yl)-1-(2-fluoroacryloyl)piperazin-2-yl)acetonitrile (9.1 mg, 13%) as a yellow solid: ¹H NMR (400 MHz, DMSO-d6) δ 10.81 (d, J=22.0 Hz, 1H), 8.76 (s, 1H), 8.08-7.80 (m, 3H), 7.75-7.68 (m, 1H), 7.59-7.47 (m, 3H), 7.47-7.39 (m, 1H), 7.36-7.25 (m, 1H), 6.71 (dd, J=16.3, 8.1 Hz, 1H), 5.45-5.16 (m, 2H), 4.87 (s, 1H), 4.29-4.08 (m, 4H), 4.05-3.81 (m, 4H), 3.80-3.66 (m, 3H), 3.57-3.34 (m, 8H), 3.24-2.79 (m, 8H), 2.73-2.62 (m, 2H), 2.31-2.23 (m, 1H), 2.16-2.06 (m, 1H), 1.91-1.80 (m, 1H), 1.72-1.54 (m, 5H); LC/MS (ESI, m/z): [(M+1)]⁺=1239.40.

### Example 4: 5-((10-(4-(4-Amino-3-(4-phenoxyphenyl)-1H-pyrazolo[3,4-d] pyrimidin-1-yl)piperidin-1-yl)decyl)oxy)-7-hydroxy-4-phenyl-2H-chromen-2-one

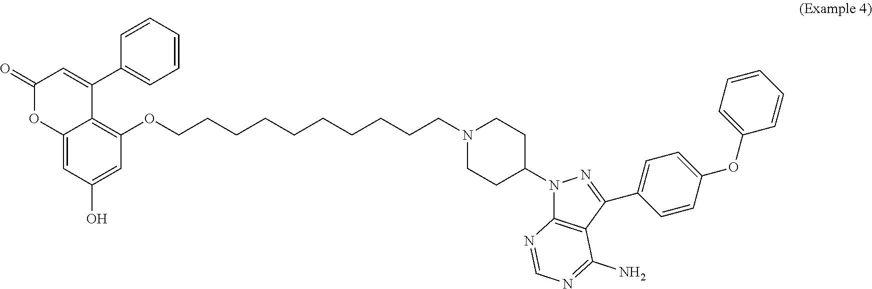 [SMILES: Nc1ncnc2c1c(-c1ccc(Oc3ccccc3)cc1)nn2C1CCN(CCCCCCCCCCOc2cc(O)cc3oc(=O)cc(-c4ccccc4)c23)CC1]

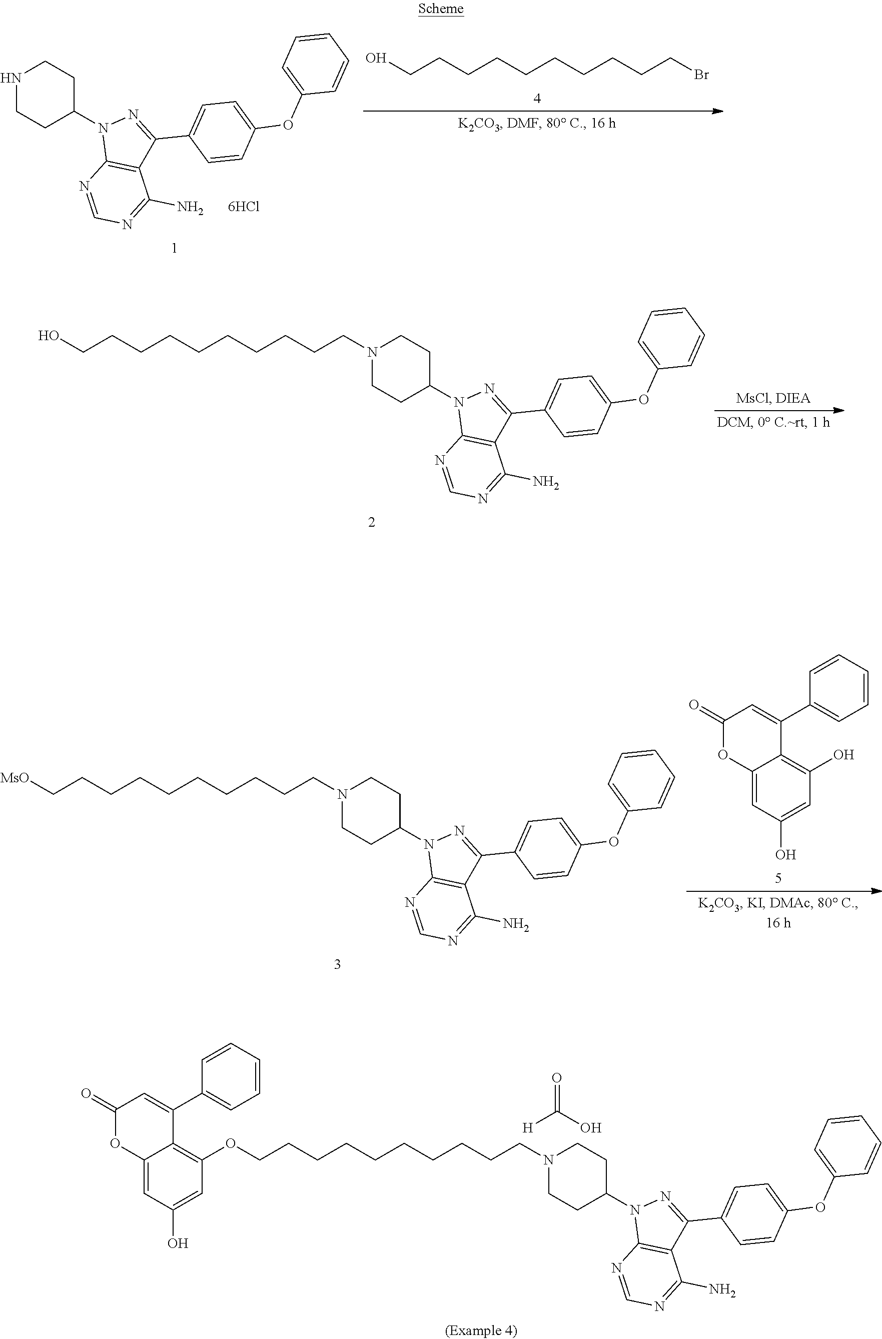

### Step 1: 10-(4-(4-Amino-3-(4-phenoxyphenyl)-1H-pyrazolo[3,4-d]pyrimidin-1-yl)piperidin-1-yl)decan-1-ol

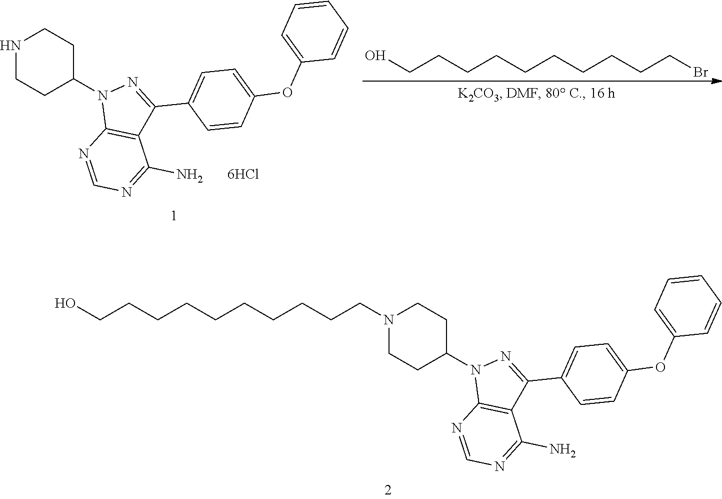

To a solution of 3-(4-phenoxyphenyl)-1-(piperidin-4-yl)pyrazolo[3,4-d]pyrimidin-4-amine (5.00 g, 12.94 mmol) in DMF (50 mL) were added 10-bromodecan-1-ol (4.60 g, 19.41 mmol) and K₂CO₃ (3.58 g, 25.88 mmol) at room temperature under a nitrogen atmosphere. The resulting mixture was stirred for 16 hours at 80° C. under a nitrogen atmosphere. Upon completion, the resulting mixture was cooled down to room temperature and was concentrated under reduced pressure. The residue was purified by silica gel column chromatography, eluted with CH₂Cl₂/MeOH (1% to 5%, v/v) to afford 10-(4-(4-Amino-3-(4-phenoxyphenyl)-1H-pyrazolo[3,4-d]pyrimidin-1-yl)piperidin-1-yl)decan-1-ol (6.0 g, 86%) as an off-white solid: ¹H NMR (400 MHz, CDCl₃) δ 8.36 (s, 1H), 7.66 (d, J=8.5 Hz, 2H), 7.40 (t, J=7.8 Hz, 2H), 7.17 (dd, J=14.3, 7.9 Hz, 3H), 7.09 (d, J=8.0 Hz, 2H), 5.70 (s, 1H), 4.78 (m, 1H), 3.65 (t, J=6.6 Hz, 3H), 3.12 (dd, J=12.0, 8.3 Hz, 2H), 2.51-2.34 (m, 4H), 2.20 (t, J=12.0 Hz, 2H), 1.55 (dd, J=13.5, 6.8 Hz, 4H), 1.38-1.28 (m, 16H); LC/MS (ESI, m/z): ((M+1))⁺=543.2.

### Step 2: 10-(4-(4-Amino-3-(4-phenoxyphenyl)-1H-pyrazolo[3,4-d]pyrimidin-1-yl)piperidin-1-yl)decyl methanesulfonate

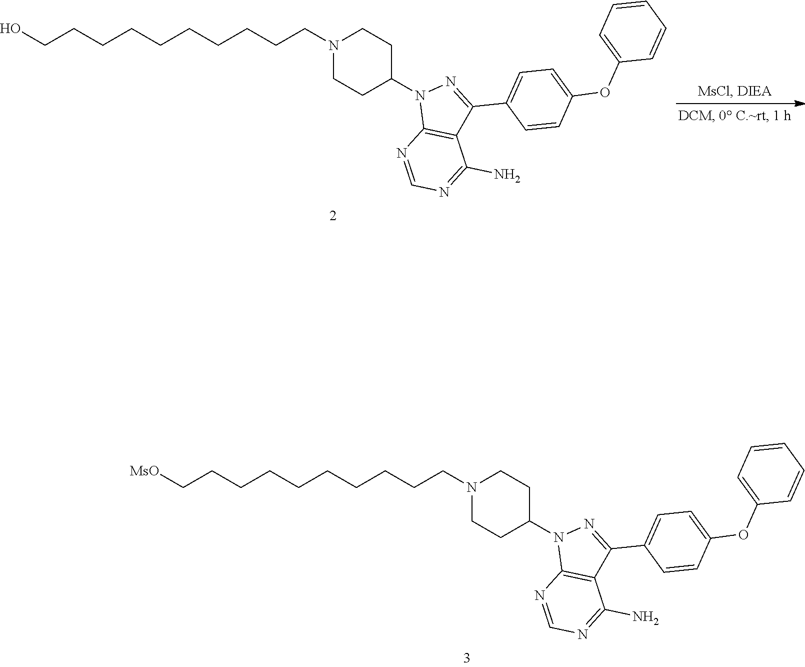 [SMILES: Nc1ncnc2c1c(-c1ccc(Oc3ccccc3)cc1)nn2C1CCN(CCCCCCCCCCO)CC1 ; CS(=O)(=O)OCCCCCCCCCCN1CCC(n2nc(-c3ccc(Oc4ccccc4)cc3)c3c(N)ncnc32)CC1]

To a solution of 10-(4-(4-amino-3-(4-phenoxyphenyl)pyrazolo[3,4-d]pyrimidin-1-yl)piperidin-1-yl)decan-1-ol (4.00 g, 7.37 mmol) in DCM (50 mL) were added mesyl chloride (1.01 g, 8.84 mmol) and DIEA (1.62 g, 12.53 mmol) at 0° C. under a nitrogen atmosphere. The resulting mixture was stirred for 4 h at room temperature under a nitrogen atmosphere. The resulting mixture was concentrated under reduced pressure. The residue was purified by silica gel column chromatography, eluted with 7%˜50% ethyl acetate in petroleum ether to afford 10-(4-(4-amino-3-(4-phenoxyphenyl)pyrazolo[3,4-d]pyrimidin-1-yl)piperidin-1-yl)decyl methanesulfonate (4.00 g, 88%) as an off-white solid: ¹H NMR (400 MHz, CDCl₃) δ 8.36 (s, 1H), 7.69-7.63 (m, 2H), 7.39 (t, J=8.0 Hz, 2H), 7.15 (dd, J=8.2, 6.2 Hz, 3H), 7.09 (d, J=8.0 Hz, 2H), 4.79 (tt, J=11.6, 4.2 Hz, 1H), 4.23 (t, J=6.6 Hz, 2H), 3.01 (s, 3H), 2.45 (dd, J=12.0, 3.7 Hz, 2H), 2.43-2.37 (m, 2H), 2.21 (t, J=11.9 Hz, 2H), 2.04 (d, J=12.6 Hz, 2H), 1.56-1.50 (m, 2H), 1.42 (s, 2H), 1.41-1.23 (m, 16H); LC/MS (ESI, m/z): ((M+18))⁺=621.2.

### Step 3: 5-((10-(4-(4-Amino-3-(4-phenoxyphenyl)-1H-pyrazolo[3,4-d]pyrimidin-1-yl)piperidin-1-yl)decyl)oxy)-7-hydroxy-4-phenyl-2H-chromen-2-one

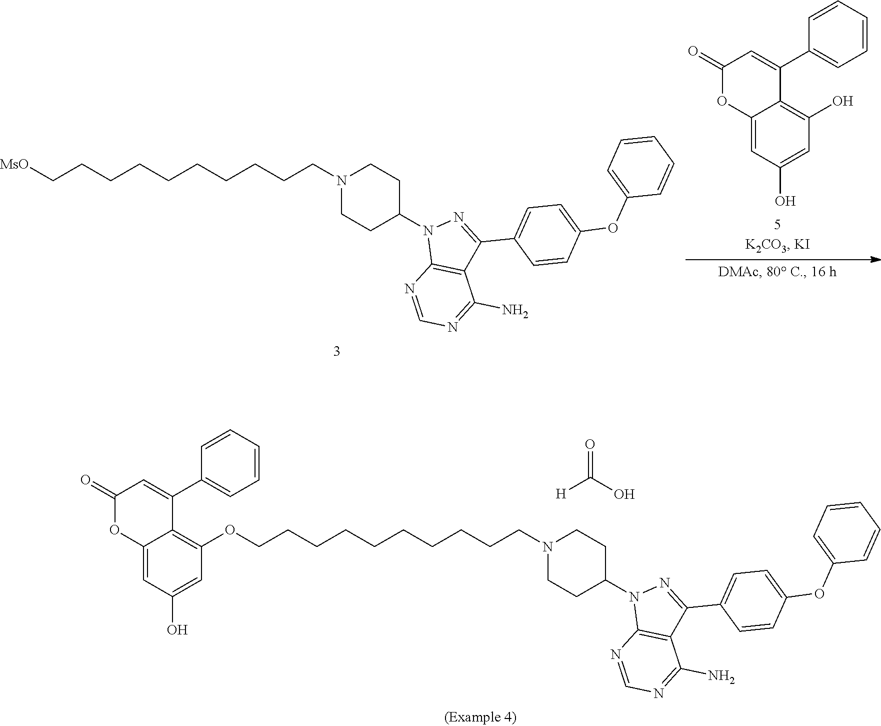

To a solution of 10-(4-(4-amino-3-(4-phenoxyphenyl)pyrazolo[3,4-d]pyrimidin-1-yl)piperidin-1-yl)decyl methanesulfonate (200 mg, 0.32 mmol) in dimethylacetamide (7 mL) were added 5,7-dihydroxy-4-phenyl-2H-chromen-2-one (122 mg, 0.48 mmol), K₂CO₃ (111 mg, 0.81 mmol) and KI (53 mg, 0.32 mmol) at room temperature under a nitrogen atmosphere. The resulting mixture was stirred for 16 h at 80° C. under a nitrogen atmosphere. Upon completion, the resulting mixture was concentrated under reduced pressure. The residue was purified by Prep-TLC (CH₂Cl₂/MeOH=10/1) and further purified by prep-HPLC with the following conditions: (Column: Xselect CSH OBD Column 30*150 mm 5 m, Eluent A: Water (plus 0.1% formic acid); Eluent B: ACN; Flow rate: 60 mL/min; Gradient: 40% B to 50% B in 8 min; Detector: UV 220/254 nm). The fractions containing desired product were collected, concentrated under reduced pressure and lyophilized to afford 5-((10-(4-(4-amino-3-(4-phenoxy phenyl)pyrazolo[3,4-d]pyrimidin-1-yl)piperidin-1-yl)decyl)oxy)-7-hydroxy-4-phenylchromen-2-one formate (38 mg, 15%) as an off-white solid: ¹H NMR (400 MHz, DMSO-d₆) δ 8.23 (d, J=6.1 Hz, 1H), 7.69-7.63 (m, 2H), 7.46-7.41 (m, 2H), 7.37 (q, J=2.9 Hz, 3H), 7.31-7.26 (m, 2H), 7.20 (d, J=7.3 Hz, 1H), 7.17-7.09 (m, 4H), 6.40 (d, J=2.2 Hz, 1H), 6.26 (d, J=2.3 Hz, 1H), 5.77 (s, 1H), 4.72-4.63 (m, 1H), 3.64 (t, J=6.1 Hz, 2H), 3.03 (d, J=10.3 Hz, 2H), 2.35 (q, J=7.1 Hz, 2H), 2.18 (m, 4H), 1.97-1.85 (m, 2H), 1.46 (s, 2H), 1.39-1.17 (m, 6H), 1.13 (s, 2H), 1.03 (d, J=10.1 Hz, 4H), 0.77 (d, J=7.8 Hz, 2H); LC/MS (ESI, m/z): ((M+1))⁺=788.45.

### Example 5: 3-(4-((10-(4-(4-Amino-3-(4-phenoxyphenyl)-1H-pyrazolo[3,4-d]pyrimidin-1-yl)piperidin-1-yl)decyl)oxy)-3,5-dibromobenzylidene)-5-iodoindolin-2-one

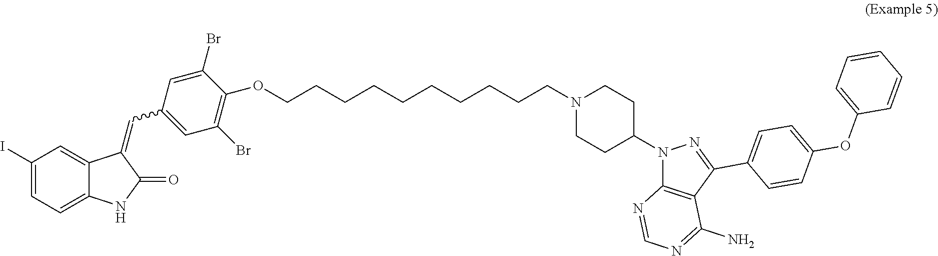 [SMILES: Nc1ncnc2c1c(-c1ccc(Oc3ccccc3)cc1)nn2C1CCN(CCCCCCCCCCOc2c(Br)cc(C=C3C(=O)Nc4ccc(I)cc43)cc2Br)CC1]

### Step: 3-(4-((10-(4-(4-Amino-3-(4-phenoxyphenyl)-1H-pyrazolo[3,4-d]pyrimidin-1-yl)piperidin-1-yl)decyl)oxy)-3,5-dibromobenzylidene)-5-iodoindolin-2-one

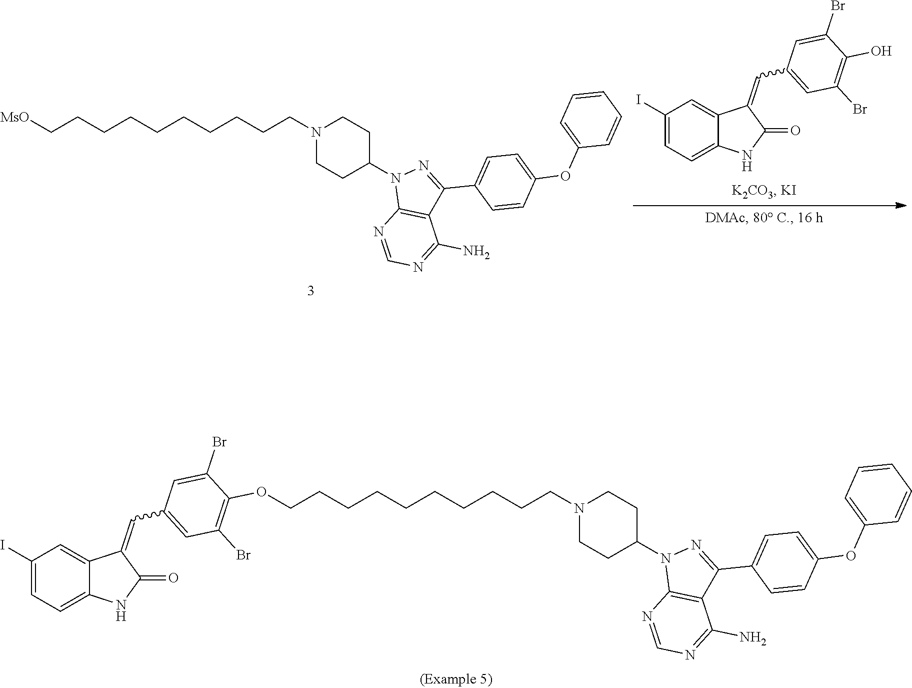

To a stirred solution of 10-(4-(4-amino-3-(4-phenoxyphenyl)pyrazolo[3,4-d]pyrimidin-1-yl)piperidin-1-yl)decyl methanesulfonate (200 mg, 0.32 mmol) in dimethylacetamide (8 mL) were added 3-((3,5-dibromo-4-hydroxyphenyl)methylidene)-5-iodo-1H-indol-2-one (201 mg, 0.39 mmol), K₂CO₃ (111 mg, 0.81 mmol) and KI (53 mg, 0.32 mmol) at room temperature under a nitrogen atmosphere. The resulting mixture was stirred for 16 hours at 80° C. under a nitrogen atmosphere. The resulting mixture was concentrated under reduced pressure and the residue was purified by Prep-TLC (CH₂Cl₂/MeOH=10/1). Further purified by Achiral SFC with the following conditions: (Column: DAICEL Dcpak P4VP, 4.6*50 mm, 3 μm; Eluent A: CO₂; Eluent B: MeOH:DCM=1:1 (plus 1% 2 M NH₃-MeOH); Flow rate: 20 mL/min; Gradient: isocratic 10% B; Detector: UV 220/254 nm.). The fractions containing desired product were collected, concentrated under reduced pressure and lyophilized to afford 3-((4-((10-(4-(4-amino-3-(4-phenoxyphenyl) pyrazolo[3,4-d]pyrimidin-1-yl)piperidin-1-yl)decyl)oxy)-3,5-dibromophenyl) methylidene)-5-iodo-1H-indol-2-one (39.5 mg, 12%) as a yellow solid: ¹H NMR (400 MHz, DMSO-d₆) δ 10.80 (d, J=22.5 Hz, 1H), 8.79 (s, 1H), 8.23 (s, 1H), 8.05-7.99 (m, 1H), 7.85 (s, 1H), 7.75-7.61 (m, 2H), 7.56 (m, 1H), 7.47-7.39 (m, 2H), 7.25-7.06 (m, 5H), 6.71 (dd, J=19.1, 8.2 Hz, 1H), 4.72-4.56 (m, 1H), 4.03 (dt, J=12.5, 6.3 Hz, 2H), 2.99 (d, J=11.0 Hz, 2H), 2.30 (d, J=7.3 Hz, 2H), 2.24-2.12 (m, 2H), 2.06 (t, J=11.6 Hz, 2H), 1.94-1.76 (m, 4H), 1.48 (dd, J=22.6, 7.2 Hz, 4H), 1.30 (s, 10H); LC/MS (ESI, m/z): ((M+1))⁺=1046.40.

### Example 6: 1-(14-(4-(4-amino-3-(4-phenoxyphenyl)-1H-pyrazolo[3,4-d]pyrimidin-1-yl) piperidin-1-yl)-3,6,9,12-tetraoxatetradecyl)-3-(3,5-dibromo-4-hydroxybenzylidene)-5-iodo indolin-2-one formate

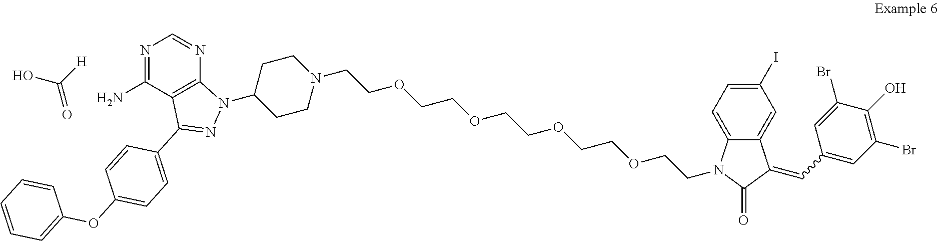 [SMILES: Nc1ncnc2c1c(-c1ccc(Oc3ccccc3)cc1)nn2C1CCN(CCOCCOCCOCCOCCN2C(=O)C(=Cc3cc(Br)c(O)c(Br)c3)c3cc(I)ccc32)CC1 ; O=CO]

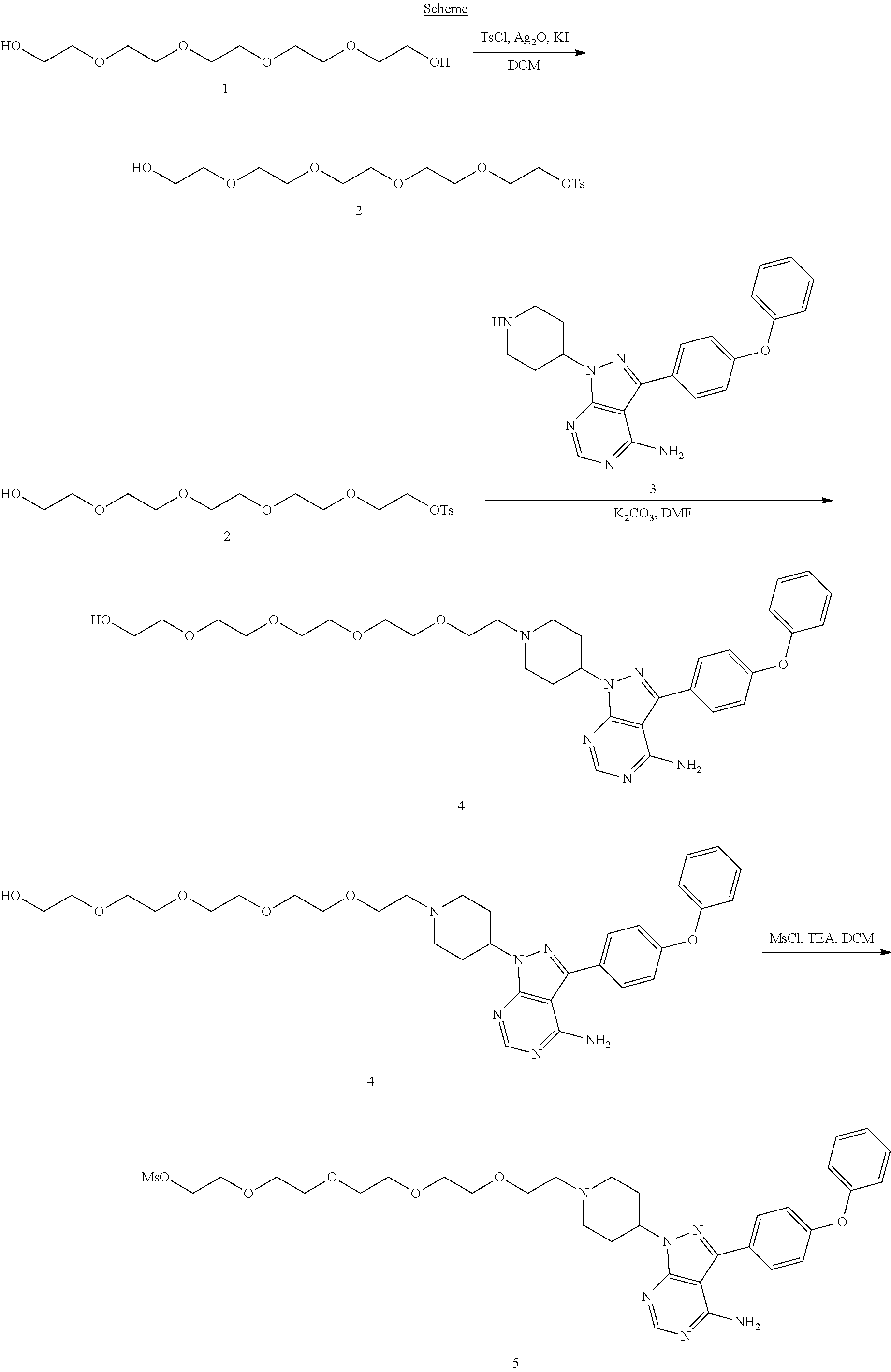

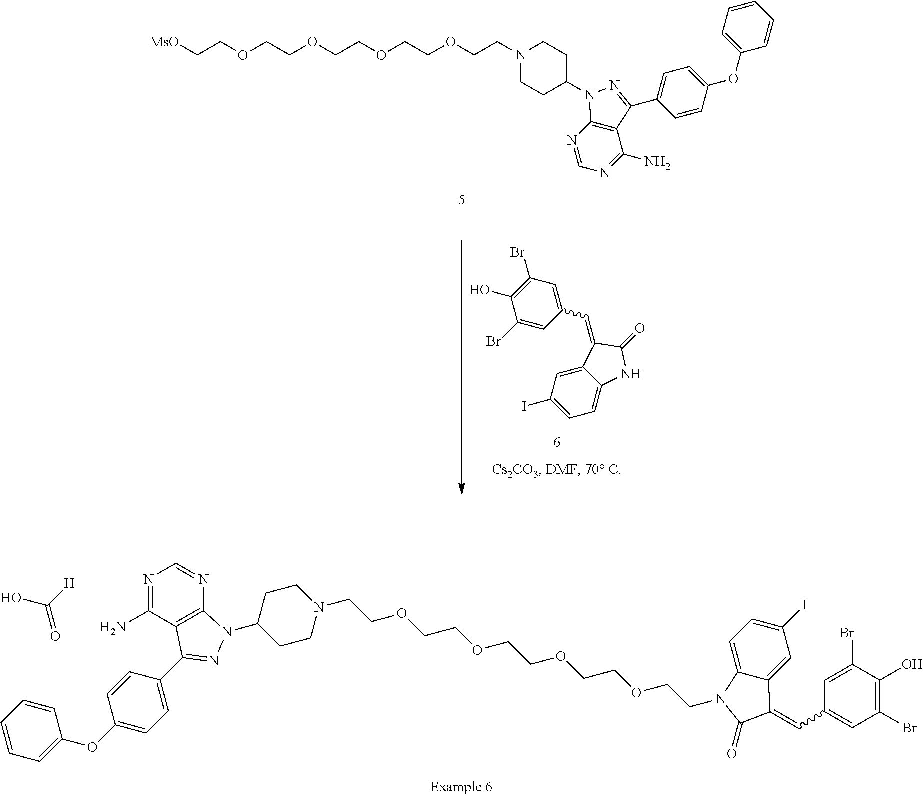 [SMILES: CS(=O)(=O)OCCOCCOCCOCCOCCN1CCC(n2nc(-c3ccc(Oc4ccccc4)cc3)c3c(N)ncnc32)CC1 ; O=C1Nc2ccc(I)cc2C1=Cc1cc(Br)c(O)c(Br)c1 ; Nc1ncnc2c1c(-c1ccc(Oc3ccccc3)cc1)nn2C1CCN(CCOCCOCCOCCOCCN2C(=O)C(=Cc3cc(Br)c(O)c(Br)c3)c3cc(I)ccc32)CC1 ; O=CO]

### Step 1: 14-hydroxy-3,6,9,12-tetraoxatetradecyl 4-methylbenzenesulfonate

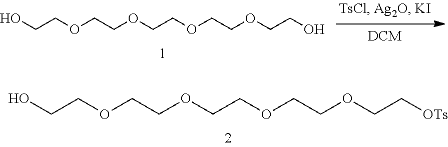 [SMILES: OCCOCCOCCOCCOCCO ; Cc1ccc(S(=O)(=O)OCCOCCOCCOCCOCCO)cc1]

To a solution of 3,6,9,12-tetraoxatetradecane-1,14-diol (8.80 g, 36.9 mmol, CAS #4792-15-8), KI (245 mg, 1.48 mmol), Ag₂O (2.57 g, 11.0 mmol) in DCM (90 mL) was added TsCl (520 mg, 7.39 mmol) at 0° C. The mixture was stirred at 25° C. for 16 hours. On completion, the reaction mixture was diluted with H₂O (100 mL), and extracted with ethyl acetate (3×150 mL). The combined organic layers were washed with brine (3×40 mL), dried over anhydrous Na₂SO₄, and filtered. The filtrate was concentrated under reduced pressure to give a residue. The residue was purified by column chromatography (SiO₂, Petroleum ether/Ethyl acetate=0/1) to give the title compound (1.50 g, 52% yield) as a colorless oil: ¹H NMR (400 MHz, CDCl₃) δ 7.92-7.70 (m, 2H), 7.35 (d, J=8.4 Hz, 2H), 4.28-4.07 (m, 2H), 3.74-3.61 (m, 15H), 3.60 (s, 4H), 2.48-2.43 (m, 3H); LC/MS (ESI, m/z): ((M+1))⁺=393.1.

### Step 2: 14-(4-(4-amino-3-(4-phenoxyphenyl)-1H-pyrazolo[3,4-d]pyrimidin-1-yl)piperidin-1-yl)-3,6,9,12-tetraoxatetradecan-1-ol

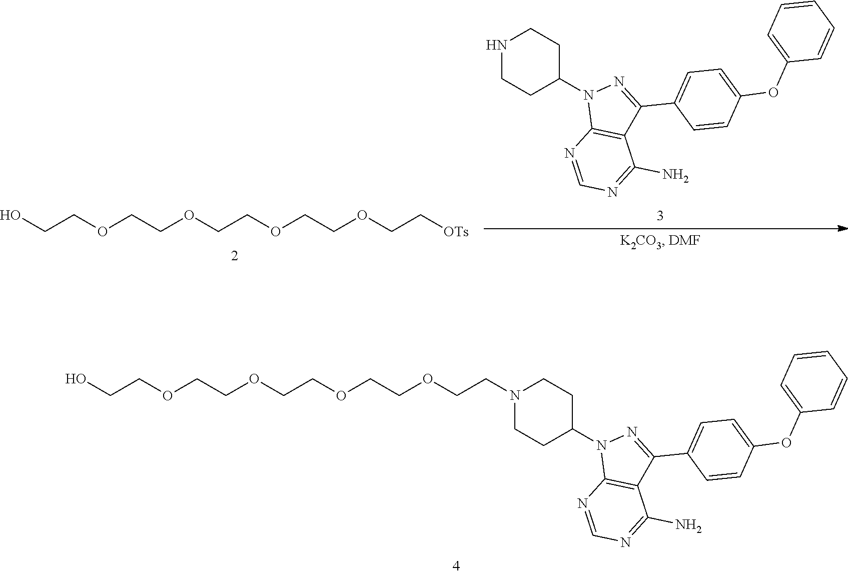 [SMILES: Nc1ncnc2c1c(-c1ccc(Oc3ccccc3)cc1)nn2C1CCNCC1 ; Cc1ccc(S(=O)(=O)OCCOCCOCCOCCOCCO)cc1 ; Nc1ncnc2c1c(-c1ccc(Oc3ccccc3)cc1)nn2C1CCN(CCOCCOCCOCCOCCO)CC1]

To a solution of 14-hydroxy-3,6,9,12-tetraoxatetradecyl 4-methylbenzenesulfonate (501 mg, 1.28 mmol) and 3-(4-phenoxyphenyl)-1-(4-piperidyl)pyrazolo[3,4-d]pyrimidin-4-amine (360 mg, 851 umol, HCl salt) in DMF (2 mL) was added K₂CO₃ (470 mg, 3.40 mmol). The mixture was stirred at 80° C. for 2 hours. On completion, the mixture was filtered and concentrated under reduced pressure. The residue was purified by prep-HPLC(column: Phenomenex C18 150*25 mm*10 um; mobile phase: [water(FA)-ACN]; B %: 10%˜40%, 8 min) to give the title compound (240 mg, 47% yield) as a colorless oil: ¹H NMR (400 MHz, DMSO-d₆) δ 8.32-8.05 (m, 1H), 7.66 (d, J=8.8 Hz, 1H), 7.53-7.38 (m, 2H), 7.24-7.03 (m, 4H), 3.75-3.67 (m, 1H), 3.57 (m, 3H), 3.51 (m, 5H), 3.47-3.36 (m, 3H), 3.32 (s, 18H), 2.28 (s, 2H), 2.18-2.00 (m, 2H); LC/MS (ESI, m/z): ((M+1))⁺=607.4.

### Step 3: 14-(4-(4-amino-3-(4-phenoxyphenyl)-1H-pyrazolo[3,4-d]pyrimidin-1-yl)piperidin-1-yl)-3,6,9,12-tetraoxatetradecyl methanesulfonate

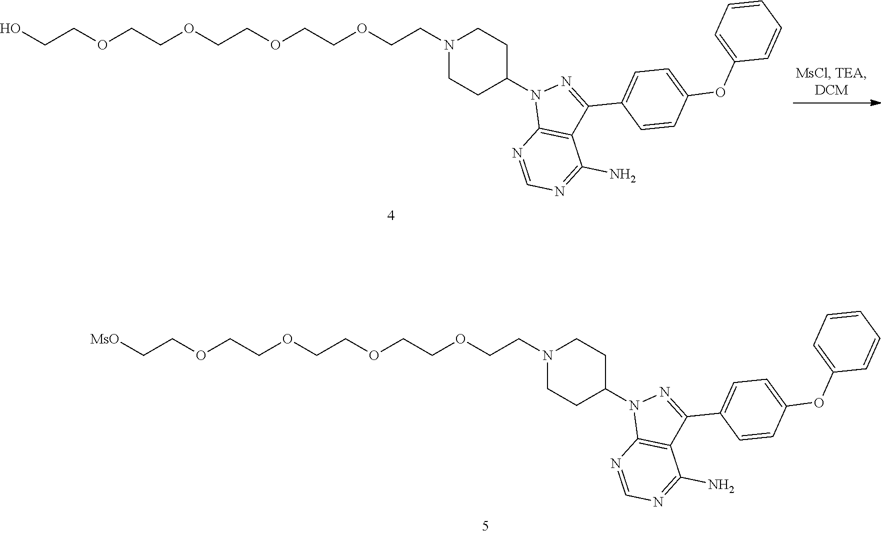 [SMILES: Nc1ncnc2c1c(-c1ccc(Oc3ccccc3)cc1)nn2C1CCN(CCOCCOCCOCCOCCO)CC1 ; CS(=O)(=O)OCCOCCOCCOCCOCCN1CCC(n2nc(-c3ccc(Oc4ccccc4)cc3)c3c(N)ncnc32)CC1]

To a solution of 14-(4-(4-amino-3-(4-phenoxyphenyl)-1H-pyrazolo[3,4-d]pyrimidin-1-yl)piperidin-1-yl)-3,6,9,12-tetraoxatetradecan-1-ol (350 mg, 576 umol) in DCM (2 mL) was added TEA (233 mg, 2.31 mmol), followed by MsCl (198 mg, 1.73 mmol) at 0° C. The mixture was stirred at 25° C. for 1 hour. On completion, the mixture was diluted with DCM (5 mL), washed with the saturated citric acid solution (3×10 mL), dried over anhydrous Na₂SO₄, filtered and concentrated under reduced pressure to give the title compound (395 mg, 99% yield) as a yellow oil: ¹H NMR (400 MHz, DMSO-d₆) δ 11.11 (s, 1H), 10.37 (s, 1H), 8.30 (s, 1H), 8.23 (s, 1H), 8.06 (d, J=8.3 Hz, 1H), 7.88 (d, J=8.2 Hz, 1H), 7.65 (d, J=8.1 Hz, 2H), 7.44 (t, J=7.6 Hz, 2H), 7.15 (q, J=10.1, 7.3 Hz, 5H), 5.12 (d, J=11.6 Hz, 1H), 4.63 (s, 1H), 4.20 (s, 2H), 3.72 (s, 2H), 3.65 (s, 3H), 3.58 (d, J=6.3 Hz, 3H), 3.02 (s, 2H), 2.86 (d, J=15.2 Hz, 1H), 2.60 (s, 3H), 2.18 (d, J=8.4 Hz, 4H), 2.04 (s, 1H), 1.86 (s, 2H); LC/MS (ESI, m/z): ((M+1))⁺=685.3.

### Step 4: 1-(14-(4-(4-amino-3-(4-phenoxyphenyl)-1H-pyrazolo[3,4-d]pyrimidin-1-yl)piperidin-1-yl)-3,6,9,12-tetraoxatetradecyl)-3-(3,5-dibromo-4-hydroxybenzylidene)-5-iodoindolin-2-one formate

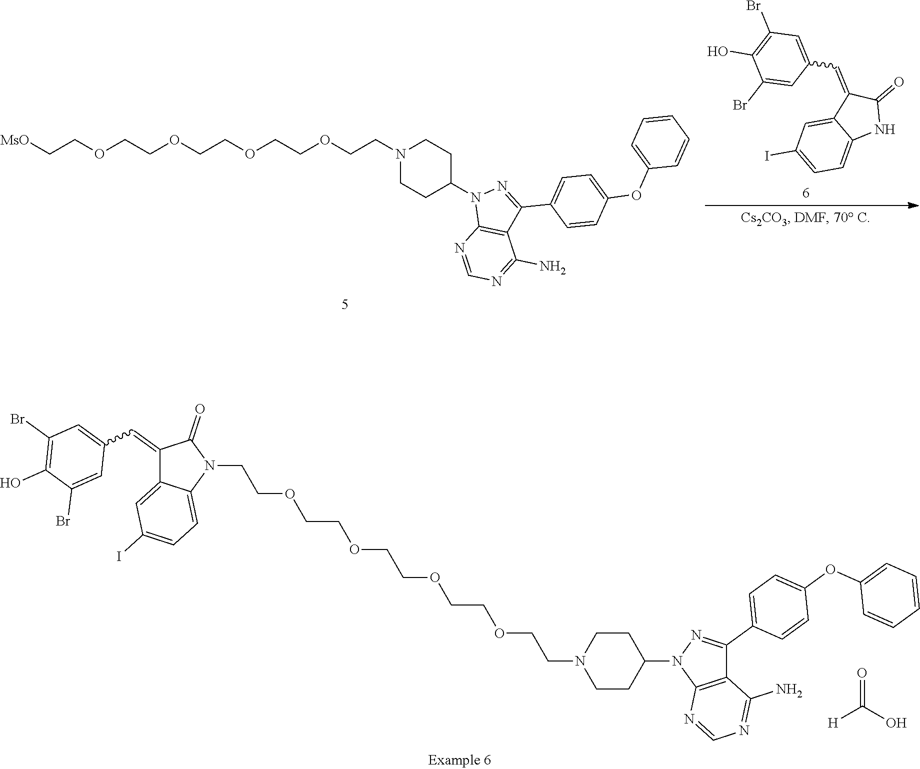 [SMILES: CS(=O)(=O)OCCOCCOCCOCCOCCN1CCC(n2nc(-c3ccc(Oc4ccccc4)cc3)c3c(N)ncnc32)CC1 ; O=C1Nc2ccc(I)cc2C1=Cc1cc(Br)c(O)c(Br)c1 ; Nc1ncnc2c1c(-c1ccc(Oc3ccccc3)cc1)nn2C1CCN(CCOCCOCCOCCOCCN2C(=O)C(=Cc3cc(Br)c(O)c(Br)c3)c3cc(I)ccc32)CC1 ; O=CO]

To a solution of 14-(4-(4-amino-3-(4-phenoxyphenyl)-1H-pyrazolo[3,4-d]pyrimidin-1-yl)piperidin-1-yl)-3,6,9,12-tetraoxatetradecyl methanesulfonate (150 mg, 219 umol) and 3-(3,5-dibromo-4-hydroxybenzylidene)-5-iodoindolin-2-one (171 mg, 328 umol) in DMF (2 mL) was added Cs₂CO₃ (142 mg, 438 umol). The mixture was degassed with N₂ for 3 times and heated to 70° C. for 16 hours under N₂. On completion, the mixture was filtered and concentrated under reduced pressure. The residue was purified by prep-TLC (Dichloromethane/Methanol=10/1) and prep-HPLC (column: Welch Ultimate C18 150*25 mm*5 um; mobile phase: [water(FA)-ACN]; B %: 32%-62%, 10.5 min) to give the title compound (14.5 mg, 5.8% yield, 98% purity) as an orange solid: ¹H NMR (400 MHz, DMSO-d₆) δ 8.88-8.68 (m, 1H), 8.31-8.20 (m, 1H), 8.13 (s, 1H), 8.08-7.87 (m, 1H), 7.82 (s, 1H), 7.65 (d, J=8.0 Hz, 2H), 7.58 (s, 1H), 7.52-7.46 (m, 1H), 7.45 (d, J=2.0 Hz, 1H), 7.43 (s, 1H), 7.41 (s, 1H), 7.37 (dd, J=1.2, 8.0 Hz, 1H), 7.18 (t, J=7.2 Hz, 1H), 7.15 (s, 1H), 7.13 (s, 2H), 7.11 (s, 1H), 6.92 (d, J=8.0 Hz, 1H), 6.82 (d, J=8.0 Hz, 1H), 4.98-4.78 (m, 1H), 3.91-3.84 (m, 2H), 3.63 (m, 2H), 3.62-3.56 (m, 3H), 3.50-3.45 (m, 6H), 3.39 (s, 10H), 2.42-2.35 (m, 2H), 2.07 (m, 2H); LC/MS (ESI, m/z): ((M+1))⁺=1109.5.

### Example 7: (3Z)-3-[[2-[2-[2-[2-[4-[4-amino-3-(4-phenoxyphenyl)pyrazolo[3,4-d]pyrimidin-1-yl]-1-piperidyl]ethoxy]ethoxy]ethoxy]ethylamino]-(3-hydroxyphenyl)methylene]-5-bromo-indolin-2-one formate

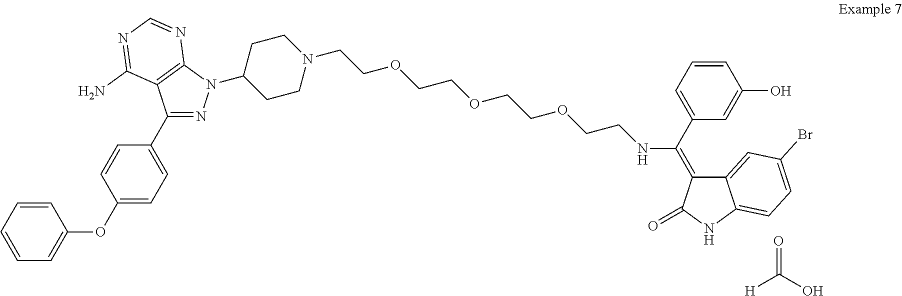

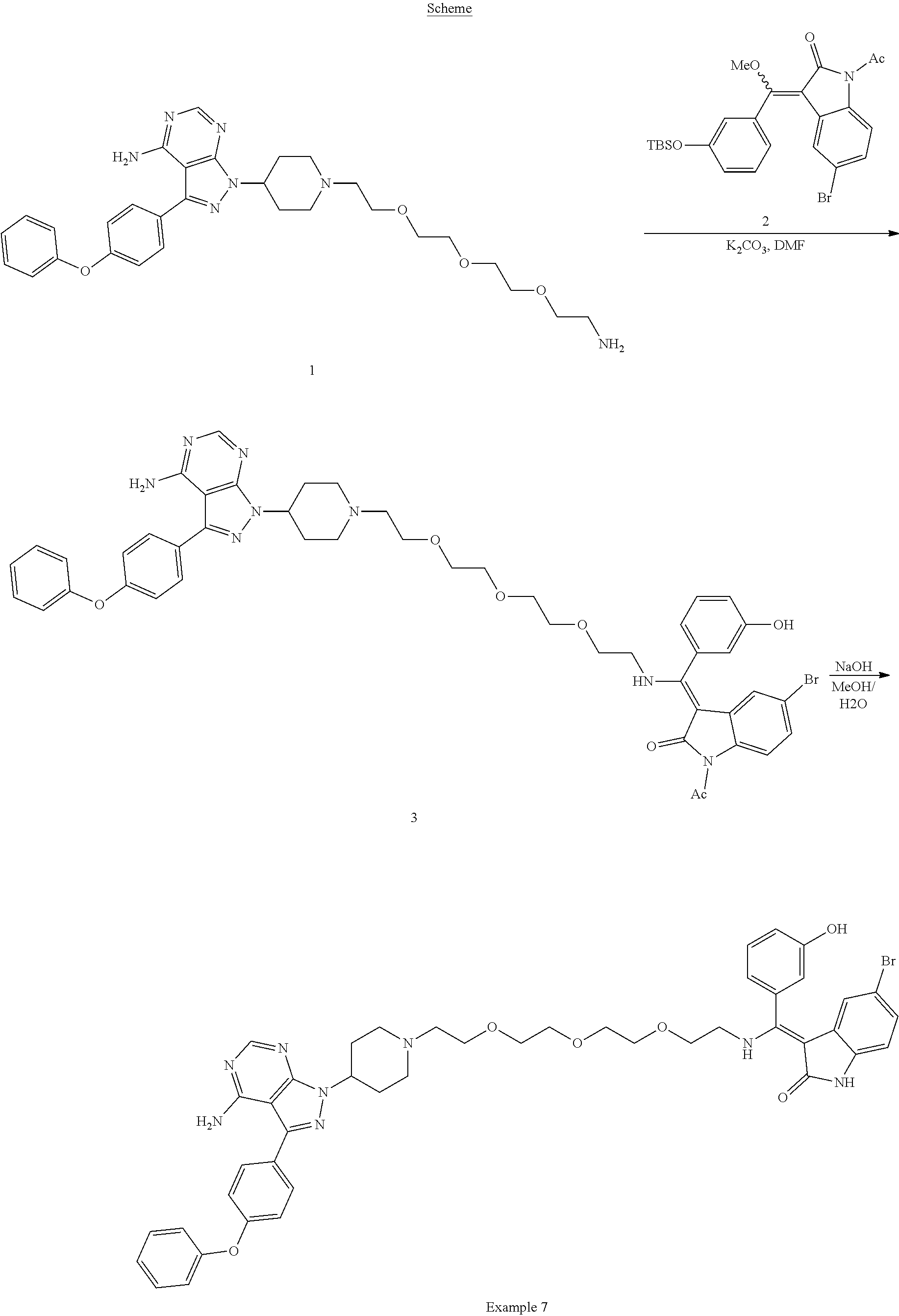

### Step 1: (3Z)-1-acetyl-3-[[2-[2-[2-[2-[4-[4-amino-3-(4-phenoxyphenyl)pyrazolo[3,4-d]pyrimidin-1-yl]-1-piperidyl]ethoxy]ethoxy]ethoxy]ethylamino]-(3-hydroxyphenyl)methylene]-5-bromo-indolin-2-one

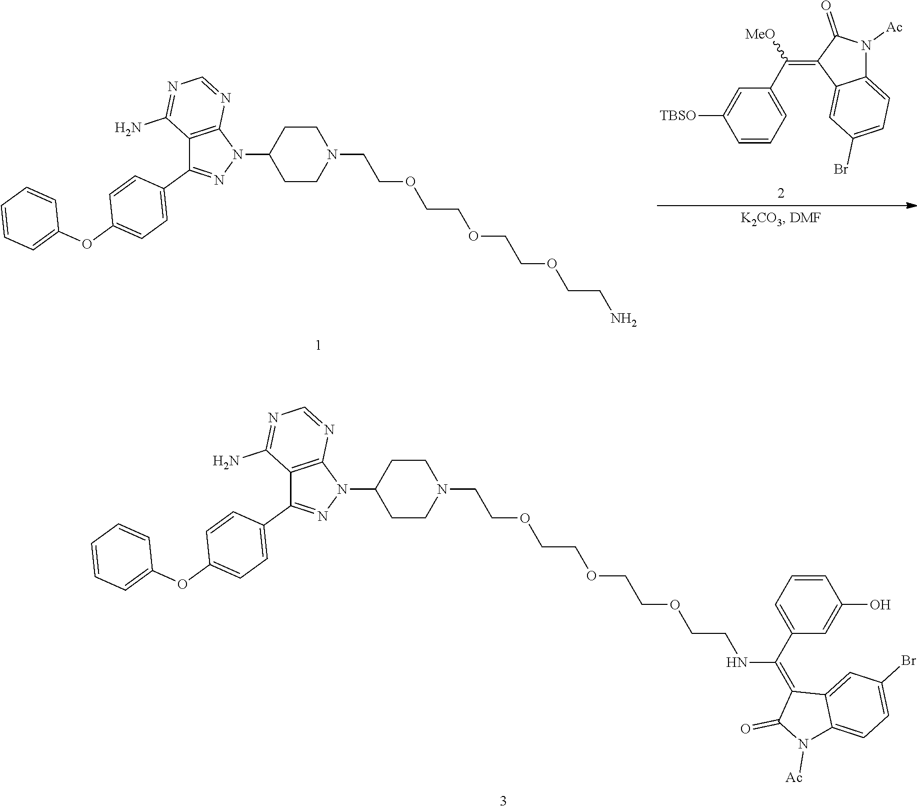 [SMILES: NCCOCCOCCOCCN1CCC(n2nc(-c3ccc(Oc4ccccc4)cc3)c3c(N)ncnc32)CC1 ; COC(=C1C(=O)N(C(C)=O)c2ccc(Br)cc21)c1cccc(O[Si](C)(C)C(C)(C)C)c1 ; CC(=O)N1C(=O)/C(=C(\NCCOCCOCCOCCN2CCC(n3nc(-c4ccc(Oc5ccccc5)cc4)c4c(N)ncnc43)CC2)c2cccc(O)c2)c2cc(Br)ccc21]

To a solution of 1-[1-[2-[2-[2-(2-aminoethoxy)ethoxy]ethoxy]ethyl]-4-piperidyl]-3-(4-phenoxyphenyl)pyrazolo [3,4-d]pyrimidin-4-amine (60 mg, 100 umol, HCl salt) and (3Z)-1-acetyl-5-bromo-3-[[3-[tert-butyl(dimethyl)silyl]oxyphenyl]-methoxy-methylene]indolin-2-one (25.2 mg, 50.1 umol, from) in DMF (1 mL) was added K₂CO₃ (13.8 mg, 100 umol). The mixture was stirred at 25° C. for 12 hours. On completion, the mixture was diluted with H₂O (10 mL), extracted with Ethyl acetate (3×15 mL), the organic layers were washed with brine (2×20 mL), dried over anhydrous Na₂SO₄, filtered and concentrated in vacuo to give the title compound (46 mg, 99% yield) as a yellow solid. LC/MS (ESI, m/z): ((M+1))⁺=919.1.

### Step 2: (3Z)-3-[[2-[2-[2-[2-[4-[4-amino-3-(4-phenoxyphenyl)pyrazolo[3,4-d]pyrimidin-1-yl]-1-piperidyl]ethoxy]ethoxy]ethoxy]ethylamino]-(3-hydroxyphenyl)methylene]-5-bromo-indolin-2-one

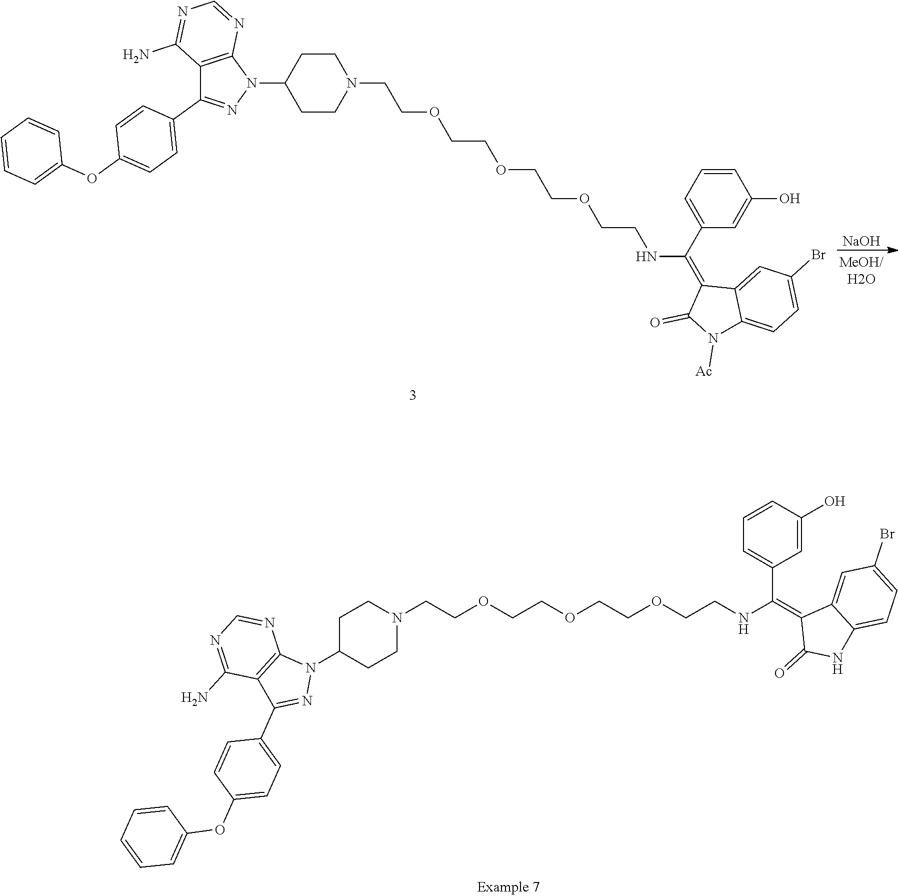

To a solution of (3Z)-1-acetyl-3-[[2-[2-[2-[2-[4-[4-amino-3-(4-phenoxyphenyl) pyrazolo[3,4-d]pyrimidin-1-yl]-1-piperidyl]ethoxy]ethoxy]ethoxy]ethylamino]-(3-hydroxy phenyl)methylene]-5-bromo-indolin-2-one (46 mg, 50.1 umol) in a mixture solvent of Methanol (1 mL) and H₂O (0.2 mL) was added NaOH (4.01 mg, 100 umol). The mixture was stirred at 25° C. for 1 hour. On completion, The mixture was diluted with H₂O (4 mL), extracted with Ethyl acetate (3×10 mL), the organic layers were washed with brine (2×10 mL), dried over anhydrous Na₂SO₄, filtered and concentrated in vacuo to give a residue. The residue was purified by prep-HPLC(column: Phenomenex luna C18 150*25 mm*10 um; mobile phase: [water(FA)-ACN]; B %: 22%-52%, 10.5 min) to give the title compound (23.7 mg, 51% yield, FA salt) as an orange solid. ¹H NMR (400 MHz, DMSO-d₆) δ 10.50 (s, 1H), 10.32-10.29 (m, 1H), 10.20-9.54 (m, 1H), 8.23 (s, 1H), 7.65 (d, J=8.4 Hz, 2H), 7.48-7.39 (m, 3H), 7.20-7.16 (m, 1H), 7.14-7.10 (m, 4H), 7.03-6.99 (m, 1H), 6.92-6.89 (m, 1H), 6.80 (d, J=7.6 Hz, 1H), 6.75 (s, 1H), 6.70 (d, J=8.0 Hz, 1H), 5.62 (d, J=1.6 Hz, 1H), 4.73-4.50 (m, 1H), 3.55-3.47 (m, 14H), 3.27-3.22 (m, 4H), 2.99 (d, J=6.0 Hz, 2H), 2.23-2.11 (m, 4H), 1.84 (s, 2H); LC/MS (ESI, m/z): ((M+1))⁺=877.2.

### Example 8: (3Z)-3-[[2-[2-[2-[4-[4-amino-3-(4-phenoxyphenyl)pyrazolo[3,4-d]pyrimidin-1-yl]-1-piperidyl]ethoxy]ethoxy]ethylamino]-(3-hydroxyphenyl)methylene]-5-bromo-indolin-2-one

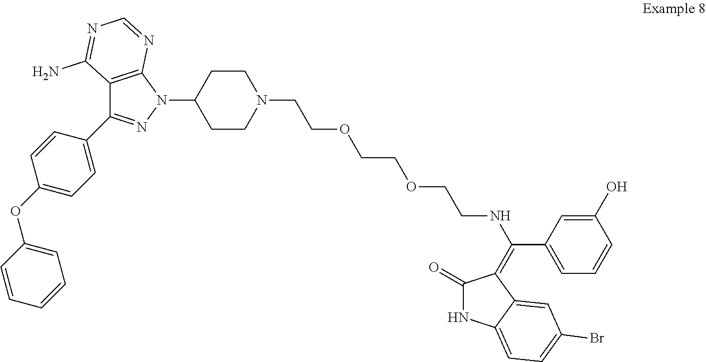 [SMILES: Nc1ncnc2c1c(-c1ccc(Oc3ccccc3)cc1)nn2C1CCN(CCOCCOCCN/C(=C2\C(=O)Nc3ccc(Br)cc32)c2cccc(O)c2)CC1]

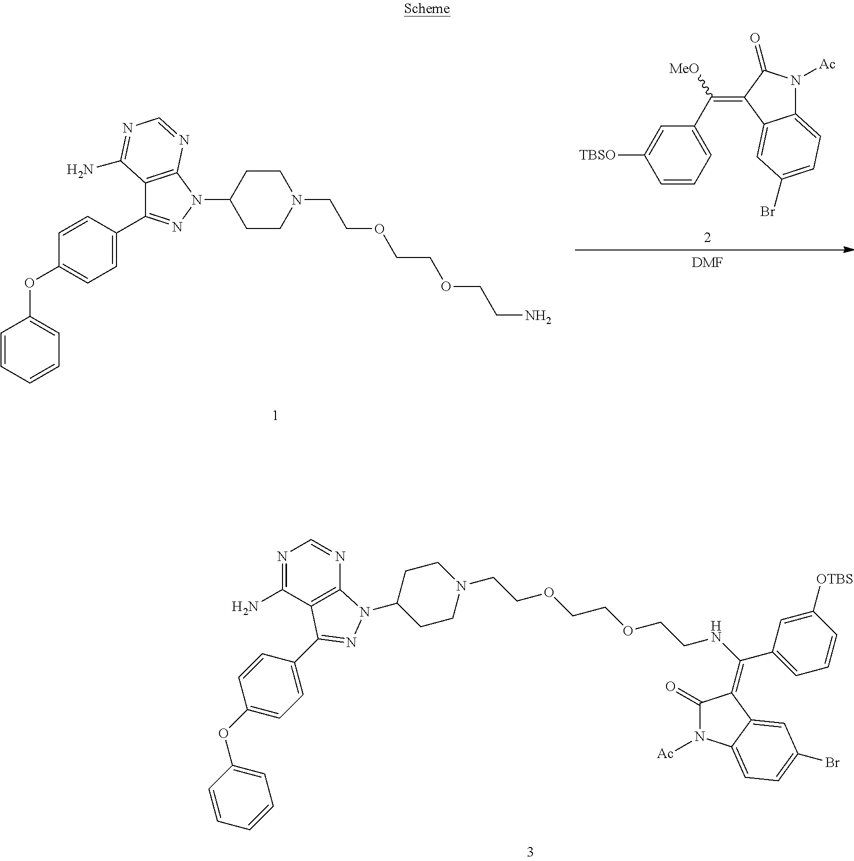

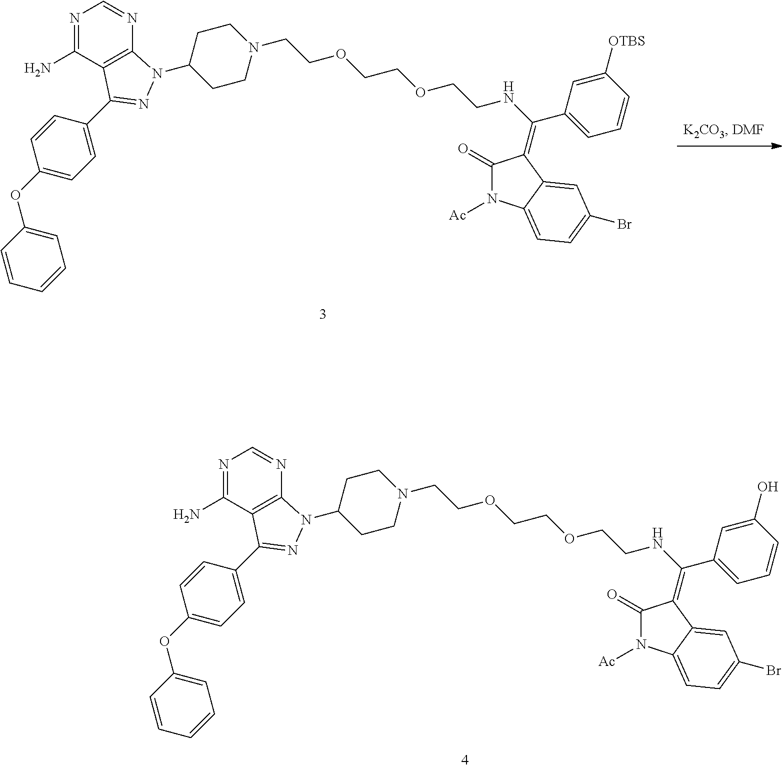

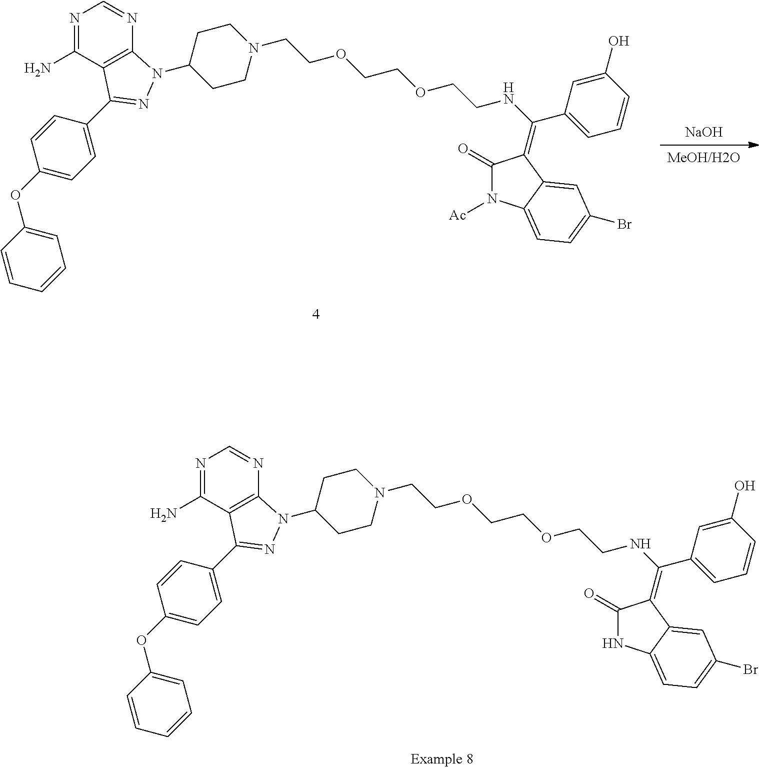 [SMILES: CC(=O)N1C(=O)/C(=C(\NCCOCCOCCN2CCC(n3nc(-c4ccc(Oc5ccccc5)cc4)c4c(N)ncnc43)CC2)c2cccc(O)c2)c2cc(Br)ccc21 ; "NaOH" ; "MeOH/H2O" ; Nc1ncnc2c1c(-c1ccc(Oc3ccccc3)cc1)nn2C1CCN(CCOCCOCCN/C(=C2\C(=O)Nc3ccc(Br)cc32)c2cccc(O)c2)CC1]

### Step 1: (3Z)-1-acetyl-3-[[2-[2-[2-[4-[4-amino-3-(4-phenoxyphenyl)pyrazolo[3,4-d]pyrimidin-1-yl]-1-piperidyl]ethoxy]ethoxy]ethylamino]-[3-[tert-butyl(dimethyl)silyl]oxyphenyl]methylene]-5-bromo-indolin-2-one

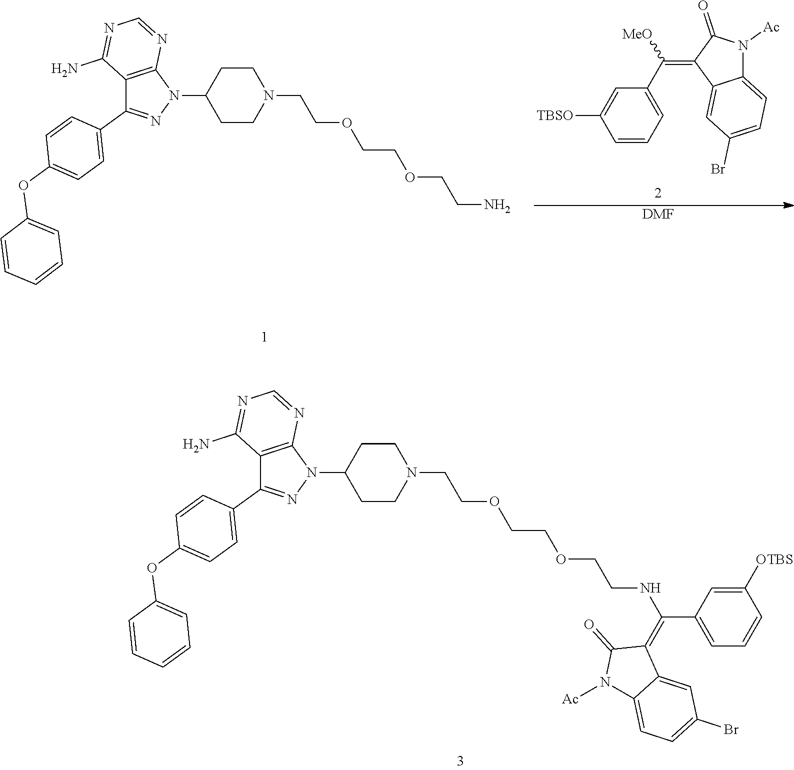

A solution of (3Z)-1-acetyl-3-[[2-[2-[2-[2-[4-[4-amino-3-(4-phenoxyphenyl)pyrazolo [3,4-d]pyrimidin-1-yl]-1-piperidyl]ethoxy]ethoxy]ethoxy]ethylamino]-[3-[tert-butyl(dimethyl) silyl]oxyphenyl] methylene]-5-bromo-indolin-2-one (41 mg, 79.2 umol,) and (3Z)-1-acetyl-5-bromo-3-[[3-[tert-butyl(dimethyl)silyl]oxyphenyl]-methoxy-methylene]indolin-2-one (19.9 mg, 39.6 umol) in DMF (1 mL) was stirred at 25° C. for 2 hours. On completion, the mixture was concentrated in vacuo to give the title compound (39 mg, 99% yield) as a yellow oil. LC/MS (ESI, m/z): ((M+1))⁺=989.1

### Step 2: (3Z)-1-acetyl-3-[[2-[2-[2-[4-[4-amino-3-(4-phenoxyphenyl)pyrazolo[3,4-d]pyrimidin-1-yl]-1-piperidyl]ethoxy]ethoxy]ethylamino]-(3-hydroxyphenyl)methylene]-5-bromo-indolin-2-one

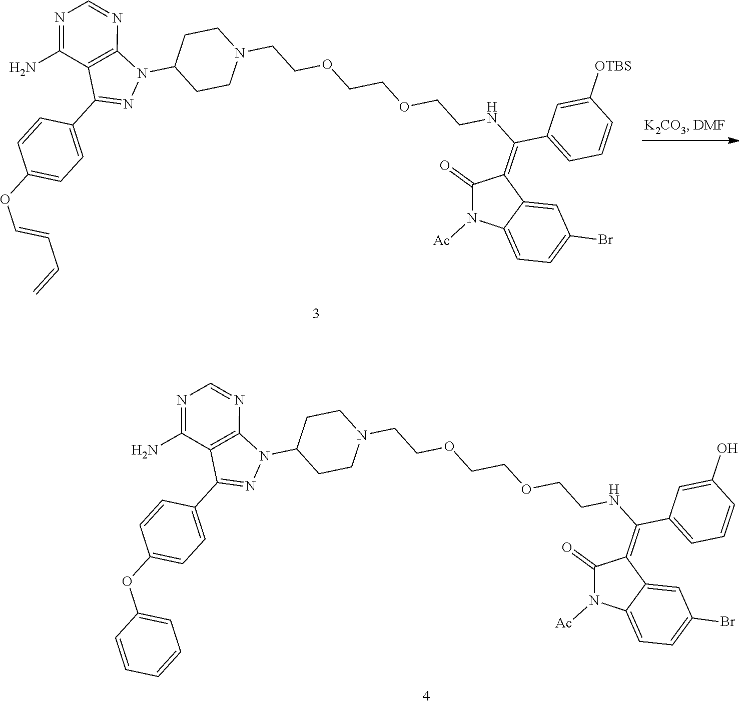 [SMILES: C=C/C=C/Oc1ccc(-c2nn(C3CCN(CCOCCOCCN/C(=C4\C(=O)N(C(C)=O)c5ccc(Br)cc54)c4cccc(O[Si](C)(C)C(C)(C)C)c4)CC3)c3ncnc(N)c23)cc1 ; CC(=O)N1C(=O)/C(=C(\NCCOCCOCCN2CCC(n3nc(-c4ccc(Oc5ccccc5)cc4)c4c(N)ncnc43)CC2)c2cccc(O)c2)c2cc(Br)ccc21]

To a solution of (3Z)-1-acetyl-3-[[2-[2-[2-[4-[4-amino-3-(4-phenoxyphenyl)pyrazolo [3,4-d]pyrimidin-1-yl]-1-piperidyl] ethoxy]ethoxy]ethylamino]-[3-[tert-butyl(dimethyl)silyl]oxyphenyl]methylene]-5-bromo-indolin-2-one (39 mg, 39.4 umol) in DMF (1 mL) was added K₂CO₃ (10.9 mg, 78.9 umol). The mixture was stirred at 25° C. for 1 hour. On completion, the mixture was filtered and concentrated in vacuo to give a residue. The residue was purified by prep-HPLC (column: Phenomenex luna C18 150*25 mm*10 um; mobile phase: [water(FA)-ACN]; B %: 5%-35%, 10.5 min) to give the title compound (13 mg, 35% yield, FA salt) as a white solid. ¹H NMR (400 MHz, DMSO-d₆) δ 10.42-10.39 (m, 1H), 10.28-9.56 (m, 1H), 8.21 (s, 1H), 7.98 (d, J=8.8 Hz, 1H), 7.67-7.62 (m, 2H), 7.50-7.39 (m, 3H), 7.23-7.09 (m, 5H), 7.08-7.00 (m, 2H), 6.85-6.77 (m, 2H), 5.60 (d, J=2.0 Hz, 1H), 4.71-4.48 (m, 1H), 3.62-3.50 (m, 10H), 3.31-3.30 (m, 2H), 2.97 (d, J=6.8 Hz, 2H), 2.67 (s, 3H), 2.52 (s, 2H), 2.19-2.10 (m, 4H), 1.82 (d, J=4.4 Hz, 2H); LC/MS (ESI, m/z): ((M+1))⁺=875.0.

### Step 3: (3Z)-3-[[2-[2-[2-[4-[4-amino-3-(4-phenoxyphenyl)pyrazolo[3,4-d]pyrimidin-1-yl]-1-piperidyl]ethoxy]ethoxy]ethylamino]-(3-hydroxyphenyl)methylene]-5-bromo-indolin-2-one

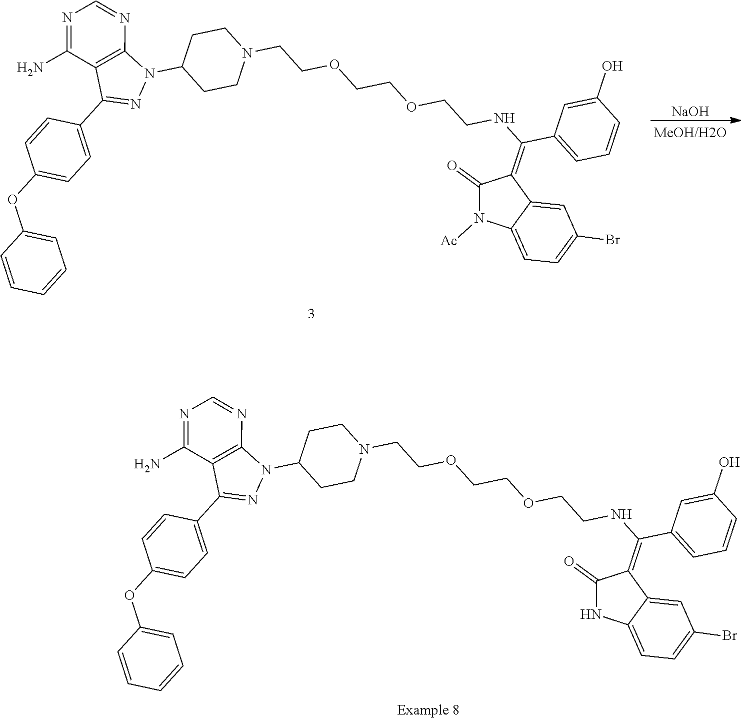

To a solution of (3Z)-1-acetyl-3-[[2-[2-[2-[4-[4-amino-3-(4-phenoxyphenyl)pyrazolo [3,4-d]pyrimidin-1-yl]-1-piperidyl] ethoxy]ethoxy]ethylamino]-(3-hydroxyphenyl)methylene]-5-bromo-indolin-2-one (13 mg, 14.1 umol, FA salt) in Methanol (1 mL) and H₂O (0.2 mL) was added NaOH (1.13 mg, 28.2 umol). The mixture was stirred at 25° C. for 1 hour. On completion, The mixture was diluted with H₂O (5 mL), extracted with Ethyl acetate (2×5 mL), the organic layers were washed with brine (2×10 mL), dried over anhydrous Na₂SO₄, filtered and concentrated in vacuo to give the title compound (7.61 mg, 64% yield) as an off-white solid. ¹H NMR (400 MHz, DMSO-d₆) δ 10.49 (s, 1H), 10.33-10.30 (m, 1H), 9.89 (s, 1H), 8.23 (s, 1H), 7.69-7.60 (m, 2H), 7.47-7.40 (m, 3H), 7.20-7.16 (m, 1H), 7.16-7.11 (m, 4H), 7.01 (dd, J=2.0, 8.0 Hz, 1H), 6.89 (dd, J=2.0, 8.2 Hz, 1H), 6.81 (d, J=7.6 Hz, 1H), 6.76 (d, J=2.0 Hz, 1H), 6.68 (d, J=8.0 Hz, 1H), 5.62 (d, J=2.0 Hz, 1H), 4.68-4.54 (m, 1H), 3.59-3.48 (m, 9H), 3.29-3.22 (m, 4H), 2.98 (d, J=6.8 Hz, 2H), 2.21-2.12 (m, 4H), 1.84 (d, J=9.2 Hz, 2H); LC/MS (ESI, m/z): ((M+1))⁺=833.0.

### C. Exemplified Compounds Reduce KRAS Levels in NCI-H2030 Cells

The following protocol was used to test the effect of exemplary compounds on KRAS levels in NCI-H2030 cells.

On Day 1, seed NCI-N2030 cells were placed into 6-well plates (3.5×105 cells/well/2 mL) and incubated for 24 hours. On Day 2, 6 μL compound solutions were added into testing wells in order to obtain compound concentrations of 3.33 μM and 10 μM—with DMSO concentrations of 0.3%. The wells were thoroughly agitated and incubated for 24 hours. On Day 3, the supernatant in the testing wells was discarded, and the testing wells were washed once with 1 mL of cold PBS. Then, 100 μL of RIPA buffer (Boston BioProducts BP-115D) was added to the testing wells with 1× complete protease inhibitor (Roche, #4693116001) and phosphatase inhibitor (Roche, #4906837001). The wells were then agitated by pipetting a few times, the testing plates were held on ice for 30 minutes to affect complete lysis of the cells. The cell lysate from the testing wells was then collected into EP tubes, and the tubes were spun at about 20000×g for 10 minutes at 4° C., before collecting the supernatant from the testing cells.

Protein concentrations in the collected supernatants were measured using BCA methods. The cell lysate was mixed with 5× NuPAGE SDS Loading Buffer and heated at 100° C. for 10 minutes in a heat block. Then 15 μL samples (15 μg) were loaded into 26-well SDS-PAGE gel (Novex, WXP81626BOX) and the gels were processed at 120 V for 1.5 hours, prior to electrotransfer to an NC membrane using a wet-transfer method with 250 mA for 1.5 hours. The resulting membrane was then blocked for 1 hours with LICOR blocking buffer (LI-COR, 927-70001), and was then incubated with a primary antibody 1:1000 (Anti-KRAS (LS-C175665)) that was previously prepared in block buffer at 4° C. overnight. Then, the membrane was washed three times with 1×TBST for 5 minutes each washing, and the membrane was incubated with a secondary antibody GAM 1:5000 (anti-mouse IgG (LI-COR, 926-68070)) for 1 hour at room temperature. The membrane was then washed three times with 1×TBST for 5 minutes each washing, and the membrane was read on LiCOR.

Table 2 below summarizes the normalized KRAS levels in NCI-H2030 cells that were treated with the exemplary compounds of Examples 1-3 for 24 hours.

TABLE 2
Normalized KRAS levels in NCI-H2030
cells treated with Compounds for 24 hours

| | 3.33 μM | 10 μM |
|---|---|---|
| Example | Compound | Compound |
| No. | Concentration | Concentration |
| 1 | 84.9% | 87.5% |
| 2 | 45.1% | 48.6% |
| 3 | 63.0% | 65.7% |

As illustrated in Table 2, compounds of the present disclosure are effective at reducing KRAS levels in NCI-H2030 cells at different compound concentrations.

### D. Exemplified Compounds Reduce BTK Levels in NAMALWA Cells

The following protocol was used to test the effect of exemplary compounds on BTK levels in NAMALWA cells.

Sample Preparation:

On Day 1, NAMALWA cells were seeded in 6 well plates at concentration of 2×10⁶/well and were incubated overnight. On Day 2, the cells were treated at indicated concentrations following 4 hours of starvation in Earle's Balanced Salt Solution (EBSS, Gibco, #24010-043). After compound treatment for 24 hours, cells were collected and samples were prepared. Fresh, ice cold 1×RIPA lysis buffer supplemented with protease and phosphatase inhibitors was then added to the cell samples, followed by incubation on ice for 30 minutes, and then cells samples were collected. Cell samples were then centrifuged at 4° C., 12,000 rpm for 15 minutes and the resulting supernatant was collected. Protein concentrations within the collected samples were then measured using BCA kit (Solarbio, PC0020), and the cell lysate was mixed with SDS loading buffer and heated at 100° C. for 5 minutes.

Western Blot:

15 μg of protein from the samples prepared above was loaded in 4-12% Bis-Tris Gels in 1×MOPS buffer, and the samples were run at 120 V for 120 min. Transfer of the resulting gels was then carried out using the Transblot system with NC membrane, and the membranes were blocked with 5% BSA in TBST for 1 hr at room temperature. Membrane hybridization was then carried out overnight at 4° C. with primary antibodies (anti-BTK, Cell Signaling Technology #8547, Rabbit, 1:1000 diluted in 1% BSA in TBST), and the resulting membranes were washed with TBST for 3×10 min at RT, and then incubated with second antibodies (Goat anti-Rabbit IgG H&L, IRDye® 800CW, LI-COR, 926-32211, 1:5000 diluted in 1% BSA in TBST) in 1% BSA for 1 hr at room temperature. After washing the resulting membranes with TBST for 10 min, 3 times at room temperatures, the membranes were imaged using a Li-COR imaging system.

Table 3 below summarizes the normalized BTK levels in NAMALWA cells treated with the exemplary compounds of Examples 6-8 for 24 hours.

TABLE 3
Normalized BTK levels in NAMALWA cells
treated with Compounds for 24 hours after 4 hours
of starvation in Earle's Balanced Salt Solution (EBSS)

| | 3.33 μM | 10 μM | 30 μM |
|---|---|---|---|
| Example | Compound | Compound | Compound |
| No. | Concentration | Concentration | Concentration |
| 6 | — | ~58.7%* | ~30.1%* |
| 7 | 67.0% | 48.2% | — |
| 8 | 83.1% | 66.3% | — |

*BTK-0020 was tested at 33.3 uM and 11.1 uM from 100 uM top concentration.

As illustrated in Table 3, compounds of the present disclosure are effective at reducing BTK levels in NAMALWA cells at different compound concentrations.

### EQUIVALENTS

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

Those skilled in the art will recognize or be able to ascertain using no more than routine experimentation, many equivalents to the specific embodiments of the disclosure described and claimed herein. Such equivalents are intended to be encompassed by the following claims.

1. A compound represented by the following structural formula:
CCB-L-(LC3B-Binder),
wherein:
CCB is a cellular component binder; and
L is a covalent bind or linker group.

2. The compound of claim 1, wherein cellular component binder binds to a cellular component selected from a protein, ion, lipid, nucleic acid, nucleotide, amino acid, particle, organelle, cellular compartment, microorganism, virus, lipid droplet, vesicle, small molecule, protein complex, protein aggregate and macromolecule.

3. The compound of claim 2, wherein the cellular component is associated with a disease.

4. The compound of claim 3, wherein the disease is cancer, a neurodegenerative disease, a metabolic disease, an infectious disease, an autoimmune disease, or an inflammatory disease.

5. The compound of claim 2, wherein the cellular component is a protein aggregate, soluble protein, midbody ring, damaged mitochodria, peroxisomes, intracellular bacteria, phagocytic membrane remnants, or viral capsid proteins.

6. The compound of claim 2, wherein the cellular component is a protein or protein complex selected from BTK, BRD4, KRAS, MYC, YAP, TAZ, CTNNB1, APP, HTT, SNCA, NRF2, MAPT, PINK1, ATG32, ESYT, PI3KC3, RAB10 and ATGL.

7. The compound of claim 2, wherein the cellular component is a protein selected from ERK5 (MAPK7); BTK; ALK; EGFR; RAF1; KRAS; MDM2; STAT3; HIF1A; NTRK1; IRAK4; AR; ABL1; KDR; CDK4; CDK6; CDK7; MAP3K11; MET; PDGFRA; ESR1; IGF1R; and TERT.

8. The compound of claim 2, wherein the cellular component is a protein aggregate.

9. The compound of claim 8, wherein the protein aggregate is amyloid precursor protein, βamyloid, IAPP, α-synuclein, PrP, prion protein Sc, Huntingtin, calcitonin, atrial natriuretic factor, apolipoprotein A1, Serum amyloid A, medin, prolactin, transthyretin, lysozyme, β-2 microglobulin, gelsolin, keratoepithelin, cystatin, immunoglobulin light chain AL or S-IBM.

10. The compound of claim 2, wherein the cellular component is KRAS, KRAS with a G12C mutation or KRAS with a G12D mutation.

11. The compound of claim 2, wherein the cellular component is BTK.

12. The compound of claim 1, wherein L is a covalent bond or a bivalent, saturated or unsaturated, straight or branched C₁₋₅₀ hydrocarbon chain, wherein 0-6 methylene units of L are independently replaced by —C(D)(H)—, —C(D)₂-, Si(R)₂—, —Si(OH)(R)—, —Si(OH)₂—, —P(O)(OR)—, —P(O)(R)—, —P(O)(NR₂)—, -Cy-, —O—, —NR—, —S—, —OC(O)—, —C(O)O—, —C(O)—, —S(O)—, —S(O)₂—, —NRS(O)₂—, —S(O)₂NR—, —NR(O)—, —C(O)NR—, —OC(O)NR—, NRC(O)O—, 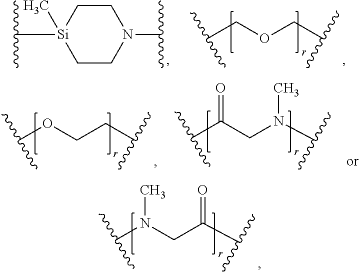
wherein:
each Cy is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur, and wherein r is 0, 1, 2, 3, 4, 5, 6, 7, 8, 9, or 10;
each R is independently hydrogen, deuterium, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or two R groups on the same atom are optionally taken together with their intervening atom to form an optionally substituted 4-11 membered saturated or partially unsaturated carbocyclic or heterocyclic monocyclic, bicyclic, bridged bicyclic, spiro, or heteroaryl ring having 0-3 heteroatoms, in addition to the atom to which they are attached, independently selected from nitrogen, oxygen, and sulfur.

13. The compound according to claim 12, wherein L is a covalent bond or a bivalent, saturated or unsaturated, straight or branched C₁₋₅₀ hydrocarbon chain, wherein 0-6 methylene units of L are independently replaced by -Cy-, —O—, —N(R)—, —C(O)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—, wherein each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur.

14. A pharmaceutical composition comprising: i) the compound of claim 1; and ii) a pharmaceutically acceptable carrier, excipient or diluent.

15. A method of treating a subject with a neurodegenerative disease, cancer, a metabolic disease, an autoimmune disease, an inflammatory disease, or infectious disease, comprising administering to the subject an effective amount of claim 1.

16. The compound of claim 2, wherein L is a covalent bond or a bivalent, saturated or unsaturated, straight or branched C₁₋₅₀ hydrocarbon chain, wherein 0-6 methylene units of L are independently replaced by —C(D)(H)—, —C(D)₂-, Si(R)₂—, —Si(OH)(R)—, —Si(OH)₂—, —P(O)(OR)—, —P(O)(R)—, —P(O)(NR₂)—, -Cy-, —O—, —NR—, —S—, —OC(O)—, —C(O)O—, —C(O)—, —S(O)—, —S(O)₂—, —NRS(O)₂—, —S(O)₂NR—, —NR(O)—, —C(O)NR—, —OC(O)NR—, NRC(O)O—, 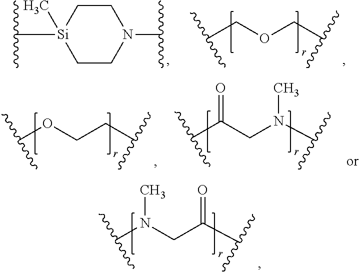
wherein:
each Cy is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur, and wherein r is 0, 1, 2, 3, 4, 5, 6, 7, 8, 9, or 10;
each R is independently hydrogen, deuterium, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or two R groups on the same atom are optionally taken together with their intervening atom to form an optionally substituted 4-11 membered saturated or partially unsaturated carbocyclic or heterocyclic monocyclic, bicyclic, bridged bicyclic, spiro, or heteroaryl ring having 0-3 heteroatoms, in addition to the atom to which they are attached, independently selected from nitrogen, oxygen, and sulfur.

17. The compound according to claim 16, wherein L is a covalent bond or a bivalent, saturated or unsaturated, straight or branched C₁₋₅₀ hydrocarbon chain, wherein 0-6 methylene units of L are independently replaced by -Cy-, —O—, —N(R)—, —C(O)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—, wherein each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur.
